(19) United States
(12) Patent Application Publication
(10) Pub. No.: US 2020/0188475 A1
(43) Pub. Date: Jun. 18, 2020

(54) REAGENTS FOR INDUCING AN IMMUNE RESPONSE

(71) Applicants: Dana-Farber Cancer Institute, Inc., Boston, MA (US); President and Fellows of Harvard College, Cambridge, MA (US); Massachusetts Institute of Technology, Cambridge, MA (US)

(72) Inventors: Ellis L. Reinherz, Lincoln, MA (US); Kyoung Joon Oh, Libertyville, IL (US); Mikyung Kim, Watertown, MA (US); Gerhard Wagner, Chestnut Hill, MA (US); Zhen-Yu J Sun, Auburndale, MA (US); Darrell J. Irvine, Arlington, MA (US); Anna Bershteyn, Somerville, MA (US)

(21) Appl. No.: 16/683,882

(22) Filed: Nov. 14, 2019

Related U.S. Application Data
(62) Division of application No. 12/680,052, filed on Sep. 27, 2010, now abandoned.
(60) Provisional application No. 60/995,708, filed on Sep. 26, 2007.

(51) Int. Cl. A61K 38/10; A61K 9/51; C07K 16/10; A61K 39/21; A61K 38/16; A61K 39/12; A61K 47/69

(52) U.S. Cl. CPC A61K 38/10; A61K 9/5153; C07K 16/1063; A61K 9/5123; A61K 39/21; A61K 2039/55555; A61K 39/12; A61K 47/6911; A61K 47/6907; A61K 47/6913; A61K 38/162

(57) ABSTRACT

The present disclosure relates to reagents (antigenic and/or immunogenic reagents) and kits that are useful in a variety of in vitro, in vivo, and ex vivo methods including, e.g., methods for inducing an immune response, or for generating an antibody, in a subject. The reagents described herein can be used in the treatment or prevention of HIV-1 infections. In addition, the disclosure provides methods and compositions useful for designing (or identifying) an agent that binds to an membrane proximal external region (MPER) of an HIV-1 gp160 polypeptide or an agent that inhibits the fusion of an HIV-1 particle to a cell.

### CROSS-REFERENCE TO RELATED APPLICATIONS

This application is a divisional and claims priority to U.S. application Ser. No. 12/680,052, filed on Sep. 2010, which is a U.S. National Stage application and claims priority of International Application No. PCT/US2008/077916, filed on Sep. 26, 2008, which claims priority of U.S. Provisional Application No. 60/995,708, filed on Sep. 26, 2007. The contents of all of the prior applications are incorporated herein by reference in their entirety.

### STATEMENT REGARDING FEDERALLY SPONSORED RESEARCH OR DEVELOPMENT

This invention was made with government support under grant numbers AI043649, AI073165, and AI037581 awarded by The National Institutes of Health and by grant number BES0348259 awarded by The National Science Foundation. The government has certain rights in the invention.

### BACKGROUND

Since the acquired immunodeficiency syndrome (AIDS) was recognized in 1981, an estimated 65 million infections and 25 million deaths have been ascribed to human immunodeficiency virus-1 (HIV-1) (Zhu et al. (2006) Nature 441:847-852). Preventative vaccination is paramount to eliminate further global HIV-1 spread. Although clinically valuable T cell-based vaccines may be developed, B cell-stimulating vaccines capable of eliciting broadly neutralizing antibodies (BNAbs) are believed to be essential for prophylaxis (Douek et al. (2006) Cell 124: 677-681 and Letvin (2006) Nat Rev Immunol 6:930-939). BNAbs will prevent entry of multiple strains of the HIV retrovirus into T cells to block viral replication as well as proviral integration into the host genome, the latter process being essential for establishing latent reservoirs of disease (Han et al. (2007) Nat Rev Microbiol 5:95-106).

### SUMMARY

This disclosure relates to, inter alia, the determination of the solution structure of the membrane proximal external region (MPER) of an HIV-1 gp160 polypeptide in a lipid environment under physiologic conditions using a combination of nuclear magnetic resonance (NMR), electron paramagnetic resonance (EPR), and surface plasmon resonance (SPR) techniques. The disclosure also relates to the discovery that the HIV-1-specific, broadly neutralizing antibody (BNAb), 4E10, upon binding to the MPER in the lipid environment, extracts key antibody epitope residues, W672 and F673, from the lipid. Both of these observations provide important implications for vaccine design strategy and HIV-1 inhibitor design, and offer insight into how BNAbs perturb viral fusion in the case of HIV-1. Accordingly, the disclosure features a variety of reagents, kits, and methods useful for, inter alia, inducing an immune response in a subject and designing (or identifying) an agent that can bind to an MPER or inhibit the fusion of an HIV-1 particle to a cell. Such agents, along with the reagents described herein, are useful in treating and/or preventing an HIV-1 infection in a subject.

In one aspect, the disclosure features a reagent comprising: a particle that is partially or completely encapsulated in lipid; and a polypeptide comprising a membrane proximal external region (MPER) of an HIV-1 gp160 polypeptide, wherein at least one amino acid residue of the MPER is embedded in the lipid.

In some embodiments, the polypeptide comprises no more than 300 (e.g., 290, 280, 270, 260, 250, 240, 230, 220, 210, 200, 190, 180, 170, 160, 150, 140, 130, 120, 110, 100, 90, 80, 70, 60, 50, 40, 30, 22, or 20) amino acids.

In some embodiments, the MPER contains, or is, the amino acid sequence X₁-L-X₂-X₃-W-X₄-X₅-X₆-W-X₇-W-X₈-X₉-I-X₁₀-X₁₁-W-L-W-Y-I-X₁₂ (SEQ ID NO:1), wherein X₁ is A, Q, G, or E; X₂ is D or S; X₃ is K, S, E, or Q; X₄ is A, S, T, D, E, K, Q, or N; X₅ is S, G, or N; X₆ is L or I; X₇ is F, N, S, or T; X₈ is F or S; X₉ is D, K, N, S, T, or G; X₁₀ is S or T; X₁₁ is N, K, S, H, R, or Q; and X₁₂ is K, E, or R. The MPER can contain, or consist of, the amino acid sequence ELDKWASLWNWFNITNWLWYIK (SEQ ID NO:2) or ALDKWASLWNWFDISNWLWYIK (SEQ ID NO:3). The MPER can contain, or consist of, any of the amino acid sequences depicted in Table 1 (e.g., SEQ ID NOS:2-34).

In some embodiments, the polypeptide can contain, or consist of, an amino acid sequence corresponding to amino acid positions 660 to 856 of the HXB2 strain HIV-1 gp160 polypeptide an amino acid sequence corresponding to amino acid positions 662 to 683 of the HXB2 strain HIV-1 gp160 polypeptide.

In some embodiments, the MPER can be flanked at the amino-terminal end, the carboxy-terminal end, or both the amino-terminal and the carboxy-terminal end by a heterologous amino acid sequence.

The lipid can be any of those described herein. The lipid can have any of the forms described herein. For example, the lipid can be a lipid monolayer or a lipid bilayer. In some embodiments, the lipid can be more than one lipid bilayer.

The particle can contain, or consist of, one or more of a polymer, a silica, a glass, a metal (e.g., gold or silver), or any of the particle materials described herein. In some embodiments, the particles can contain, or consist of, more than one of any of the materials described herein. In some embodiments, the particle can be magnetic, encoded, or both magnetic and encoded. The particle can contain, or consist of, a therapeutic, diagnostic, or prophylactic agent such as any of those described herein. The particle can be bioresorbable or biodegradable.

The particle, or the reagent itself, can be a microparticle or a nanoparticle.

In some embodiments, at least one (e.g., two, three, four, five, six, seven, eight, nine, 10, 11, 12, 13, 14, 15, 16, 17, 18, 19, or 20 or more) amino acid(s) of the MPER is not embedded in the lipid. The at least one amino acid of the MPER can correspond to position 671, 674, 677, or 680 of the HXB2 strain HIV-1 gp160 polypeptide.

In some embodiments, the reagent can contain at least one additional polypeptide such as a targeting polypeptide or a dendritic cell activating polypeptide. The targeting polypeptide can target the reagent to an antigen presenting cell such as a dendritic cell or a macrophage. The at least one polypeptide can contain a T helper epitope such as any of those described herein.

In some embodiments, the reagent can contain one or more additional therapeutic, diagnostic, or prophylactic agents. The one or more additional therapeutic agents can be immune modulators such as adenosine receptor inhibitors, HIF-1α inhibitors, or adjuvants. The one or more agents can be lipophilic and/or consist of embedded in the lipid.

In some embodiments, the MPER can be a fragment of a Group M HIV-1 gp160 polypeptide. In some embodiments, the MPER can be fragment of a Clade, A, Clade B, Clade C, or Clade D HIV-1 gp160 polypeptide.

In some embodiments, the reagent can be detectably labeled. For example, the lipid, the polypeptide, and/or the particle can be labeled. The detectable label can be a fluorescent label, a luminescent label, a radioactive label, or an enzymatic label.

In another aspect, the disclosure features a pharmaceutical composition comprising any of the reagents described herein and a pharmaceutically acceptable carrier.

In another aspect, the disclosure features a pharmaceutical solution comprising any of the reagents described herein in a pharmaceutically acceptable carrier.

In yet another aspect, the disclosure features a method for inducing an immune response, or a method for generating/producing an antibody, in a subject. The method includes the step of administering to a subject a composition comprising lipid and a polypeptide consisting of a membrane proximal external region (MPER) of an HIV-1 gp160 polypeptide, wherein at least one amino acid residue of the MPER is embedded in the lipid.

In another aspect, the disclosure features a method for inducing an immune response, or a method for generating/producing an antibody, in a subject. The method includes the step of administering to a subject a composition comprising: a particle encapsulated in lipid; and an immunogen, wherein all or part of the immunogen is embedded in the lipid. The immunogen can be a molecule or an immunogenic fragment thereof that is expressed on the surface of (i) a cell; (ii) a microorganism; or (iii) a cell that is infected with a microorganism. The microorganism and cell can be any of those described herein.

In yet another aspect, the disclosure features a method for inducing an immune response, or a method for generating/producing an antibody, in a subject, the method comprising administering to a subject any of the reagents described herein.

In some embodiments of any of the above methods, the subject can be a mammal such as a human. The subject can have, be suspected of having, or consist of at risk of developing an HIV-1 infection.

In some embodiments, any of the above methods can also include the step of after administering the reagent, determining whether an immune response in the subject has occurred.

In some embodiments, any of the above methods can also include the step of administering to the subject one or more anti-HIV-1 agents. The one or more anti-HIV-1 agents can be selected from the group consisting of HIV-1 protease inhibitors, HIV-1 integrase inhibitors, HIV-1 reverse transcriptase inhibitors, HIV-1 fusion inhibitors, and antibodies specific for HIV-1 (e.g., HIV-1 specific neutralizing antibodies).

In some embodiments, any of the above methods can also include the step of determining whether the subject has an HIV-1 infection. The determining can occur before and/or after administering the reagent to the subject.

In some embodiments, any of the methods described above can also include the step of administering an adjuvant to the subject.

In another aspect, the disclosure features (i) an isolated antibody generated by any of the above methods for generating/producing an antibody in a subject and (ii) an isolated cell that produces the antibody.

In yet another aspect, the disclosure features a kit comprising: any of the reagents described herein; and optionally instructions for administering the reagent to a subject. The kit can also include one or more pharmaceutically acceptable carriers or diluents.

In another aspect, the disclosure features an article of manufacture comprising: a container; and a composition contained within the container, wherein the composition comprises an active ingredient for inducing an immune response in a mammal, wherein the active ingredient comprises any of the reagents described herein, and wherein the container has a label indicating that the composition is for use in inducing an immune response in a mammal. The label can further indicate that the composition is to be administered to a mammal having, or at risk of developing, an HIV-1 infection. In some embodiments, the article of manufacture can also contain instructions for administering the composition to the mammal. The composition can be dried or lyophilized.

In yet another aspect, the disclosure features a method for designing an agent that interacts with a membrane proximal external region (MPER) of an HIV-1 gp160 polypeptide. The method can include the steps of: providing a three-dimensional model of a composition comprising a membrane proximal external region (MPER) of an HIV-1 gp160 polypeptide and lipid, wherein at least one amino acid of the MPER is embedded in the lipid; and performing computer fitting analysis to design an agent that interacts with the MPER. The method can also include the step of determining whether the agent interacts with the MPER. The method can also include the step of determining the three-dimensional structure of the composition. The three-dimensional structure can be a solution structure or a crystal structure. The method can also include the step of obtaining the agent.

In some embodiments, the three-dimensional model of the composition can contain the structural coordinates of an atom selected from the group consisting of atoms of amino acids L669 to W680 according to FIG. 25, ± a root mean square deviation from the conserved backbone of atoms of the amino acids of not more than 1.5 Å (e.g., not more than 1.0 Å or not more than 0.5 Å).

In some embodiments, the three-dimensional model of the composition can contain the complete structural coordinates of the amino acids according to FIG. 25, ±a root mean square deviation from the conserved backbone of atoms of the amino acids of not more than 1.5 Å (e.g., not more than 1.0 Å or not more than 0.5 Å).

The lipid can be any described herein and can have any form described herein, e.g., a lipid bilayer, a lipid monolayer, or a lipid micelle. In some embodiments, the lipid can be in the form of more than one lipid bilayer.

In some embodiments, the agent can inhibit the fusion of an HIV-1 particle to a cell. In some embodiments, the method can include the step of determining if the agent inhibits the fusion of an HIV-1 particle to a cell.

In yet another aspect, the disclosure features an agent designed by the above methods.

In another aspect, the disclosure features a method for identifying a potential inhibitor of the fusion of an HIV-1 particle to a cell. The method includes the steps of: generating a three dimensional model of composition using the relative structural coordinates of the amino acids of FIG. 25, ± a root mean square deviation from the conserved backbone atoms of the amino acids of not more than 1.5 Å (e.g., not more than 1.0 Å or not more than 0.5 Å), wherein the composition comprises lipid and a membrane proximal external region (MPER) of an HIV-1 gp160 polypeptide and wherein at least one amino acid of the MPER is embedded in the lipid; employing the three-dimensional model to design or select a potential inhibitor of the fusion of an HIV-1 particle to a cell; and synthesizing or obtaining the potential inhibitor.

In another aspect, the disclosure features a solution comprising a composition comprising: a polypeptide consisting of a membrane proximal external region (MPER) of an HIV-1 gp160 polypeptide; and lipid, wherein at least one amino acid of the MPER is embedded in the lipid. The three-dimensional structure can be a solution structure or a crystal structure. The three-dimensional structure can be determined by NMR.

In some embodiments, the MPER can contain, or consist of, the amino acid residues 662 to 682 of FIG. 25.

In some embodiments, the MPER can be unlabeled, ¹⁵N-labeled, or ¹⁵N and ¹³C labeled.

In some embodiments, the secondary structure of the MPER can contain two alpha helices. A first alpha helix can contain, or consist of, amino acid residues 662 to 672 of the HXB2 strain gp160 polypeptide and a second alpha helix can contain, or consist of, amino acids 675 to 682 of the HXB2 strain gp160 polypeptide. The two alpha helices can be joined by a hinge region. For example, the hinge region can contain, or consist of, amino acids 673 and 674 of the HXB2 strain gp160 polypeptide.

In some embodiments, the MPER can have the structure defined by the relative structural coordinates according to FIG. 25, ± a root mean square deviation from the conserved backbone atoms of the amino acids of not more than 1.5 Å (e.g., not more than 1.0 Å or not more than 0.5 Å).

In some embodiments, the MPER can have the structure defined by the relative structural coordinates of an atom selected from the group consisting of atoms of amino acids L669 to W680 according to FIG. 25, ±a root mean square deviation from the conserved backbone of atoms of the amino acids of not more than 1.5 Å (e.g., not more than 1.0 Å or not more than 0.5 Å).

The lipid can be any described herein and in any form such as a lipid monolayer, a lipid bilayer, or a form comprising more than one lipid bilayer.

In yet another aspect, the disclosure features a method for identifying an agent capable of extracting one or more amino acid residues of a membrane proximal external region (MPER) of an HIV-1 gp160 polypeptide from lipid. The method includes the steps of providing a composition comprising lipid and an MPER of an HIV-1 gp160 polypeptide, wherein one or more amino acids of the MPER are embedded in the lipid; contacting the composition with a candidate agent; and detecting whether one or more amino acids of the MPER are extracted from the lipid, wherein the extraction of one or more amino acids from the lipid in the presence of the candidate compound indicates that the candidate agent is capable of extracting one or more amino acid residues of an MPER from lipid. The detecting can comprise nuclear magnetic resonance spectroscopy or electron paramagnetic spectrometry. The detecting can include measuring membrane immersion depth data on a spin-labeled MPER peptide. The method can also include determining whether a conformational change occurred at one or more specific residues of the MPER. The method can also include the step of determining the structure of the MPER bound to the candidate agent in a lipid environment. The method can also include the step of determining whether the candidate agent inhibits the fusion of an HIV-1 particle to a cell.

“Polypeptide” and “protein” are used interchangeably and mean any peptide-linked chain of amino acids, regardless of length or post-translational modification.

As used herein, a “membrane proximal external region” or “MPER” of an HIV-1 gp160 polypeptide is a region corresponding to amino acid positions 662 to 683 of the HXB2 strain HIV-1 gp160 polypeptide depicted in SEQ ID NO:37. “Corresponding to” means that (i) an MPER present in an HIV-1 gp160 polypeptide other than the HXB2 strain HIV-1 polypeptide does not, per se, have to occur exactly at amino acid positions 662 to 683 of the other HIV-1 gp160 polypeptide and (ii) that the amino acid sequence of the MPER does not have to be a sequence identical to the MPER of an HXB2 strain gp160 polypeptide of SEQ ID NO:37. That is, an MPER can occur at, e.g., positions 660 to 681 of another HIV-1 gp160 polypeptide such as the ADA strain HIV-1 gp160 polypeptide depicted in SEQ ID NO:38 or any other HIV-1 Group (e.g., Group M) or Clade (e.g., Clades A, B, C, or D). All that is required is that the MPER sequence corresponding to amino acid positions 662 to 683 of SEQ ID NO:37 is at least 50 (e.g., at least 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100) % identical to amino acid sequence of 662 to 683 of SEQ ID NO:37 when the two sequences are aligned for optimal homology.

Also included are MPER that have a sequence that has not more than 20 (e.g., not more than one, two, three, four, five, six, seven, eight, nine, 10, 11, 12, 13, 14, 15, 16, 17, 18, or 19) conservative amino acid substitutions so long as the sequence is at least 50 (e.g., at least 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100) % identical to the MPER of the HXB2 strain HIV-1 gp160 polypeptide.

Suitable algorithms and computational methods for determining sequence identify between two polypeptide sequences are known in the art and include programs such as, but not limited to, Clustal W (The European Bioinformatics Institute (EMBL-EBI), BLAST-Protein (National Center for Biotechnology Information (NCBI), United States National Institutes of Health), and PSAlign (University of Texas A&M; Sze et al. (2006) Journal of Computational Biology 13:309-319).

Any of the polypeptides (e.g., the polypeptides containing an MPER) or polypeptide immunogens described herein can consist of, or include, the full-length, wild-type forms of the polypeptides. For example, an HIV-1 gp160 polypeptide can consist of, or be, a full-length HIV-1 gp160 polypeptide (e.g., a full-length HXB2 strain HIV-1 gp160 polypeptide SEQ ID NO:37).

The disclosure also provides (i) biologically active variants and (ii) biologically active fragments or biologically active variants thereof, of the wild-type, full-length polypeptides. Biologically active variants of full-length, mature, wild-type proteins or fragments of the proteins can contain additions, deletions, or substitutions. Proteins with substitutions will generally have not more than 50 (e.g., not more than one, two, three, four, five, six, seven, eight, nine, ten, 12, 15, 20, 25, 30, 35, 40, or 50) conservative amino acid substitutions. A conservative substitution is the substitution of one amino acid for another with similar characteristics. Conservative substitutions include substitutions within the following groups: valine, alanine and glycine; leucine, valine, and isoleucine; aspartic acid and glutamic acid; asparagine and glutamine; serine, cysteine, and threonine; lysine and arginine; and phenylalanine and tyrosine. The non-polar hydrophobic amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Any substitution of one member of the above-mentioned polar, basic or acidic groups by another member of the same group can be deemed a conservative substitution. By contrast, a non-conservative substitution is a substitution of one amino acid for another with dissimilar characteristics.

Deletion variants can lack one, two, three, four, five, six, seven, eight, nine, ten, 11, 12, 13, 14, 15, 16, 17, 18, 19, or 20 amino acid segments (of two or more amino acids) or non-contiguous single amino acids.

Additions (addition variants) include fusion proteins containing: (a) full-length, wild-type polypeptides or fragments thereof containing at least five amino acids; and (b) internal or terminal (C or N) irrelevant or heterologous amino acid sequences. In the context of such fusion proteins, the term “heterologous amino acid sequences” refers to an amino acid sequence other than (a). A fusion protein containing a peptide described herein and a heterologous amino acid sequence thus does not correspond in sequence to all or part of a naturally occurring protein. A heterologous sequence can be, for example a sequence used for purification of the recombinant protein (e.g., FLAG, polyhistidine (e.g., hexahistidine), hemagluttanin (HA), glutathione-S-transferase (GST), or maltose-binding protein (MBP)). Heterologous sequences can also be proteins useful as diagnostic or detectable markers, for example, luciferase, green fluorescent protein (GFP), or chloramphenicol acetyl transferase (CAT). In some embodiments, the fusion protein contains an antibody or antigen binding fragment there of (see below). In some embodiments, the fusion protein contains a signal sequence from another protein. In some embodiments, the fusion protein can contain a carrier (e.g., KLH) useful, e.g., in eliciting an immune response (e.g., for antibody generation; see below). In some embodiments, the fusion protein can contain one or more linker moieties (see below). Heterologous sequences can be of varying length and in some cases can be a longer sequences than the full-length target proteins to which the heterologous sequences are attached.

A “fragment” as used herein, refers to a segment of the polypeptide that is shorter than a full-length, immature protein. Fragments of a protein can have terminal (carboxy or amino-terminal) and/or internal deletions. Generally, fragments of a protein will be at least four (e.g., at least five, at least six, at least seven, at least eight, at least nine, at least 10, at least 12, at least 15, at least 18, at least 25, at least 30, at least 35, at least 40, at least 50, at least 60, at least 65, at least 70, at least 75, at least 80, at least 85, at least 90, or at least 100 or more) amino acids in length.

Biologically active fragments or biologically active variants of any of the targeting polypeptides or toxic polypeptides described herein have at least 25% (e.g., at least: 30%; 40%; 50%; 60%; 70%; 75%; 80%; 85%; 90%; 95%; 97%; 98%; 99%; 99.5%, or 100% or even greater) of the activity of the wild-type, full-length polypeptide. In the case of a targeting polypeptide, the relevant activity is the ability of the targeting polypeptide to bind to the target of interest (e.g., a target cell, a target tissue, or a target molecule or macromolecule complex).

Depending on their intended use, the polypeptides (e.g., targeting polypeptides or immunogenic polypeptides), biologically active fragments, or biologically active variants thereof can be of any species, such as, e.g., fungus, protozoan, bacterium, virus, nematode, insect, plant, bird, fish, reptile, or mammal (e.g., a mouse, rat, rabbit, hamster, gerbil, dog, cat, goat, pig, cow, horse, whale, monkey, or human). In some embodiments, biologically active fragments or biologically active variants include immunogenic and antigenic fragments of the proteins. An immunogenic fragment is one that has at least 25% (e.g., at least: 30%; 40%; 50%; 60%; 70%; 75%; 80%; 85%; 90%; 95%; 97%; 98%; 99%; 99.5%, or 100% or even more) of the ability of the relevant full-length, wild-type protein to stimulate an immune response (e.g., an antibody response or a cellular immune response) in an animal of interest. An antigenic fragment of a protein is one having at least 25% (e.g., at least: 30%; 40%; 50%; 60%; 70%; 75%; 80%; 85%; 90%; 95%; 97%; 98%; 99%; 99.5%, or 100% or even greater) of the ability of the relevant full-length, wild-type protein to be recognized by an antibody specific for the protein or a T cell specific to the protein.

As used herein, “encapsulated” means to separate (as a barrier) one substance from another by enveloping or coating one of the substances. For example, a particle that is encapsulated by lipid can be directly coated with the lipid (that is, physical contact between the surface of the particle and the lipid) or a particle can be enveloped by the lipid (e.g., a lipid bilayer) such that the encapsulated particle or part of the particle does not physically touch the lipid. It is understood that a particle can be partially (e.g., 5, 10, 15, 20, 25, 30,35, 40, 45, 50, 60, 65, 70, 75, 80, 85, 90, 95, or 99%) or completely encapsulated by lipid. Thus, a partially encapsulated particle is one that is not completely surrounded by lipid.

“Structural coordinates” are the Cartesian coordinates corresponding to an atom's spatial relationship to other atoms in a molecule or molecular complex. Structural coordinates can be obtained using x-ray crystallography techniques or NMR techniques, or can be derived using molecular replacement analysis or homology modeling. Various software programs allow for the graphical representation of a set of structural coordinates to obtain a three dimensional representation of a molecule or molecular complex. The structural coordinates of the structures described herein can be modified from the original set provided in FIG. 25 by mathematical manipulation, such as by inversion or integer additions or subtractions. As such, it is recognized that the structural coordinates of the present invention are relative, and are in no way specifically limited by the actual x, y, z coordinates of FIG. 25.

As used herein, “root mean square deviation” is the square root of the arithmetic mean of the squares of the deviations from the mean, and is a way of expressing deviation or variation from the structural coordinates described herein. The present disclosure includes all embodiments comprising conservative substitutions of the noted amino acid residues resulting in same structural coordinates within the stated root mean square deviation.

As used herein, a T cell can be, e.g., a CD4⁺ T cell, a CD8⁺ T cell, a helper T cell, or a cytotoxic T cell.

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can also be used in the practice or testing of the present invention. All publications, patent applications, patents and other references mentioned herein are incorporated by reference in their entirety. The materials, methods, and examples disclosed herein are illustrative only and not intended to be limiting.

Other features and advantages of the invention, e.g., methods for inducing an immune response in a subject, will be apparent from the following description, from the drawings and from the claims.

### BRIEF DESCRIPTION OF THE DRAWINGS

FIGS. 1A-1D depict the NMR structure of the MPER in a DPC micelle. FIG. 1A is a stereo ribbon diagram of the MPER of an HXB2 strain gp160 polypeptide. FIG. 1B is a sequential plot of NMR constraints showing the α-helical pattern at the N-terminal and mixed 3₁₀ and α-helical pattern at the C-terminal end of MPER peptide. FIG. 1C is an ensemble of 17 MPER NMR structure models superimposed by backbone atoms (light trace) of the N-terminal segment (dark trace; left), or the C-terminal segment (dark trace; right). FIG. 1D is a ribbon diagram depicting the placement of the MPER peptide on the micelle surface (light-shaded spheres at the bottom). The darker sphere represents the lipid acyl-chain region.

FIGS. 2A-2F depict MPER analysis by electron paramagnetic resonance (EPR): EPR spectra, accessibility parameters, immersion-depth and overall topology. FIG. 2A is EPR spectra of R1 side chains in MPER peptides bound to large unilamellar vesicles of POPC/POPG (at a 4:1 ratio, w/w). Spectra were obtained in the absence and presence of 4E10 antibody twice in excess to the peptide. Characteristic features of highly mobile spectra (E662R1, W670R1 and W678R1) and highly immobile one (N667R1) are indicated by arrows and by an arrow head, respectively. The vertical dotted lines indicate the approximate region of some spectra where the immobile components are increasing upon 4E10 binding. Scan width (abscissa) was 100 Gauss. Generation of the R1 side chain by the reaction of the methanethiosulfonate nitroxide spin label with the cysteine residue is shown in the inset. FIG. 2B depicts the accessibility parameters Π(O₂) and Π(NiEDDA) for R1 residues in MPER peptides bound to POPC/POPG vesicles as a function of residue number. Air oxygen and 5 mM NiEDDA were used to measure the accessibility parameters, Π(O₂) (top panel) and Π(NiEDDA) (bottom panel), respectively. The positions of Π(O₂) maxima and corresponding positions in Π(NiEDDA) are marked with vertical dotted lines. FIG. 2C depicts the immersion-depth of the lipid-facing R1 residues of MPER bound to POPC/POPG (4:1, w/w) vesicles. Average values of 2-3 independent measurements are reported with standard deviation. Depth values larger than 0 Å and between 0 and −5 Å correspond to acyl chain region and headgroup region in the membrane, respectively. The depths of lipid-facing R1 residues were fitted with membrane surface-bound helical models for the N-terminal (residues 667-673, dotted curve) and C-terminal (residues 676-682, solid curve) helices as described in FIG. 3. FIG. 2D depicts helical wheel diagrams for N- (residues 662-673) and C-terminal (residues 674-682) helices of the membrane-bound MPER. The open square, shaded triangle, or filled circle represents a R1 residue exposed to aqueous phase, buried in the lipid headgroup region, or in the acyl chain region, respectively. The topological location of the residue in parentheses was not determined. FIG. 2E depicts the membrane immersion depth for R1 residues in membrane-bound/4E10-bound MPER peptide. The depths of the indicated R1 residues in the MPER peptides bound to the POPC/POPG vesicles were measured in the presence of equimolar 4E10 antibody. Residues showing the largest depth change upon 4E10 binding are indicated with asterisks. FIG. 2F is a topological model of MPER peptide in the membrane. The tilted N-terminal helix (residues 662-672) is linked to the C-terminal helix (residues 676-682) lying almost parallel to the membrane surface. Residues 673-675 serve as a linker.

FIGS. 3A-3F depict the tilts and rotational orientation of the N- and C-terminal helices of the MPER. FIG. 3A is a representation of positions of a R1 side chain in cylindrical coordinates adapted from Oh et al. (2005) J Biol Chem 280, 753-767. N represents the amino acid residue number; N₀, the residue at which the helical axis intercepts the surface of the lipid bilayers that is the interface between the lipid head group and the hydrocarbon chain; r, the length of the nitroxide arm; θ₀, the rotational orientation angle of the residue N₀ vector with respect to the membrane normal; ω, the helix tilting angle; and p, the helical pitch, 5.41 Å, for 3.6 residues rise for a turn in an a-helix. The equation for the immersion depth of a spin label on a tilted helix is shown inset (Oh et al., supra). FIGS. 3B and 3C depict the tilting angle and rotational orientation of the N-terminal helix (residues 662-672). The best fitting curve for the depths of the residues 667R1-672R1 in the N-terminal helix was obtained with ω=16.5° (±5), N₀=665.8 (±0.1) and θ₀=176° (±5). The dotted arrow in B represents helical axis drawn from the N to the C terminus, which is ˜15° tilted away from the membrane surface for the N-terminal helix. The dotted vertical arrow in FIG. 3C represents the direction of the greatest depth viewed from the helical axis. FIGS. 3D and 3E depict the tilting angle and rotational orientation of the C-terminal helix (residues 676-683). The best fitting curve for the depths of the residues 676R1-683R1 in the C-terminal helix (FIG. 2C, solid curve) was obtained with ω=2.9° (±5), N₀=671.1 (±0.1) and θ₀=139° (±5), where residue Y681R1 deviated considerably perhaps due to the alternative conformations of the spin label. Dotted arrows in FIGS. 3D and 3E represent the same as defined in FIG. 3B and FIG. 3C, respectively. The angles (θ) between the membrane normal vector and the radial vectors for residues 669 in FIG. 3C and 682 in FIG. 3D, viewed from the helical axis, are 141° and 149°, respectively. A value of r=7.5 Å (±0.5) was assumed in all the data fittings (Oh et al., supra). FIG. 3F is a model of MPER in the membrane. The tilted N-terminal helix (residues 662-672) is linked to the C-terminal helix (residues 676-682) lying almost parallel to the membrane surface. Residues 673-675 serve as a linker.

FIG. 4 depicts a comparison of MPER peptide in DPC micelle and bicelle NMR ¹⁵N-HSQC spectra of MPER peptide in DPC micelle (light shade) and DHPC/DMPC bicelle (q=DMPC:DHPC=0.3) (dark shade) taken at 35° C. The peak shifts are comparable to the small bending of a membrane peptide in different lipid environment (Chou et al. (2002) J Am Chem Soc 124, 2450-2451).

FIGS. 5A-5C depict the sequence conservation within the MPER segment of HIV-1 envelope proteins. FIG. 5A is a space-filled model of the HxB2 MPER peptide on a micelle (48 Å diameter). FIG. 5B depicts Shannon entropy is plotted for each residue from 975 HIV-1 sequences with variability on the Y-axis (0=no variability at a given position; 4.322=all 20 amino acids permitted at that position). The insert shows variability over the entire gp160 proteins from these same viral isolates. Open circles represent regions of conservation in gp160 comparable to that of the MPER segment (darkened circle) and correspond to amino acid residues (from left to right) 85-117, β1-α1 elements buried within the inner domain; 187-222, V2-β3-β4 largely buried segments; 230-258, [symbol] A β6-β8, [symbol] B, mostly buried within the inner domain; 512-534, fusion peptide; 553-590, the N leucine zipper; and 684-705, the TM segment abutting the MPER. Analyses were performed using a window size of 20 residues and with the X-axis showing amino acid position of the window start. FIG. 5C is a graphical representation of amino acids patterns within sequence alignments using WebLogo (University of Berkeley, Calif.).

FIGS. 6A-6C depict the sequence variability of the MPER peptide. FIG. 6A is a phylogenetic tree of a set of HIV-1 envelope sequences representing a variety of group M clades and their geographic isolates plus a single representative for each of the groups O and N. FIG. 6B depicts the sequence logos of major HIV-1 groups. FIG. 6C depicts the sequence logos for subgroups (clades) of the HIV-1 group M. CRF=circulating recombinant forms.

FIGS. 7A-7D depict the binding of 2F5 and 4E10 antibodies to the membrane bound spin-labeled peptides. FIGS. 7A and 7B are a pair of binding curves of membrane-bound/spin-labeled peptides for 2F5 (FIG. 7A) and 4E10 (FIG. 7B) peptides. FIG. 7C depicts the residual binding of 2F5 and 4E10. FIG. 7D is a ratio of 4E10 residual binding to that of 2F5. In A and B, the binding curves of 2F5 and 4E10 antibodies were recorded as described in Example 1. The initial 40 second plateau of the curves in FIGS. 7A and 7B corresponds to the washing step after loading the peptides (2 μM) to the liposome-loaded chip. Antibodies (20 μg/ml) were loaded to the peptide/liposomes chip at ˜2040 second for 3 min and washed for 2 and a half minutes. In FIG. 7C, the average RU values of the residual binding of 2F5 (shaded bars) and 4E10 (black bars) relative to the corresponding buffer baselines at the last 10 seconds in FIGS. 7A and 7B are shown in pairs for the indicated peptides in C. In D, the ratio of the last 10 second average RU for 4E10 to that of 2F5 shown in C are presented for the indicated peptides. The binding curves for 662R1, 664R1, 665R1, 667R1, 668R1 and 683R1 (see the italicized letters for the dotted lines in FIGS. 7A and 7B were obtained separately from the rest of the samples. The 4E10/2F5 binding ratio of the control wild type MPER peptide for this data set was different from the value shown in FIG. 7D. The ratios were therefore scaled to give the same 4E10 to 2F5 binding ratio for the wild type peptide. The 4E10/2F5 binding ratios for peptides spin-labeled at residues 662 and 664-667, which are critical for 2F5 binding, are shown above the corresponding bars ‘Wt’ and ‘672A673A’ stand for MPER peptides with no or double alanine substitution mutations in the sequence, respectively. The spin labeled peptides had a wild type sequence except the cysteine residue substitution at the position of the indicated spin label.

FIGS. 8A-8D depict the sequence-specific 4E10 antibody binding to the MPER peptide bound to the POPC/POPG (4:1, w/w) membrane. FIG. 8A is an EPR spectra for the membrane-bound MPER peptide containing 677R1 in the presence of 4E10 at various ratios as indicated. FIG. 8B is an EPR spectra for the membrane-bound MPER peptide containing 677R1 in the presence of control human IgG at various ratios as indicated. FIG. 8C is an EPR spectra for the membrane-bound MPER peptide containing 677R1 and double alanine substitutions (672A673A677R1) in the presence of 4E10 at various ratios as indicated. FIG. 8D is an EPR spectra for the membrane-bound MPER peptide containing 677R1 and double alanine substitutions (672A673A677R1) in the presence of control human IgG at various ratios as indicated. The arrow in FIG. 8A indicates a relatively mobile population of the spin label 677R1, which decreases only upon 4E10 binding to an MPER peptide with a wild type sequence but not with 672A673A double mutations. The dotted lines show a region in the spectra where an immobile population of the spin label increases upon 4E10 binding. LUV (large unilamellar vesicles) consisting of POPG and POPC at 4:1 w/w ratio, prepared as described in Example 1. Spectra of 100 Gauss scan for varying peptide to antibody ratios are overlayed after normalization to the same area by double integration.

FIGS. 9A-9E depict the conformational change in MPER induced by 4E10. FIG. 9A is a ¹⁵N-TROSY-HSQC spectrum containing free and Fab-bound HxB2 MPER peptide. FIG. 9B depicts the normalized (sqrt((ΔHcs)²+(ΔNcs/5)²) in ppm) MPER amide chemical shift changes upon 4E10 binding. FIG. 9C depicts the relative signal reduction of amide peaks with 250 ms cross-saturation showing MPER residues involved in 4E10 interaction. FIGS. 9D and 9E are models for MPER peptide in complex with 4E10 antibody as viewed from the side (FIG. 9D) and membrane face (FIG. 9E). In FIG. 9D, the orientation of uncomplexed MPER is shown for comparison.

FIGS. 10A-10B are a pair of bar graphs depicting NMR derived shifts of MPER peptide upon 4E10 binding. FIG. 10A is a comparison of Cα chemical shift changes of MPER peptide upon 4E10 binding. Chemical shift indexes (Wishart and Sykes (1994) J Biomol NMR 4, 171-180) larger than 1.0 are indicative of an alpha-helical conformation. The residues W670 and N671 appear to be in extended (beta-strand) conformation. FIG. 10B depicts the peak intensities of amide peaks from 4E10-bound MPER relative to the unbound MPER (in slow exchange) in the same NMR sample. The weak peaks (N671 to L679) are a result of combination of slow mobility and fast relaxation.

FIGS. 11A-11C are an assessment of BNAb with membrane and MPER. FIG. 11A depicts the critical role of N671 for 4E10 binding to MPER/liposomes as evaluated using BIAcore. Control (HXB2) MPER and single amino acid variants are shown. 2F5 reactivity for each variant was equivalent to the HXB2 control. FIG. 11B depicts the isothermal titration calorimetry (ITC) result of injecting 250 mM of MPER peptide with virion membrane-like liposome into 10 mM 4E10 Fab at 25° C. The enthalpy change is −25.0 kcal/mole of Fab molecule and the binding constant is 1.0 μM from fitting results, yielding a large positive entropic energy change of (−TDS)=16.9 kcal/mole. FIG. 11C depicts the binding of BNAbs 4E10, 2F5 and Z13e1 to synthetic virion membrane-bound MPER (virion membrane/MPER)(black) and virion membrane alone (insert).

FIGS. 12A-12E depict the synthesis of lipid-enveloped nanoparticles. FIG. 12A depicts the chemical structures of PLGA and several lipids used in the preparation of lipid-enveloped nanoparticles. FIG. 12B depicts the diameters of lipid-coated PLGA particles obtained as a function of processing conditions, as determined by dynamic light scattering. FIG. 12C depicts the fluorescence from rhodamine-conjugated lipid incorporated in lipid-enveloped microparticles. FIGS. 12D and 12E are a pair of unstained cryo-electron microscopy images of lipid-enveloped particles, illustrating surface lipids. The right panel is magnified view of left panel inset. Arrows highlight evidence for bilayer formation at the surface of the enveloped nanoparticles.

FIGS. 13A-13D show that lipid-enveloped PLGA particles taken up by dendritic cells and can be functionalized with targeting ligands. FIG. 13A depicts DiD-labeled lipid-enveloped nanoparticles 150 nm in diameter (1 mg/mL) that were incubated with the murine dendritic cell line DC2.4 for different times at 37° C. and then analyzed by flow cytometry to detect nanoparticle fluorescence in the cells. FIGS. 13B and 13C depict lipid-enveloped microparticles containing 1 mole % biotin-PEG-DSPE lipid (FIG. 13B) or non-biotinylated control particles (FIG. 13C) were stained with Alexa fluor 488-conjugated streptavidin (lower panel) and visualized by confocal microscopy (upper panel, rhodamine-lipid fluorescence). FIG. 13D depicts the antibody conjugation to maleimide-functionalized nanoparticles: Maleimide-bearing or control lipid-enveloped microparticles were mixed with thiolated antibody or control non-thiolated Alexafluor 488-labeled antibody at pH 7.4, then centrifuged and washed to remove unbound antibody. Average fluorescence intensities around individual particles were then quantified by confocal microscopy for each condition. Surface fluorescence similar to the streptavidin coupling shown in (FIG. 13B) was only observed when maleimide-bearing particles (Mal-particles) were incubated with thiolated antibody (Ab-SH).

FIG. 14 depicts a schematic of an exemplary lipid-enveloped nanoparticle described herein.

FIGS. 15A-15E show that an MPER spontaneously adsorbs to lipid-enveloped PLGA particles. FIGS. 15A and 15B depict the confocal fluorescence imaging of lipid-enveloped PLGA microparticles (FIG. 15A) or lipid-enveloped particles incubated with 10 μM FITC-MPER peptide for 30 min at 4° C. (FIG. 15B). Particles were labeled by incorporation of DiD lipid dye. Clear MPER binding to the surfaces of the particles is observed in (FIG. 15B). FIGS. 15C and 15D depict the nanoparticle capture-on-cells assay used to quantify FITC-MPER binding to lipid-enveloped nanoparticles. DC2.4 murine dendritic cells were surface-biotinylated, stained with streptavidin, and then incubated with lipid-enveloped nanoparticles containing biotinylated lipids in their surface layer. FIG. 15C depicts the use of confocal microscopy to show that the biotinylated nanoparticles (DiD lipid component of the nanoparticles) specifically bound to streptavidin-decorated cells. FIG. 15D is a flow cytometry analysis of biotinylated lipid-enveloped nanoparticles bound to cells, control filtered FITC-MPER solution, or FITC-MPER-coated biotinylated nanoparticles bound to cells revealed strong MPER binding to the lipid-enveloped nanoparticles. FIG. 15E is a fluorescence emission spectrum from lipid-enveloped or bare PLGA nanoparticles incubated with 10 μM FITC-MPER (excited at 450 nm) for 1 hour at 37° C. following washing to remove unbound MPER. A strong fluorescence peak in the FITC emission range from adsorbed MPER is detected on lipid-enveloped nanoparticles, but no fluorescence is detected from bare PLGA nanoparticles. (lipid-env NP data is offset by 1×10⁵ fluorescence units for clarity).

FIGS. 16A-16D show that the broadly neutralizing Ab, 4E10, recognizes MPER peptide adsorbed to lipid-enveloped PLGA micro- and nano-particles. Lipid-enveloped PLGA microparticles in the absence of MPER (FIG. 16A) or MPER-coated particles (FIG. 16B) were stained with neutralizing antibody 4E10, followed by secondary staining with Alexafluor 488-conjugated secondary antibody (green fluorescence that appears white in the black and white figure), and visualized by confocal microscopy. Red fluorescence, which appears grey in the black and white figure: DiD in particles. FIGS. 16C and 16D are fluorescence emission spectra of dilute lipid-enveloped nanoparticle suspensions excited with 647 nm light: untreated lipid-enveloped nanoparticles (FIG. 16C) or MPER-coated lipid-enveloped nanoparticles (FIG. 16D) were stained with 4E10 and Alexa 647-conjugated secondary antibody, and fluorescence was measured in the emission range for the secondary antibody.

FIGS. 17A-17E show that nanoparticles are transported to lymph nodes and taken up by dendritic cells and B cells following intradermal immunization. Mice were injected intradermally (i.d.) with 2 mg polystyrene nanoparticles (200 nm diam.); cells recovered from lymph nodes after 48 hours were stained and analyzed by flow cytometry. FIG. 17A shows that particles were clearly detected in ˜3% of cells in the draining lymph nodes, but none in the control contralateral node. FIG. 17B shows that of particle containing cells, ˜40% were CD11c⁺ DCs. FIG. 17D shows that CD11c+ cells internalized substantial amounts of particles. FIG. 17E is an analysis of particle uptake by non-CD11c+ cells; the major population was comprised of CD11c−B220+ B cells.

FIGS. 18A-18B depict the encapsulation of iron oxide in the core of lipid-enveloped PLGA nanoparticles. FIG. 18A is a cryo-electron microscopy image of iron oxide particles (10 nm mean diameter, small dark spots within each nanoparticle in the micrograph) encapsulated in the core of lipid-enveloped PLGA nanoparticles. FIG. 18B depicts the magnetic separation of iron oxide-loaded nanoparticles: lipid-enveloped nanoparticles loaded with iron oxide have a brownish tinge (left); when placed near a bar magnet the particles accumulate against the wall of the vial, clarifying the solution (right).

FIGS. 19A-19C are an EPR analysis of MPER association with lipid-enveloped nanoparticles. MPER peptide (residues 662-683, spin-labeled at N677) was mixed with DOPC/DOPG lipid-enveloped nanoparticles or DOPC/DOPG liposomes at a 300:1 lipid headgroup:MPER mole ratio. FIG. 19A is an EPR spectra for spin-labeled MPER peptides adsorbed to lipid-enveloped PLGA nanoparticles. FIG. 19B is an EPR spectra for spin-labeled MPER peptides adsorbed to liposomes. FIG. 19C is an EPR spectra for spin-labeled MPER peptides adsorbed to ‘bare’ PLGA nanoparticles lacking a lipid skin. “No Ab” denotes MPER spectra in absence of antibody, “4E10” in (FIG. 19A) and (FIG. 19B) denotes the spectra obtained when MPER-adsorbed particles/liposomes were mixed with a 2-fold molar excess of 4E10 antibody relative to MPER.

FIG. 20 depicts the targeting ligand conjugation chemistry for antibody and flagellin coupling to lipid-enveloped nanoparticles.

FIG. 21 depicts the concept of nanoparticle-mediated adenosine receptor/HIF-1α inhibitor delivery.

FIG. 22 depicts the structures of exemplary adenosine receptor inhibitors: caffeine and DMS-DEX.

FIG. 23 depicts the immigration of PLGA-lipid-coated, DiD-labeled nanoparticles to lymph nodes after uptake and transport by dermal dendritic cells. Mice were injected intradermally (i.d.) with 1 mg of lipid-enveloped nanoparticles (200 nm diameter). Lymph nodes from the injected (regional) side and control (contralateral) side were removed 48 hours after injection, stained with mAbs specific for CD11b, CD11c, and B220 antigens.

FIG. 24 is an illustration of a computer system for use in the methods described herein.

FIG. 25 provides the atomic structural coordinates, in Protein Data Bank (PDB) format, for 17 models of the residue sections 662-683 (the MPER) of the HXB2 strain gp160 polypeptide in a 2 DPC micelle, as determined by NMR spectroscopy.

### DETAILED DESCRIPTION

The disclosure features, inter alia, reagents (antigenic and/or immunogenic reagents) that are useful in a variety of in vitro, in vivo, and ex vivo methods. For example, the reagents are useful in methods for inducing an immune response, or for generating an antibody, in a subject. Antigenic reagents containing a membrane proximal external region (MPER) of an HIV-1 gp160 polypeptide, are useful in inducing humoral immunity, and cellular immunity in some embodiments, against HIV-1 and can be used in the treatment or prevention of HIV-1 infections.

Also featured herein are methods, compositions, and kits useful for inducing an immune response (or generating an antibody) in a subject (e.g., a mammal) and in the treatment and/or prevention of a variety of disorders such as microbial infections (e.g., an HIV-1 infection).

In addition, the disclosure provides methods and compositions useful for designing (or identifying) an agent that binds to an MPER of an HIV-1 gp160 polypeptide or an agent that inhibits the fusion of an HIV-1 particle to a cell.

### Reagents

The reagents (antigenic and/or immunogenic reagents) described herein contain: a particle encapsulated in lipid and a polypeptide. The polypeptide contains, or consists of, an MPER of an HIV-1 gp160 polypeptide and at least one amino acid residue of the MPER is embedded in the lipid.

In some embodiments, the MPER can contain, or be, the following amino acid sequence: X₁-L-X₂-X₃-W-X₄-X₅-X₆-W-X₇-W-X₈-X₉-I-X₁₀-X₁₁-W-L-W-Y-I-X₁₂ (SEQ ID NO:1). X₁ can be A, Q, G, or E; X₂ can be D or S; X₃ can be K, S, E, or Q; X₄ can be A, S, T, D, E, K, Q, or N; X₅ can be S, G, or N; X₆ can be L or I; X₇ can be F, N, S, or T; X₈ can be F or S; X₉ can be D, K, N, S, T, or G; X₁₀ can be S or T; X₁₁ can be N, K, S, H, R, or Q; and X₁₂ can be K, E, or R.

In some embodiments, the MPER can contain, or be, any of the amino acid sequences depicted in Table 1.

TABLE 1

| HIV-1 Taxons | Amino Acid Sequence | SEQ ID NO: |
|---|---|---|
| HXB2 | ELDKWASLWNWFNITNWLWYIK | 2 |
| HV1B1 | ELDKWASLWNWFNITNWLWYIK | 2 |
| HV1B8 | ELDKWASLWNWFNITNWLWYIK | 2 |
| HV1BN | ELDKWASLWNWFNITNWLWYIK | 2 |
| HV1BR | ELDKWASLWNWFNITNWLWYIK | 2 |
| HV1H2 | ELDKWASLWNWFNITNWLWYIK | 2 |
| HV1H3 | ELDKWASLWNWFNITNWLWYIK | 2 |
| HV1LW | ELDKWASLWNWFNITNWLWYIK | 2 |
| HV1SC | ELDKWASLWNWFNITNWLWYIK | 2 |
| ADA | ALDKWASLWNWFDISNWLWYIK | 3 |
| HV197 | ALDKWASLWNWFDISNWLWYIK | 3 |
| HV1VI | ALDKWASLWNWFDISNWLWYIK | 3 |
| HV190 | ALDKWASLWTWFDISHWLWYIK | 4 |
| HV193 | ALDKWASLWNWFDITQWLWYIK | 5 |
| HV196 | ALDKWASLWNWFDITKWLWYIK | 6 |
| HV19N | ALDKWASLWNWFDISNWLWYIR | 7 |
| HV1ZH | ALDKWANLWNWFDISNWLWYIK | 8 |
| HV1A2 | ELDKWASLWNWFSITNWLWYIK | 9 |
| HV1W1 | ELDKWASLWNWFSITNWLWYIK | 9 |
| HV1S3 | ELDKWASLWNWFSITNWLWYIR | 10 |
| HV1B9 | ELDKWASLWNWFDITNWLWYIR | 11 |
| HV1MN | ELDKWASLWNWFDITNWLWYIK | 12 |
| HV1W2 | ELDKWASLWNWFDITNWLWYIK | 12 |
| HV1EL | ELDKWASLWNWFSITQWLWYIK | 13 |
| HV1Z2 | ELDKWASLWNWFNITQWLWYIK | 14 |
| HV1Z6 | ELDKWASLWNWFNITQWLWYIK | 14 |
| HV1ND | ELDKWASLWNWFSITKWLWYIK | 15 |
| HV1Z8 | QLDKWASLWNWFSITKWLWYIK | 16 |
| HV1JR | ELDKWASLWNWFGITKWLWYIK | 17 |
| HV1MA | ELDKWASLWNWFSISKWLWYIR | 18 |
| HV1MV | ELDKWASLWNWFSISKWLWYIR | 18 |
| HV1AN | ELDEWASIWNWLDITKWLWYIK | 19 |
| HV1MF | ELDEWASLWNWFDITKWLWYIK | 20 |
| HV1Y2 | ELDQWASLWNWFDITKWLWYIK | 21 |
| HV1S1 | ELDKWASLWNWFDISKWLWYIK | 22 |
| HV1RH | ELDKWANLWNWFDITQWLWYIR | 23 |
| HV1ET | ALDKWENLWNWFNITNWLWYIK | 24 |
| HV1S2 | ALDKWTNLWNWFNISNWLWYIK | 25 |
| HV1S9 | ALDKWTNLWNWFNISNWLWYIK | 25 |
| HV1V9 | ALDKWANLWNWFSITNWLWYIR | 26 |
| HV1J3 | GLDKWASLWNWFTITNWLWYIR | 27 |
| HV1OY | ELDKWAGLWSWFSITNWLWYIR | 28 |
| HV1KB | ALDKWDSLWNWFSITKWLWYIK | 28 |
| HV1MP | ALDKWDSLWSWFSITNWLWYIK | 29 |
| HV1M2 | ALDKWDNLWNWFSITRWLWYIE | 30 |
| HV192 | ALDKWQNLWTWFGITNWLWYIK | 31 |
| HV1YF | ELDQWDSLWSWFGITKWLWYIK | 32 |
| HV1C4 | QLDKWASLWTWSDITKWLWYIK | 33 |

In some embodiments, the polypeptide can be an MPER-containing fragment of a Group M HIV-1 gp160 polypeptide. In some embodiments, the polypeptide can be an f-containing fragment of a Clade A, B, C, or D HIV-1 gp160 polypeptide.

In some embodiments, the polypeptide can be an MPER-containing fragment of an HXB2 strain HIV-1 gp160 polypeptide. An exemplary HXB2 strain HIV-1 gp160 polypeptide is as follows: MRVKEKYQHLWRWGWRWGTMLLGMLMICSATEKLWVTVYYGVPVWKEATTTLFCA SDAKAYDTEVHNVWATHACVPTDPNPQEVVLVNVTENFNMWKNDMVEQMHEDIISL WDQSLKPCVKLTPLCVSLKCTDLKNDTNTNSSSGRMIMEKGEIKNCSFNISTSIRGKVQK EYAFFYKLDIIPIDNDTTSYKLTSCNTSVITQACPKVSFEPIPIHYCAPAGFAILKCNNKTF NGTGPCTNVSTVQCTHGIRPVVSTQLLLNGSLAEEEVVIRSVNFTDNAKTIIVQLNTSVEI NCTRPNNNTRKRIRIQRGPGRAFVTIGKIGNMRQAHCNISRAKWNNTLKQIASKLREQF GNNKTIIFKQSSGGDPEIVTHSFNCGGEFFYCNSTQLFNSTWFNSTWSTEGSNNTEGSDTI TLPCRIKQIINMWQKVGKAMYAPPISGQIRCSSNITGLLLTRDGGNSNNESEIFRPGGGD MRDNWRSELYKYKVVKIEPLGVAPTKAKRRVVQREKRAVGIGALFLGFLGAAGSTMG AASMTLTVQARQLLSGIVQQQNNLLRAIEAQQHLLQLTVWGIKQLQARILAVERYLKD QQLLGIWGCSGKLICTTAVPWNASWSNKSLEQIWNHTTWMEWDREINNYTSLIHSLIEE SQNQQEKNEQELLELDKWASLWNWFNITNWLWYIKLFIMIVGGLVGLRIVFAVLSIVNR VRQGYSPLSFQTHLPTPRGPDRPEGIEEEGGERDRDRSIRLVNGSLALIWDDLRSLCLFSY HRLRDLLLIVTRIVELLGRRGWEALKYWWNLLQYWSQELKNSAVSLLNATAIAVAEGT DRVIEVVQGACRAIRHIPRRIRQGLERILL (SEQ ID NO:37). In some embodiments, the polypeptide can contain, or be, the amino acid sequence corresponding to amino acid positions 660 to 856 of the HXB2 strain HIV-1 gp160 polypeptide (SEQ ID NO:37). In some embodiments, the polypeptide can contain, or be, the amino acid sequence corresponding to amino acid positions 662 to 856 of the HXB2 strain HIV-1 gp160 polypeptide (SEQ ID NO:37). In some embodiments, the polypeptide can contain, or be, the amino acid sequence corresponding to amino acid positions 662 to 683 of the HXB2 strain HIV-1 gp160 polypeptide (SEQ ID NO:37).

In some embodiments, the polypeptide can be an MPER-containing fragment of an ADA strain HIV-1 gp160 polypeptide. An exemplary ADA strain HIV-1 gp160 polypeptide is as follows: MRVKEKYQHLWRWGWKWGTMLLGILMICSATEKLWVTVYYGVPVWKEATTTLFCAS DAKAYDTEVHNVWATHACVPTDPNPQEVVLENVTENFNMWKNNMVEQMHEDIISLW DQSLKPCVKLTPLCVTLNCTDLRNVTNINNSSEGMRGEIKNCSFNITTSIRDKVKKDYAL FYRLDVVPIDNDNTSYRLINCNTSTITQACPKVSFEPIPIHYCTPAGFAILKCKDKKFNGT GPCKNVSTVQCTHGIRPVVSTQLLLNGSLAEEEVVIRSSNFTDNAKNIIVQLKESVEINCT RPNNNTRKSIHIGPGRAFYTTGEIIGDIRQAHCNISRTKWNNTLNQIATKLKEQFGNNKTI VFNQSSGGDPEIVMHSFNCGGEFFYCNSTQLFNSTWNFNGTWNLTQSNGTEGNDTITLP RIKQIINMWQEVGKAMYAPPIRGQIRCSSNITGLILTRDGGTNSSGSEIFRPGGGDMRDN WRSELYKYKVVKIEPLGVAPTKAKRRVVQREKRAVGTIGAMFLGFLGAAGSTMGAASI TLTVQARLLLSGIVQQQNNLLRAIEAQQHLLQLTVWGIKQLQARVLALERYLRDQQLLG IWGCSGKLICTTAVPWNASWSNKTLDMIWDNMTWMEWEREIENYTGLIYTLIEESQNQ QEKNEQDLLALDKWASLWNWFDISNWLWYIKIFIMIVGGLIGLRIVFTVLSIVNRVRQG YSPLSFQTHLPAPRGPDRPEGIEEEGGDRDRDRSVRLVDGFLALFWDDLRSLCLFSYHRL RDLLLIVARIVELLGRRGWEVLKYWWNLLQYWSQELRNSAVSLLNATAIAVAEGTDRV IEVVQRIYRAILHIPTRIRQGLERLLL (SEQ ID NO:38). In some embodiments, the polypeptide can be a full-length, HIV-1 gp160 polypeptide such as, but not limited to, SEQ ID NO:37 or SEQ ID NO:38.

In some embodiments, the polypeptide can contain less than 500 (e.g., less than 490, 480, 470, 460, 450, 440, 430, 420, 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 29, 28, 27, 26, 25, 24, 23, 22, 21, or 20) amino acids.

In some embodiments, at least two (e.g., at least three, at least four, at least five, at least six, at least seven, at least eight, or at least nine or more) amino acid residues of the MPER can be embedded in the lipid. In some embodiments, no more than 10 (e.g., no more than nine, eight, seven, six, five, four, three, two, or one) amino acid residues can be embedded in the lipid. The amino acids that are embedded in the lipid can be those corresponding to, e.g., L669, W670, W672, F673, I675, W678, L679, Y681, I682, or K683 of the HXB2 strain HIV-1 gp160 polypeptide.

In some embodiments, at least one (e.g., at least two, at least three, at least four, at least five, at least six, at least seven, at least eight, at least nine, at least 10, at least 11, at least 12, at least 13, at least 14, at least 15, or at least 20 or more) amino acid residue(s) of the MPER is/are not embedded in the lipid. The amino acid residue not embedded in the lipid can be one corresponding to position 671, 674, 677, or 680 of the HXB2 strain HIV-1 gp160 polypeptide.

In some embodiments, the MPER can be flanked at the amino-terminus, the carboxy-terminus, or both the amino-terminus and the carboxy-terminus by a heterologous amino acid sequence. A heterologous sequence can be any of those described above.

The polypeptide containing the MPER can be naturally occurring or recombinant. For example, a natural or recombinant polypeptide containing an MPER can be isolated from a cell, from a viral particle (e.g., an HIV-1 viral particle), or from a medium in which a cell or virus is cultured, using standard techniques (see Sambrook et al., Molecular Cloning: A Laboratory Manual Second Edition vol. 1, 2 and 3. Cold Spring Harbor Laboratory Press: Cold Spring Harbor, N.Y., USA, November 1989; the disclosure of which is incorporated herein by reference in its entirety). Methods for isolating a polypeptide from one or more unwanted components (e.g., other biomolecules) are known in the art and include, e.g., liquid chromatography (e.g., HPLC), affinity chromatography (e.g., metal chelation or immunoaffinity chromatography), ion-exchange chromatography, hydrophobic-interaction chromatography, precipitation, or differential solubilization.

Smaller polypeptides containing an MPER, e.g., polypeptides that are less than 200 (e.g., less than 175, less than 150, less than 125, less than 100, less than 90, less than 80, less than 70, or less than 60) amino acids can be chemically synthesized by standard chemical means.

A particle component of any of the reagents described herein can be composed of a variety of materials or a combination of materials depending on the particular application. For example, a particle can contain, or consist of, a natural or synthetic material or an organic or inorganic material. For example, a particle can contain a polymer, a resin, carbon, latex, a metal, a glass, or combinations of any of the foregoing. Polymeric materials include, e.g., polystyrene, polyethylene, polyvinyltoluene, polyvinyl chloride, poly(lactic-co-glycolic acid) (PLGA), or an acrylic polymer. Polymers can be composed of any of the following monomers: divinyl benzene, trivinyl benzene, divinyl toluene, trivinyl toluene, triethylenglycol dimethacrylate, tetraethylenglycol dimethacrylate, allylmethacrylate, diallylmaleate, triallylmaleate, or 1, 4-butanediol diacrylate. Polymeric materials also include polysaccharides such as dextran or inorganic oxides such as alumina or silica. Polymeric materials can be bioresorbable, e.g., a polyester or polycaprolactone, polyhydroxybutyrate, poly(beta-amino esters), polylactide, or polycarbonates. In some embodiments, the particle can contain, or consist of, a magnetic metal such as magnetite (Fe₃O₄), maghemite (γFe₂O₃), or greigite (Fe₃S₄). The particle can be superparamagnetic or single-domain (i.e., with a fixed magnetic moment). In some embodiments, the particles can contain non-magnetic metals (e.g., gold or silver) or any of a variety of metal salts (e.g., cadmium sulfide). The particles can contain one or more (e.g., two three, four, five, six, seven, eight, nine, 10, 11, 12, 13, 14, 15, 20, 25, 30, or more) of any of the above described suitable materials.

In some embodiments, the particles can be “quantum dots,” which are semiconductor nanostructures such as colloidal semiconductor nanocrystals (see, e.g., Reed et al. (1988) Phys Rev Lett 60 (6): 535-537; Reed (1993) Scientific American 268 (1): 118; Murray et al. (1993) J Am Chem Soc 115: 8706-15; Buhro et al. (2003) Nature materials 2 (3): 138-9; and Shim et al. (2000) Nature 407 (6807): 981-3).

In some embodiments, particles can be encoded. That is, each particle can include a unique code (such as a bar code, luminescence code, fluorescence code, a nucleic acid code, and the like). The code is embedded (for example, within the interior of the particle) or otherwise attached to the particle in a manner that is stable through processes such as, e.g., lipid encapsulation, purification, and/or dilution or suspension in a pharmaceutically acceptable carrier. The code can be provided by any detectable means, such as by holographic encoding, by a fluorescence property, color, shape, size, weight, light emission, quantum dot emission and the like to identify particle and thus the capture probes immobilized thereto. Encoding can also be the ratio of two or more dyes in one particle that is different than the ratio present in another particle. For example, the particles may be encoded using optical, chemical, physical, or electronic tags. Examples of such coding technologies are optical bar codes fluorescent dyes, or other means.

Encoded particles, like magnetic particles, are useful for, e.g., separating a mixture of different particles or different reagents (e.g., reagents with different polypeptides; see below), tracking the localization of a reagent in a subject, or determining whether a reagent has fused with, or been endocytosed, by a cell. A particle can be both encoded and magnetic.

In some embodiments, a particle can consist of, or contain, a therapeutic, diagnostic, or prophylactic agent. That is, the particle can be, e.g., a medicament that is co-delivered to a cell along with the polypeptide of the reagent. Generally, any chemical compound to be administered to a subject may be incorporated into the particles. For example, an agent can be a small molecule, a nucleic acid (e.g., DNA, an RNA (such as anti-sense RNA, an siRNA, or a miRNA), or a protein. The agent can be, or contain, e.g., a HIF la inhibitor or an adenosine receptor inhibitor. The agent can be, e.g., an antibiotic, an anti-viral agent (see anti-HIV-1 agent), an anesthetic, a steroidal agent, an anti-inflammatory agent, an anti-neoplastic agent, an antigen, an antibody, a decongestant, an antihypertensive, a sedative, an anti-cholinergic, an analgesic, an anti-depressant, an anti-psychotic, a polypeptide containing a T helper epitope such as any of those described herein, a β-adrenergic blocking agent, a diuretic, a vasoactive agent, an anti-inflammatory agent, or a nutritional agent (e.g., a vitamin such as vitamin A, B, C, or D). For example, the particles can include one or more agents selected from the group consisting of: (i) drugs that act at synaptic and neuroeffector junctional sites (e.g., acetylcholine, methacholine, pilocarpine, atropine, scopolamine, physostigmine, succinylcholine, epinephrine, norepinephrine, dopamine, dobutamine, isoproterenol, albuterol, propranolol, or serotonin); (ii) drugs that act on the central nervous system (e.g., clonazepam, diazepam, lorazepam, benzocaine, bupivacaine, lidocaine, tetracaine, ropivacaine, amitriptyline, fluoxetine, paroxetine, valproic acid, carbamazepine, bromocriptine, morphine, fentanyl, naltrexone, or naloxone); (iii) drugs that modulate inflammatory responses (e.g., aspirin, indomethacin, ibuprofen, naproxen, steroids, cromolyn sodium, or theophylline); (iv) drugs that affect renal and/or cardiovascular function (e.g., furosemide, thiazide, amiloride, spironolactone, captopril, enalapril, lisinopril, diltiazem, nifedipine, verapamil, digoxin, isordil, dobutamine, lidocaine, quinidine, adenosine, digitalis, mevastatin, lovastatin, simvastatin, or mevalonate); (v) drugs that affect gastrointestinal function (e.g., omeprazole or sucralfate); (vi) antibiotics (e.g., tetracycline, clindamycin, amphotericin B, quinine, methicillin, vancomycin, penicillin G, amoxicillin, gentamicin, erythromycin, ciprofloxacin, doxycycline, streptomycin, gentamicin, tobramycin, chloramphenicol, isoniazid, fluconazole, or amantadine); (vii) anti-cancer agents (e.g., cyclophosphamide, methotrexate, fluorouracil, cytarabine, mercaptopurine, vinblastine, vincristine, doxorubicin, bleomycin, mitomycin C, hydroxyurea, prednisone, tamoxifen, cisplatin, or decarbazine); (viii) immunomodulatory agents (e.g., interleukins, interferons, GM-CSF, TNFα, TNFβ, cyclosporine, FK506, azathioprine, steroids); (ix) drugs acting on the blood and/or the blood-forming organs (e.g., interleukins, G-CSF, GM-CSF, erythropoietin, heparin, warfarin, or coumarin); or (x) hormones (e.g., growth hormone (GH), prolactin, luteinizing hormone, TSH, ACTH, insulin, FSH, CG, somatostatin, estrogens, androgens, progesterone, gonadotropin-releasing hormone (GnRH), thyroxine, triiodothyronine); hormone antagonists; agents affecting calcification and bone turnover (e.g., calcium, phosphate, parathyroid hormone (PTH), vitamin D, bisphosphonates, calcitonin, fluoride).

In some embodiments, a particle can contain, or consist of, a combination of two or more therapeutic, diagnostic, or prophylactic agents. For example, a particle can contain, or consist of, at least two (e.g., at least three, four, five, six, seven, eight, nine, 10, 11, 12, 13, 14, or 15 or more) therapeutic, diagnostic, or prophylactic agents.

Generally, a particle described herein has a spherical shape. However, a particle can be, e.g., oblong or tube-like. In some embodiments, e.g., a crystalline form particle, the particle can have polyhedral shape (irregular or regular) such as a cube shape. In some embodiments, a particle can be amorphous.

In some embodiments, the particle or particle mixture can be substantially spherical, substantially oblong, substantially tube-like, substantially polyhedral, or substantially amorphous. By “substantially” is meant that the particle, or the particle mixture, is more than 30 (e.g., 35, 40, 45, 50, 55, 60, 65, 70, 75, 80, 85, 90, 95, 96, 97, 98, or 99 or more) % of a given shape.

In some embodiments, the diameter of the particle can be between about 1 nm to about 1000 nm or larger. For example, a particle can be at least about 1 nm to about 1000 nm (e.g., at least about two, three, four, five, six, seven, eight, nine, 10, 15, 20, 25, 30, 35, 40, 45, 50, 75, 100, 150, 200, 250, 300, 325, 350, 375, 400, 425, 450, 475, 500, 525, 550, 575, 600, 625, 650, 675, 700, 725, 750, 775, 800, 825, 850, 875, 900, 925, 950, 975, or 1000 nm). In some embodiments, a particle can be not more than 1000 nm (e.g., not more than 975, 950, 925, 900, 875, 850, 825, 800, 775, 750, 725, 700, 675, 650, 625, 600, 575, 550, 525, 500, 475, 450, 425, 400, 375, 350, 325, 300, 275, 250, 225, 200, 175, 150, 125, 100, 75, 50, 45, 40, 35, 30, 25, 20, 15, 10, or five nm) in diameter (or at its longest straight dimension).

Where the particles (the particle core of the lipid-encapsulated particle) are in a dispersion of a plurality of particles, the size distribution can have a standard deviation of no more than about 35% (e.g., one, two, three, four, five, six, seven, eight, nine, 10, 15, 20, 25, 30, or 35%) of the average diameter of the plurality of particles.

The particles described herein can be porous or substantially without pores. Pores in a particle (e.g., a nanoparticle) can be of any size that is less than the diameter (or longest straight dimension) of the particle. For example, pores in a nanoparticle can average about 0.2, 0.5, one, two, three, four, five, six, seven, eight, nine, 10, 20, 50, 60, 70, 80, 90, or 100 nm in size.

In some embodiments, the particles can be bioresorbable and/or biodegradable.

In some embodiments, the particles can be solid. As used herein, “solid” with regard to a particle means that at least a portion of a particle is solid at room temperature and atmospheric pressure. However, a solid particle can include portions of liquid and/or entrapped solvent. In some embodiments, a particle can be completely solid at room temperature and atmospheric pressure.

In some embodiments, the particles can be hollow. The hollow cavity can be filled with, e.g., any of the additional polypeptides or therapeutic, diagnostic, or prophylactic agents described herein.

Methods for preparing a particle are included in the accompanying Examples and known in the art. For example, a polymer nanoparticle can be formed by dispersion polymerization, emulsion polymerization, condensation polymerization, cationic polymerization, ring opening polymerization, anionic polymerization, living free radical (i.e., atom transfer radical, nitroxide mediated), and free radical addition polymerization (see, e.g., European Patent No. EP1411076 and U.S. Pat. No. 7,112,369, the disclosures of each of which are incorporated by reference in their entirety). Additional methods for preparing a particle (e.g., a magnetic, encoded, polymeric, or silicate particle) are described in, e.g., U.S. Patent Publication Nos. 20030029590 and 20070051815; International Patent Publication No. WO/2003/010091; and U.S. Pat. Nos. 7,106,513 and 6,384,104; the disclosures of each of which are incorporated by reference in their entirety.

A wide variety of particles can be obtained from commercial sources such as G.Kisker GbR (Germany), Spherotech (Lake Forest, Ill.), and microParticles GmbH (Germany).

Any lipid including surfactants and emulsifiers known in the art is suitable for use in the reagents described herein. The lipids can be natural or synthetic or a combination of both. The lipids can be altered, e.g., chemically altered. Lipids can be, e.g., phospholipids, a glycolipid, a sphingolipid, or a sterol such as cholesterol. In some embodiments, the lipids can contain a glycerol or sphingosine core such as a glycolipid or phospholipid. In some embodiments, lipids can be amphipathic.

Suitable lipids for use in the reagents described herein include those set forth in the accompanying Examples as well as many known in the art. Thus, useful lipids include, e.g., phosphoglycerides; phosphatidylcholines; dipalmitoyl phosphatidylcholine (DPPC); dioleyloxypropyltriethylammonium (DOTMA); 1,2-dioleoyl-sn-glycero-3-phosphatidylcholine (DOPC); 1,2-dioleoyl-sn-glycero-3-phosphatidylethanolamine (DOPE); 1,2-dioleoyl-sn-glycero-3-[phospho-rac-(1-glycerol)] (DOPG); egg sphingomyelin (SM); 1-Palmitoyl-2-oleoyl-sn-glycero-3-phosphocholine (POPC); 1-palmitoyl-2-oleoyl-sn-glycero-3-[phospho-rac-(1-glycerol)] (POPG); 1-palmitoyl-2-oleoyl-sn-glycero-3-phosphotempocholine (PC tempo); 1-palmitoyl-2-stearoyl(5-doxyl)-sn-glycero-3-phosphocholine (5-doxyl PC); 1-palmitoyl-2-stearoyl(7-doxyl)-sn-glycero-3-phosphocholine (7-doxyl PC); 1-palmitoyl-2-stearoyl(10-doxyl)-sn-glycero-3-phosphocholine (10-doxyl PC); 1-palmitoyl-2-stearoyl(12-doxyl)-sn-glycero-3-phosphocholine (12-doxyl PC); dioleoylphosphatidylcholine; diacylglycerol; diacylglycerolsuccinate; diphosphatidyl glycerol (DPPG); hexanedecanol; fatty alcohols such as polyethylene glycol (PEG); polyoxyethylene-9-laury-1 ether; a surface active fatty acid, such as palmitic acid or oleic acid; fatty acids; fatty acid amides; sorbitan trioleate (Span 85) glycocholate; surfactin; a poloxomer; a sorbitan fatty acid ester such as sorbitan trioleate; lecithin; lysolecithin; phosphatidylserine; phosphatidylinositol; sphingomyelin; phosphatidylethanolamine (cephalin); cardiolipin; phosphatidic acid; cerebrosides; dicetylphosphate; dipalmitoylphosphatidylglycerol; stearylamine; dodecylamine; hexadecyl-amine; acetyl palmitate; glycerol ricinoleate; hexadecyl sterate; isopropyl myristate; tyloxapol; poly(ethylene glycol)5000-phosphatidylethanolamine; and phospholipids. The lipid can be positively charged, negatively charged, or neutral. Phospholipids include, e.g., negatively charged phosphatidyl inositol, phosphatidyl serine, phosphatidyl glycerol, phosphatic acid, diphosphatidyl glycerol, poly(ethylene glycol)-phosphatidyl ethanolamine, dimyristoylphosphatidyl glycerol, dioleoylphosphatidyl glycerol, dilauryloylphosphatidyl glycerol, dipalmitotylphosphatidyl glycerol, di stearyloylphosphatidyl glycerol, dimyristoyl phosphatic acid, dipalmitoyl phosphatic acid, dimyristoyl phosphitadyl serine, dipalmitoyl phosphatidyl serine, phosphatidyl serine, or combinations of any of the foregoing. Zwitterionic phospholipids include, but are not limited to, phosphatidyl choline, phosphatidyl ethanolamine, sphingomyeline, lecithin, lysolecithin, lysophatidylethanolamine, cerebrosides, dimyristoylphosphatidyl choline, dipalmitotylphosphatidyl choline, di stearyloylphosphatidyl choline, dielaidoylphosphatidyl choline, dioleoylphosphatidyl choline, dilauryloylphosphatidyl choline, 1-myristoyl-2-palmitoyl phosphatidyl choline, 1-palmitoyl-2-myristoyl phosphatidyl choline, 1-palmitoyl-phosphatidyl choline, 1-stearoyl-2-palmitoyl phosphatidyl choline, dimyristoyl phosphatidyl ethanolamine, dipalmitoyl phosphatidyl ethanolamine, brain sphingomyelin, dipalmitoyl sphingomyelin, distearoyl sphingomyelin, or combinations of any of the foregoing.

In some embodiments, the lipid can comprise a monoglyceride, diglyceride, or triglyceride of at least one C₄ to C₂₄ carboxylic acid. The carboxylic acid can be saturated or unsaturated and can be branched or unbranched. For example, the lipid can be a monoglyceride of a C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, or C₂₄ carboxylic acid. The carboxylic acid can be saturated or unsaturated and branched or unbranched. The carboxylic acid can be covalently linked to any one of the three glycerol hydroxyl groups or an amino group of sphingosine. In another example, the lipid can be a diglyceride of C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, or C₂₄ carboxylic acids. The two carboxylic acids can be the same or different, and the carboxylic acids can be covalently linked to any two of the three glycerol hydroxyl groups. In a further example, the lipid can be a triglyceride of C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, or C₂₄ carboxylic acids. The three carboxylic acids can be the same, two of the carboxylic acid can be the same, or all three can be different. That is, the triglyceride can comprise, e.g., two fatty acids having the same chain length and another of a different chain length or can comprise three fatty acids having the same chain length.

In some embodiments, the lipid can contain a monoglyceride, diglyceride, or triglyceride of at least one saturated, even-numbered, unbranched natural fatty acid with a chain length of C₈ to C₁₈. For example, the lipid can be a triglyceride of C₈, C₁₀, C₁₂, C₁₄, C₁₆, or C₁₈ carboxylic acids.

Sterols include, but are not limited to, cholesterol, cholesterol derivatives, cholesteryl esters, vitamin D, phytosterols, ergosterol, or steroid hormones. Examples of cholesterol derivatives include, but are not limited to, cholesterol-phosphocholine, cholesterolpolyethylene glycol, and cholesterol-SO₄. Phytosterols can be, e.g., sitosterol, campesterol, and stigmasterol. Salt forms of organic acid derivatives of sterols can also be used and are described in, e.g., U.S. Pat. No. 4,891,208, the disclosure of which is incorportated herein by reference in its entirety.

Derivatized lipids can also be used in the reagents described herein. Derivatized lipids, or derivatized lipids in combination with non-derivatized lipids, can be used to alter one or more pharmacokinetic properties of the reagents. In some embodiments, the derivatized lipids of the reagents include a labile lipid-polymer linkage, such as a peptide, amide, ether, ester, or disulfide linkage, which can be cleaved under selective physiological conditions, such as in the presence of peptidase or esterase enzymes or reducing agents. Such linkages allow for the attainment of high blood levels for several hours after administration as described in, e.g., U.S. Pat. No. 5,356,633, the disclosure of which is incorporated herein by reference in its entirety. The surface charge of the lipid portion of the reagent can also be altered. Thermal or pH release characteristics can be built into the reagent by, e.g., incorporating thermal sensitive or pH sensitive lipids as a component of the lipid portion (e.g., dipalmitoyl-phosphatidylcholine:distearyl phosphatidylcholine (DPPC:DSPC) based mixtures). Use of thermal or pH sensitive lipids can also allow for controlled degradation of the lipid portion of the reagent.

The lipid portion of the reagent can adopt any of a variety of conformations depending on, e.g., the intended application and/or the type of solvent in which the reagent is present. For example, the lipid can be multilamellar or unilamellar. In some embodiments, the particle can be encapsulated with a multilamellar lipid membrane such as a lipid bilayer. In some embodiments, the particle can be encapsulated with a unilamellar lipid membrane such as a micelle. In some embodiments, a particle can be encapsulated by more than one lipid bilayer.

In some embodiments, the lipid portion of the reagent can include more than one (e.g., two, three, four, five, six, seven, eight, nine, 10, 11, 12, 15, 20, 22, 25, 27, 30, 32, 35, 37, 40, 45, or 50 or more) different types of lipid. In embodiments where the lipid forms a bilayer, a lipid combination can include one or more sterols such as cholesterol.

In some embodiments, the lipid portion of the reagent can be all or a part of a lipid bilayer from a cell or a microorganism. For example, the lipid can include all or part of the lipid envelope of a virus such as HIV-1.

In some embodiments, the diameter of a reagent can be, e.g., at least about 10 nm to about 2000 nm (e.g., at least about 15, 20, 25, 30, 35, 40, 45, 50, 75, 100, 125, 150, 175, 200, 225, 250, 250, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1125, 1150, 1175, 1200, 1250, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1925, 1950, or 1975 nm). In some embodiments, a reagent can be not more than 2000 nm (e.g., not more than 1975, 1950, 1925, 1900, 1875, 1850, 1825, 1800, 1775, 1750, 1725, 1700, 1675, 1650, 1625, 1600, 1575, 1550, 1525, 1500, 1475, 1450, 1425, 1400, 1375, 1350, 1325, 1300, 1275, 1250, 1225, 1200, 1175, 1150, 1125, 1100, 1000, 975, 950, 925, 900, 875, 850, 825, 800, 775, 750, 725, 700, 675, 650, 625, 600, 575, 550, 525, 500, 475, 450, 425, 400, 375, 350, 325, 300, 275, 250, 225, 200, 175, 150, 125, 100, 75, 50, 45, 40, 35, 30, 25, 20, 15, 10, or five nm) in diameter (or at its longest straight measurement).

In some embodiments, the reagent (or the particle component of the reagent) can be a nanoparticle, i.e., a particle with at least one dimension that is less than 100 nm.

In some embodiments, the reagent (or the particle component of the reagent) can be a microparticle, i.e., a particle with at least one dimension that is between 0.1 and 11 μm. That is, a microparticle can be about 100 (e.g., 200, 300, 400, 500, 600, 700, 800, 900, 1000, 1200, 1300, 1400, 1500, 1600, 1700, 1800, 1900, 2000, 2200, 2400, 2600, 2800, 3000, 3200, 3400, 3600, 3800, 4000, 4200, 4400, 4600, 4800, 5000, 5200, 5400, 5600, 5800, 6000, 7200, 7400, 7600, 7800, 8000, 8200, 8400, 8600, 8800, 9000, 9200, 9400, 9600, 9800, 10000, 10250, 10500, 10750, or 11000) nm.

Any of the reagents described herein can also include at least one (e.g., two, three, four, five, six, seven, eight, nine, 10, 11, 15, 20, 25, or 30 or more) additional polypeptide(s). The one or more additional polypeptide(s) can be, e.g., a targeting polypeptide, a therapeutic polypeptide, a dendritic cell activating polypeptide, or a microbial polypeptide such as a polypeptide from a virus (e.g., HIV-1), bacterium, or protozoan. Examples of microbes from which polypeptides can be derived are described below.

Targeting polypeptides, as used herein, are polypeptides that target the reagents described herein to specific tissues (e.g., to a lymph node) or cells (e.g., to an antigen presenting cell or other immune cell), or where in vitro, specific isolated molecules or molecular complexes. Targeting polypeptides can be, e.g., an antibody or antigen binding fragment thereof or a ligand for a cell surface receptor. An antibody (or antigen-binding fragment thereof) can be, e.g., a monoclonal antibody, a polyclonal antibody, a humanized antibody, a fully human antibody, a single chain antibody, a chimeric antibody, or an Fab fragment, an F(ab′)₂fragment, an Fab′ fragment, an Fv fragment, or an scFv fragment of an antibody. Antibody fragments that include Fc regions (with or without antigen-binding regions) can also be used to target the reagents to Fc receptor-expressing cells (e.g., antigen presenting cells such as interdigitating dendritic cells). A ligand for a cell surface receptor can be, e.g., a chemokine, a cytokine (e.g., Interleukins 1, 2, 3, 4, 5, 6, 7, 8, 9, 10, 11, 12, 13, 14, 15, or 16), or a death receptor ligand (e.g., FasL or TNFα).

The therapeutic polypeptide can be, or contain, a T helper epitope such as, but not limited to, a PADRE (SEQ ID NO:41) epitope or a TT-Th universal T helper cell epitope. In some embodiments, the T helper epitope can contain, or consist of, one or more (e.g., two, three, four, five, six, seven, eight, nine, 10, 11, 12, 13, 14, 15, 20, 25, or 30 or more) polypeptides or peptide fragments thereof from microorganisms (e.g., an infectious microorganism such as HIV-1) that are capable of specifically binding to a particular MHC Class II alleles. In this way, the reagents can be antigenically customized to a particular subject or group of subjects based on their MHC Class II allele status.

In some embodiments, a reagent can contain a polypeptide that targets the reagent to an antigen presenting cell such as a dendritic cell or a macrophage.

In some embodiments, the reagents can contain a polypeptide consisting of the MPER and one or more additional HIV-1 polypeptides such as, e.g., full-length gp160, gp41, gp120, Rev, Nef, Tat, Vif, Vpr, protease, integrase, reverse transcriptase, or fragments or variants of any of the foregoing.

Any of the reagents described herein can also include one or more additional therapeutic or prophylactic agents. The agents can be, e.g., an immune modulator or any of those described above. The agents can be lipophilic and can be embedded within the lipid. The immune modulator can be a ligand for a Toll Receptor or an adjuvant such as any of those described herein. Ligands for Toll Receptors include any of a variety of microbial molecules (e.g., proteins, nucleic acids, or lipids) such as, but not limited to, triacyl lipopeptides, OspA, Porin PorB, peptidoglycan, lipopolysaccharide (LPS), hemagglutinin, flavolipin, unmethylated CpG DNA, flagellin, lipoarabinomannan, or zymosan. Additional Toll Receptor ligands are described in, e.g., Gay et al. (2007) Annual Review of Biochemistry 76:141-165, the disclosure of which is incorporated herein by reference in its entirety.

Any of the reagents described herein can also include one or more (e.g., two or more, three or more, four or more, five or more, six or more, seven or more, eight or more, nine or more, or 10 or more) detectable labels. Any component of the reagent can be detectably labeled. For example, a polypeptide, a particle (e.g., an encoded particle), or lipid can be detectably labeled. The type and nature of the detectable label can vary in, e.g., the component of the reagent that is labeled and the specific application. Generally, a detectable label includes, but is not limited to, an enzyme (e.g., horseradish peroxidase, alkaline phosphatase, β-galactosidase, or acetylcholinesterase), a fluorescent material (e.g., umbelliferone, fluorescein, fluorescein isothiocyanate, rhodamine, dichlorotriazinylamine fluorescein, dansyl chloride, allophycocyanin (APC), or phycoerythrin), a luminescent material (e.g., europium, terbium), a bioluminescent material (e.g., luciferase, luciferin, or aequorin), or a radionuclide (e.g., ³³P, ³²P, ¹⁵N, ¹³C, or ³H).

The disclosure also features a plurality or mixture of two or more (e.g., three, four, five, six, seven, eight, nine, 10, 11, 12, 15, 20, 25, 30, 35, or 40 or more) of any of the reagents described herein (i.e., a plurality or mixture of different reagents). The plurality can contain reagents that differ from one another by any of a variety of characteristics including, e.g., particle, lipid, or polypeptide composition. For example, the plurality can contain a first reagent with a metal particle core, a second reagent with a polymer particle core, and a third reagent with a glass particle core. In another example, the plurality can contain a first reagent comprising a lipid monolayer-encapsulated particle and a second reagent comprising a lipid bilayer-encapsulated particle. In yet another example, the plurality can contain a first reagent containing a polypeptide with a first MPER sequence and a second reagent containing a polypeptide with a second MPER sequence. The plurality can also contain reagents that different from one another by therapeutic agent. For example, a plurality can contain a first reagent that comprises an analgesic and a second reagent comprising an immune modulator.

It is understood that the plurality can contain two or more different reagents in various ratios. For example, 20% of a plurality of reagents can be a first reagent, 30% of the plurality a second reagent, and 50% of the plurality a third reagent.

Where the reagent (the particle core of the lipid-encapsulated particle) is in a dispersion of a plurality of reagents, the size distribution can have a standard deviation of no more than about 35% (e.g., one, two, three, four, five, six, seven, eight, nine, 10, 15, 20, 25, 30, or 35%) of the average diameter of the plurality of reagents. In some embodiments, the reagents can have a mean diameter of less than 50 (e.g., less than 45, 40, 35, 30, 25, 20, 15, 10) nm.

Methods for encapsulating a particle in lipid are known in the art and described in the accompanying Examples. One exemplary method for encapsulating a particle in lipid is a reverse phase evaporation (see, e.g., Huang et al. (2005) Biol. Pharm. Bull. 28(2) 387-390). Briefly, a lipid mixture (e.g., a mixture of any of the lipids described herein) is dissolved in a solvent such as hexane and chloroform. A particle suspension is then mixed with the lipid solution to form an emulsion. The emulsion is dried under vacuum to remove the organic solvent. Optionally, the resulting suspension can be sonicated and/or passed through a filter membrane. The suspension can also be subjected to centrifugation to separate lipid encapsulated particles from free particles.

Additional methods for encapsulating a particle in lipid are described in, e.g., Winter et al. (2006) Magnetic Resonance in Medicine 56(6):1384-1388 and Kunisawa et al. (2005) Journal of Controlled Release 105:344-353, the disclosures of each of which are incorporated by reference in their entirety.

In some embodiments, the reagents described herein can be frozen, lyophilized, or immobilized and stored under appropriate conditions, which allow the reagents to retain activity (e.g., the ability to induce an immune response in a subject).

### Pharmaceutical Compositions Containing the Reagents

Any of the reagents described herein can be incorporated into pharmaceutical compositions. Such compositions typically include a reagent and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. A reagent can be formulated as a pharmaceutical composition in the form of a syrup, an elixir, a suspension, a powder, a granule, a tablet, a capsule, a lozenge, a troche, an aqueous solution, a cream, an ointment, a lotion, a drop, a gel, a nasal spray, an emulsion, etc. Supplementary active compounds (e.g., one or more anti-microbial agents such an anti-HIV-1 agents) can also be incorporated into the compositions.

A pharmaceutical composition is generally formulated to be compatible with its intended route of administration. Examples of routes of administration include oral, rectal, and parenteral, e.g., intravenous, intramuscular, intradermal, subcutaneous, inhalation, transdermal, or transmucosal. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The compositions can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contamination by microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of contamination by microorganisms can be achieved by various antibacterial and antifungal agents, for example, parabens, chlorobutanol, phenol, ascorbic acid, thimerosal, and the like. In many cases, it will be desirable to include isotonic agents, for example, sugars, polyalcohols such as manitol, sorbitol, sodium chloride in the composition. Prolonged absorption of the injectable compositions can be facilitated by including in the composition an agent that delays absorption, for example, aluminum monostearate and gelatin.

Sterile injectable solutions can be prepared by incorporating the reagents in the required amount in an appropriate solvent with one or a combination of ingredients enumerated above, as required, followed by filtered sterilization. Generally, dispersions are prepared by incorporating the reagent into a sterile vehicle which contains a basic dispersion medium and the required other ingredients from those enumerated above. In the case of sterile powders for the preparation of sterile injectable solutions, the methods of preparation can include vacuum drying or freeze-drying which yields a powder of the active ingredient plus any additional desired ingredient from a previously sterile-filtered solution thereof.

Aqueous solutions suitable for oral use can be prepared by dissolving the active component in water and adding suitable colorants, flavors, stabilizers, and thickening agents as desired. Aqueous suspensions suitable for oral use can also be made by dispersing the finely divided active component in water with viscous material, such as natural or synthetic gums, resins, methylcellulose, sodium carboxymethylcellulose, and other well-known suspending agents.

For administration by inhalation, the reagents are delivered in the form of an aerosol spray from pressured container or dispenser which contains a suitable propellant, e.g., a gas such as carbon dioxide, or a nebulizer.

A reagent suitable for topical administration can be formulated as, e.g., a cream, a spray, a foam, a gel, an ointment, or a salve.

Systemic administration can also be achieved by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the reagents are formulated into ointments, salves, gels, or creams as generally known in the art.

The reagents can also be prepared in the form of suppositories (e.g., with conventional suppository bases such as cocoa butter and other glycerides) or retention enemas for rectal delivery.

In some embodiments, oral or parenteral compositions can be formulated in dosage unit form for ease of administration and uniformity of dosage. Dosage unit form, as used herein, refers to physically discrete units formulated as unitary dosages for the subject to be treated; each unit containing a predetermined quantity of reagent calculated to produce the desired therapeutic effect in association with the required pharmaceutical carrier. Dosage units can also be accompanied by instructions for use.

Any of the pharmaceutical compositions described herein can be included in a container, pack, or dispenser together with instructions for administration as described in subsequent sections.

### Methods for Inducing an Immune Response

The disclosure also features a variety of methods for inducing an immune response (or methods for producing an antibody; also see below) in a subject.

One exemplary method for inducing an immune response in a subject includes the step of administering to a subject a composition comprising: a particle encapsulated in lipid; and an immunogen. All or part of the immunogen can be embedded in the lipid. The immunogen can be, e.g., a molecule (e.g., a polypeptide or a nucleic acid) or an immunogenic or antigenic fragment thereof that is expressed on the surface of (i) a cell; (ii) a microorganism; or (iii) a cell infected with a microorganism.

Microorganisms include, e.g., bacteria, fungus (e.g., yeast), protozoa, and virus. Examples of bacteria (e.g., gram-negative or gram-positive bacteria) include, but are not limited to, Staphylococcus epidermidis, Staphylococcus warneri, Staphylococcus saprophyticus, Staphylococcus xylosus, Staphylococcus cohnii, Staphylococcus simulans, Staphylococcus hominus, Staphylococcus haemolyticus, Staphylococcus aureus, Streptococcus milleri, Streptococcus pneumoniae, Streptococcus spp. Streptococcus GroupG, Enterococcus faecium, Streptococcus faecalis, Echererichia coli, Kledsiella oxytoca, Klebsiella pneumoniae, Enterobacter cloaeae, Enterobacter aerogenes, Citrobacter freundii, Proteus mirabilis, Serratia marcesens, Psudomonas aeruginosa, Stenotrophomonas maltophilia, Legionella pneumophila, or Burkholderia cepacia. Fungi (e.g., moulds or yeasts) include, e.g., Candida albicans, Candida glabrata, Aspergillus fumigatus, Cryptococcus neoformans, or pneumocystis carinii. Protozoa (e.g., infectious protozoa) include, e.g., Entamoeba histolytica, Giardia lamblia, Trypanosoma brucei, Toxoplasma gondii, or Plasodium. Viruses can include, e.g., herpes simplex virus (HSV), retroviruses such as human immunodeficiency virus (e.g., HIV-1), papillomaviruses (e.g., HPV), Epstein-Barr virus (EBV), rotaviruses, papovaviruses, parvoviruses, phage, influenza virus, pox viruses, and filoviruses.

A cell infected with a microorganism can be a prokaryotic cell (e.g., a bacterial cell) or a eukaryotic cell (e.g., a yeast cell, a nematode cell, an insect cell, a bird cell, a mammalian cell (e.g., a mouse cell, a rat cell, a guinea pig cell, a horse cell, a cow cell, a pig cell, a goat cell, a donkey cell, a monkey cell, or a human cell)). In some embodiments, a cell can be a cancer cell such as, but not limited to, a lung cancer cell, a breast cancer cell, a colon cancer cell, a pancreatic cancer cell, a renal cancer cell, a stomach cancer cell, a liver cancer cell, a bone cancer cell, a hematological cancer cell, a neural tissue cancer cell, a thyroid cancer cell, an ovarian cancer cell, a testicular cancer cell, a prostate cancer cell, a cervical cancer cell, a vaginal cancer cell, or a bladder cancer cell.

A cell infected with an microorganism is considered “infected” even if the microorganism is dormant or only a microorganismal genome remains in the cell. For example, a cell harboring an integrated retroviral genome or partial retroviral genome (or a viral episome) can be considered to be infected with the virus, even though the virus encoded by the genome is not actively replicating. In some embodiments, the integrated retroviral genome does not include endogenous retroviral genomes.

Another exemplary method for inducing an immune response in a subject includes the step of administering to a subject a composition comprising lipid and a polypeptide, wherein the polypeptide consists of an MPER of an HIV-1 gp160 polypeptide and wherein at least one amino acid of the MPER is embedded in the lipid.

With respect to the above methods, the particle and lipid can be any of those described herein.

Yet another exemplary method for inducing an immune response in a subject includes the step of administering to a subject any of the reagents described herein (or any of the pharmaceutical compositions containing a reagent described herein).

Any of the above methods can also be, e.g., methods for treating or preventing a condition (e.g., an infection such as an HIV-1 infection) in a subject. When the terms “prevent,” “preventing,” or “prevention” are used herein in connection with a given treatment for a given condition, they mean that the treated subject either does not develop a clinically observable level of the condition at all (e.g., the subject does not exhibit one or more symptoms of the condition or, in the case of an infection, the subject does not develop a detectable level of the microorganism), or the condition develops more slowly and/or to a lesser degree (e.g., fewer symptoms or a lower amount of a microorganism in or on the subject) in the subject than it would have absent the treatment. These terms are not limited solely to a situation in which the subject experiences no aspect of the condition whatsover. For example, a treatment will be said to have “prevented” the condition if it is given during exposure of a subject to a stimulus (e.g., an infectious agent) that would have been expected to produce a given manifestation of the condition, and results in the subject's experiencing fewer and/or milder symptoms of the condition than otherwise expected. A treatment can “prevent” an infection (e.g., an HIV-1 infection) when the subject displays only mild overt symptoms of the infection. “Prevention” does not imply that there must have been no penetration of, or fusion with, any cell by the infecting microorganism (e.g., an HIV-1).

Generally, a reagent or immunogenic/antigenic composition delivered to the subject will be suspended in a pharmaceutically-acceptable carrier (e.g., physiological saline) and administered orally, rectally, or parenterally, e.g., injected intravenously, subcutaneously, intramuscularly, intrathecally, intraperitoneally, intrarectally, intravaginally, intranasally, intragastrically, intratracheally, or intrapulmonarily (see below).

Administration can be by periodic injections of a bolus of the pharmaceutical composition or can be uninterrupted or continuous by intravenous or intraperitoneal administration from a reservoir which is external (e.g., an IV bag) or internal (e.g., a bioerodable implant, a bioartificial organ, or a colony of implanted reagent production cells). See, e.g., U.S. Pat. Nos. 4,407,957, 5,798,113 and 5,800,828, each incorporated herein by reference in their entirety.

The dosage required depends on the choice of the route of administration; the nature of the formulation; the nature or severity of the subject's illness; the immune status of the subject; the subject's size, weight, surface area, age, and sex; other drugs being administered; and the judgment of the attending medical professional. Suitable dosages for inducing an immune response are in the range of 0.000001 to 10 mg of the reagent or antigenic/immunogenic composition per kg of the subject. Wide variations in the needed dosage are to be expected in view of the variety of reagents and the differing efficiencies of various routes of administration. For example, nasal or rectal administration may require higher dosages than administration by intravenous injection. Variations in these dosage levels can be adjusted using standard empirical routines for optimization as is well understood in the art. Administrations can be single or multiple (e.g., 2-, 3-, 4-, 6-, 8-, 10-, 20-, 50-,100-, 150-, or more fold).

In order to optimize therapeutic efficacy (the efficacy of the reagent to induce an immune response in a subject), the reagents can be first administered at different dosing regimens. The unit dose and regimen depend on factors that include, e.g., the species of mammal, its immune status, the body weight of the mammal.

The frequency of dosing for a pharmaceutical composition (e.g., a pharmaceutical composition containing a reagent or an immunogenic/antigenic composition) is within the skills and clinical judgement of medical practitioners (e.g., doctors or nurses). Typically, the administration regime is established by clinical trials which may establish optimal administration parameters. However, the practitioner may vary such administration regimes according to the subject's age, health, weight, sex and medical status.

In some embodiments, a pharmaceutical composition can be administered to a subject at least two (e.g., three, four, five, six, seven, eight, nine, 10, 11, 12, 15, or 20 or more) times. For example, a pharmaceutical composition can be administered to a subject once a month for three months; once a week for a month; once a year for three years, once a year for five years; once every five years; once every ten years; or once every three years for a lifetime.

In some embodiments, the reagent can be administered with an immune modulator such as a Toll Receptor ligand or an adjuvant (see above).

As defined herein, a “therapeutically effective amount” of a reagent is an amount of the reagent that is capable of producing an immune response in a treated subject. A therapeutically effective amount of a reagent (i.e., an effective dosage) includes milligram, microgram, nanogram, or picogram amounts of the reagent per kilogram of subject or sample weight (e.g., about 1 nanogram per kilogram to about 500 micrograms per kilogram, about 1 microgram per kilogram to about 500 milligrams per kilogram, about 100 micrograms per kilogram to about 5 milligrams per kilogram, or about 1 microgram per kilogram to about 50 micrograms per kilogram).

As defined herein, a “prophylatically effective amount” of a reagent is an amount of the reagent that is capable of producing an immune response against an infectious agent (e.g., a infectious microorganism) in a treated subject, which immune response is capable of preventing the infection of a subject by an infectious agent or is able to substantially reduce the chance of a subject being productively infected with the infectious agent if the subject comes into contact with it. A prophylatically effective amount of a reagent (i.e., an effective dosage) includes milligram, microgram, nanogram, or picogram amounts of the reagent per kilogram of subject or sample weight (e.g., about 1 nanogram per kilogram to about 500 micrograms per kilogram, about 1 microgram per kilogram to about 500 milligrams per kilogram, about 100 micrograms per kilogram to about 5 milligrams per kilogram, or about 1 microgram per kilogram to about 50 micrograms per kilogram).

The subject can be any animal capable of an immune response to an antigen such as, but not limited to, a mammal, e.g., a human (e.g., a human patient) or a non-human primate (e.g., chimpanzee, baboon, or monkey), mouse, rat, rabbit, guinea pig, gerbil, hamster, horse, a type of livestock (e.g., cow, pig, sheep, or goat), a dog, cat, or a whale. The subject can be one having, suspected of having, or at risk of developing an HIV-1 infection.

As used herein, a subject “at risk of developing an HIV-1 infection” is a subject in a high risk HIV-1 exposure group, e.g., an intravenous drug user, a subject engaged in promiscuous sexual behavior, a subject receiving a blood transfusion, a homosexual male, an ethnic minority person (e.g., an African-American person), a subject at risk of needle-stick injuries such as a medical professional, or a child borne of a mother with an HIV-1 infection (i.e., in utero transmission or transmission during childbirth). From the above it will be clear that subjects “at risk of developing an HIV-1 infection” are not all the subjects within a species of interest.

A subject “suspected of having an HIV-1 infection” is one having one or more symptoms of an HIV-1 infection. Symptoms of an HIV-1 infection are well-known to those of skill in the art and include, without limitation, rapid weight loss; dry cough; recurring fever or profuse night sweats; profound and unexplained fatigue; swollen lymph glands in the armpits, groin, or neck; diarrhea; white spots or unusual blemishes on the tongue, in the mouth, or in the throat; pneumonia; red, brown, pink, or purplish blotches on or under the skin or inside the mouth, nose, or eyelids; memory loss; depression; or other neurological disorders.

In some embodiments, the method can also include determining if an immune response occurred in a subject after administering the reagent to the subject. Suitable methods for determining whether an immune response occurred in a subject include use of immunoassays to detect, e.g., the presence of antibodies specific for a polypeptide of the reagent in a biological sample from the subject. For example, after the administration of the reagent to the subject, a biological sample (e.g., a blood sample) can be obtained from the subject and tested for the presence of MPER-specific antibodies. Briefly, an MPER polypeptide (or an MPER polypeptide wherein at least one amino acid of the polypeptide is embedded in lipid) bound to a well of an assay plate can be contacted with the biological sample under conditions that allow the binding of an anti-MPER antibody, if present in the biological sample, to the MPER polypeptide. The well is then washed, e.g., with PBS to remove any unbound material. Next, a secondary antibody that is specific for the anti-MPER antibody and that bears a detectable label (e.g., any of those described above) is contacted with well. Unbound secondary antibody can be removed by an additional wash step. The presence or amount of signal produced by the detectable label indicates that presence or amount of anti-MPER antibodies in the biological sample.

In some embodiments, the methods can also include the step of determining whether a subject has an HIV-1 infection. Suitable methods and kits useful for such a determination are known in the art and can be qualitative or quantitative. For example, a medical practitioner can diagnose a subject as having an HIV-1 infection when the subject exhibits two or more symptoms of an HIV-1 infection such as any of those described herein. The HIV-1 status of a subject can also be determined by enzyme immunoassay to detect HIV-1 specific antibodies or by, e.g., RT-PCR to detect one or more nucleic acids from HIV-1 (e.g., a viral RNA). In some embodiments, a subject can self-test for an HIV-1 infection using, e.g., a Home Access Express HIV-1 Test System manufactured by Home Access Health Corporation (Hoffman Estates, Ill.).

A reagent or pharmaceutical composition thereof described herein can be administered to a subject as a combination therapy with another treatment, e.g., an anti-HIV-1 agent such as an HIV-1 protease inhibitor, an HIV-1 integrase inhibitor, an HIV-1 reverse transcriptase inhibitor, an HIV-1 fusion inhibitor, or an antibody that neutralizes an HIV-1 particle. For example, the combination therapy can include administering to the subject (e.g., a human patient) one or more additional agents that provide a therapeutic benefit to the subject who has, or is at risk of developing, (or suspected of having) an HIV-1 infection. Thus, the reagent or pharmaceutical composition and the one or more additional agents can be administered at the same time. Alternatively, the reagent can be administered first in time and the one or more additional agents administered second in time. The one or more additional agents can be administered first in time and the reagent administered second in time. The reagent can replace or augment a previously or currently administered therapy. That is, compositions that are determined not to produce a humoral immune response against HIV-1 or a neutralizing HIV-1 antibody response can be replaced with one or more of the reagents described herein. Administration of the previous therapy can also be maintained. The two therapies can be administered in combination.

In some instances, when the subject is administered a reagent or pharmaceutical composition thereof, the first therapy is halted. The subject can be monitored for a first pre-selected result, e.g., the production of a neutralizing antibody response or an improvement in or loss of one or more symptoms of an HIV-1 infection). In some cases, where the first pre-selected result is observed, treatment with the reagent is decreased or halted.

The reagent can also be administered with a treatment for one or more symptoms of a disease (e.g., an HIV-1 infection). For example, the reagent can be co-administered (e.g., at the same time or by any combination regimen described above) with, e.g., an analgesic or an antibiotic.

Ex Vivo Approaches. An ex vivo strategy can involve contacting cells obtained from the subject with any of the reagents or immunogenic/antigenic compositions described herein. The contacted cells are then returned to the subject. The cells can be any of a wide range of types including, without limitation, bone marrow cells, macrophages, monocytes, dendritic cells, T cells (e.g., T helper cells, CD4⁺ cells, CD8⁺ cells, or cytotoxic T cells), or B cells. Alternatively, cells (e.g., antigen presenting cells), obtained from a subject of the same species other than the subject (allogeneic) can be contacted with the reagents (or immunogenic/antigenic compositions) and administered to the subject.

The ex vivo methods include the steps of harvesting cells from a subject (or a subject of the same species as the subject), culturing the cells, contacting them with any of the reagents (or immunogenic/antigenic compositions described herein), and administering the cells to the subject.

### Methods for Producing an Antibody

Methods of producing an antibody specific for an immunogen (e.g., a polypeptide containing an MPER of an HIV-1 gp160 polypeptide) are described herein are known in the art. For example, methods for generating antibodies or antibody fragments specific for a polypeptide of a reagent described herein can be generated by immunization, e.g., using an animal, or by in vitro methods such as phage display. A polypeptide that includes all or part of a target polypeptide (e.g., all or part of a polypeptide containing an MPER) can be used to generate an antibody or antibody fragment.

A polypeptide can be used to prepare antibodies by immunizing a suitable subject, (e.g., rabbit, goat, mouse, or other mammal such as a human) with the peptide. An appropriate immunogenic preparation can contain, for example, any of the reagents described herein. The preparation can further include an adjuvant, such as Freund's complete or incomplete adjuvant, alum, RIBI, or similar immunostimulatory agent. Adjuvants also include, e.g., cholera toxin (CT), E. coli heat labile toxin (LT), mutant CT (MCT) (Yamamoto et al. (1997) J. Exp. Med. 185:1203-1210) and mutant E. coli heat labile toxin (MLT) (Di Tommaso et al. (1996) Infect. Immunity 64:974-979). MCT and MLT contain point mutations that substantially diminish toxicity without substantially compromising adjuvant activity relative to that of the parent molecules. Immunization of a suitable subject with an immunogenic peptide preparation (e.g., any of the reagents described herein) induces a polyclonal anti-peptide antibody response.

The antibodies described herein can be polyclonal or monoclonal, and the term “antibody” is intended to encompass both polyclonal and monoclonal antibodies. An antibody that specifically binds to a polypeptide described herein is an antibody that binds the polypeptide, but does not substantially bind other molecules in a sample.

The disclosure also provides immunologically active portions (or fragments) of immunoglobulin molecules (i.e., molecules that contain an antigen binding site that specifically bind to the polypeptide (e.g., the polypeptide containing the MPER sequence). Examples of immunologically active portions of immunoglobulin molecules include Fab fragments, F(ab′)₂ fragments, Fab′ fragments, Fv fragments, or scFv fragments of antibodies.

The anti-peptide antibody can be a monoclonal antibody or a preparation of polyclonal antibodies. The term monoclonal antibody, as used herein, refers to a population of antibody molecules that contain only one species of an antigen binding site capable of immunoreacting with the polypeptide. A monoclonal antibody composition thus typically displays a single binding affinity for a particular polypeptide with which it immunoreacts.

Polyclonal anti-peptide antibodies can be prepared as described above by immunizing a suitable subject with a polypeptide immunogen (e.g., a reagent described herein containing an MPER). The anti-peptide antibody titer in the immunized subject can be monitored over time by standard techniques, such as with an enzyme linked immunosorbent assay (ELISA) using immobilized peptide. If desired, the antibody molecules directed against the peptide can be isolated from the mammal (e.g., from the blood) and further purified by techniques such as protein A chromatography to obtain the IgG fraction. At an appropriate time after immunization, e.g., when the anti-peptide antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), or the EBV-hybridoma technique (Cole et al. (1985), Monoclonal Antibodies and Cancer Therapy, Alan R. Liss, Inc., pp. 77-96). Any of the many well known protocols used for fusing lymphocytes and immortalized cell lines can be applied for the purpose of generating an anti-peptide monoclonal antibody (see, e.g., Current Protocols in Immunology, supra; Galfre et al. (1977) Nature 266:55052; R. H. Kenneth, in Monoclonal Antibodies: A New Dimension In Biological Analyses, Plenum Publishing Corp., New York, N.Y. (1980); and Lerner (1981) Yale J. Biol. Med., 54:387-402, the disclosures of each of which are incorporated by reference in their entirety).

As an alternative to preparing monoclonal antibody-secreting hybridomas, a monoclonal anti-peptide antibody can be identified and isolated by screening a recombinant combinatorial immunoglobulin library (e.g., an antibody phage display library) with a peptide described herein to isolate immunoglobulin library members that bind the peptide.

An anti-peptide antibody (e.g., a monoclonal antibody) can be used to isolate the peptide by techniques such as affinity chromatography or immunoprecipitation. Moreover, an anti-peptide antibody can be used to detect the peptide in screening assays described herein. An antibody can optionally be coupled to a detectable label such as any of those described herein or a first or second member of a binding pair (e.g., streptavidin/biotin or avidin/biotin), the second member of which can be conjugated to a detectable label.

Non-human antibodies to a target polypeptide (e.g., an MPER of an HIV-1 gp160 polypeptide) can also be produced in non-human host (e.g., a rodent) and then humanized, e.g., as described in U.S. Pat. No. 6,602,503, EP 239 400, U.S. Pat. No. 5,693,761, and U.S. Pat. No. 6,407,213, the disclosures of each of which are incorporated by reference in their entirety.

EP 239 400 (Winter et al.) describes altering antibodies by substitution (within a given variable region) of their CDRs for one species with those from another. CDR-substituted antibodies can be less likely to elicit an immune response in humans compared to true chimeric antibodies because the CDR-substituted antibodies contain considerably less non-human components. See Riechmann et al., 1988, Nature 332, 323-327; Verhoeyen et al., 1988, Science 239, 1534-1536, the disclosures of each of which is incorporated by reference in their entirety. Typically, CDRs of a murine antibody are substituted into the corresponding regions in a human antibody by using recombinant nucleic acid technology to produce sequences encoding the desired substituted antibody. Human constant region gene segments of the desired isotype (e.g., gamma I for CH and kappa for CL) can be added and the humanized heavy and light chain genes can be co-expressed in mammalian cells to produce soluble humanized antibody.

WO 90/07861 describes a process that includes choosing human V framework regions by computer analysis for optimal protein sequence homology to the V region framework of the original murine antibody, and modeling the tertiary structure of the murine V region to visualize framework amino acid residues that are likely to interact with the murine CDRs. These murine amino acid residues are then superimposed on the homologous human framework. See also U.S. Pat. Nos. 5,693,762; 5,693,761; 5,585,089; and 5,530,101. Tempest et al., 1991, Biotechnology 9, 266-271 use, as standard, the V region frameworks derived from NEWM and REI heavy and light chains, respectively, for CDR-grafting without radical introduction of mouse residues. An advantage of using the Tempest et al. approach to construct NEWM and REI based humanized antibodies is that the three dimensional structures of NEWM and REI variable regions are known from x-ray crystallography and thus specific interactions between CDRs and V region framework residues can be modeled.

Non-human antibodies can be modified to include substitutions that insert human immunoglobulin sequences, e.g., consensus human amino acid residues at particular positions, e.g., at one or more (e.g., at least five, ten, twelve, or all) of the following positions: (in the framework of the variable domain of the light chain) 4L, 35L, 36L, 38L, 43L, 44L, 58L, 46L, 62L, 63L, 64L, 65L, 66L, 67L, 68L, 69L, 70L, 71L, 73L, 85L, 87L, 98L, and/or (in the framework of the variable domain of the heavy chain) 2H, 4H, 24H, 36H, 37H, 39H, 43H, 45H, 49H, 58H, 60H, 67H, 68H, 69H, 70H, 73H, 74H, 75H, 78H, 91H, 92H, 93H, and/or 103H (according to the Kabat numbering). See, e.g., U.S. Pat. No. 6,407,213, the disclosure of which is incorporated herein by reference in its entirety.

Fully human monoclonal antibodies that bind to a target polypeptide (e.g., a polypeptide containing an MPER of a HIV-1 gp160 polypeptide) can be produced, e.g., using in vitro-primed human splenocytes, as described by Boerner et al., 1991, J. Immunol., 147, 86-95. They may be prepared by repertoire cloning as described by Persson et al., 1991, Proc. Nat. Acad. Sci. USA, 88: 2432-2436 or by Huang and Stollar, 1991, J. Immunol. Methods 141, 227-236; also U.S. Pat. No. 5,798,230, the disclosures of each of which are incorporated herein by reference in their entirety. Large nonimmunized human phage display libraries may also be used to isolate high affinity antibodies that can be developed as human therapeutics using standard phage technology (see, e.g., Vaughan et al, 1996; Hoogenboom et al. (1998) Immunotechnology 4:1-20; and Hoogenboom et al. (2000) Immunol Today 2:371-8; US 2003-0232333, the disclosures of each of which are incorporated by reference in their entirety).

As used herein, an “immunoglobulin variable domain sequence” refers to an amino acid sequence that can form the structure of an immunoglobulin variable domain. For example, the sequence may include all or part of the amino acid sequence of a naturally-occurring variable domain. For example, the sequence may omit one, two or more N- or C-terminal amino acids, internal amino acids, may include one or more insertions or additional terminal amino acids, or may include other alterations. In one embodiment, a polypeptide that includes an immunoglobulin variable domain sequence can associate with another immunoglobulin variable domain sequence to form a target binding structure (or “antigen binding site”), e.g., a structure that interacts with a target polypeptide (e.g., a polypeptide containing an MPER of an HIV-1 gp160 polypeptide).

The VH or VL chain of the antibody can further include all or part of a heavy or light chain constant region, to thereby form a heavy or light immunoglobulin chain, respectively. In one embodiment, the antibody is a tetramer of two heavy immunoglobulin chains and two light immunoglobulin chains. The heavy and light immunoglobulin chains can be connected by disulfide bonds. The heavy chain constant region typically includes three constant domains, CH1, CH2 and CH3. The light chain constant region typically includes a CL domain. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

One or more regions of an antibody can be human, effectively human, or humanized. For example, one or more of the variable regions can be human or effectively human. For example, one or more of the CDRs, e.g., heavy chain (HC) CDR1, HC CDR2, HC CDR3, light chain (LC) CDR1, LC CDR2, and LC CDR3, can be human. Each of the light chain CDRs can be human. HC CDR3 can be human. One or more of the framework regions (FR) can be human, e.g., FR1, FR2, FR3, and FR4 of the HC or LC. In some embodiments, all the framework regions are human, e.g., derived from a human somatic cell, e.g., a hematopoietic cell that produces immunoglobulins or a non-hematopoietic cell. In one embodiment, the human sequences are germline sequences, e.g., encoded by a germline nucleic acid. One or more of the constant regions can be human, effectively human, or humanized. In another embodiment, at least 70, 75, 80, 85, 90, 92, 95, or 98% of the framework regions (e.g., FR1, FR2, and FR3, collectively, or

FR1, FR2, FR3, and FR4, collectively) or the entire antibody can be human, effectively human, or humanized. For example, FR1, FR2, and FR3 collectively can be at least 70, 75, 80, 85, 90, 92, 95, 98, or 99% identical to a human sequence encoded by a human germline segment. In some embodiments, to humanize a murine antibody, one or more regions of a mouse Ig loci can be replaced with corresponding human Ig loci (see, e.g., Zou et al. (1996) The FASEB Journal Vol 10, 1227-1232; Popov et al. (1999) J. Exp. Med. 189(10) 1611-1619; and Nicholson et al. (1999) J. Immunol. 6898-6906; the disclosures of each of which are incorporated by reference in their entirety.

An “effectively human” immunoglobulin variable region is an immunoglobulin variable region that includes a sufficient number of human framework amino acid positions such that the immunoglobulin variable region does not elicit an immunogenic response in a normal human. An “effectively human” antibody is an antibody that includes a sufficient number of human amino acid positions such that the antibody does not elicit an immunogenic response in a normal human.

A “humanized” immunoglobulin variable region is an immunoglobulin variable region that is modified such that the modified form elicits less of an immune response in a human than does the non-modified form, e.g., is modified to include a sufficient number of human framework amino acid positions such that the immunoglobulin variable region does not elicit an immunogenic response in a normal human. Descriptions of “humanized” immunoglobulins include, for example, U.S. Pat. No. 6,407,213 and U.S. Pat. No. 5,693,762, the disclosures of each of which are incorporated herein by reference in their entirety. In some cases, humanized immunoglobulins can include a non-human amino acid at one or more framework amino acid positions.

All or part of an antibody can be encoded by an immunoglobulin gene or a segment thereof. Exemplary human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 kDa or 214 amino acids) are encoded by a variable region gene at the NH2-terminus (about 110 amino acids) and a kappa or lambda constant region gene at the COOH-terminus. Full-length immunoglobulin “heavy chains” (about 50 kDa or 446 amino acids), are similarly encoded by a variable region gene (about 116 amino acids) and one of the other aforementioned constant region genes, e.g., gamma (encoding about 330 amino acids).

The term “antigen-binding fragment” of a full length antibody refers to one or more fragments of a full-length antibody that retain the ability to specifically bind to a target of interest (i.e., a polypeptide containing an MPER sequence). Examples of binding fragments encompassed within the term “antigen-binding fragment” of a full length antibody include: (i) a Fab fragment, a monovalent fragment consisting of the VL, VH, CL and CH1 domains; (ii) a F(ab′)2 fragment, a bivalent fragment including two Fab fragments linked by a disulfide bridge at the hinge region; (iii) a Fd fragment consisting of the VH and CH1 domains; (iv) a Fv fragment consisting of the VL and VH domains of a single arm of an antibody; (v) a dAb fragment (Ward et al., (1989) Nature 341:544-546), which consists of a VH domain; and (vi) an isolated complementarity determining region (CDR) that retains functionality. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFᵥ). See e.g., Bird et al. (1988) Science 242:423-426; and Huston et al. (1988) Proc. Natl. Acad. Sci. USA 85:5879-5883, the disclosures of each of which are incorporated herein by reference in their entirety.

It is understood that an antibody produced by a method described above (e.g., an antibody specific for an MPER polypeptide) can be used to treat and or prevent an HIV-1 infection in a subject.

### Structures and Methods for Identifying an Agent

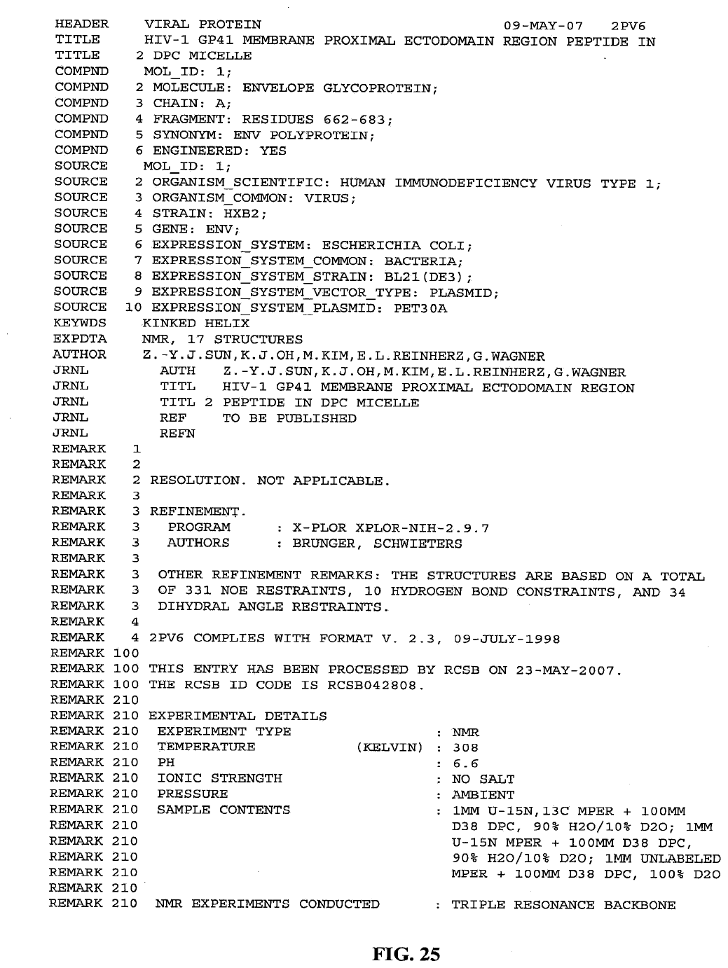

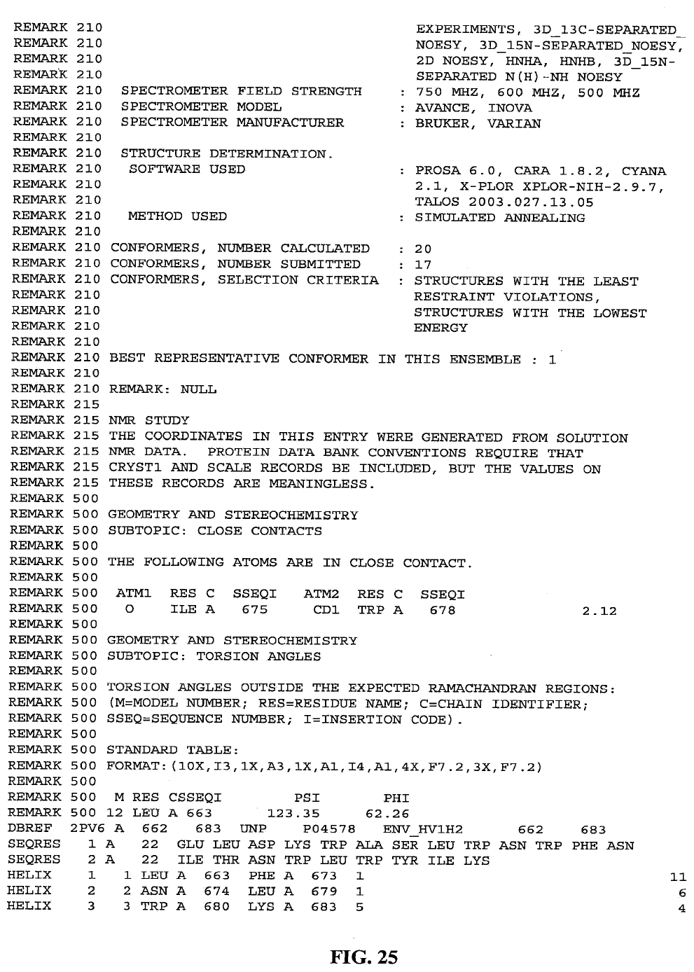

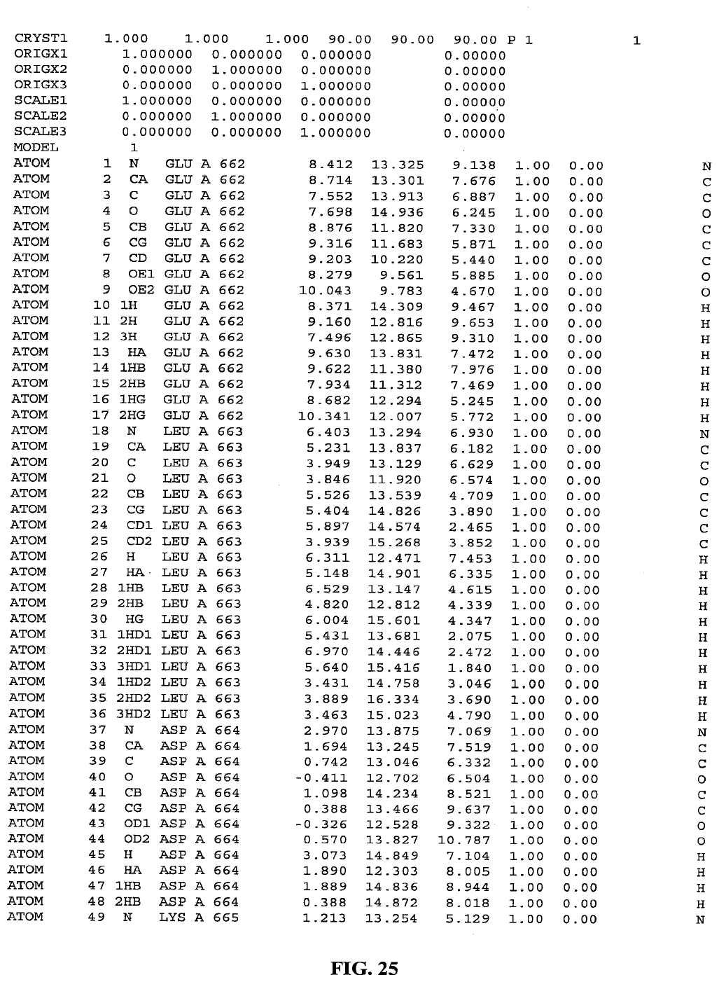

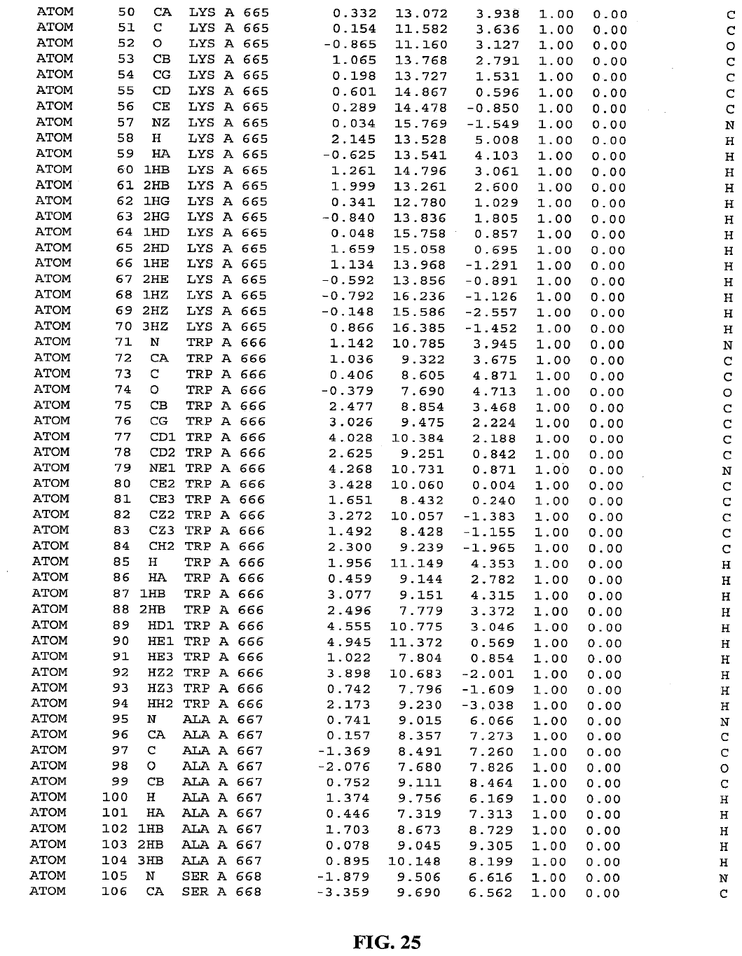

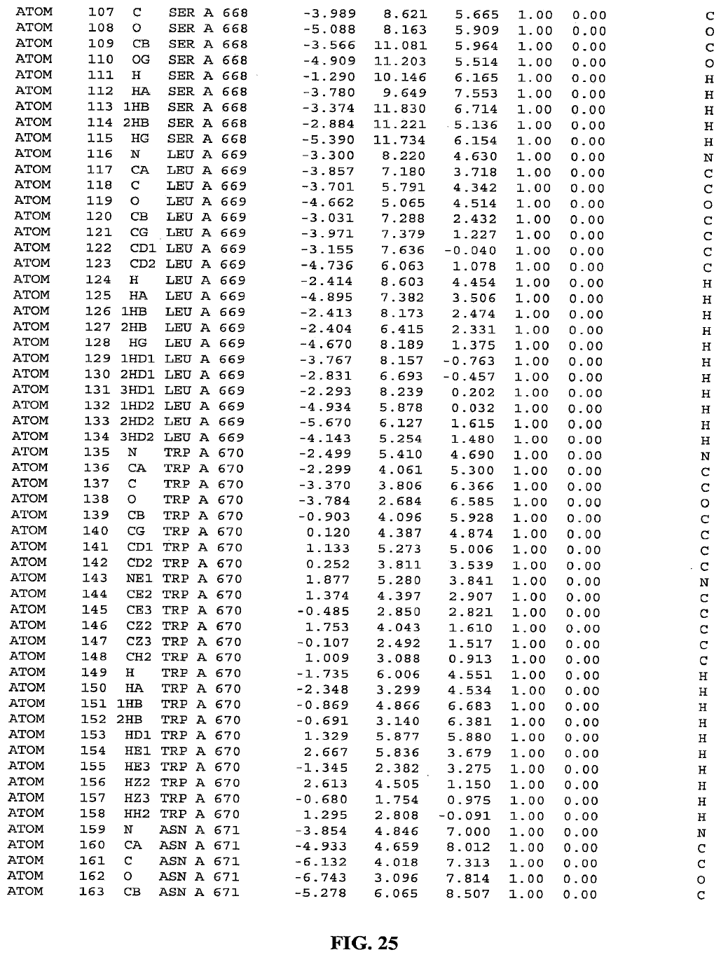

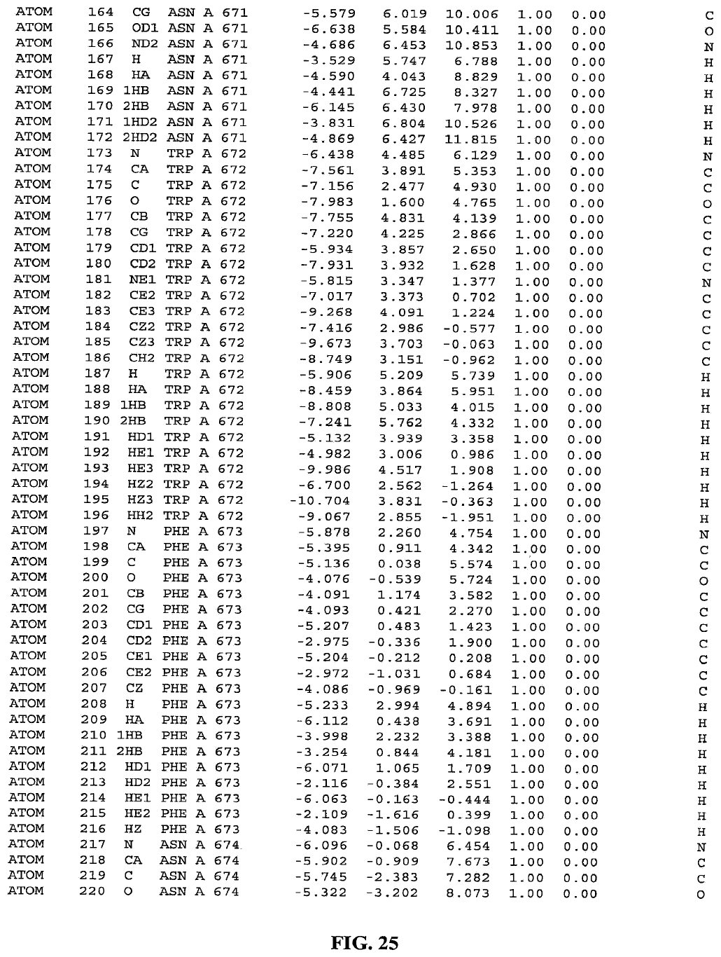

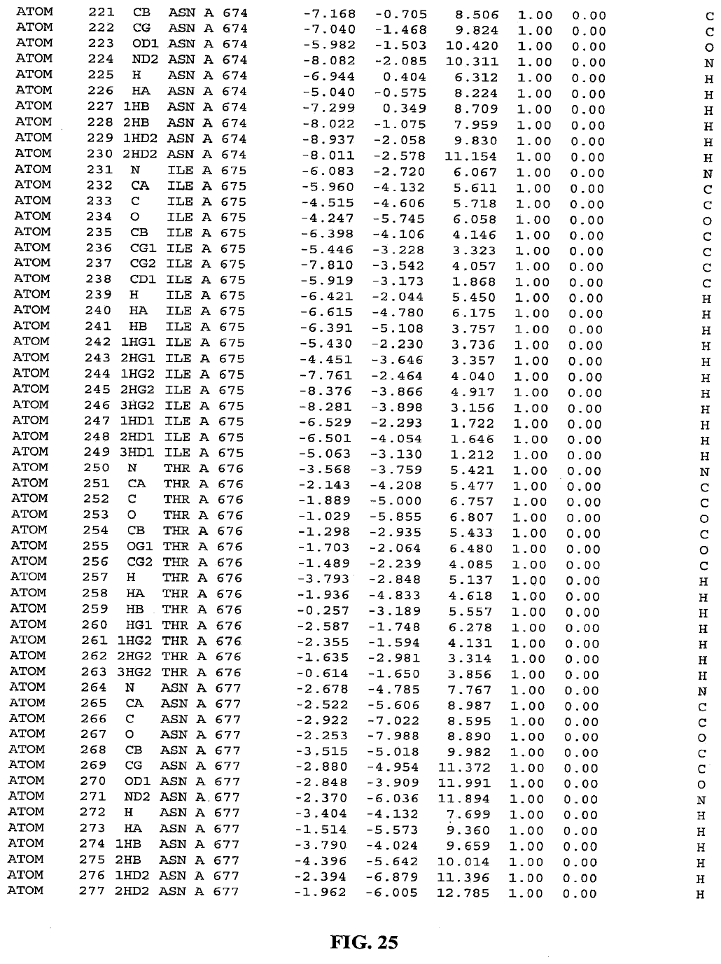

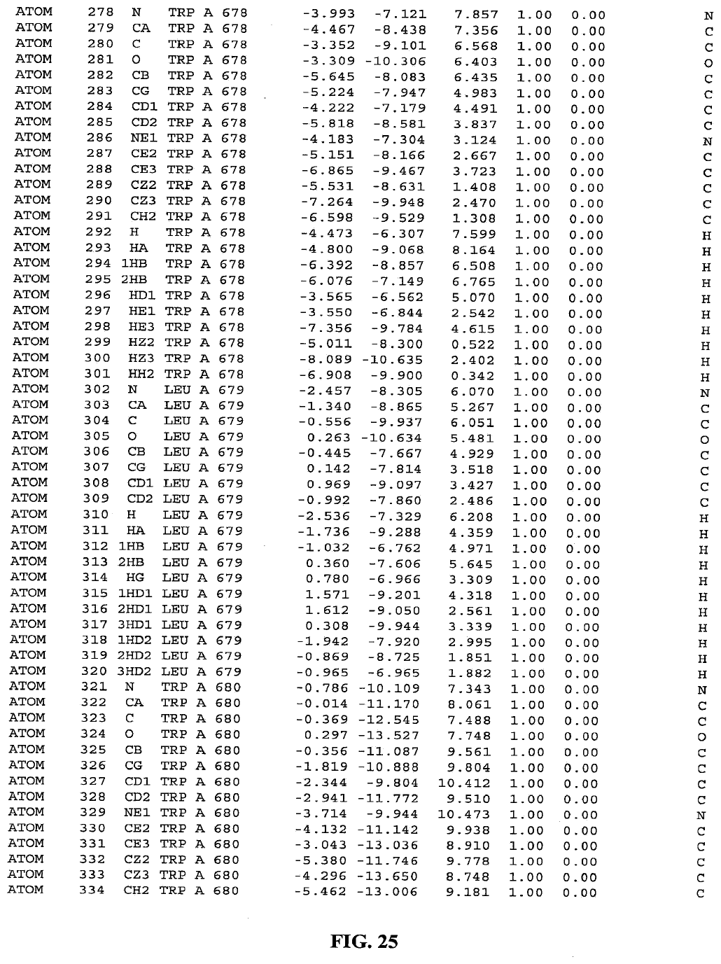

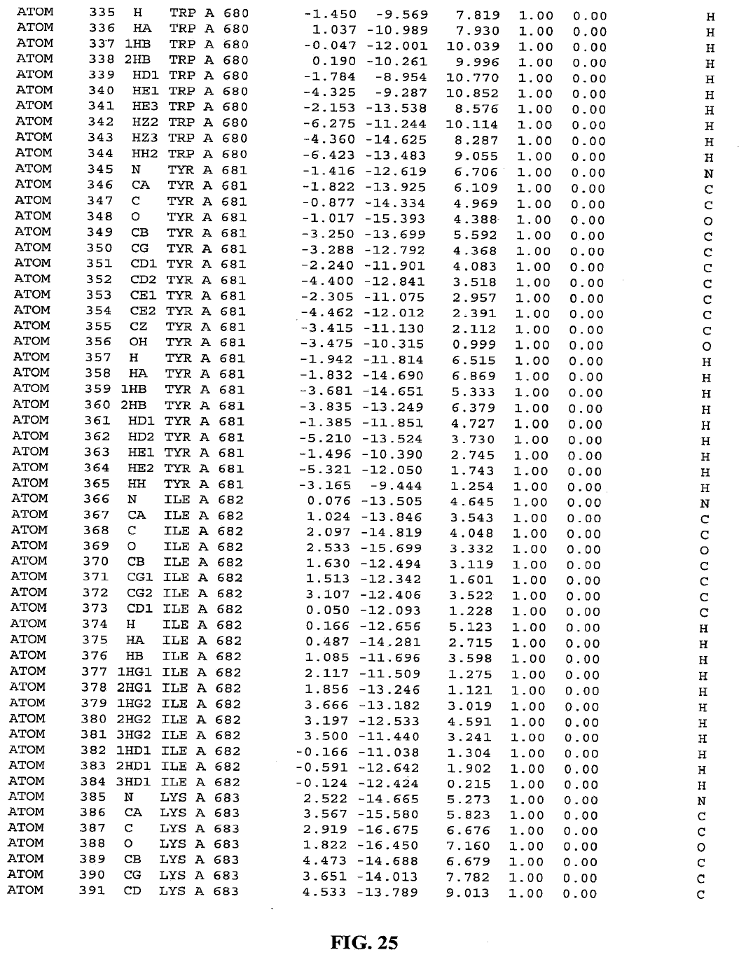

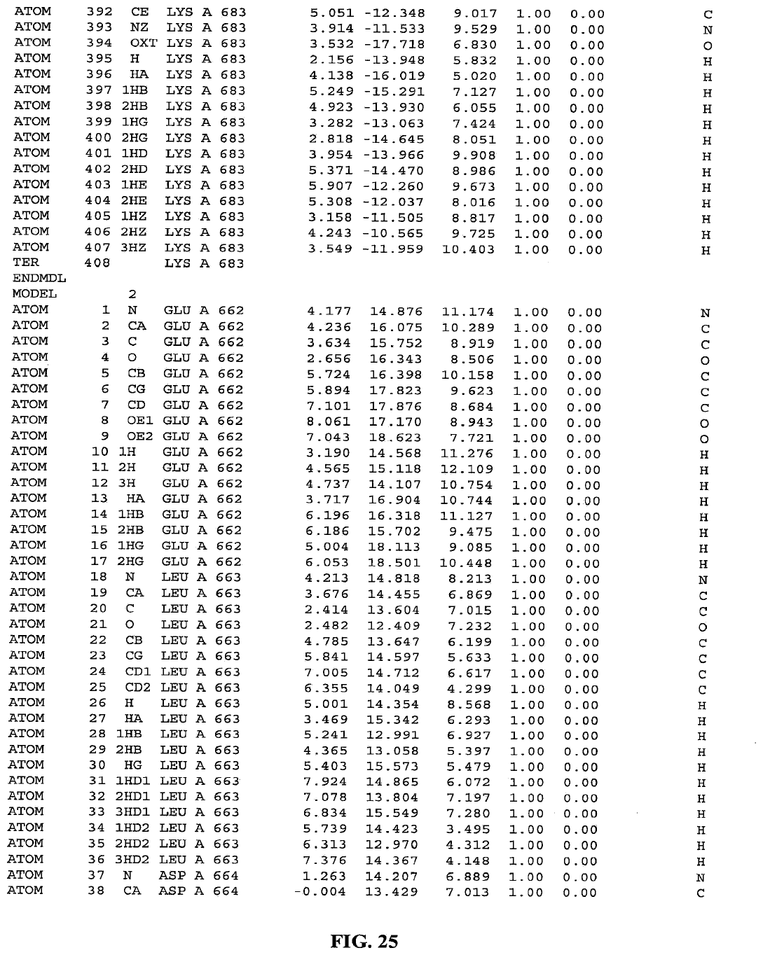

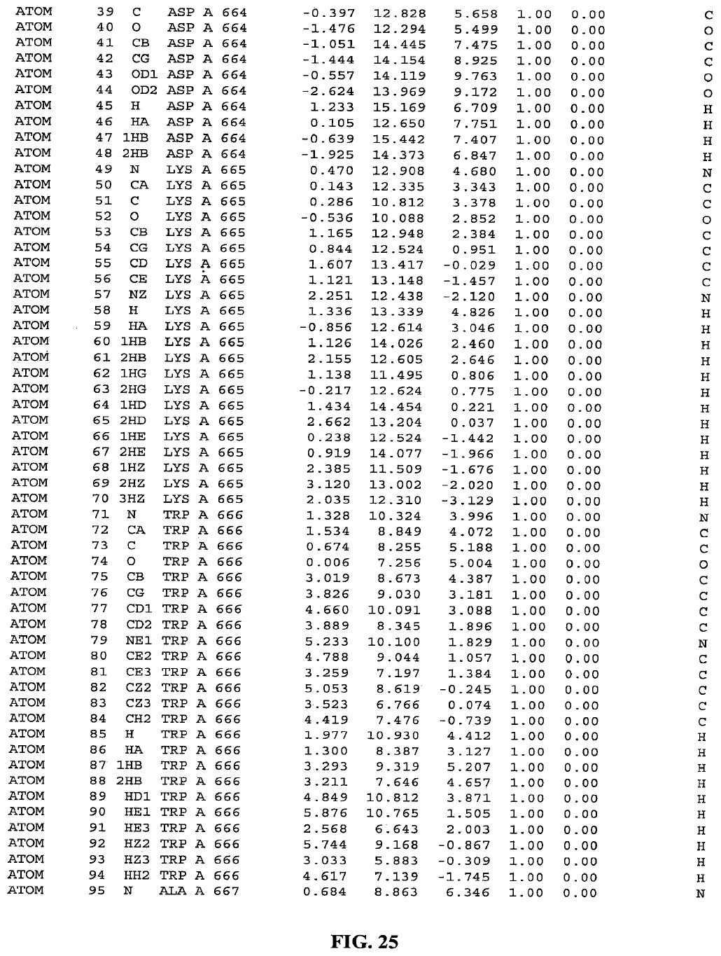

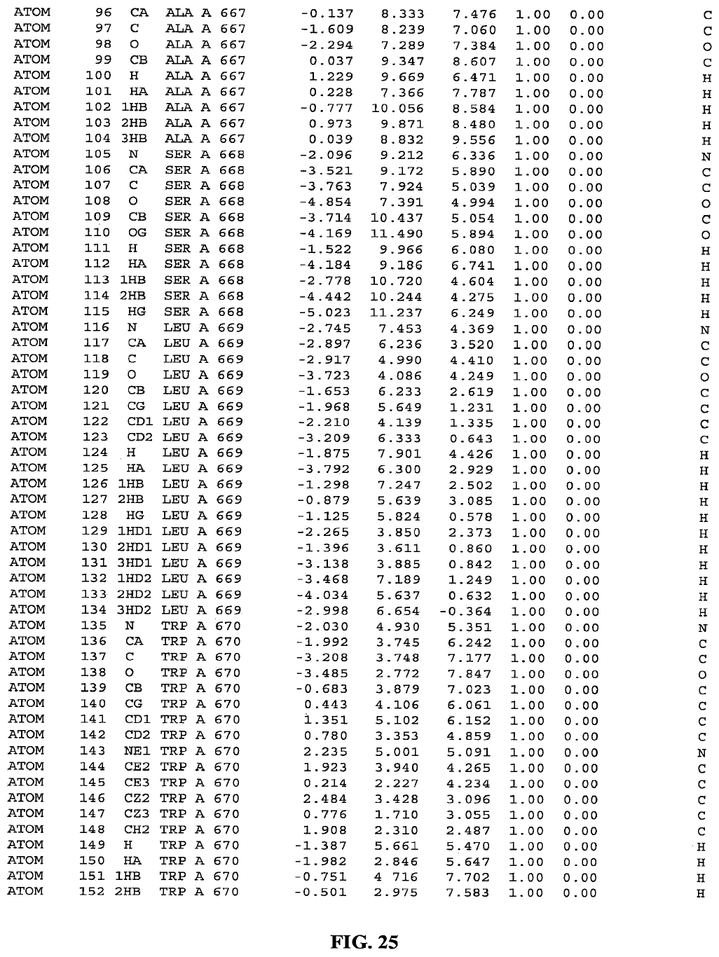

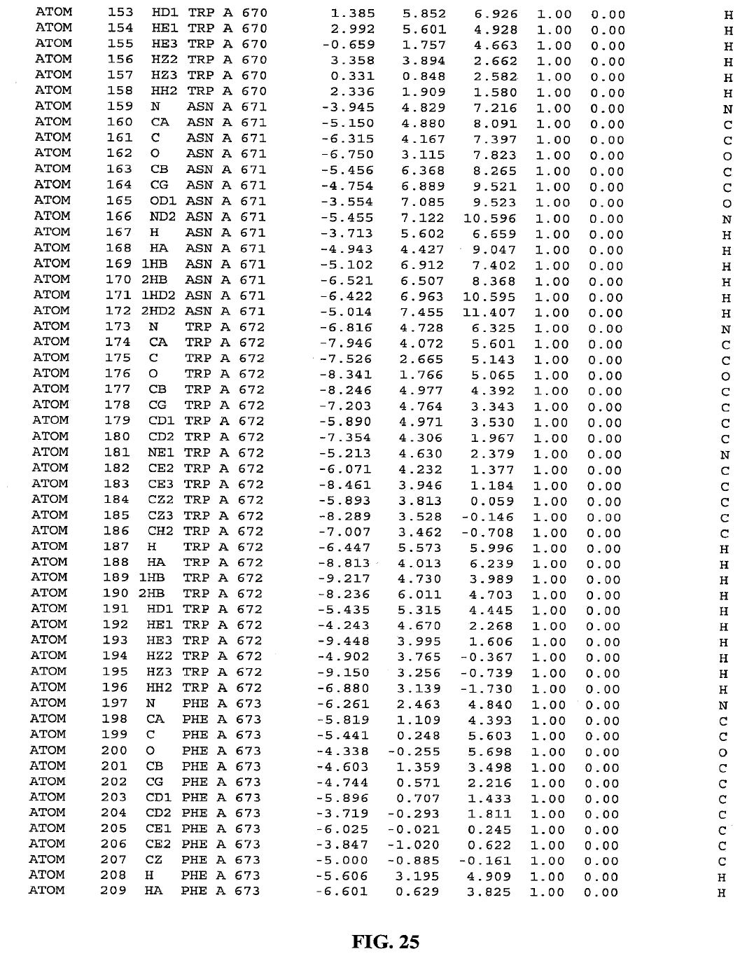

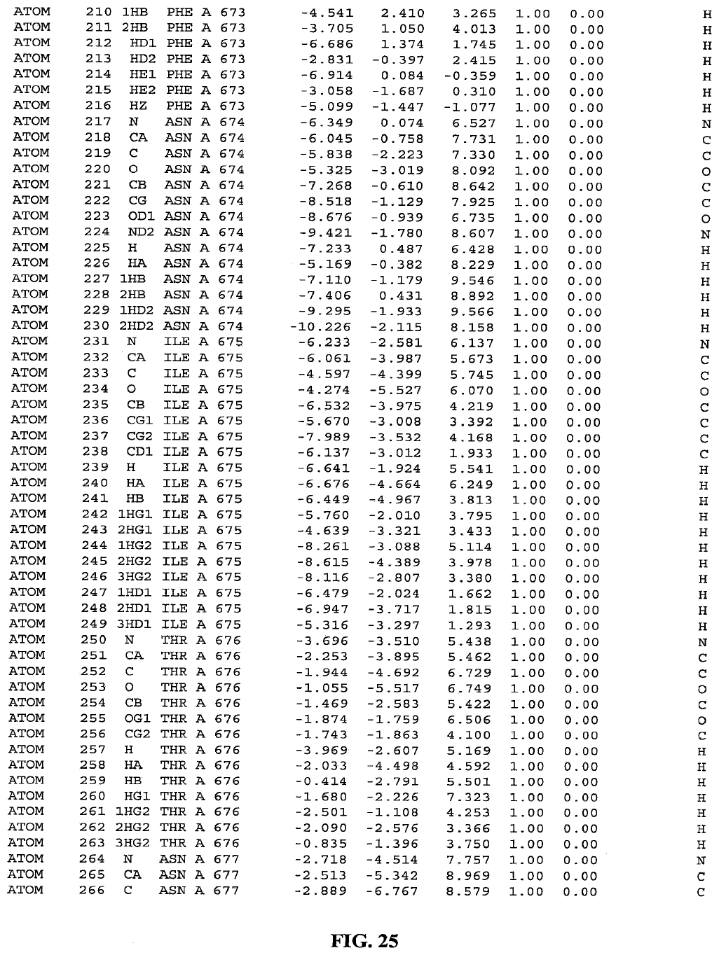

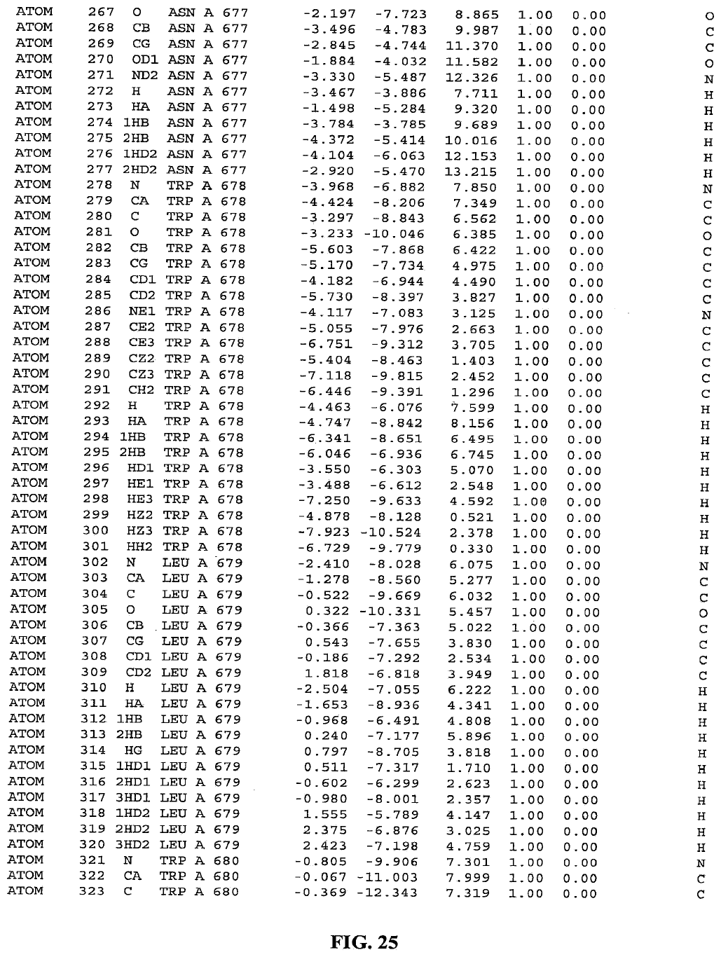

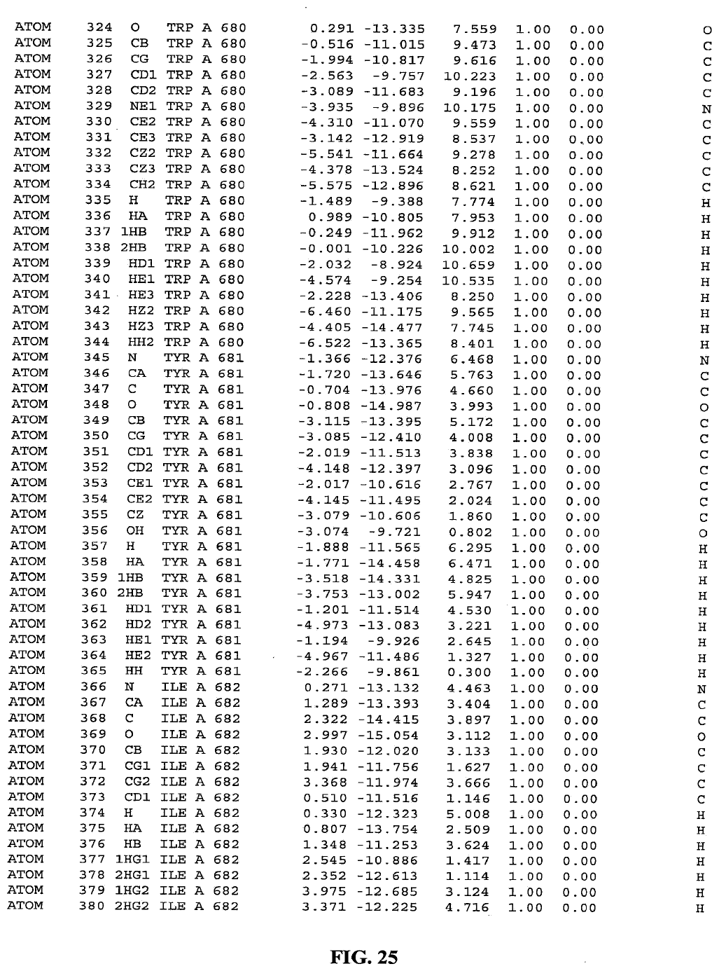

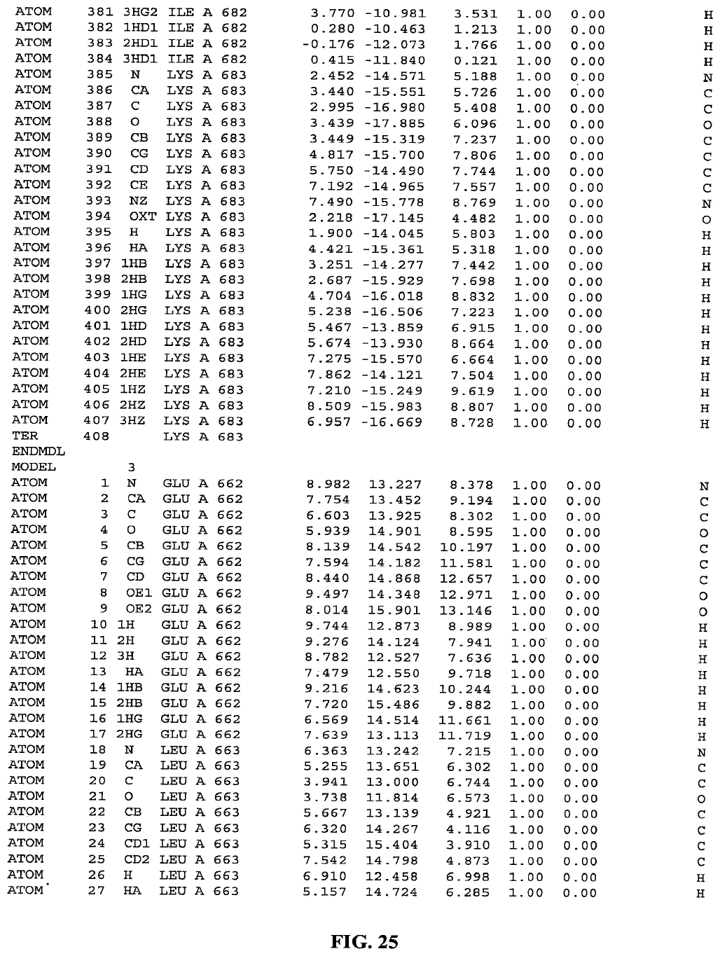

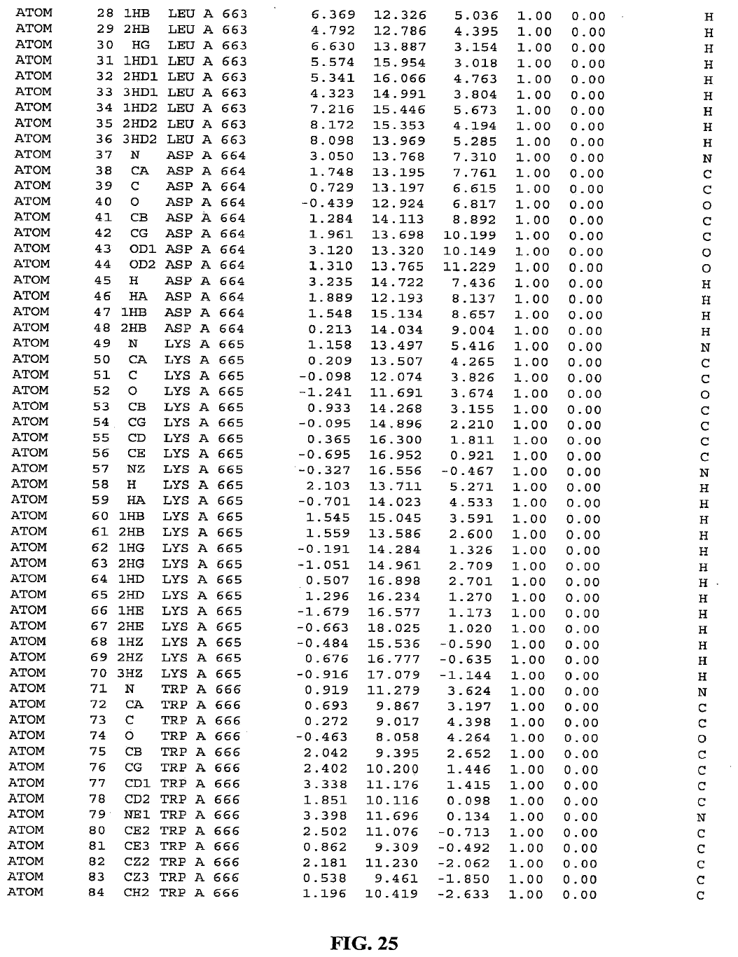

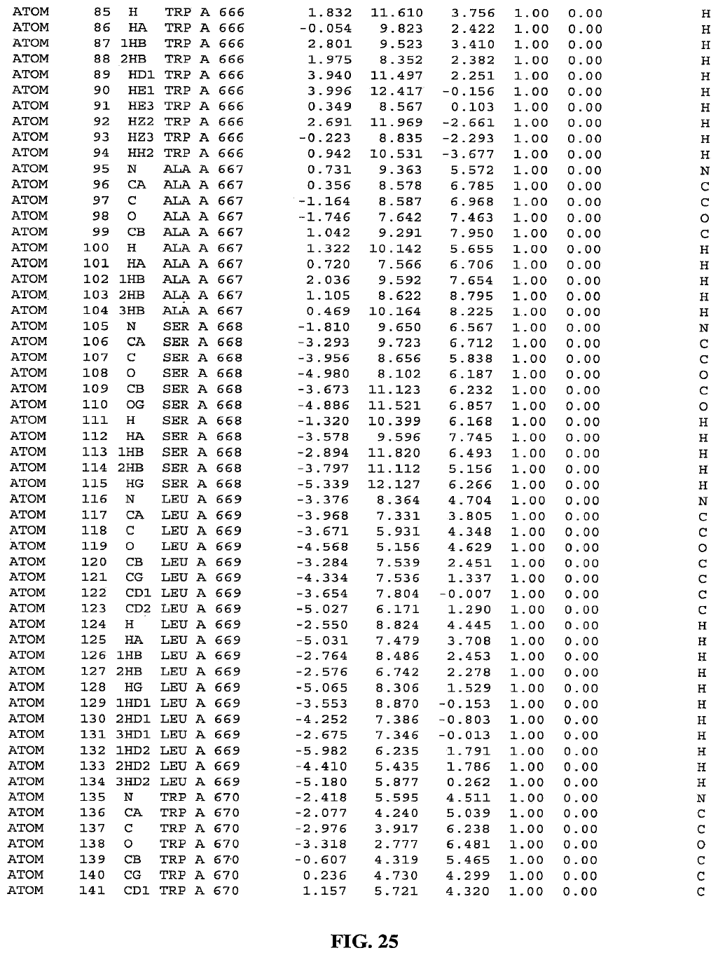

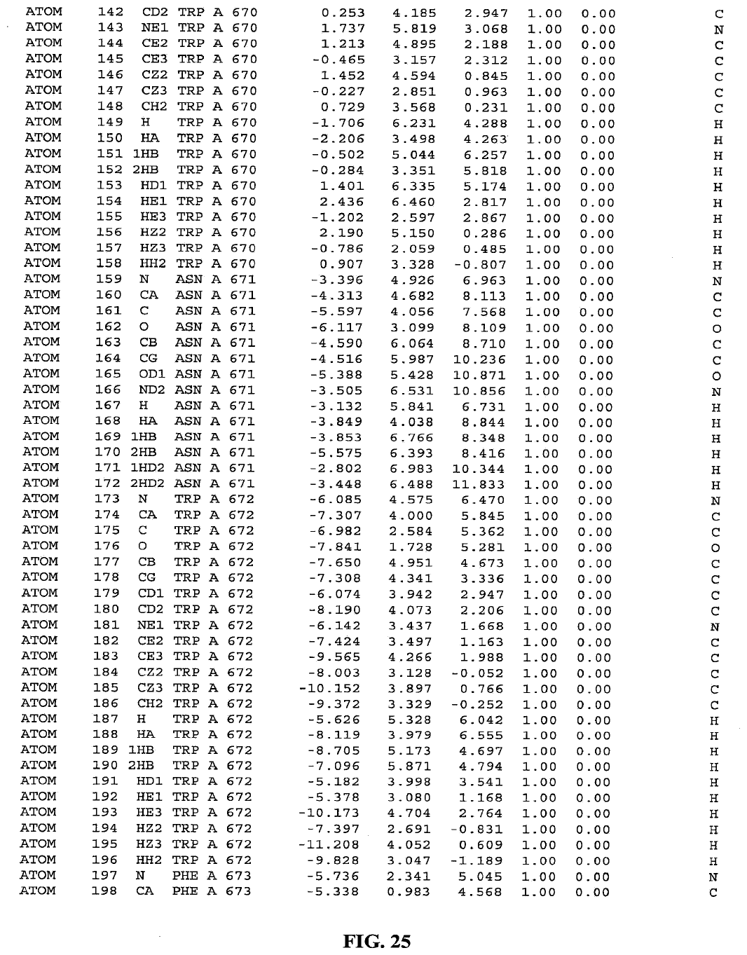

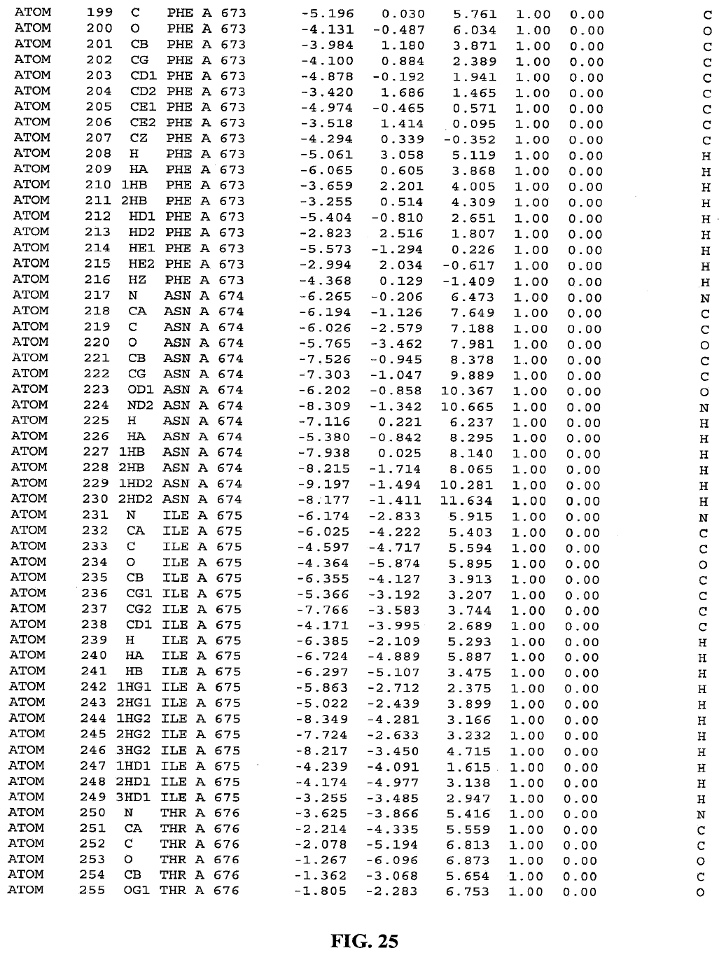

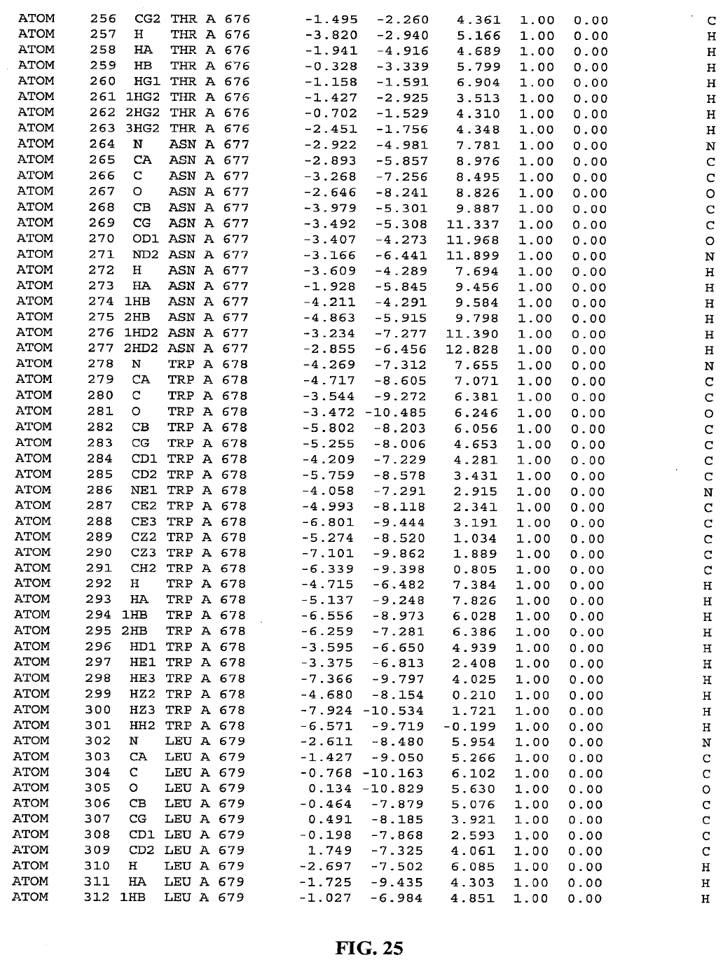

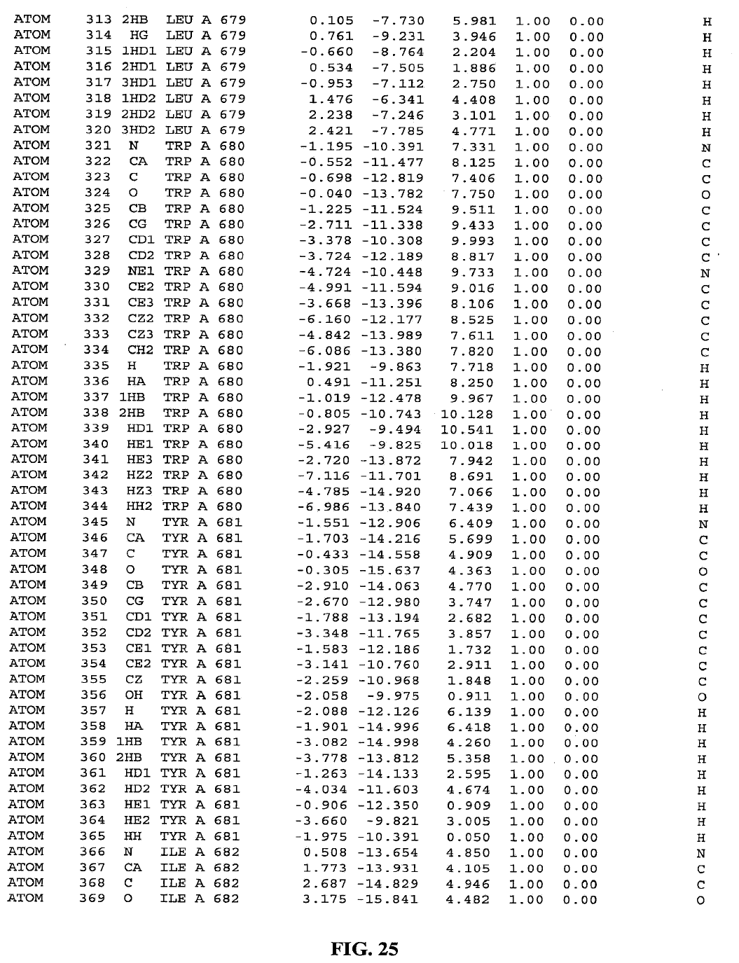

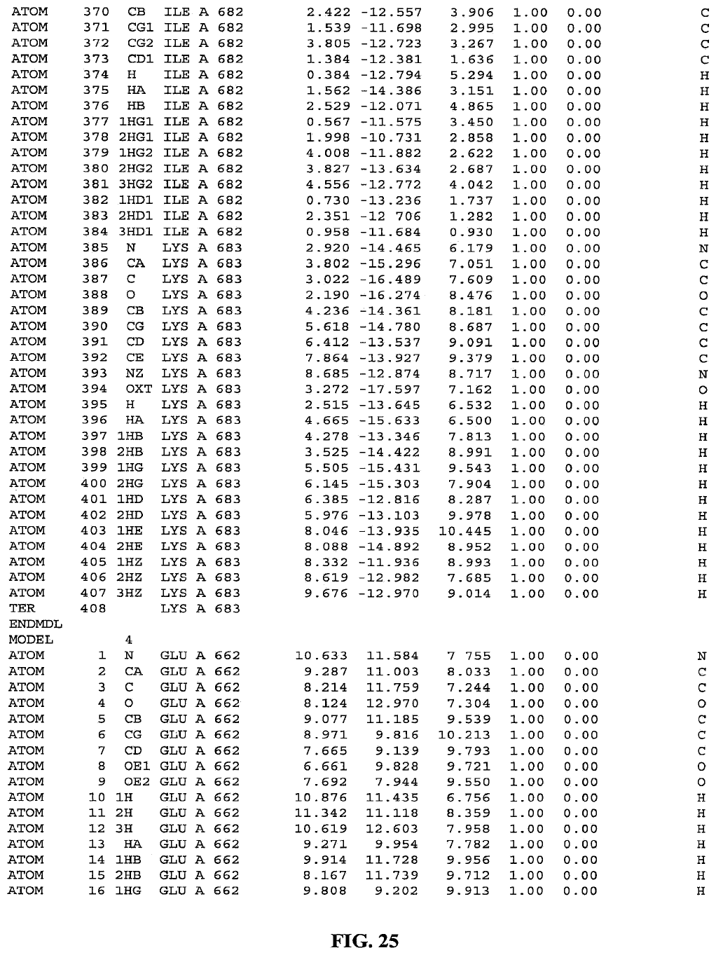

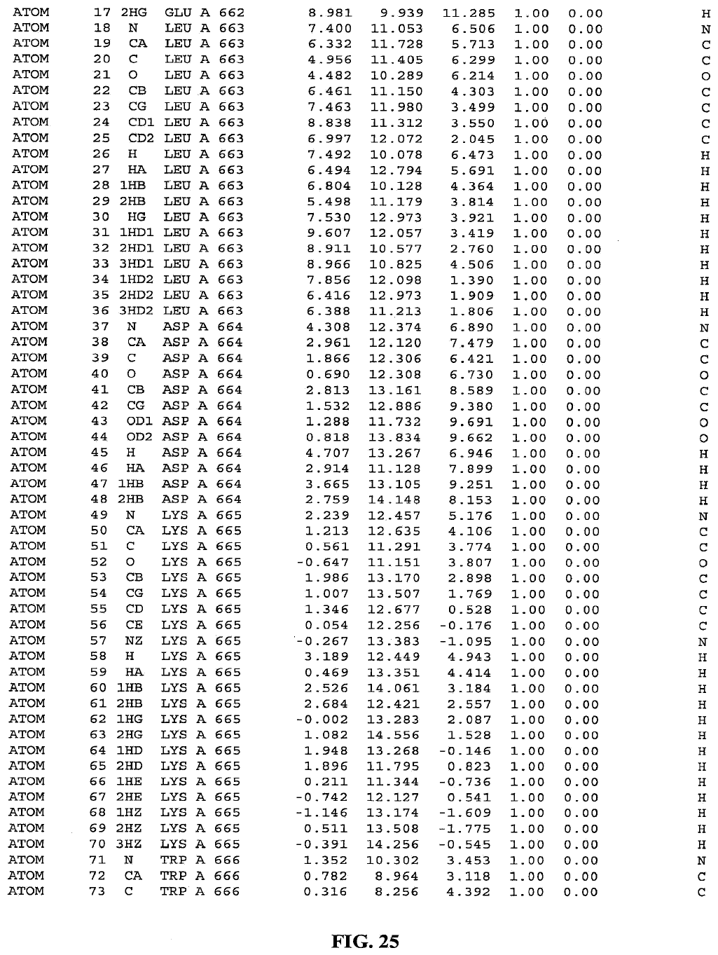

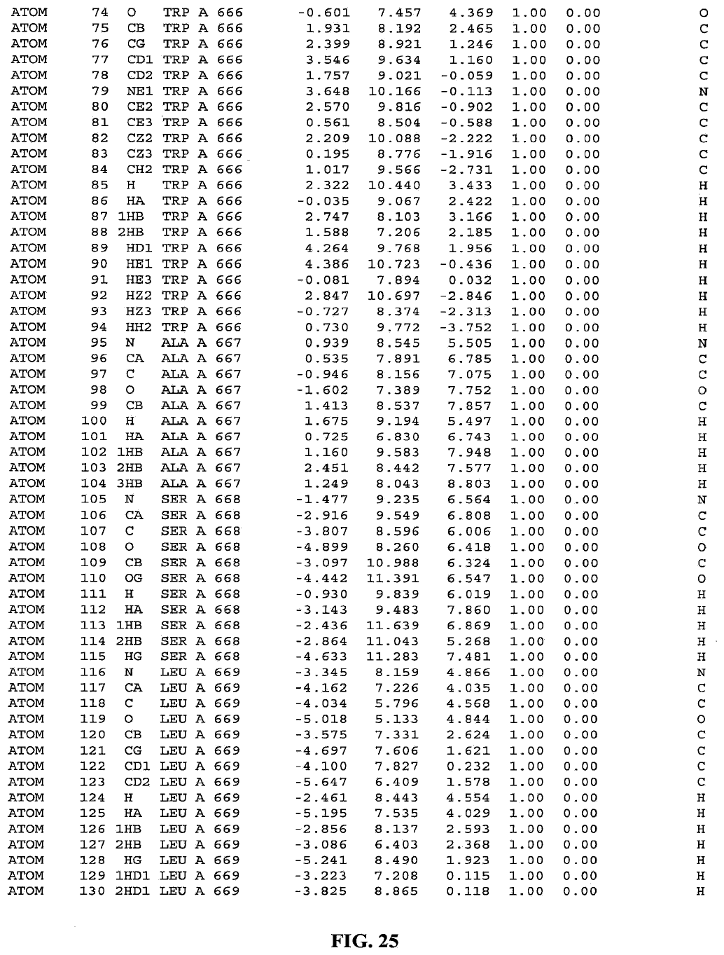

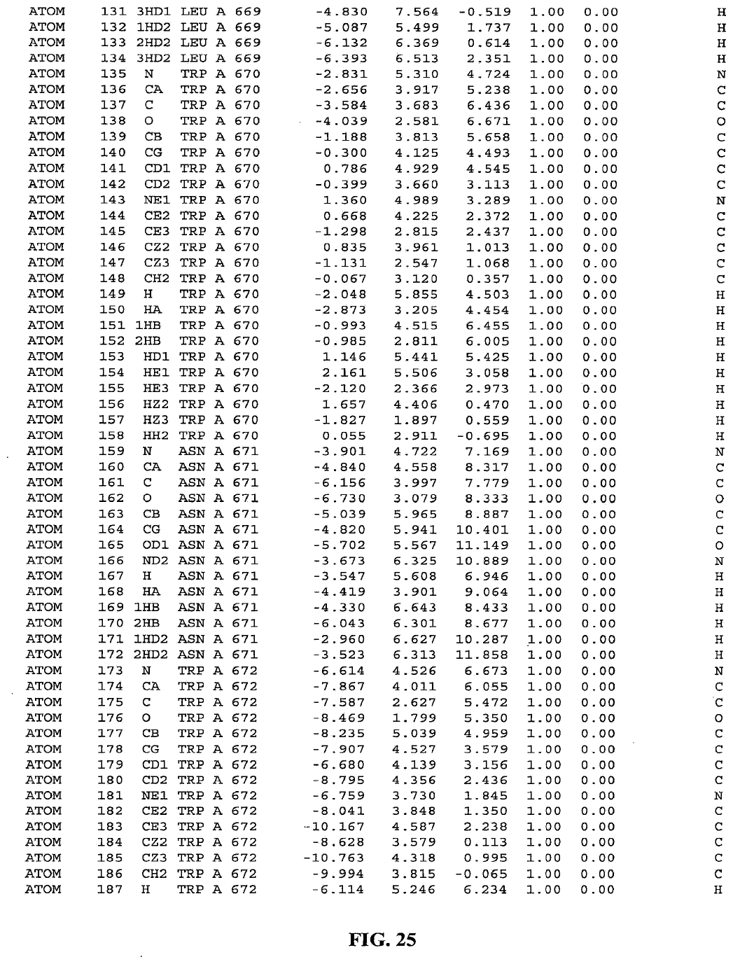

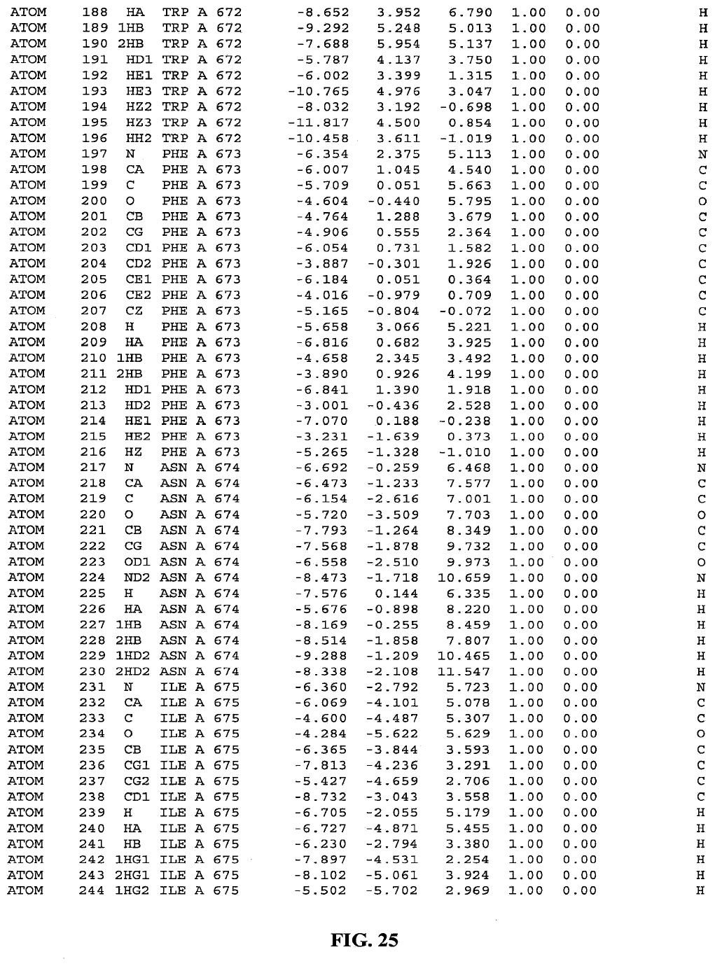

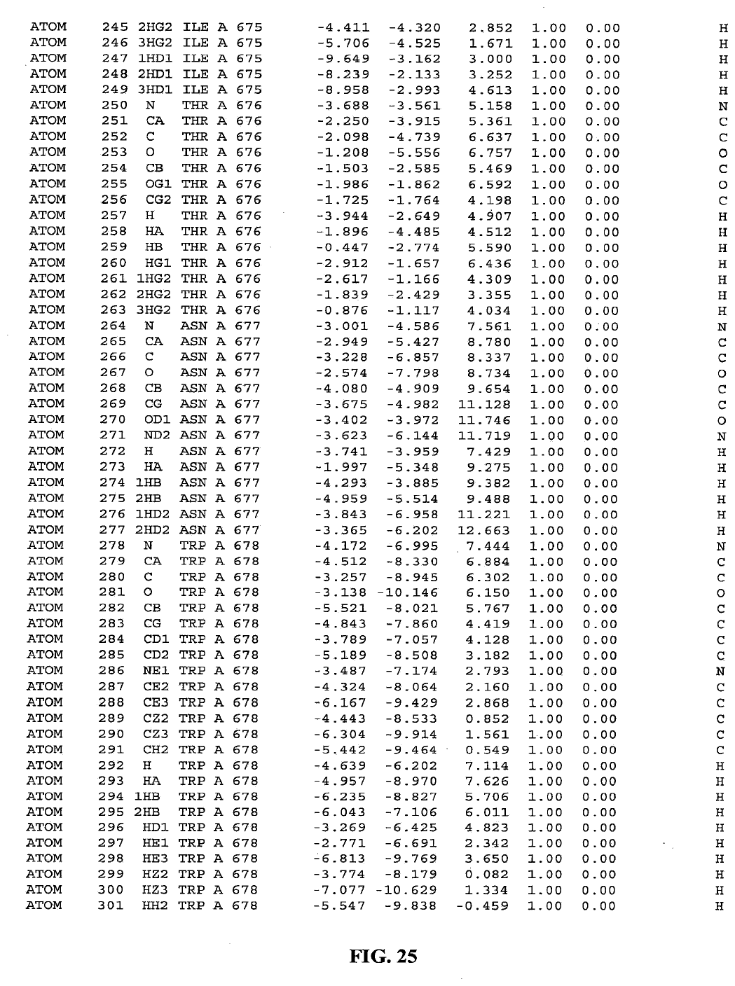

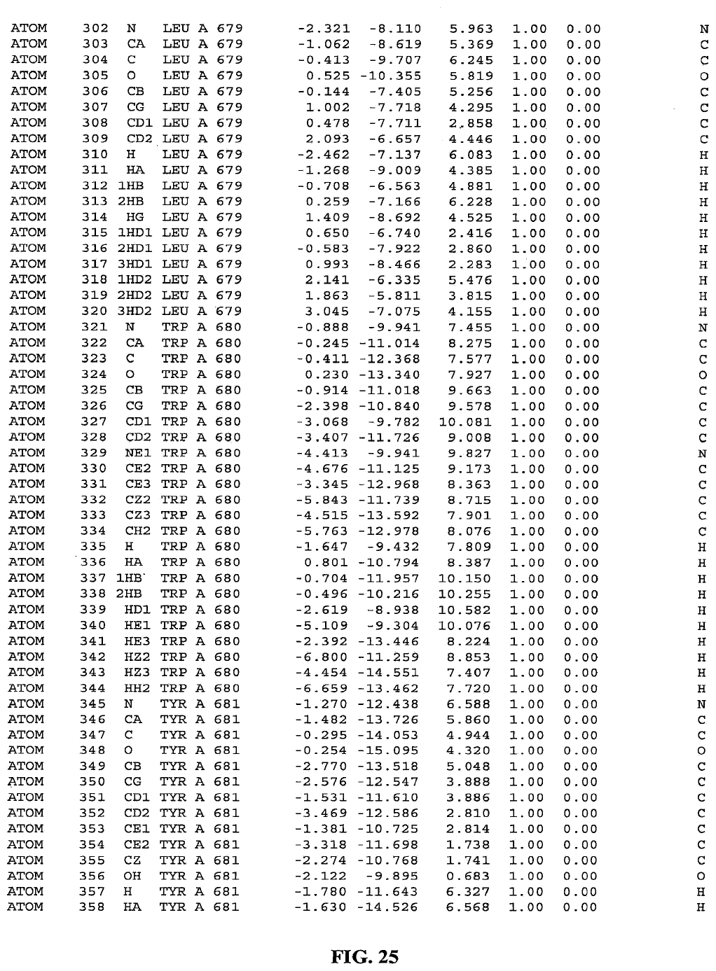

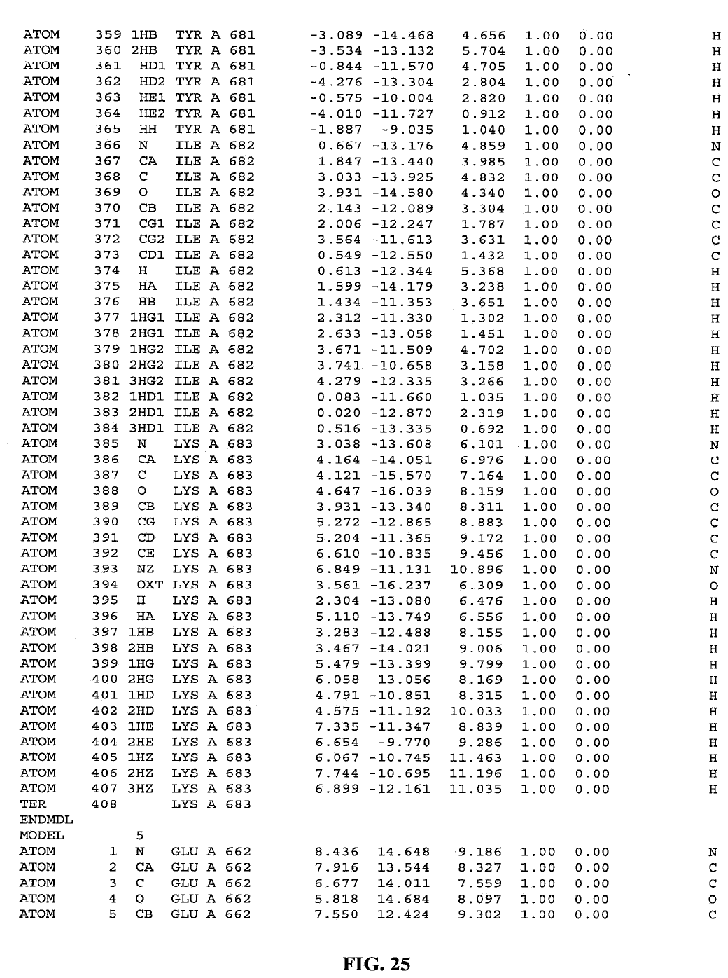

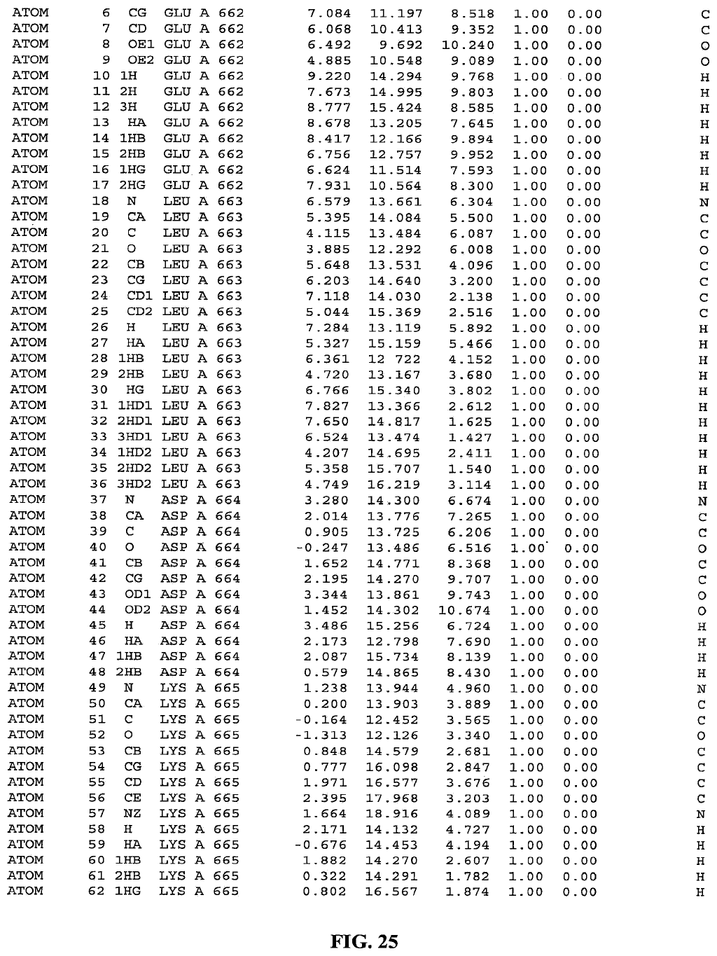

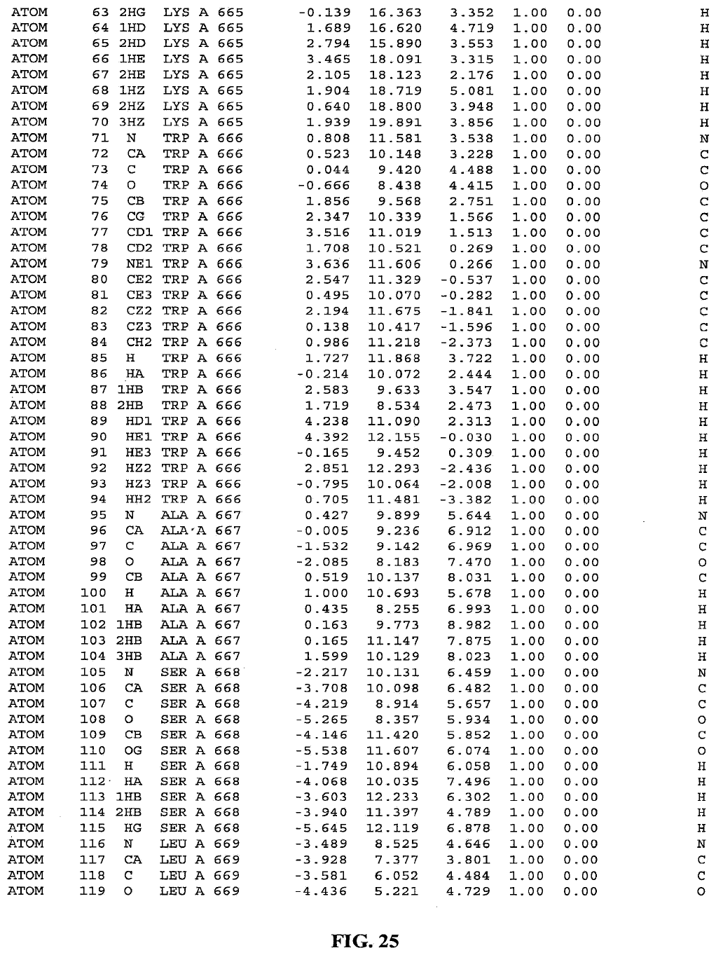

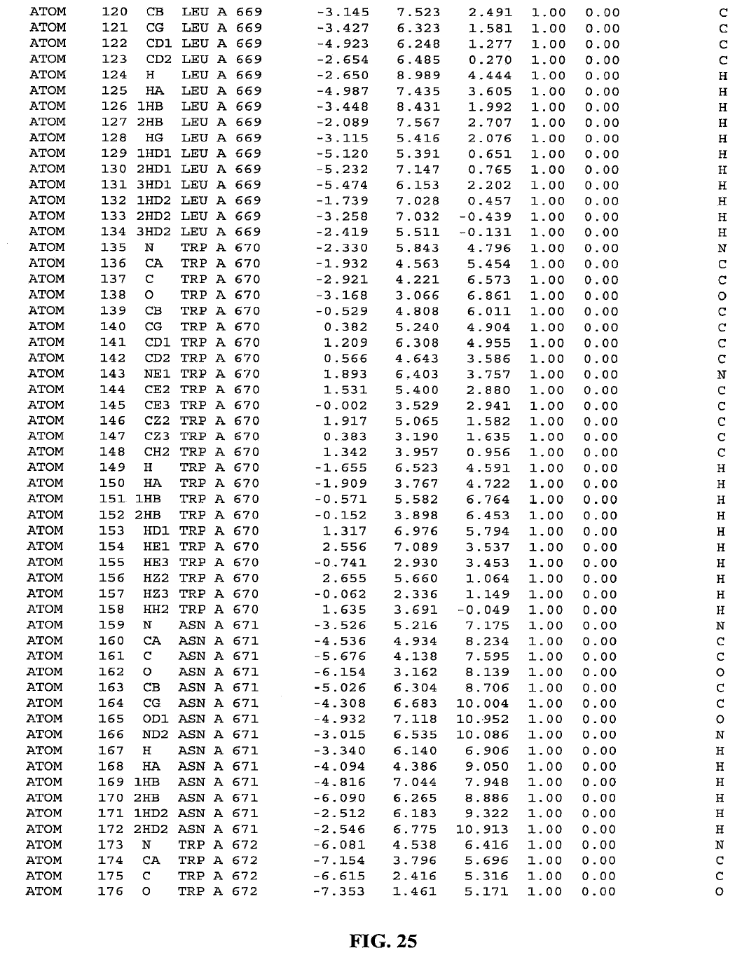

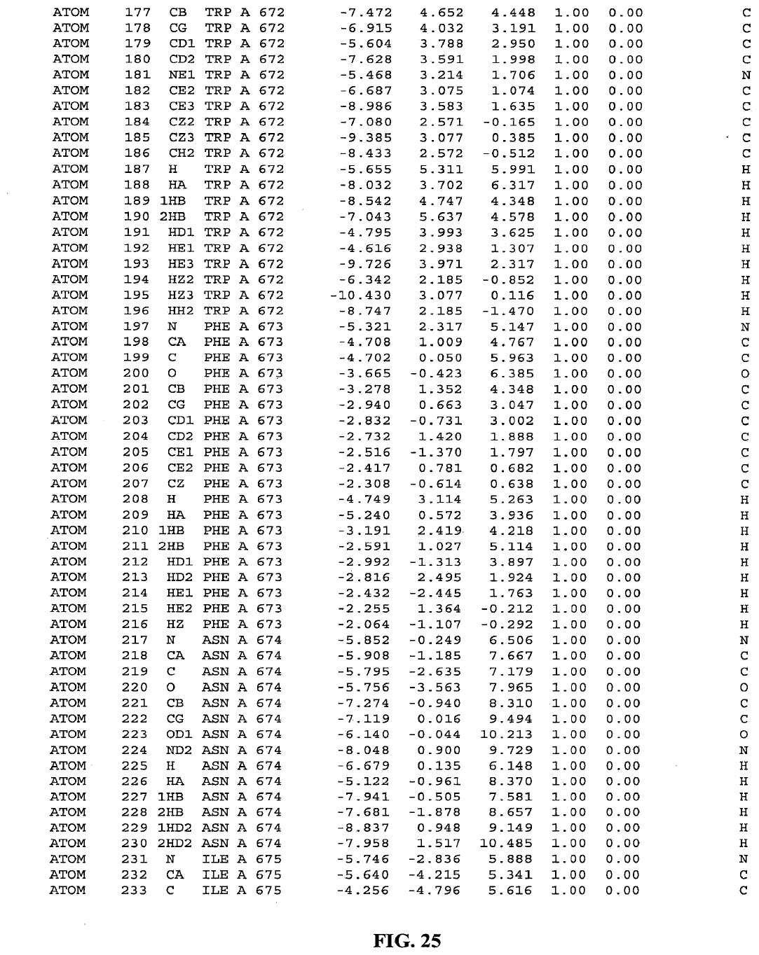

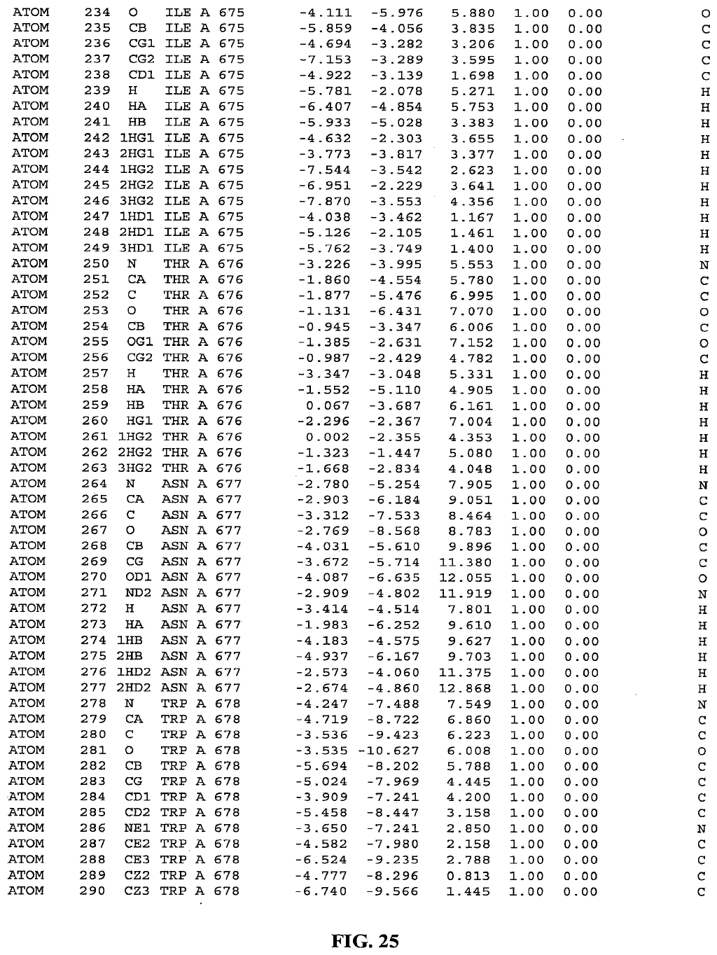

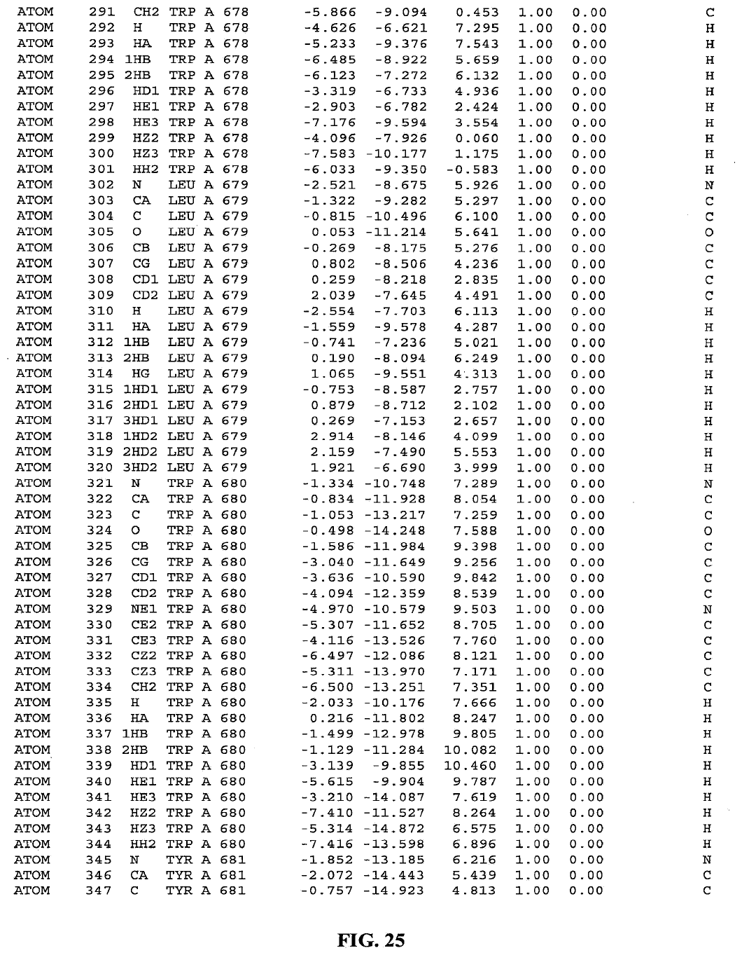

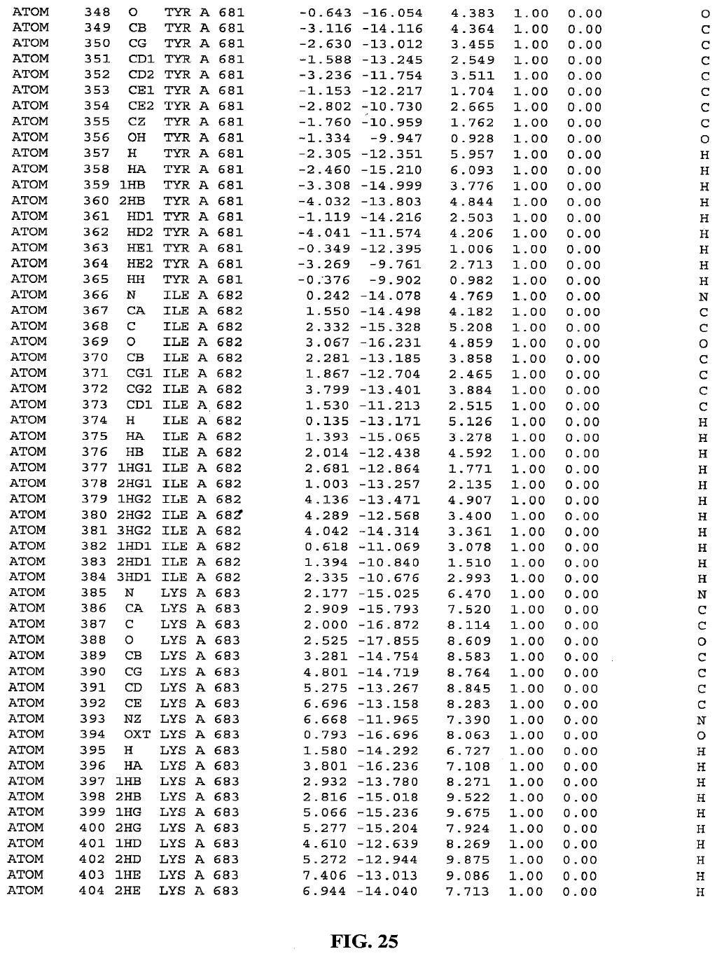

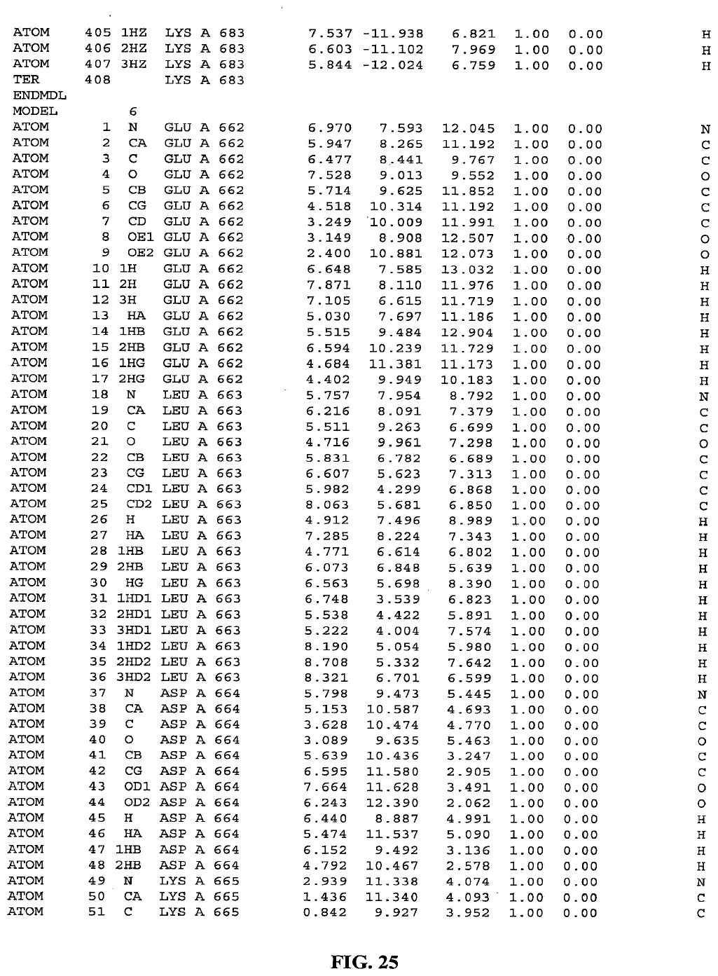

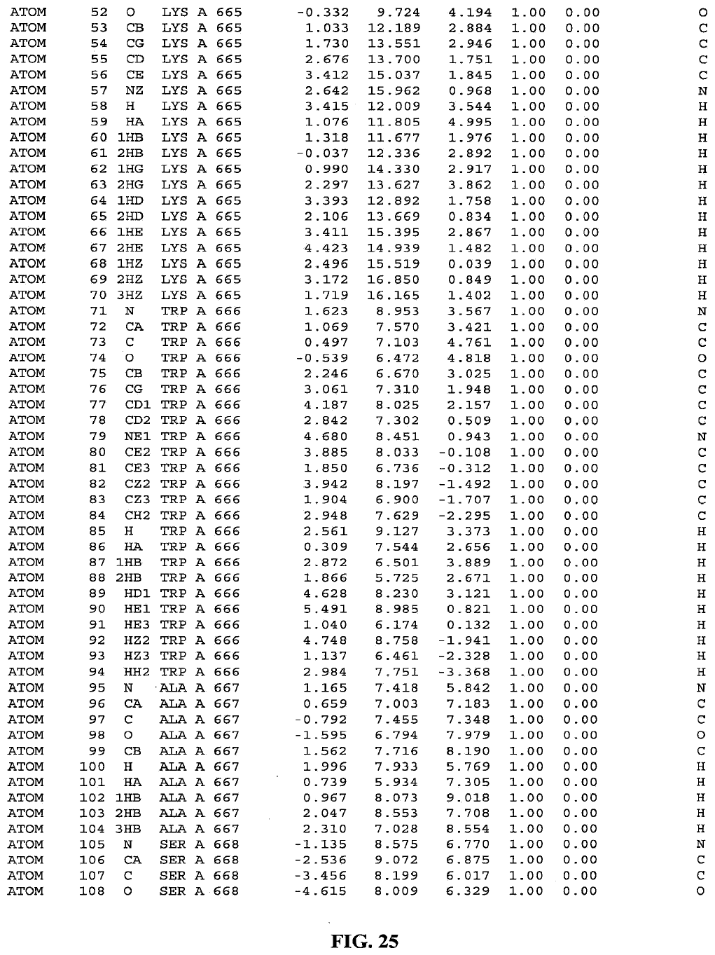

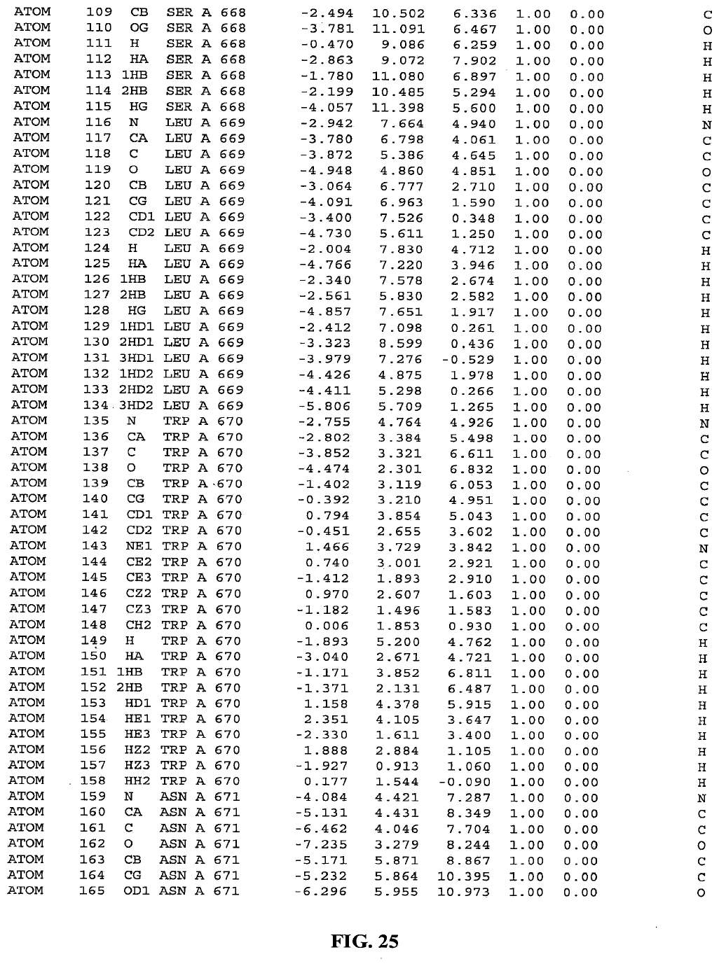

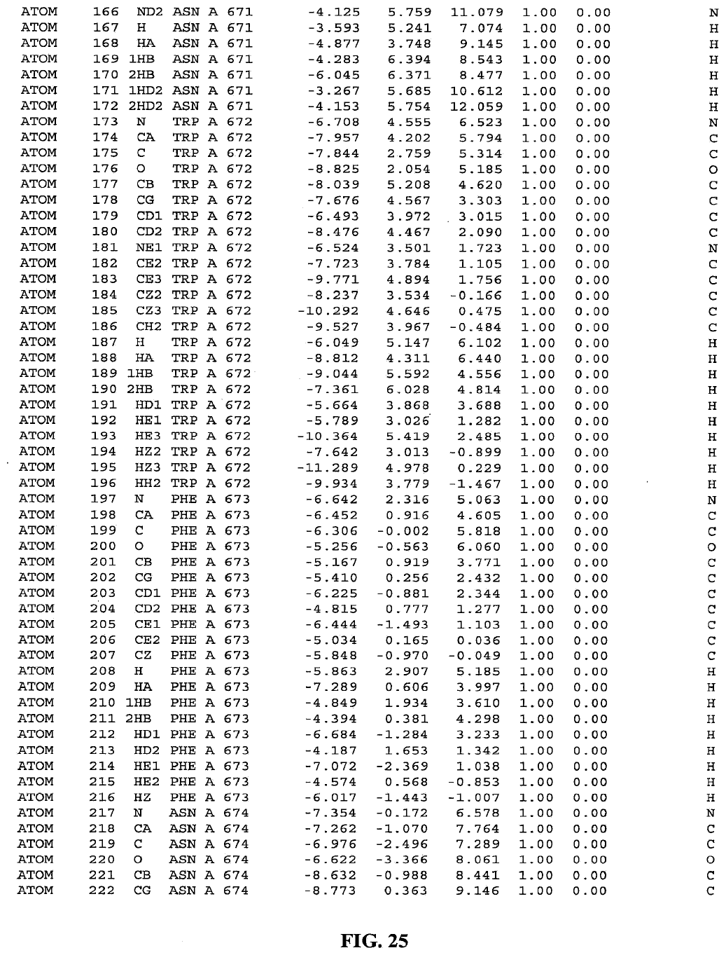

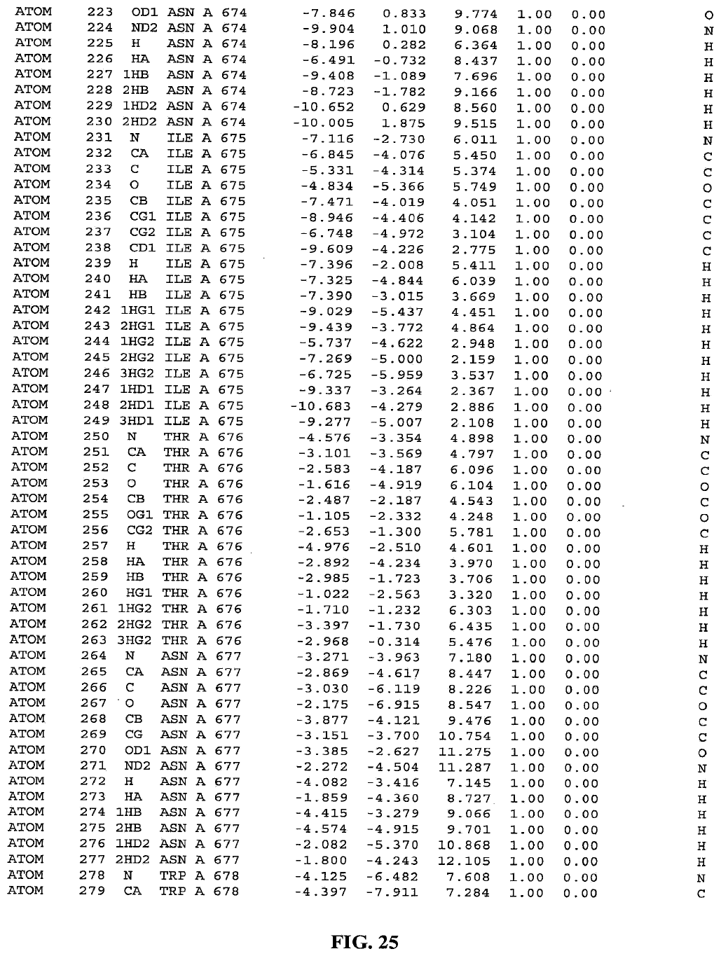

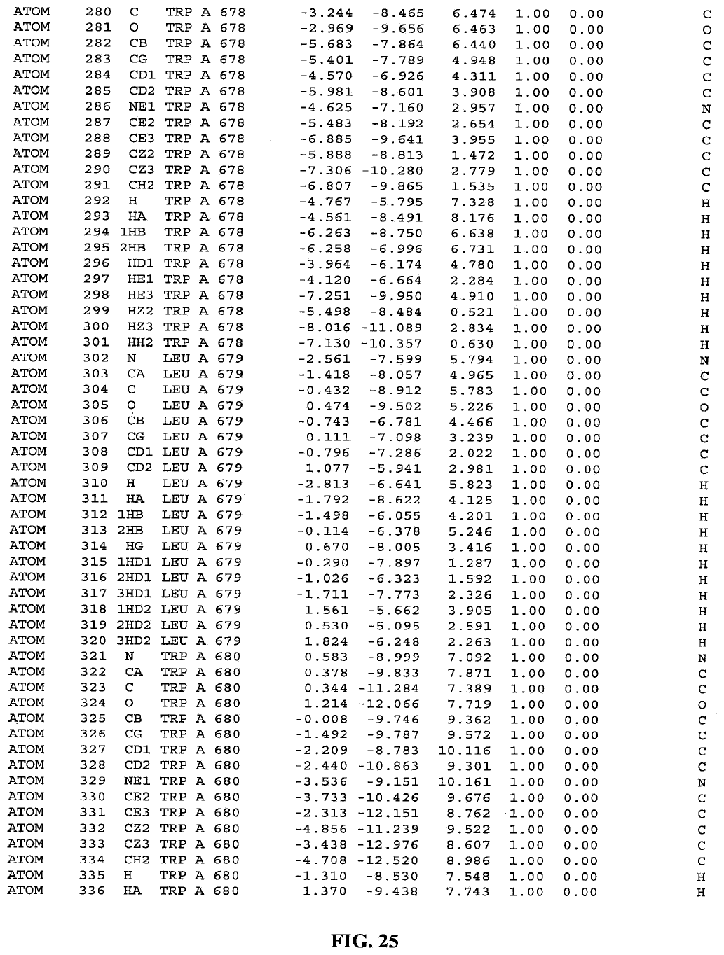

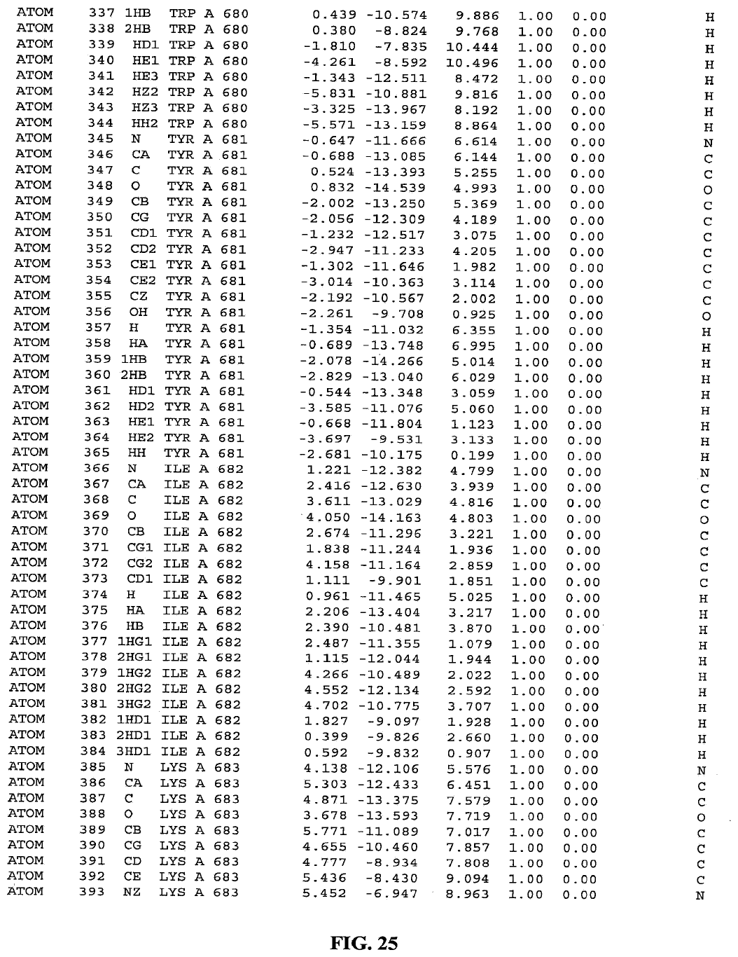

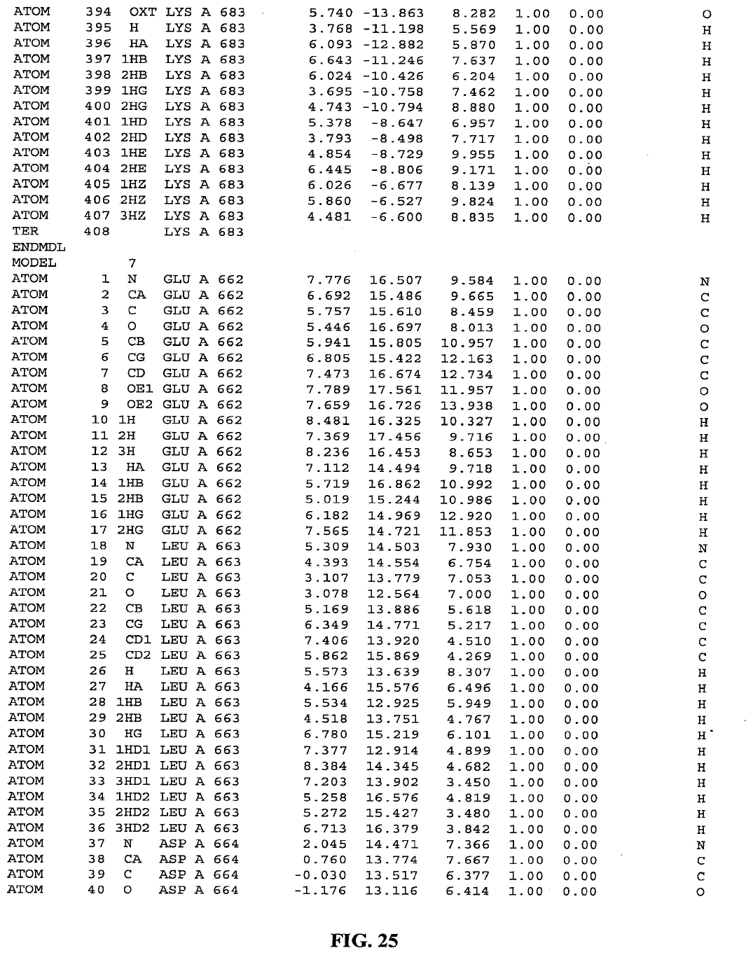

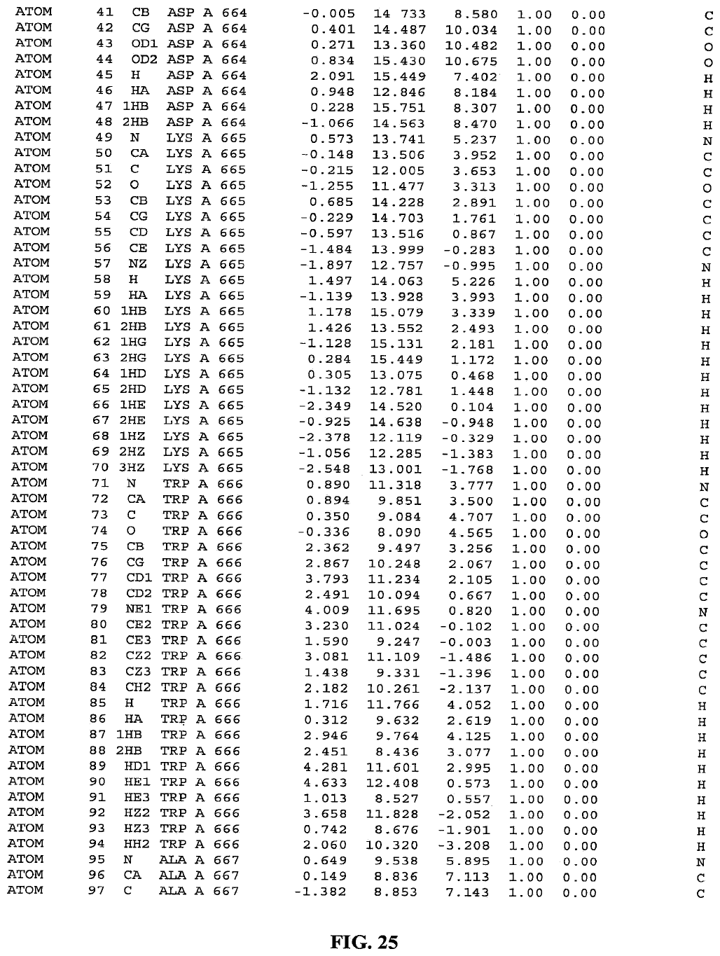

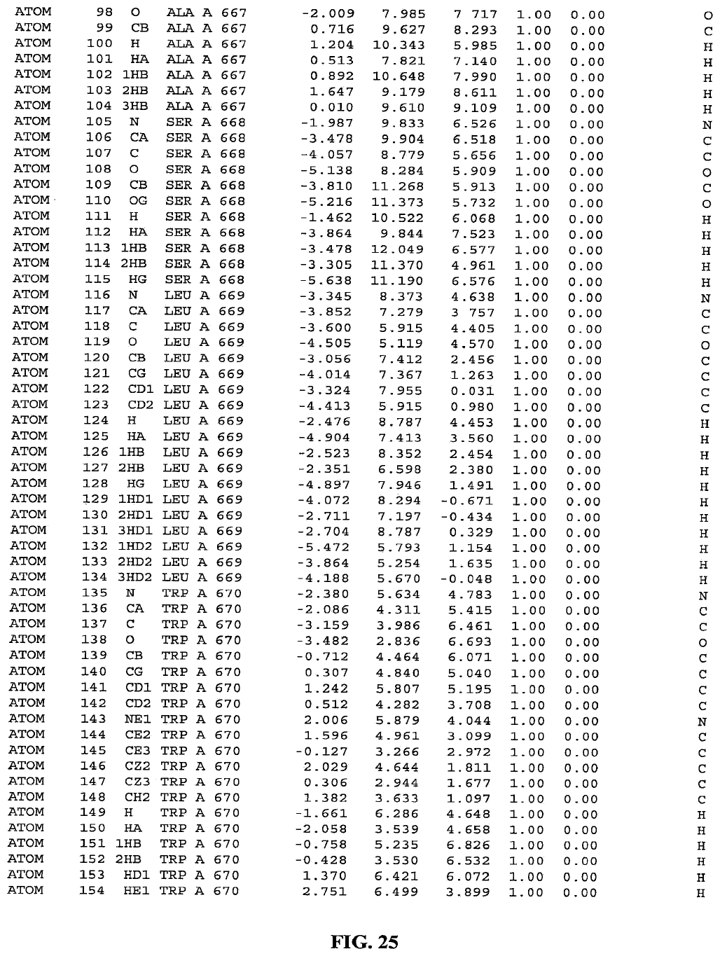

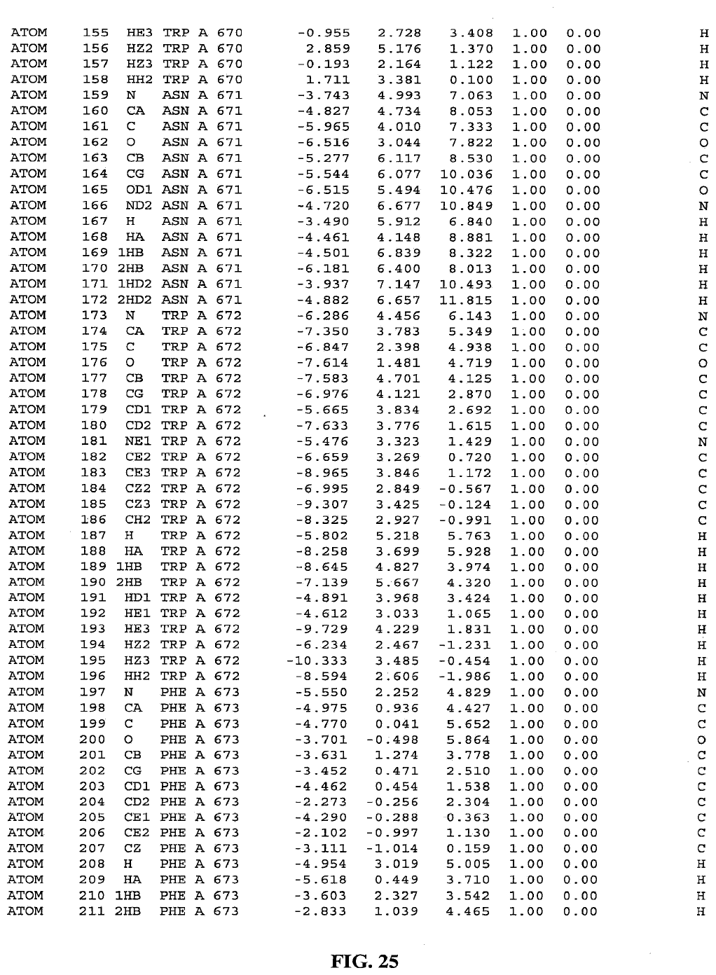

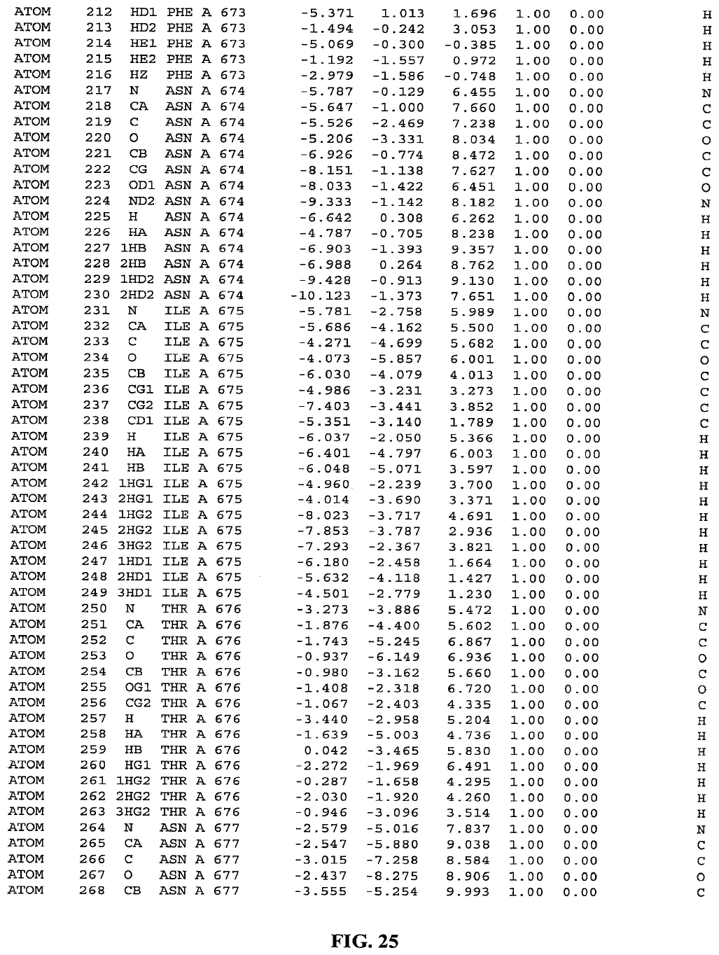

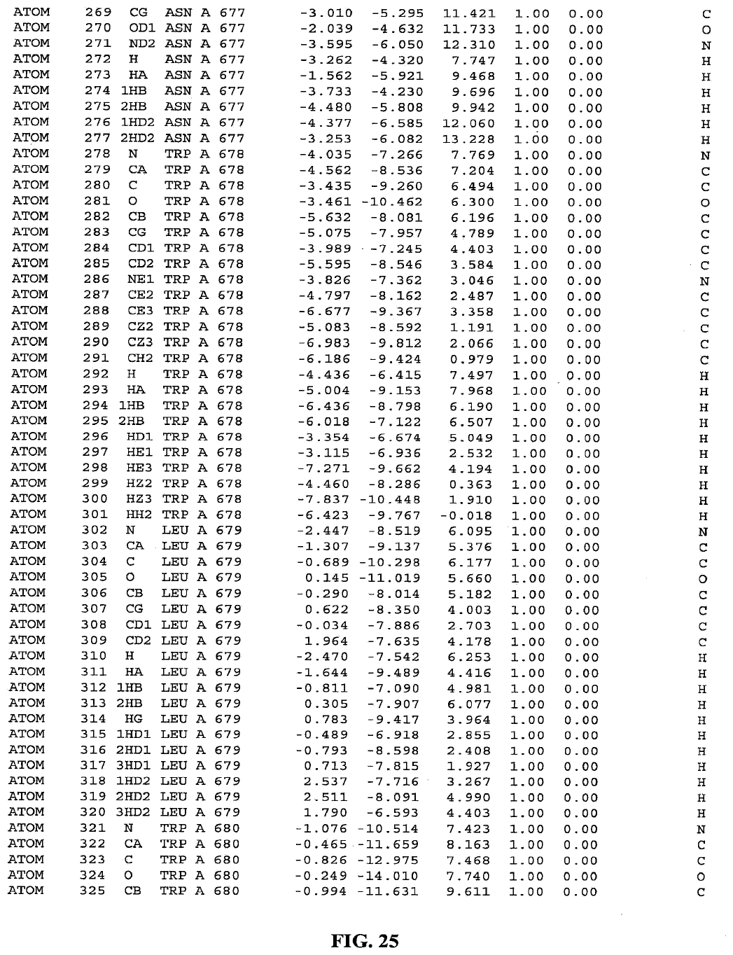

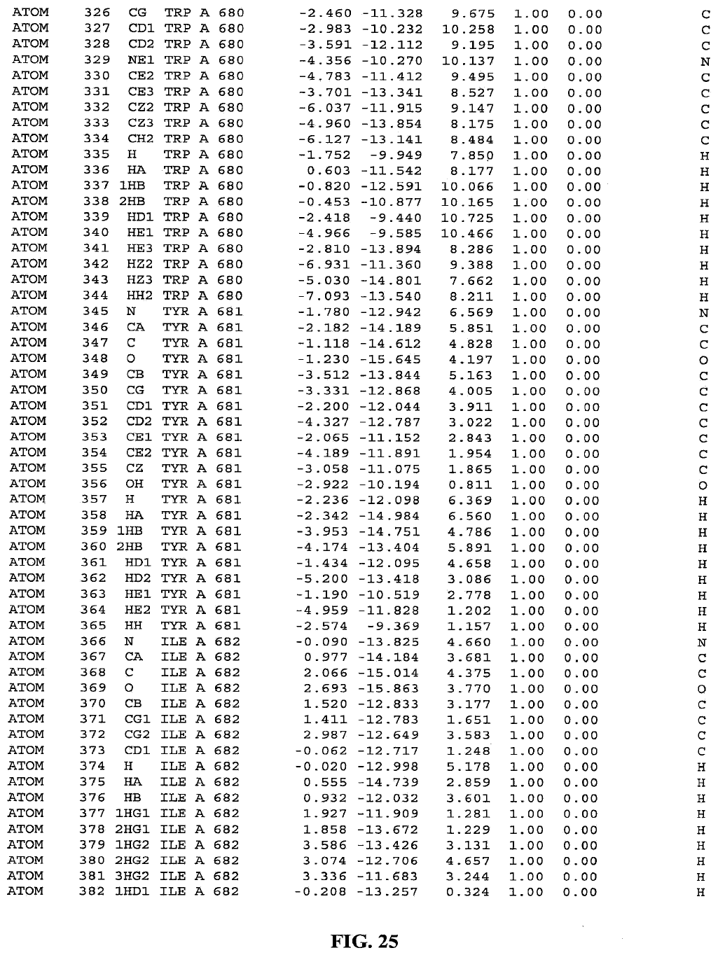

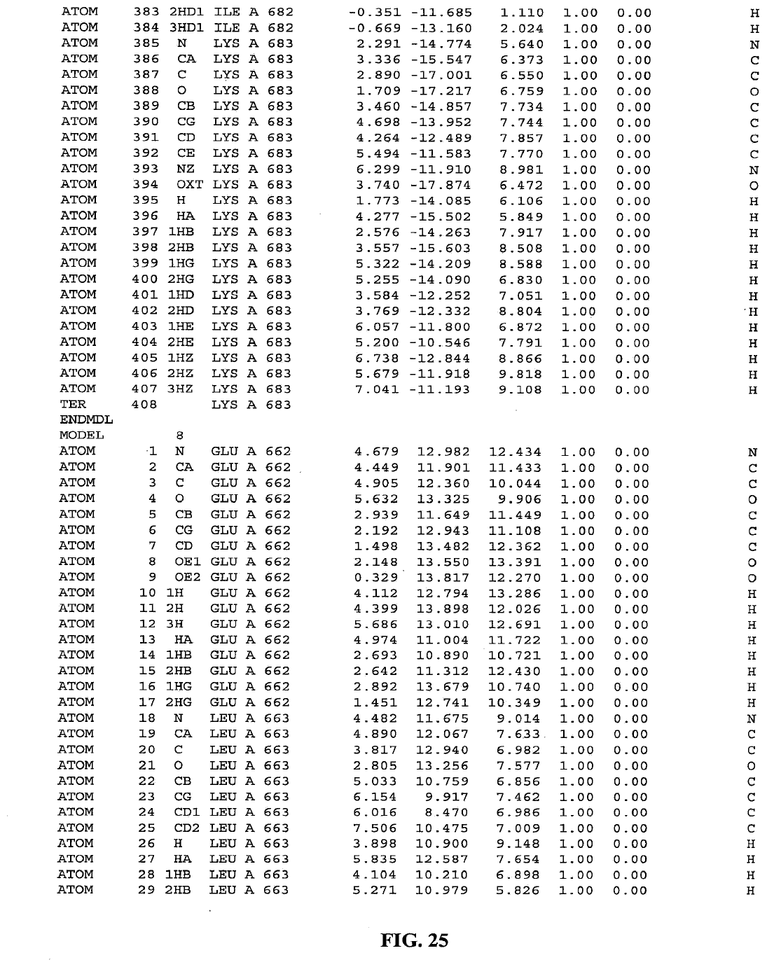

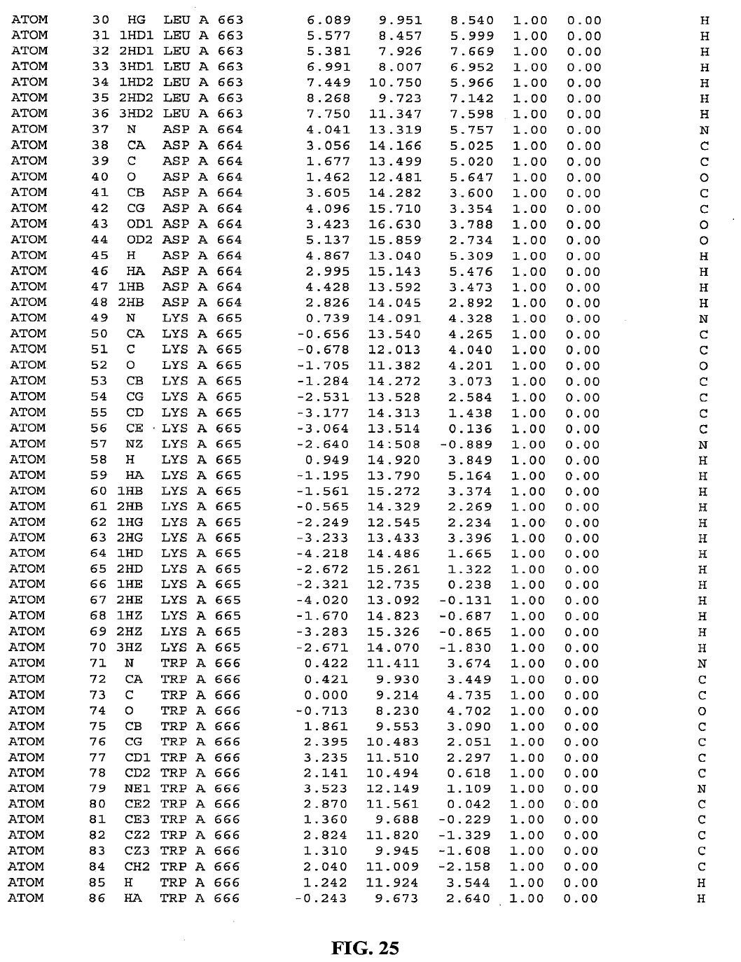

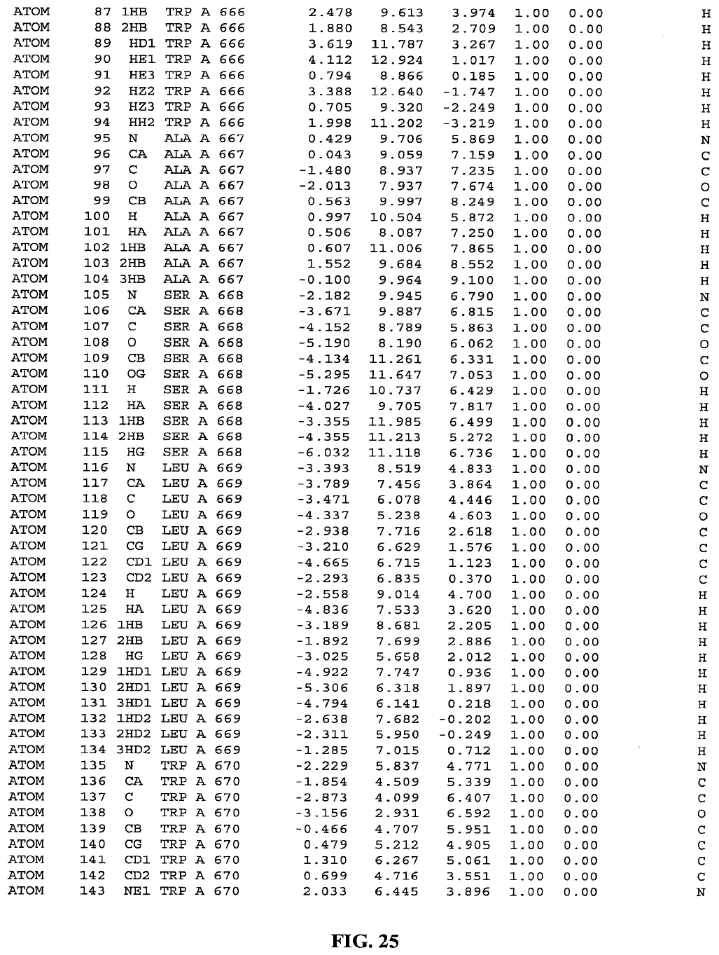

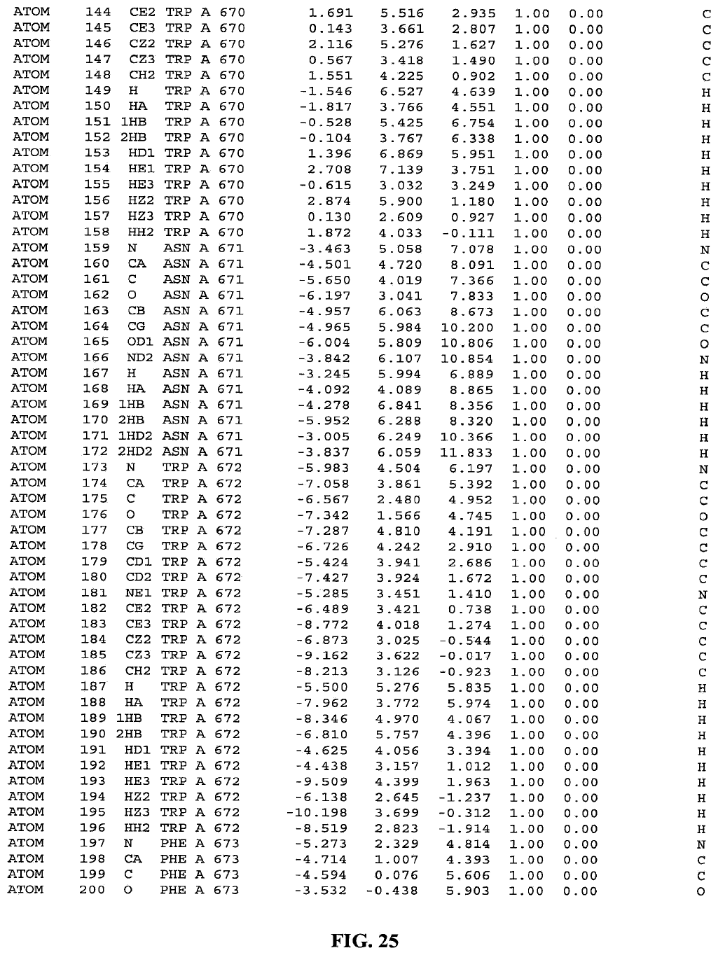

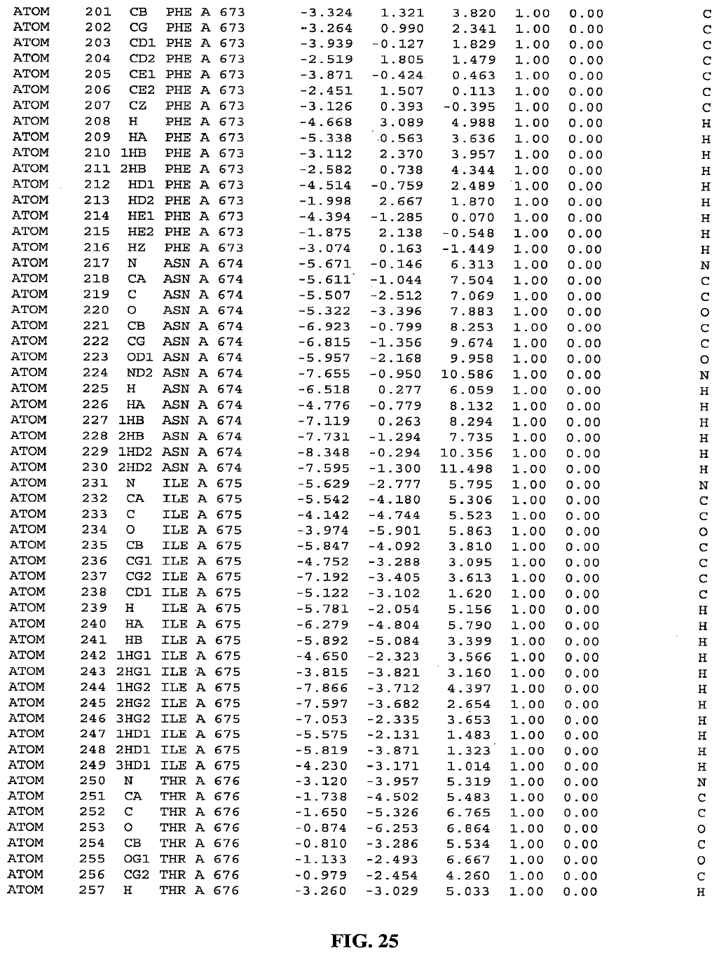

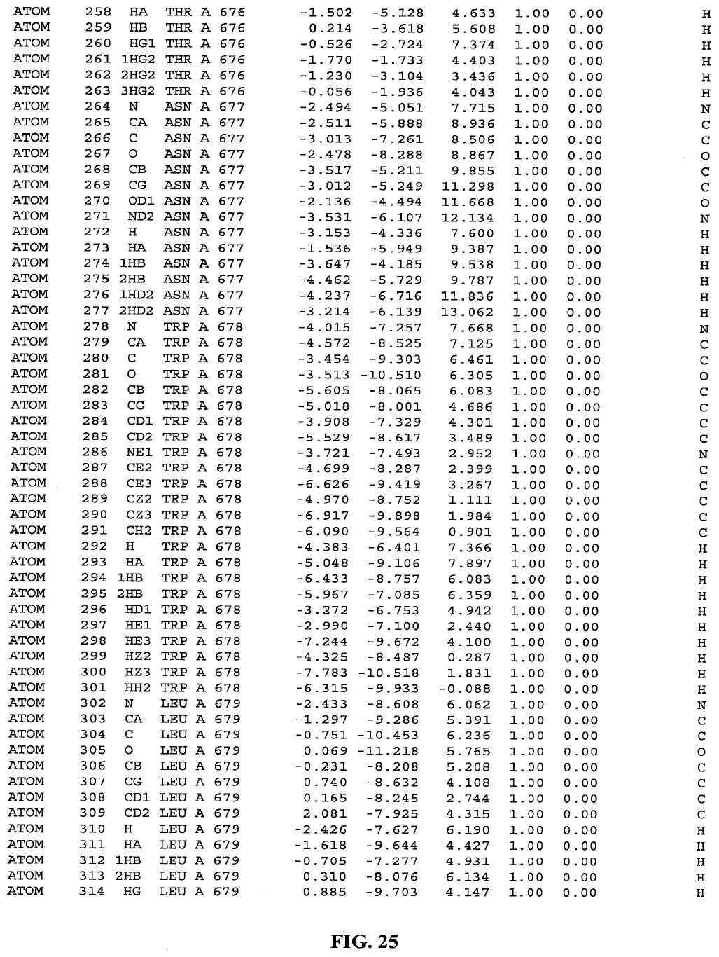

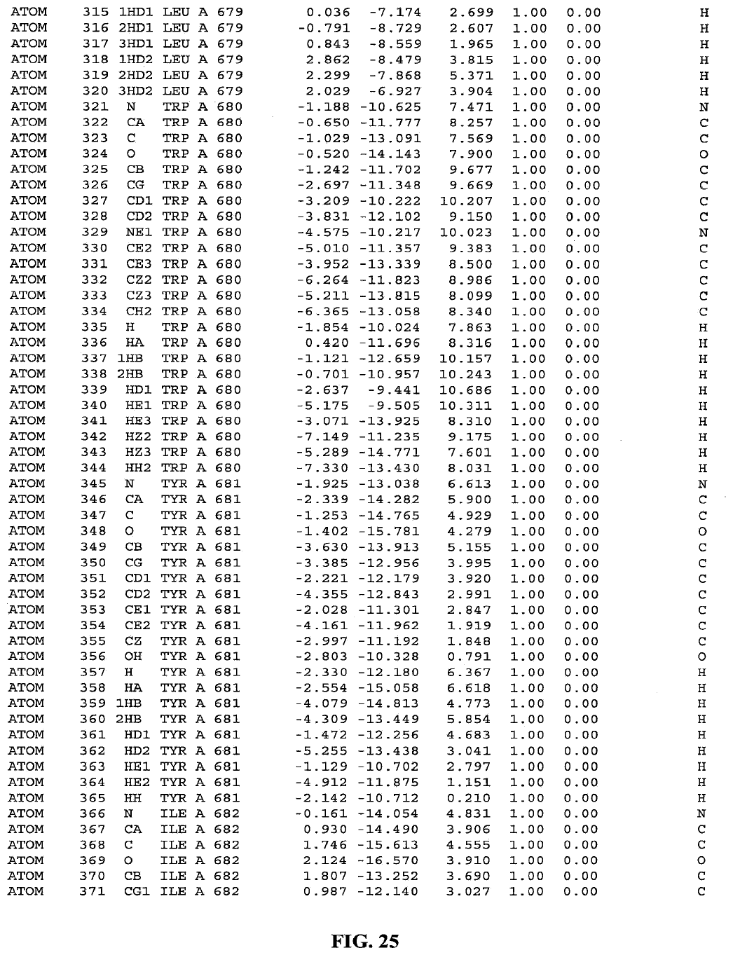

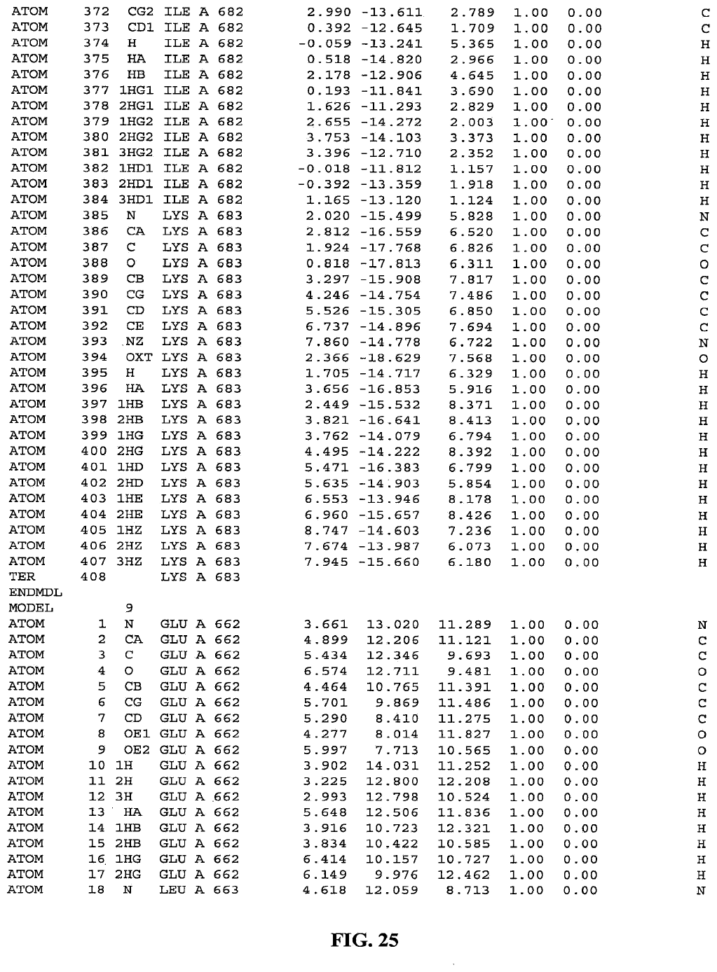

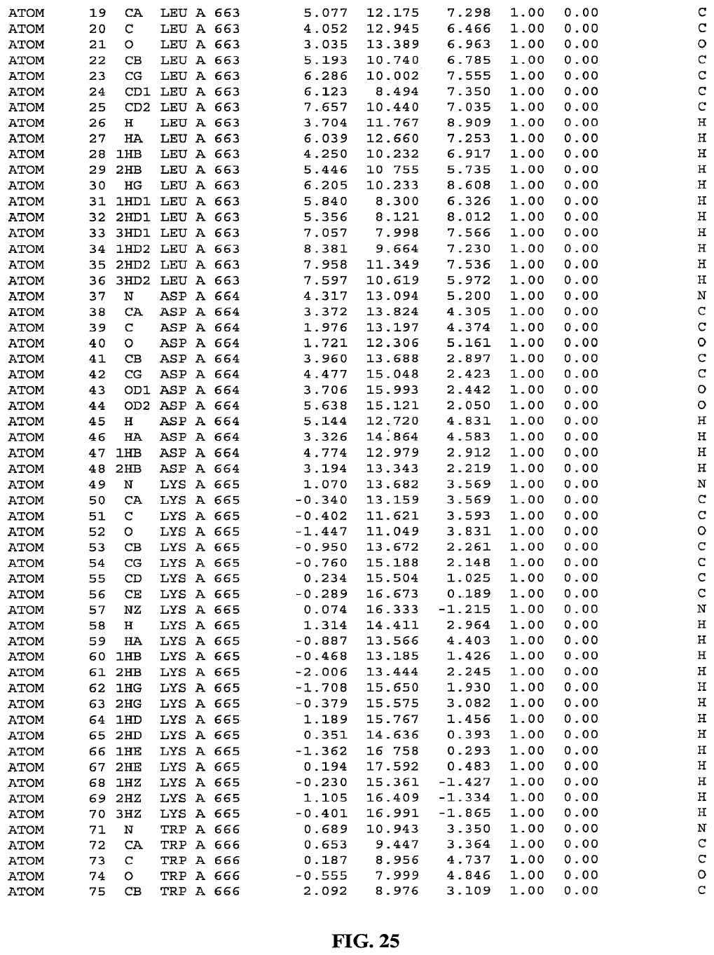

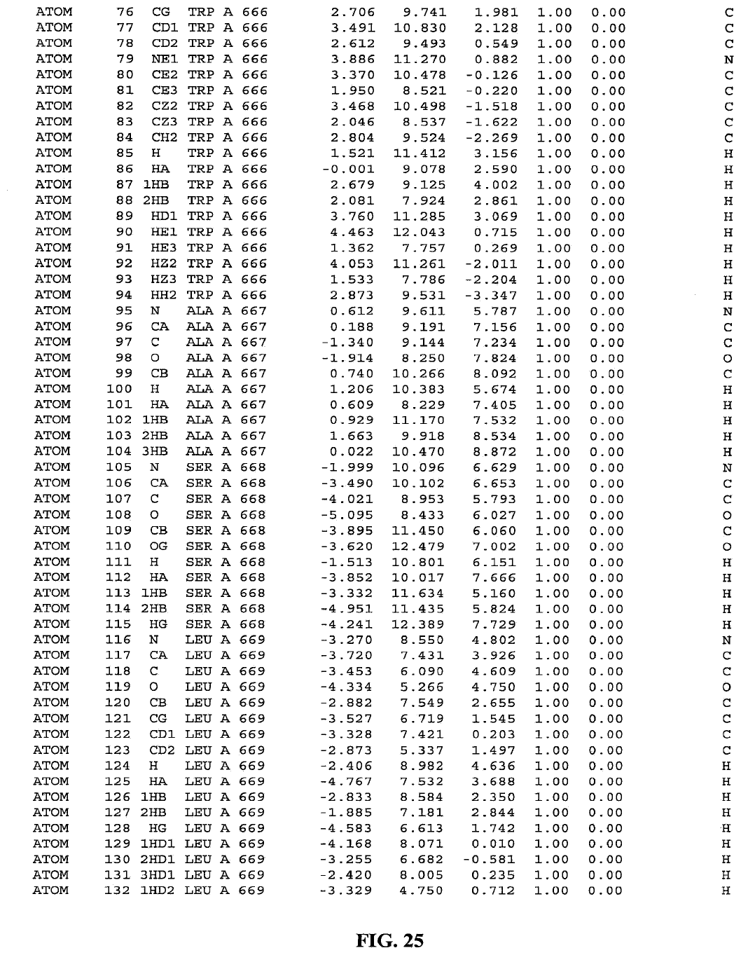

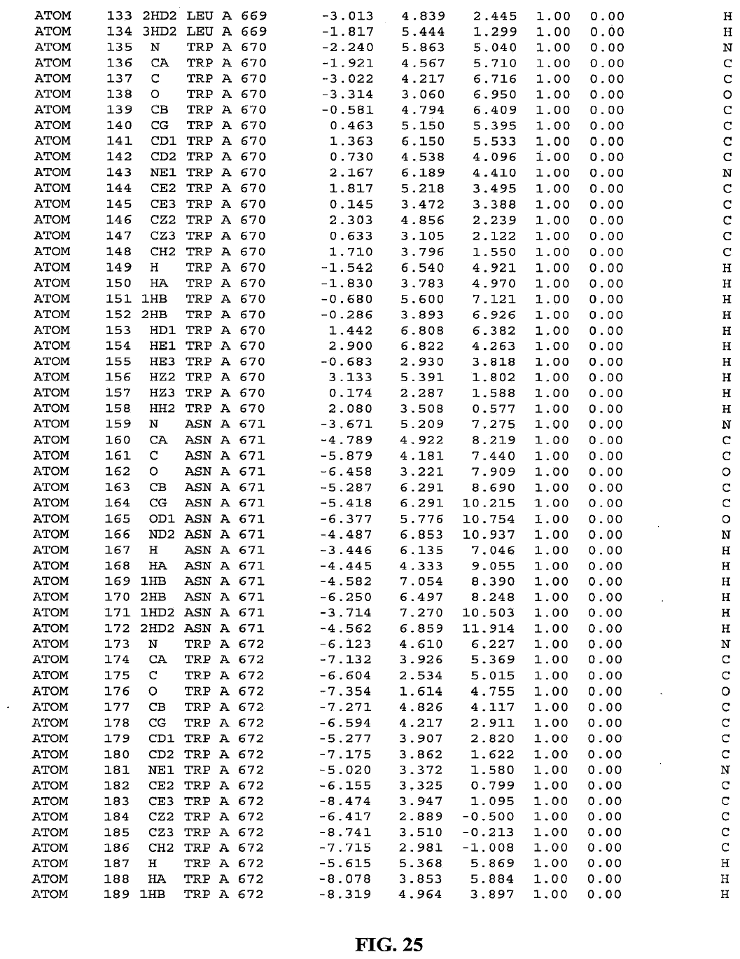

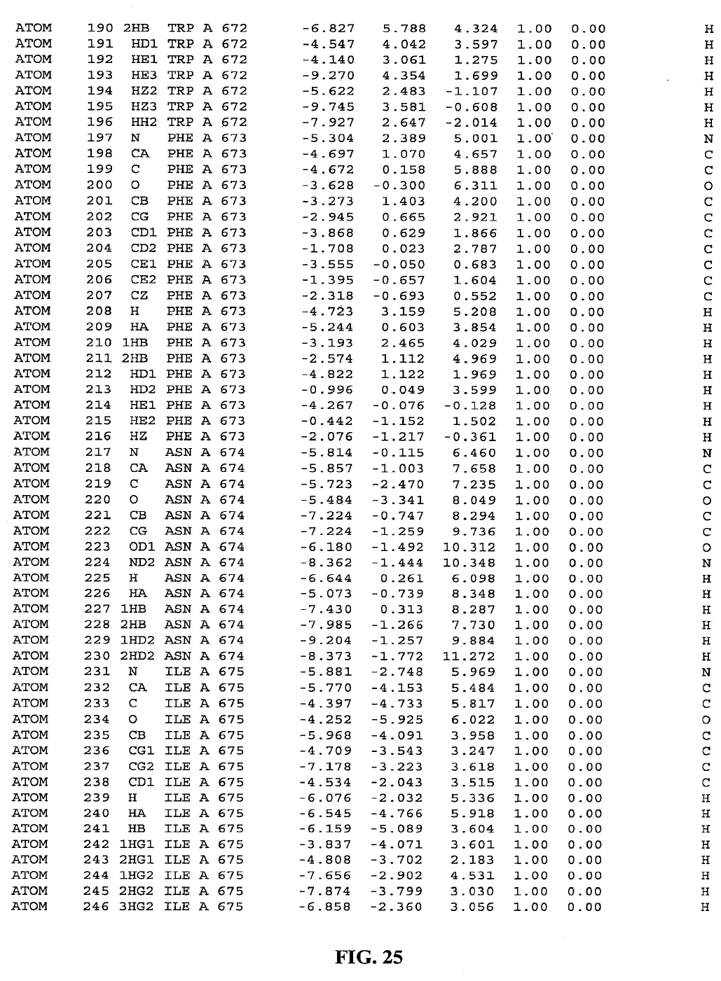

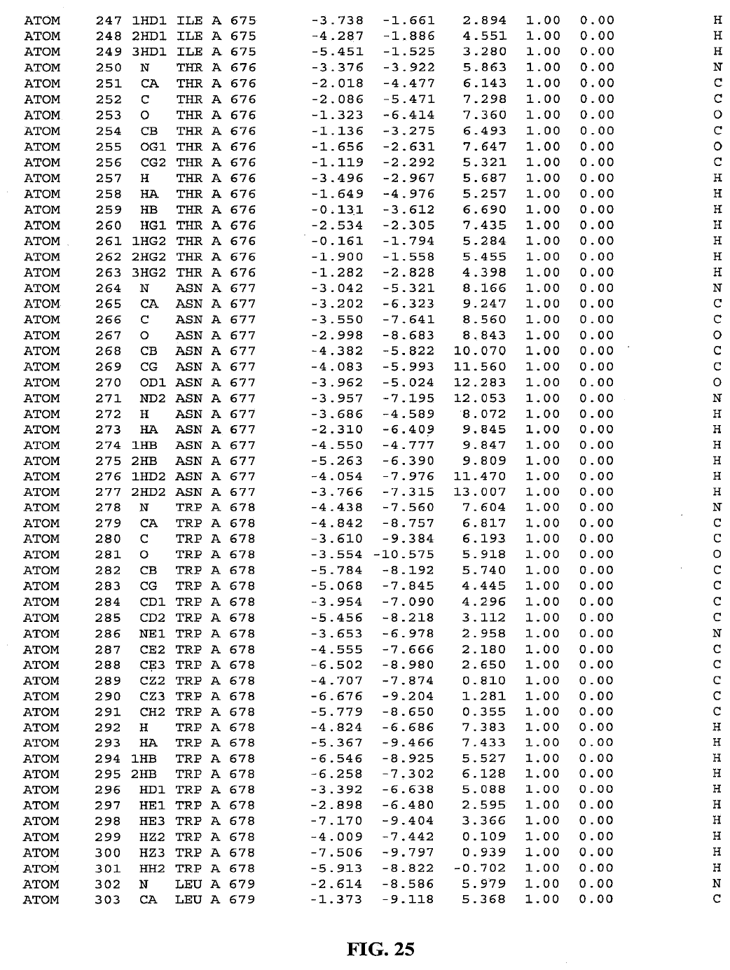

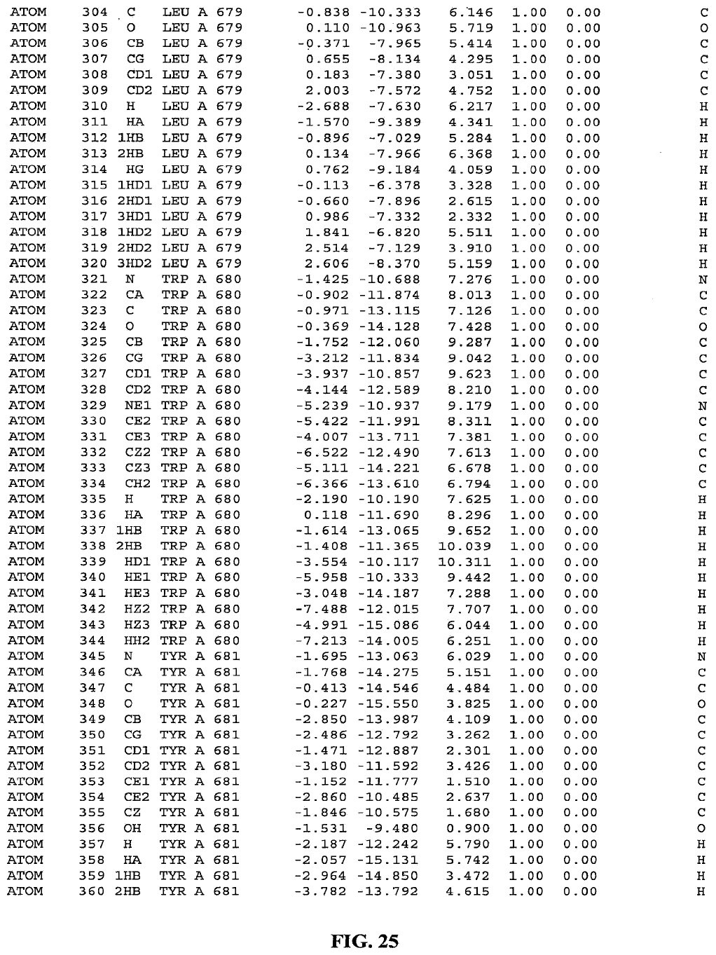

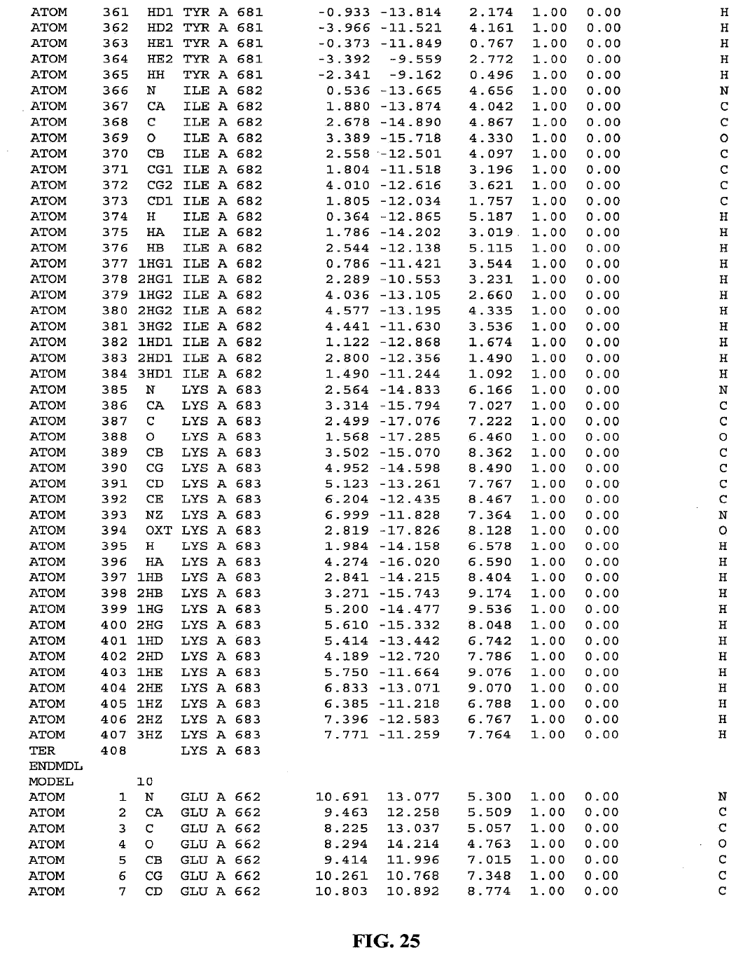

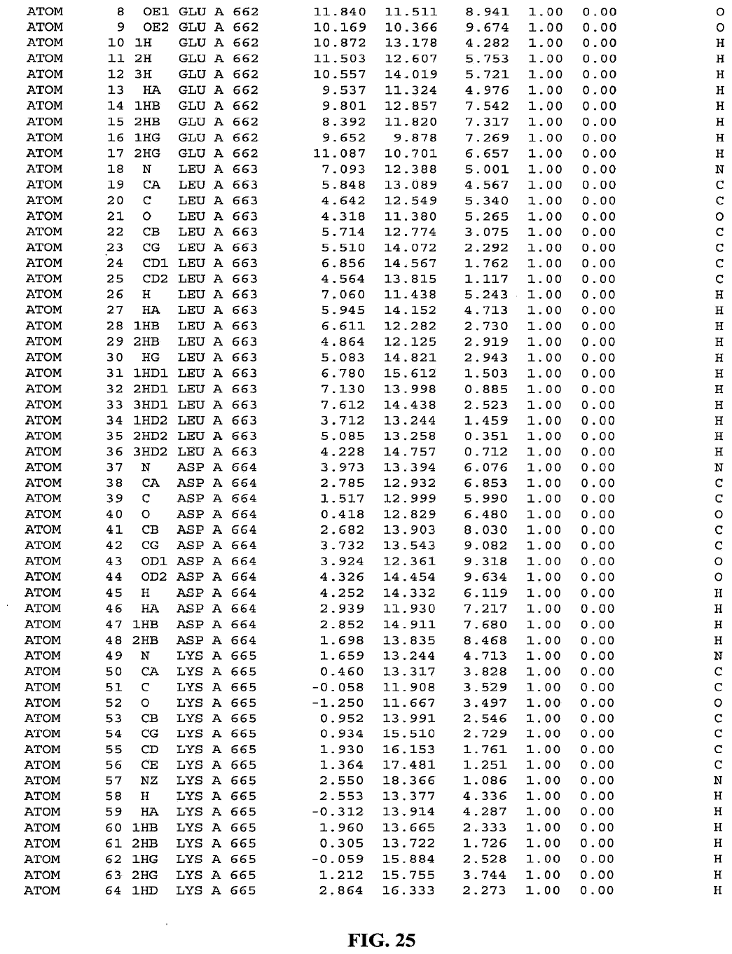

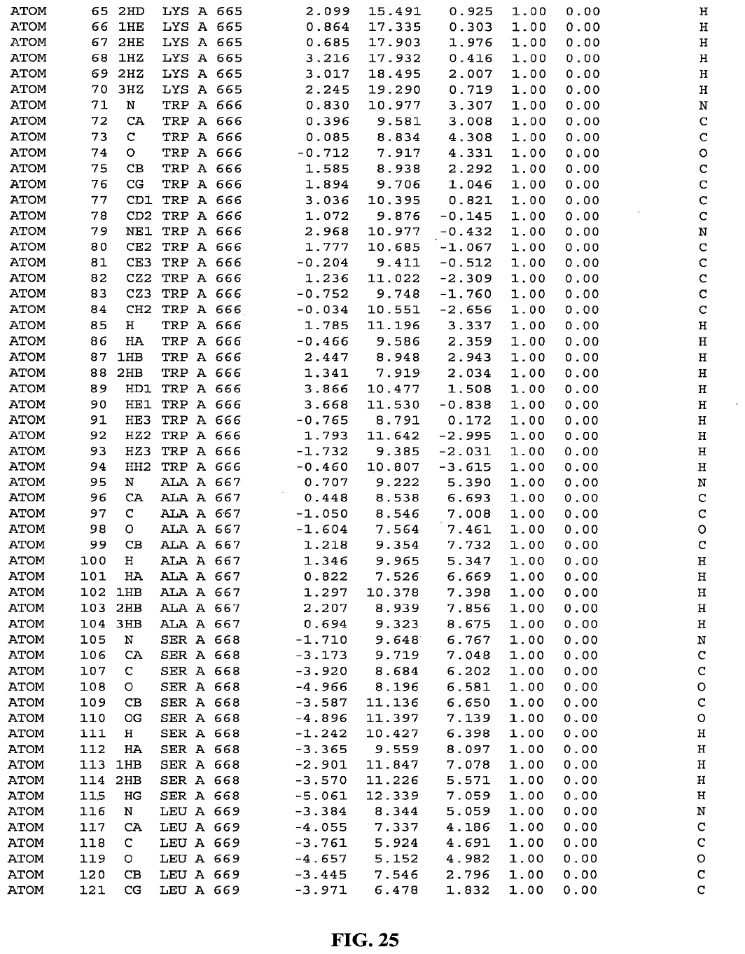

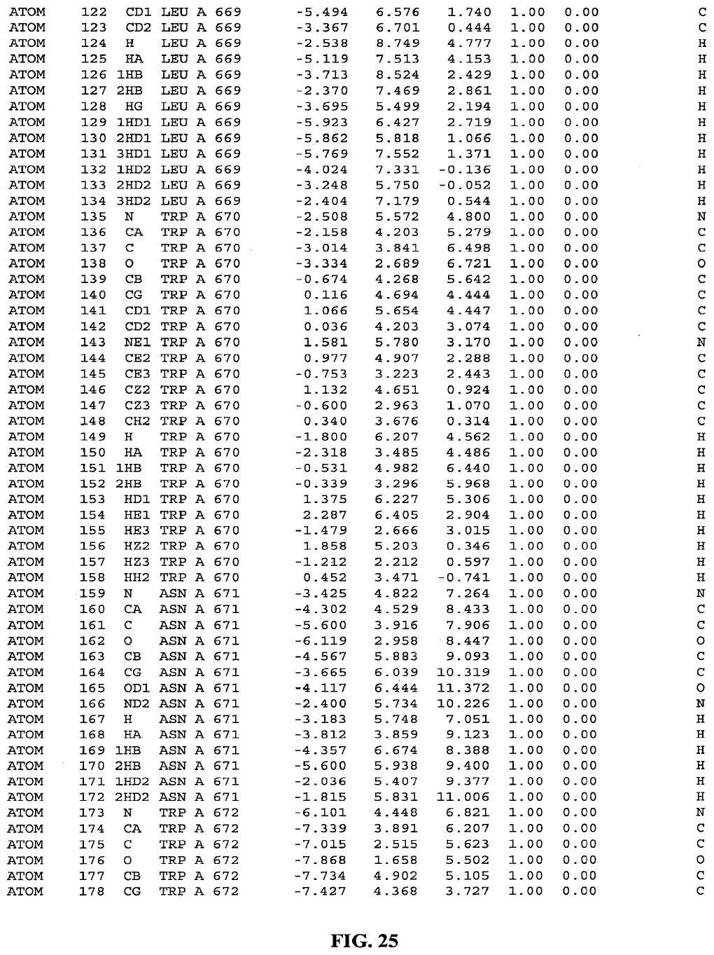

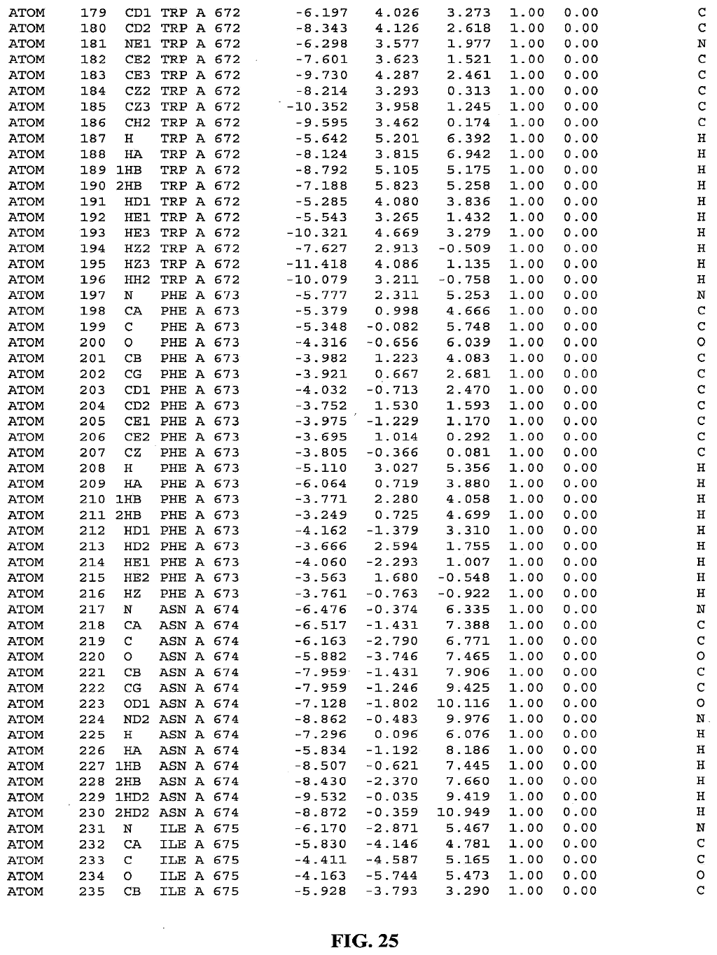

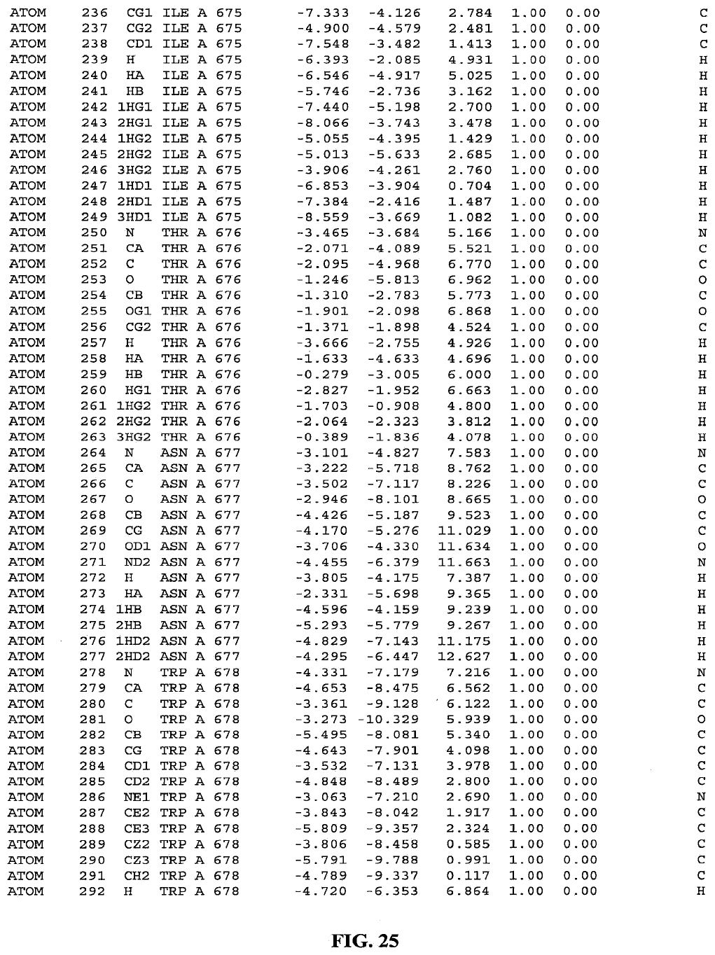

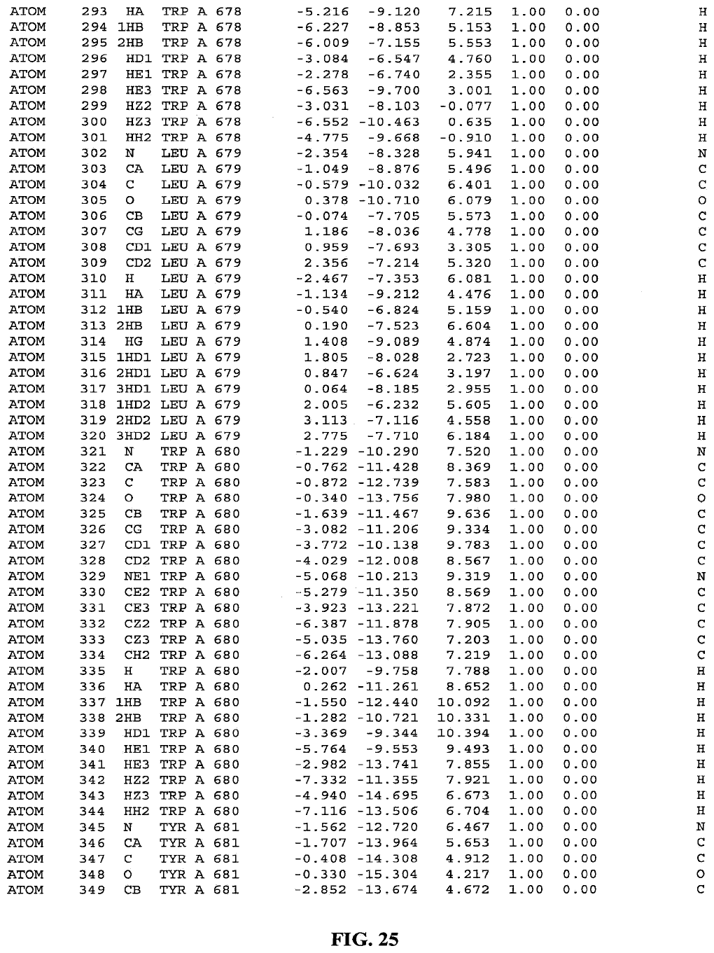

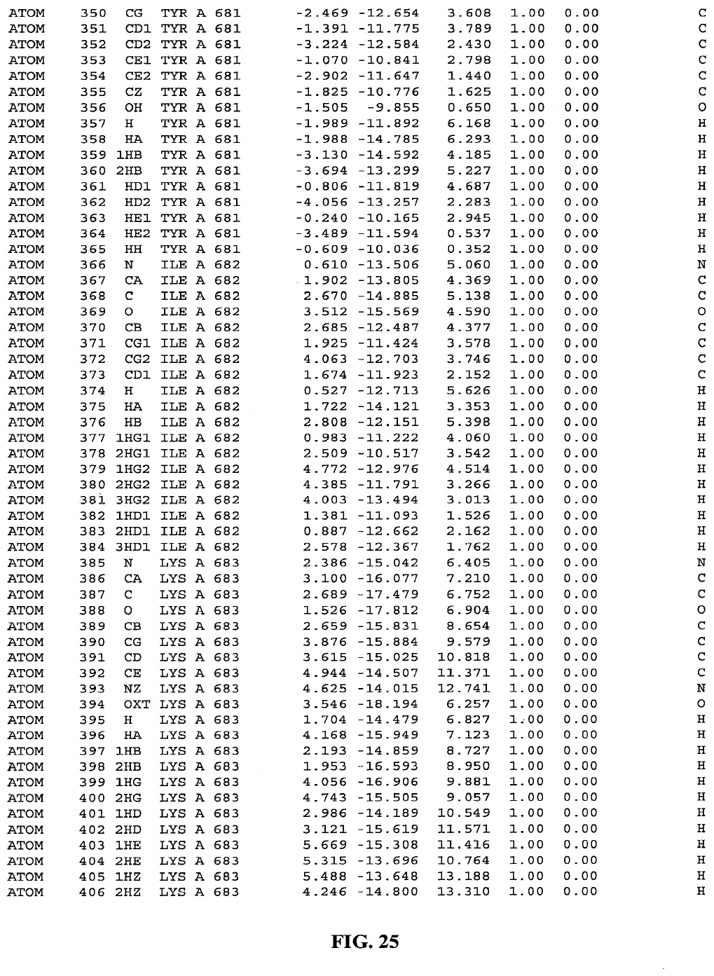

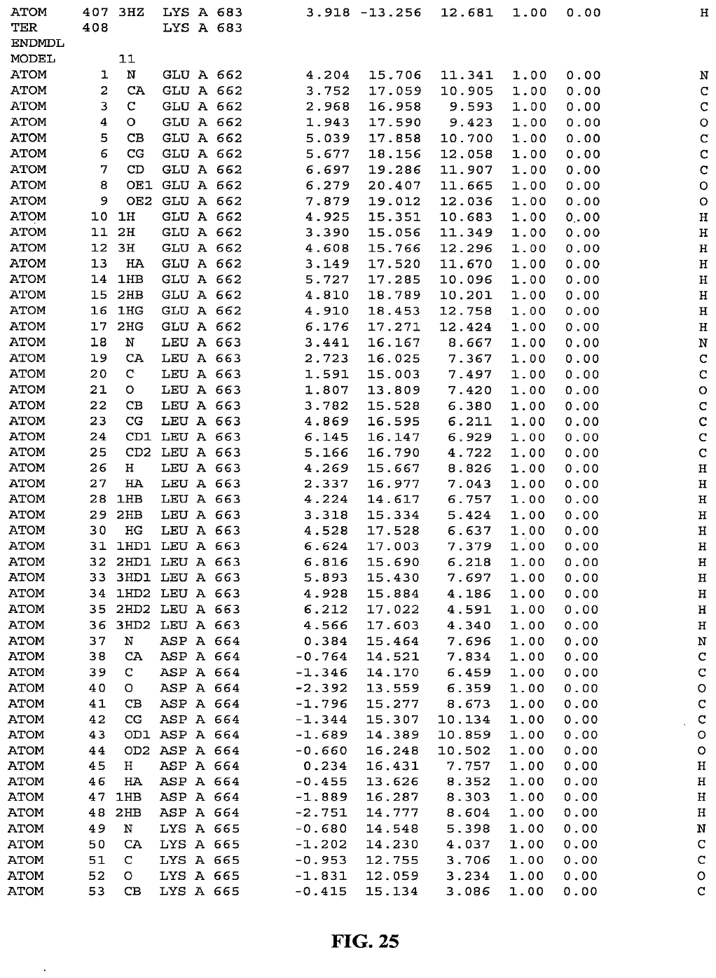

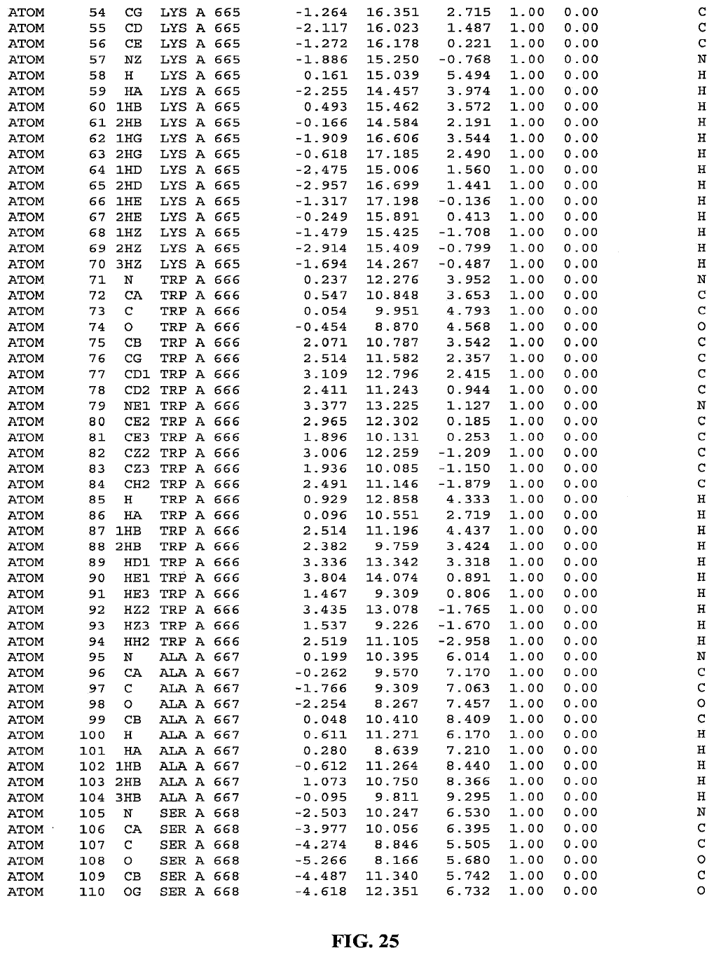

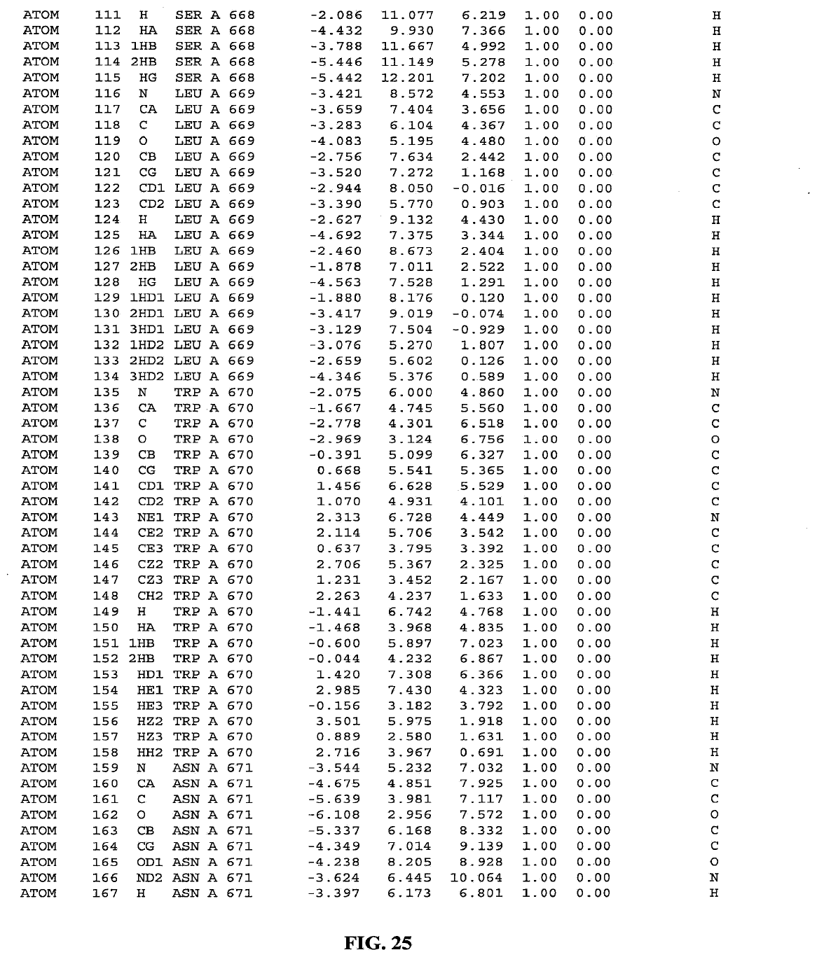

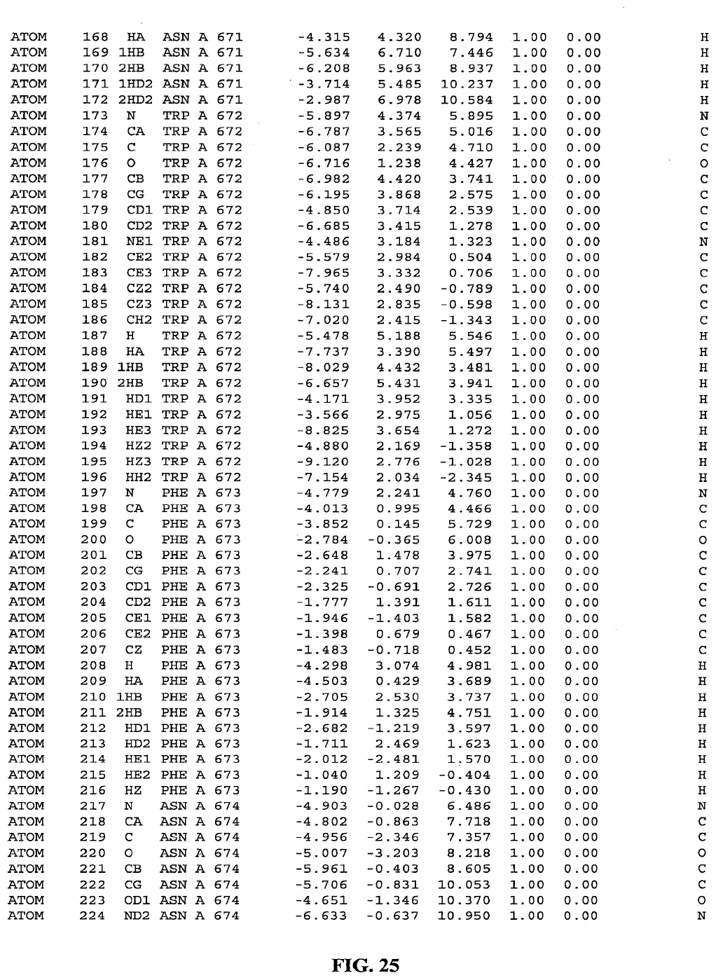

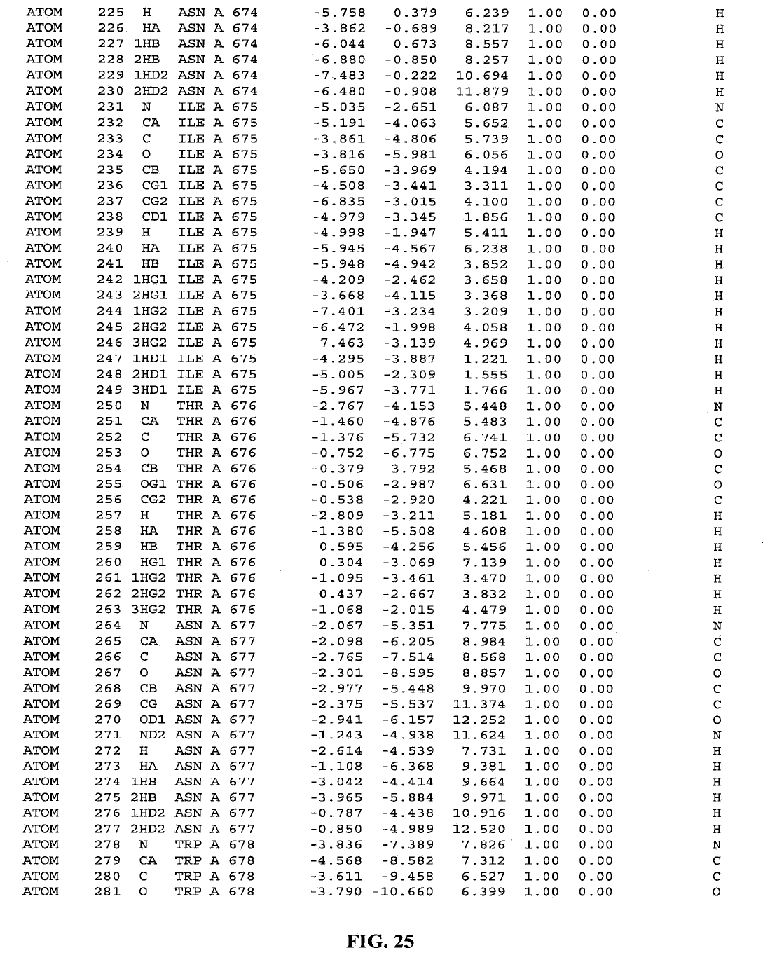

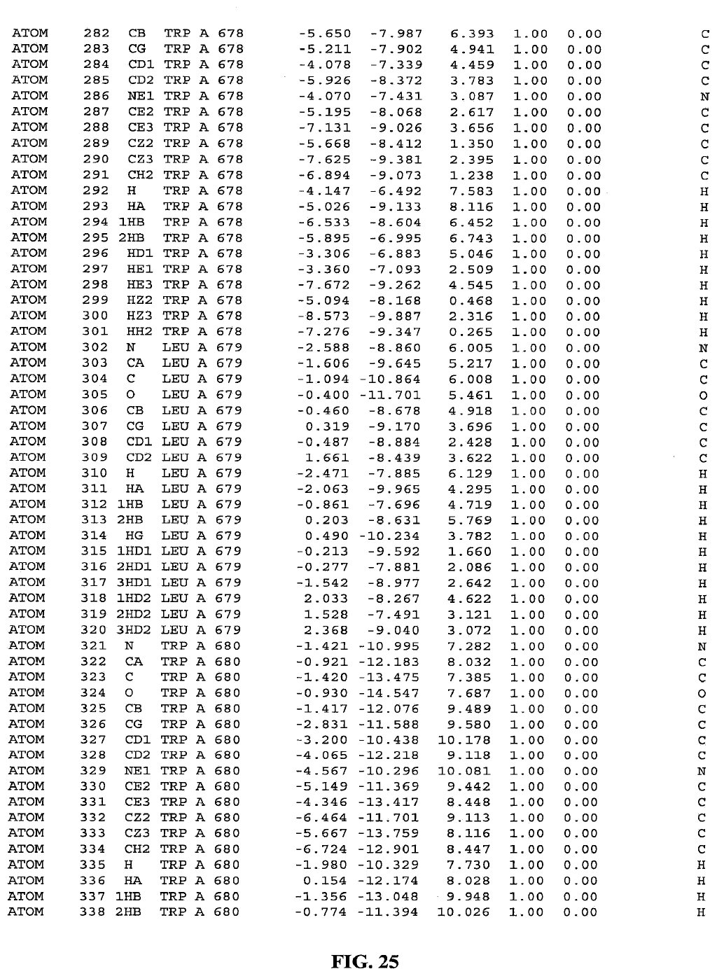

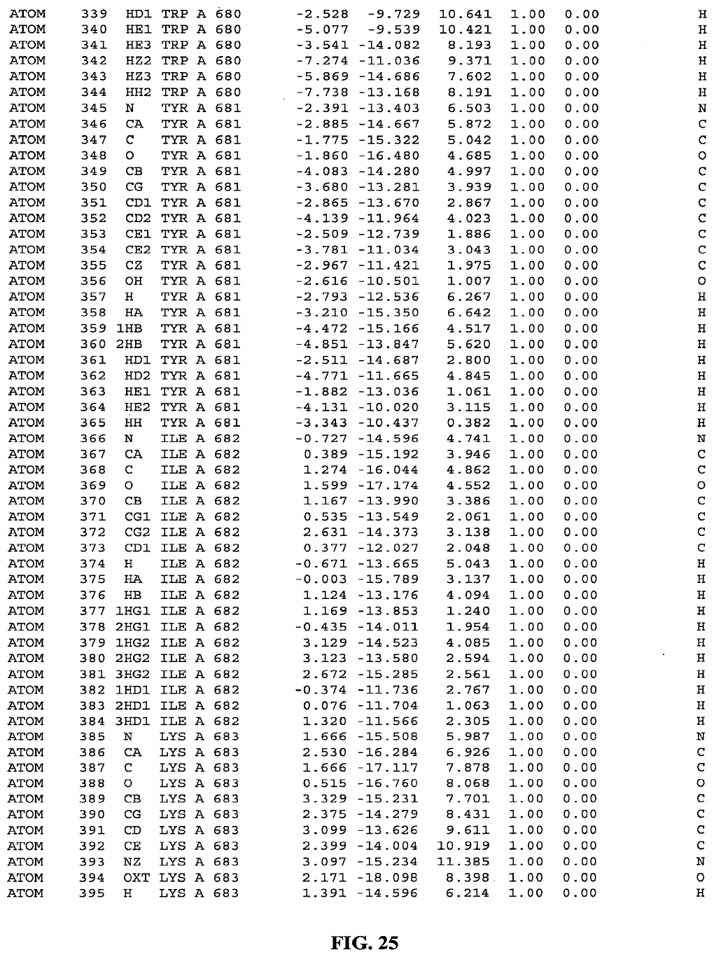

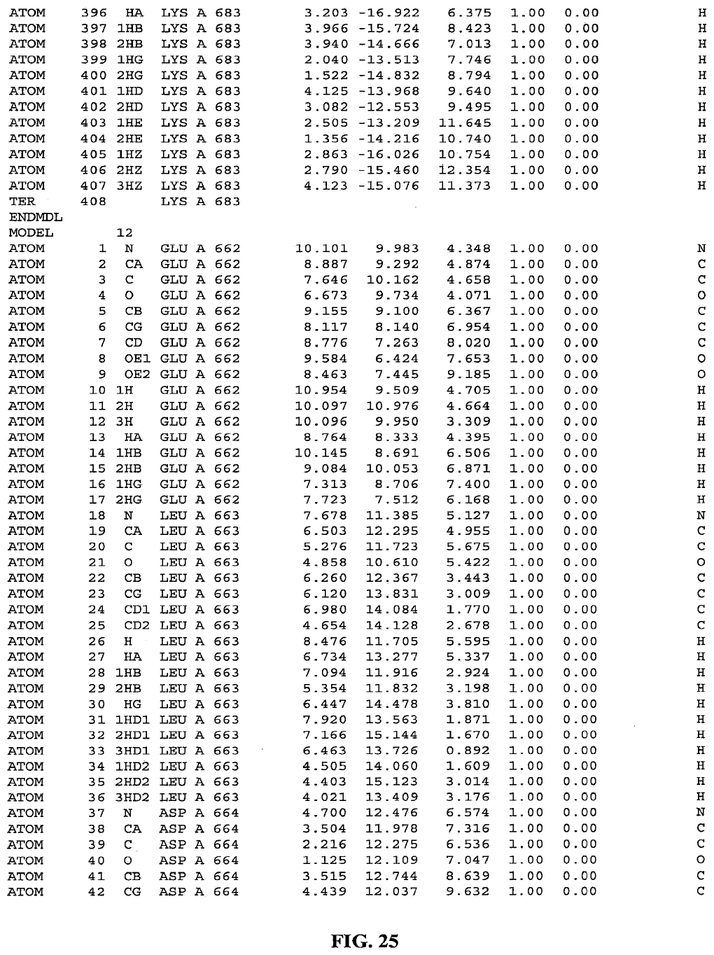

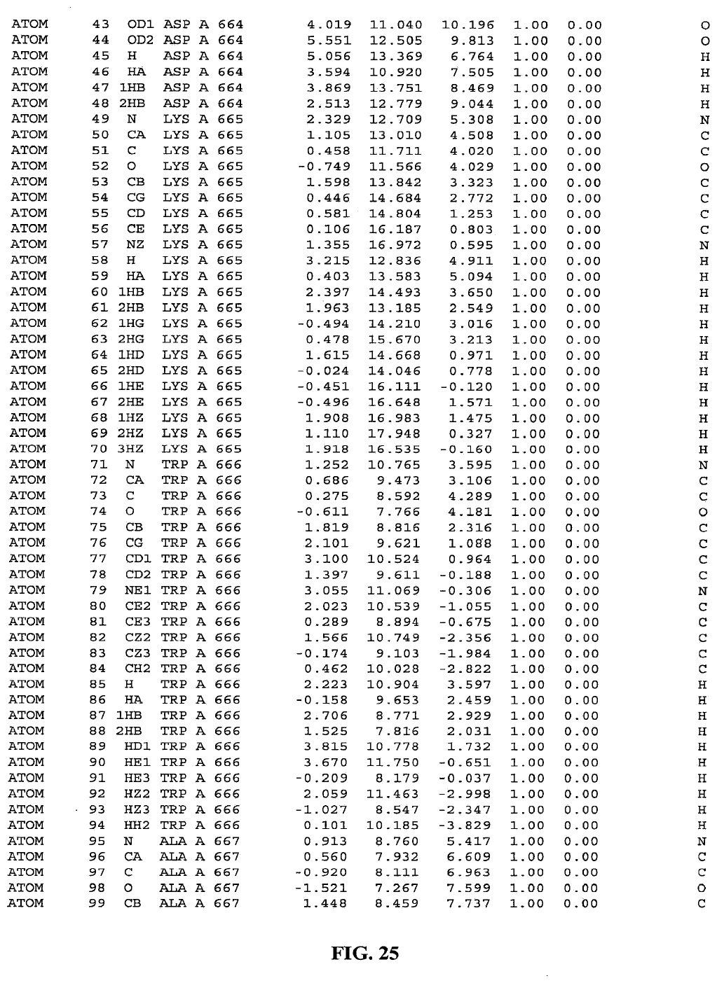

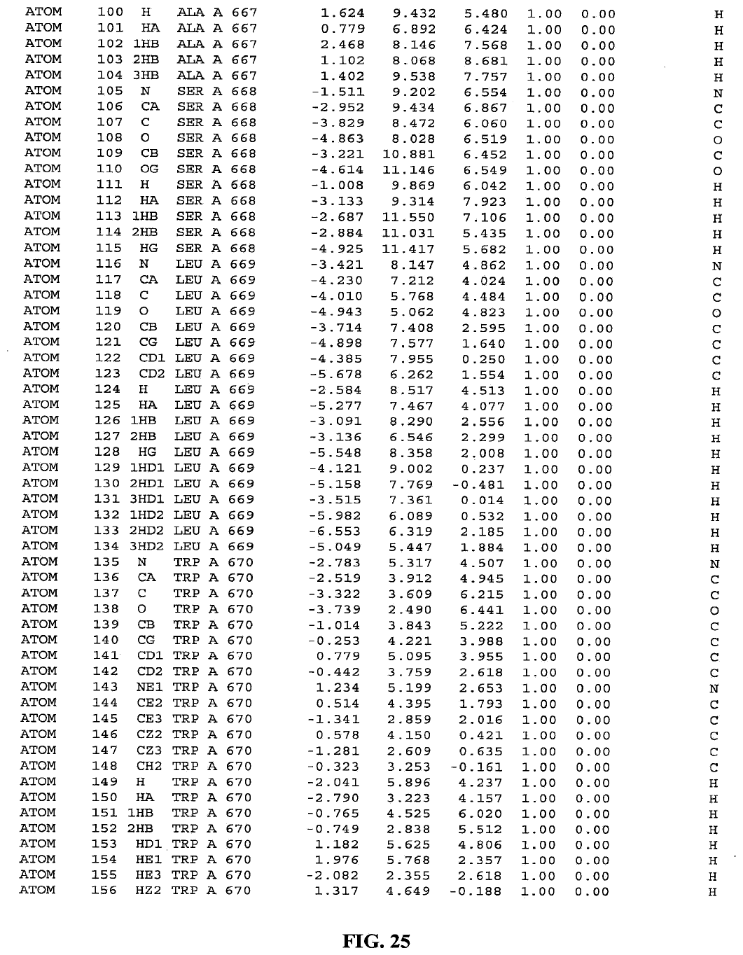

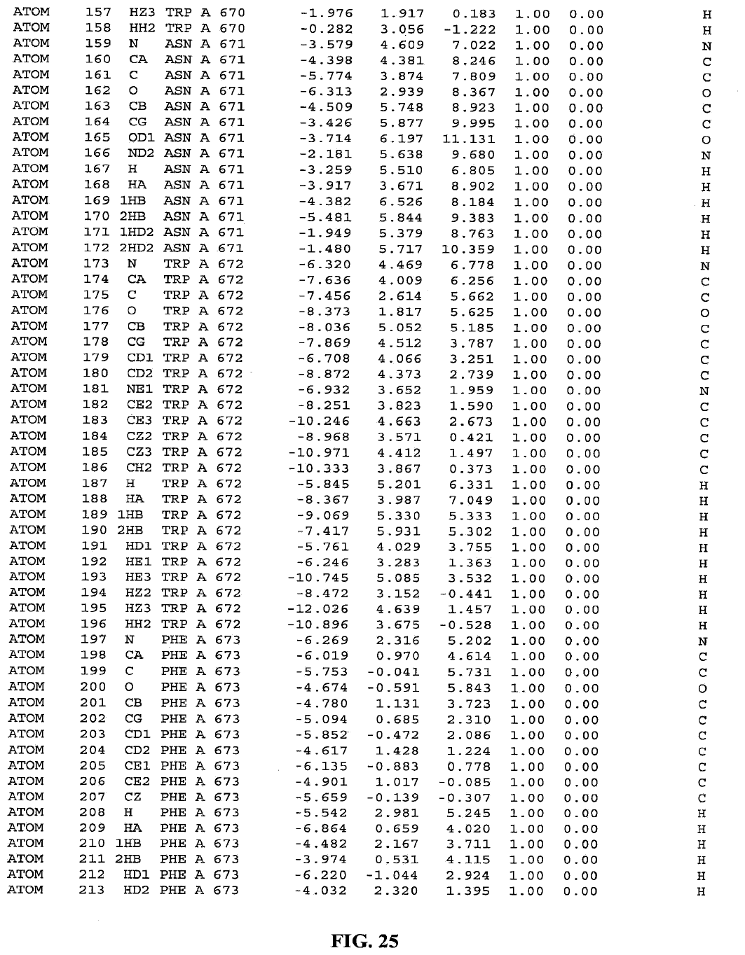

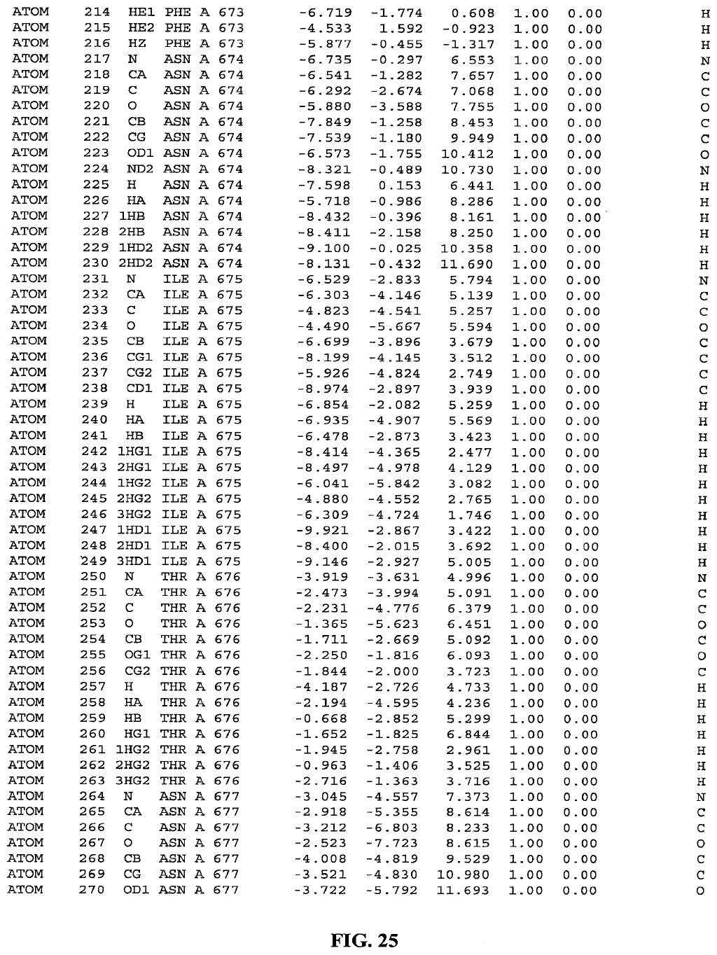

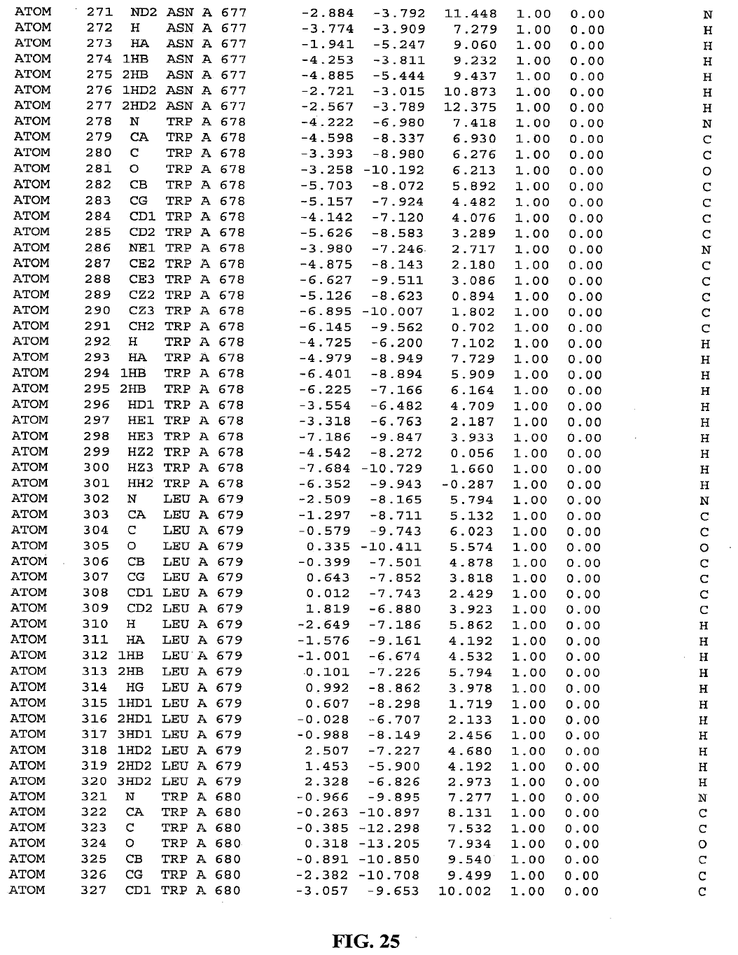

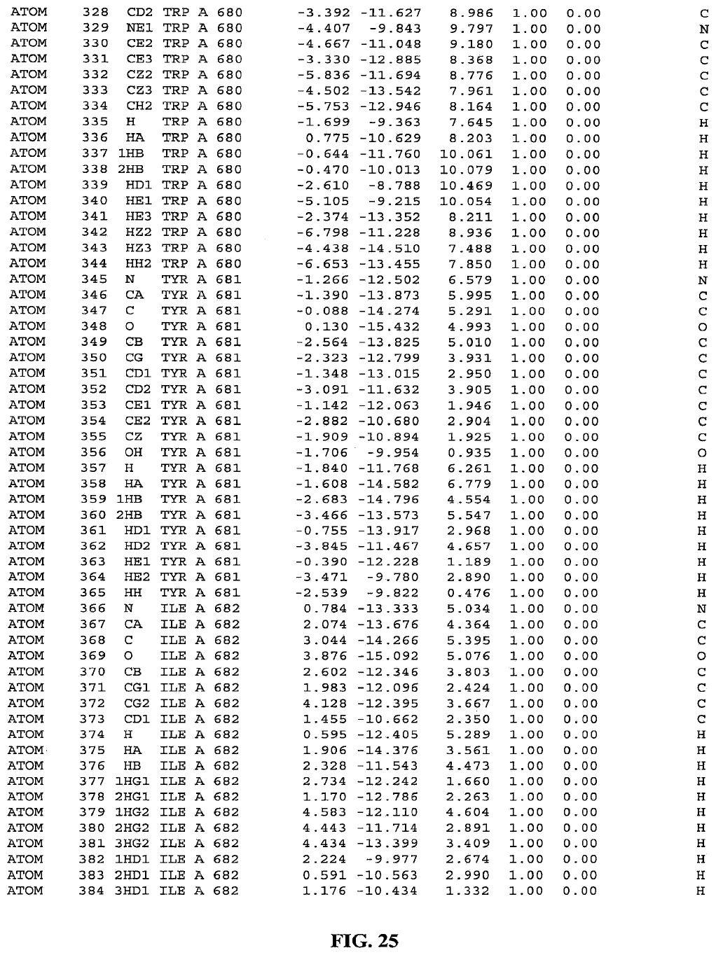

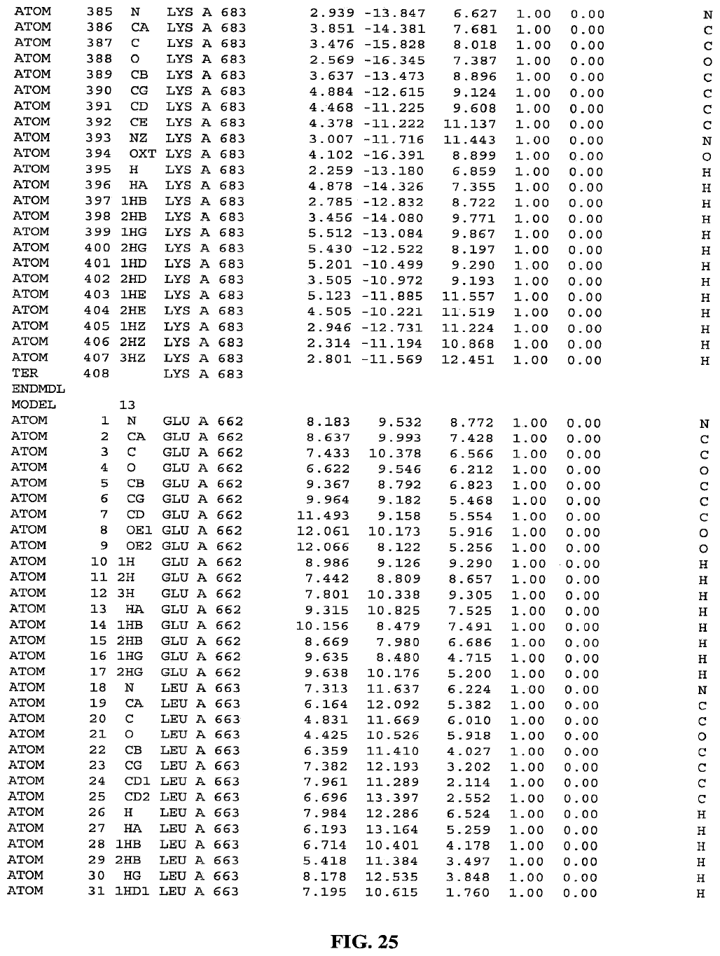

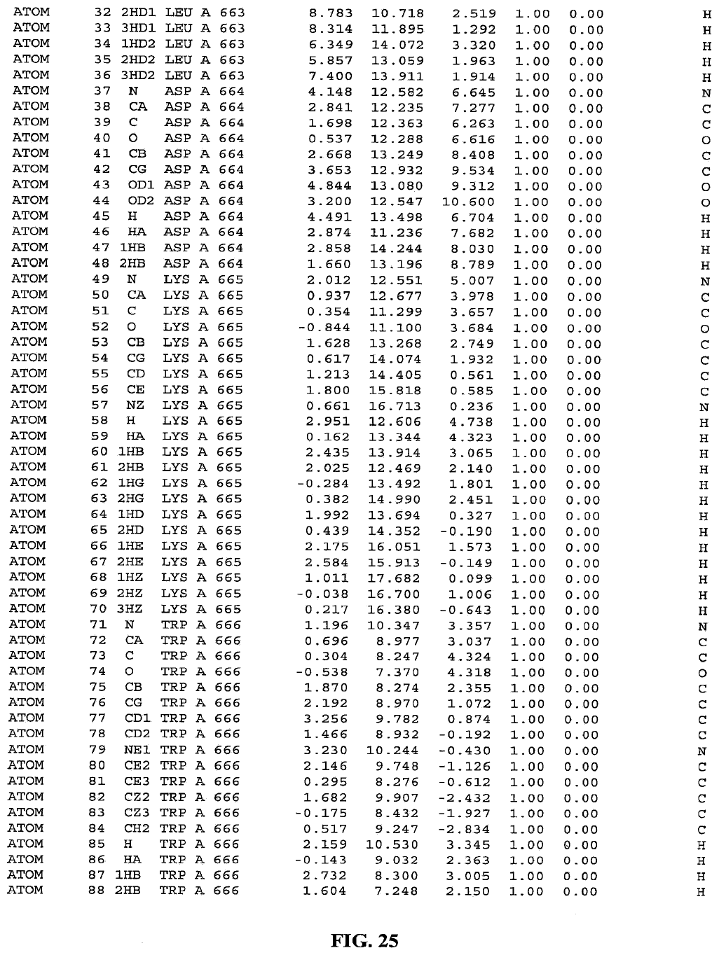

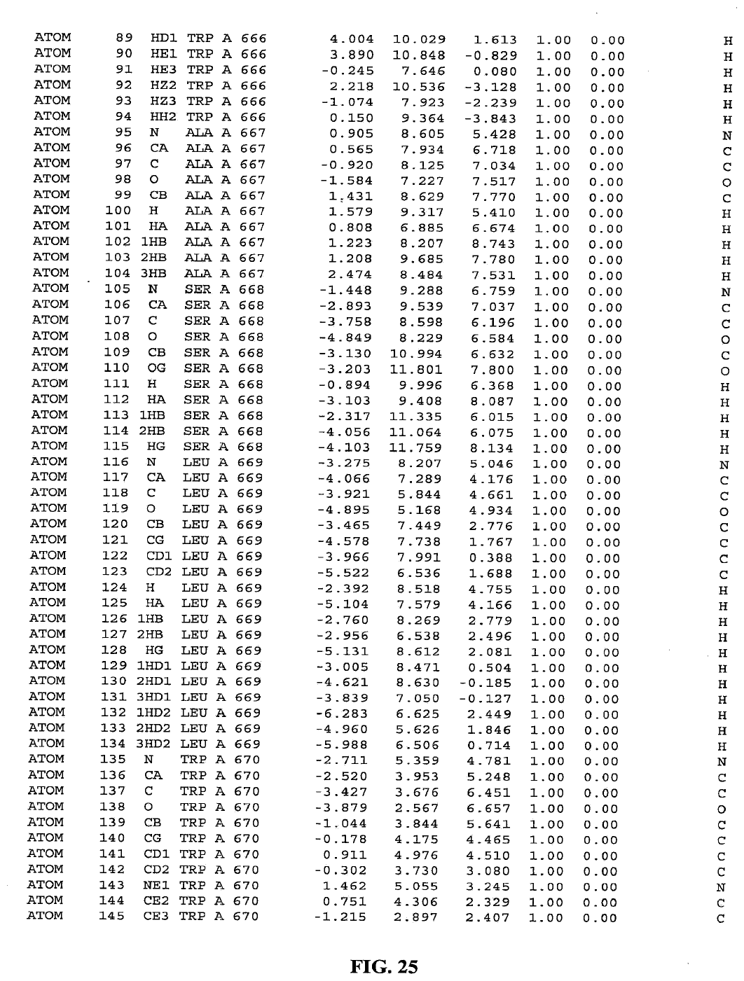

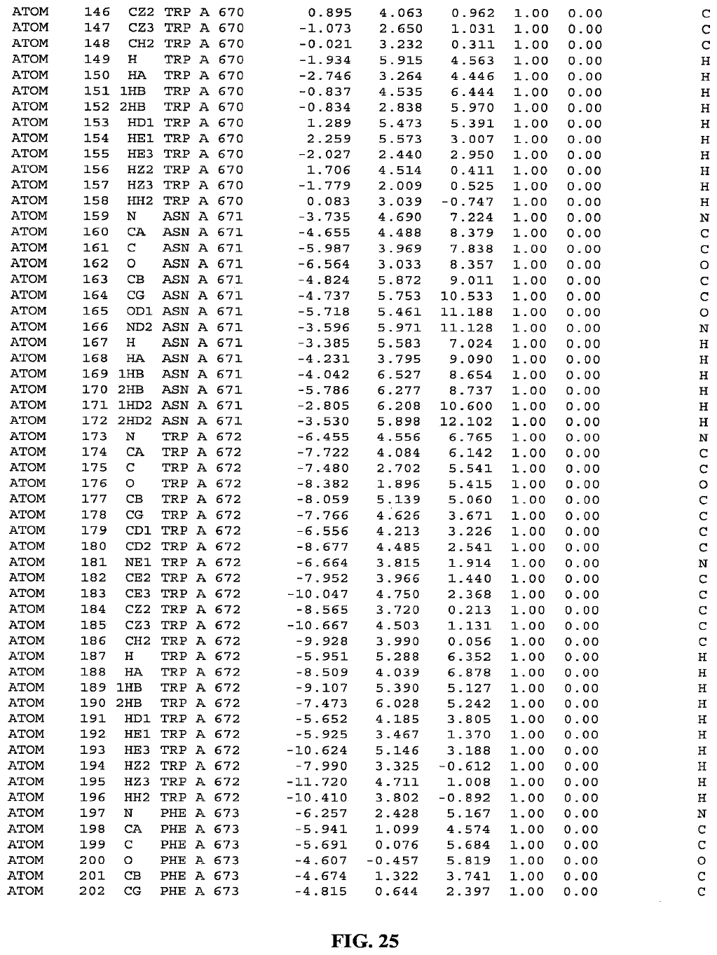

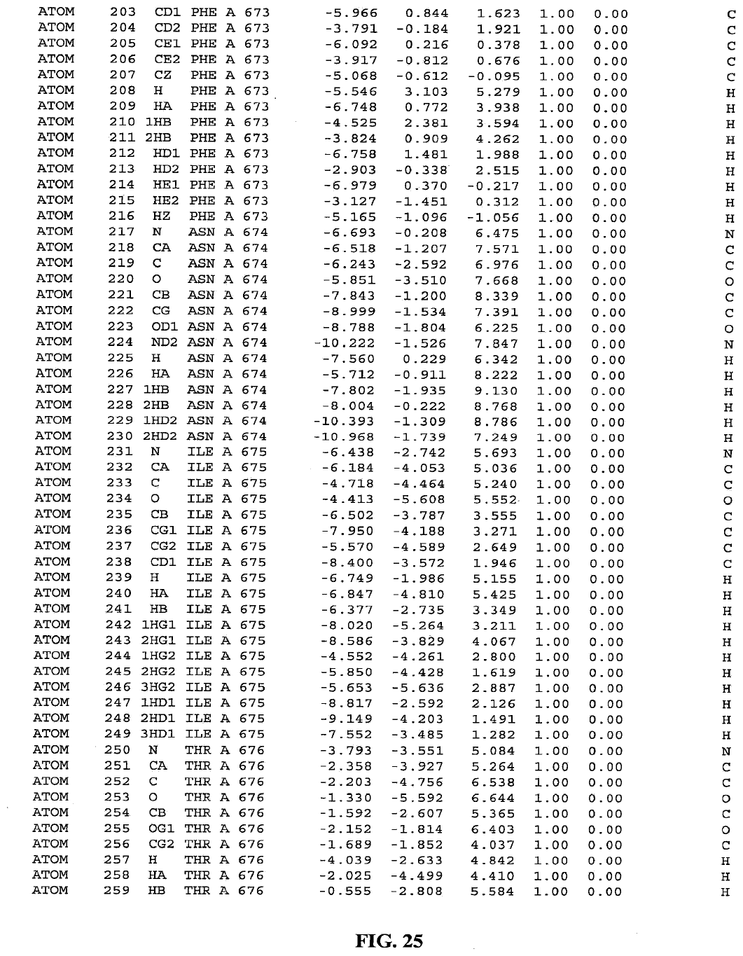

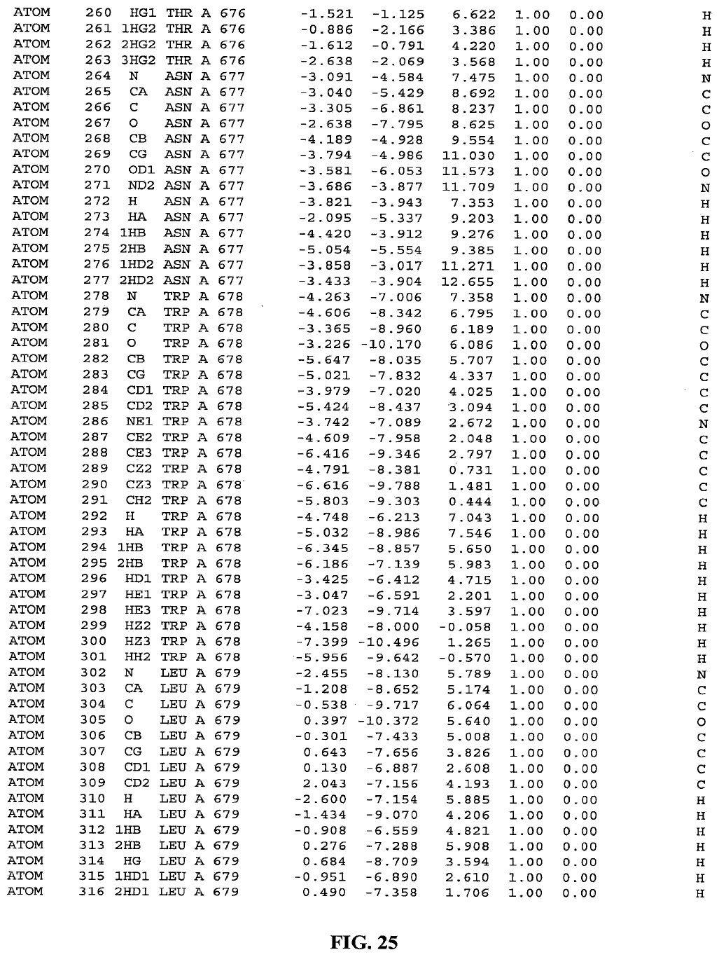

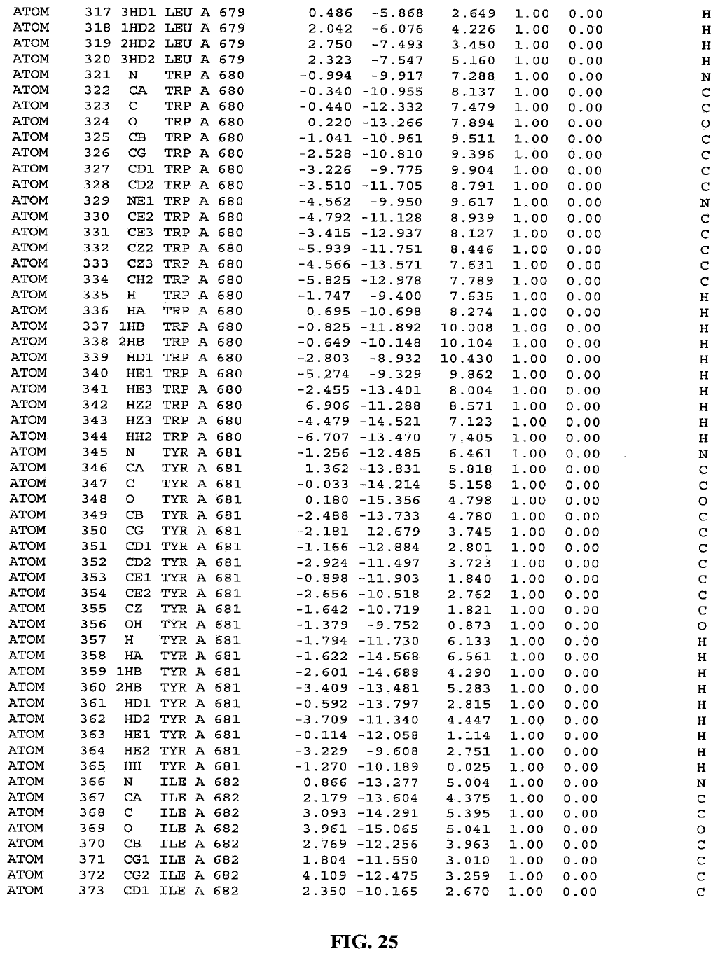

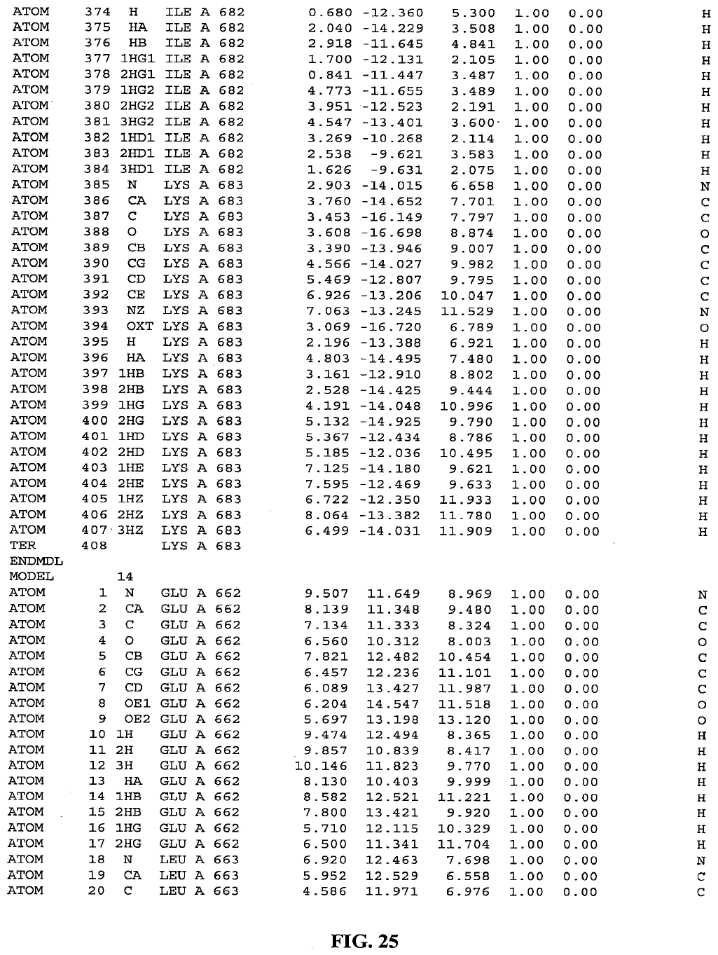

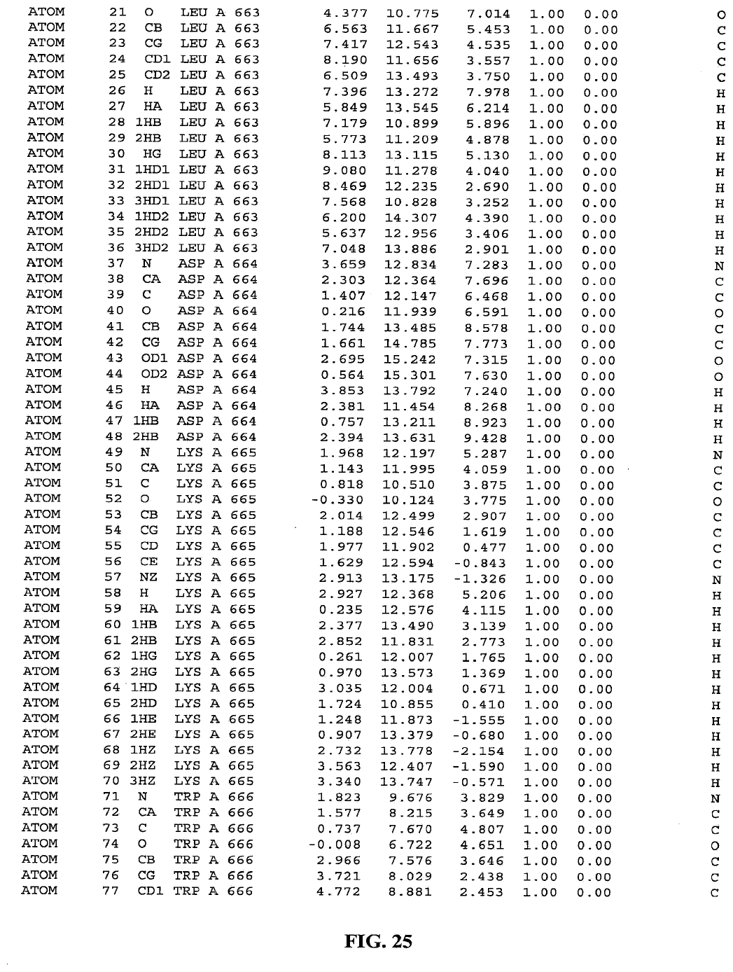

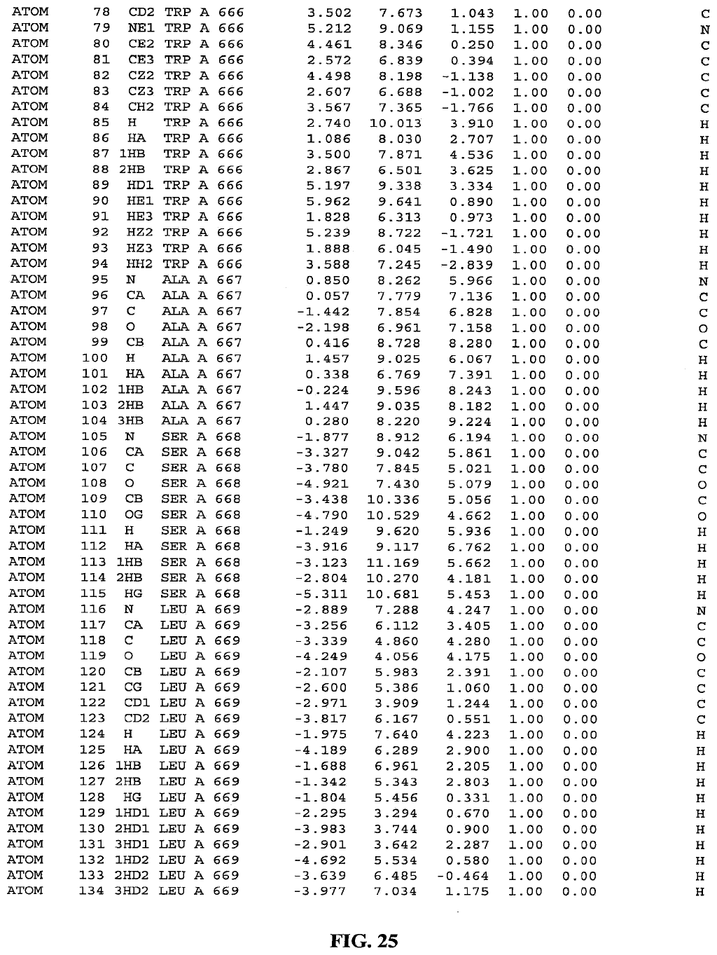

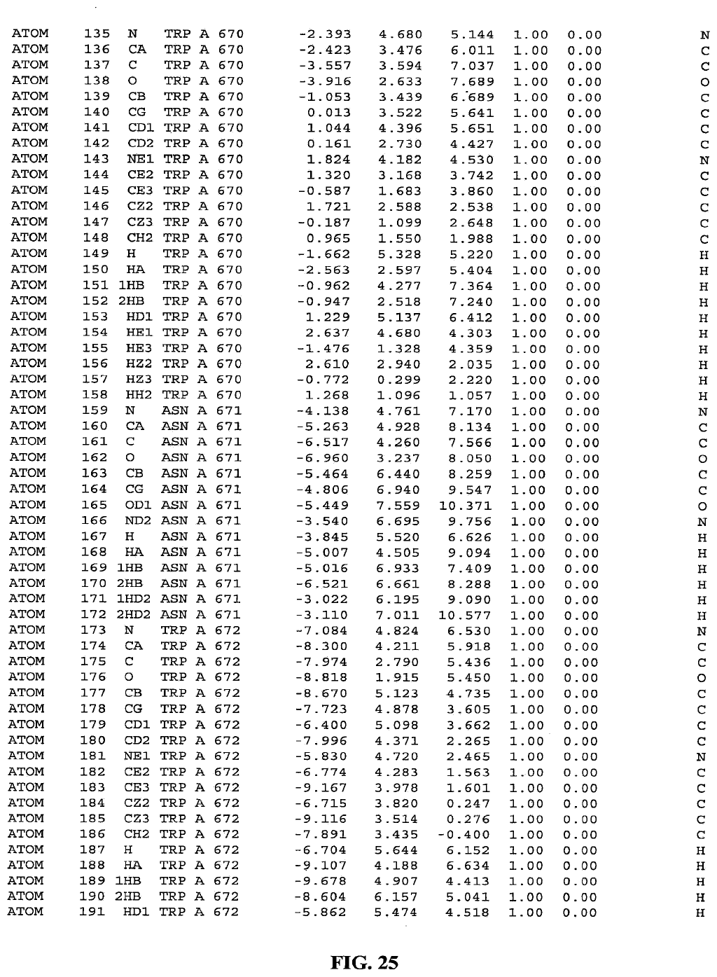

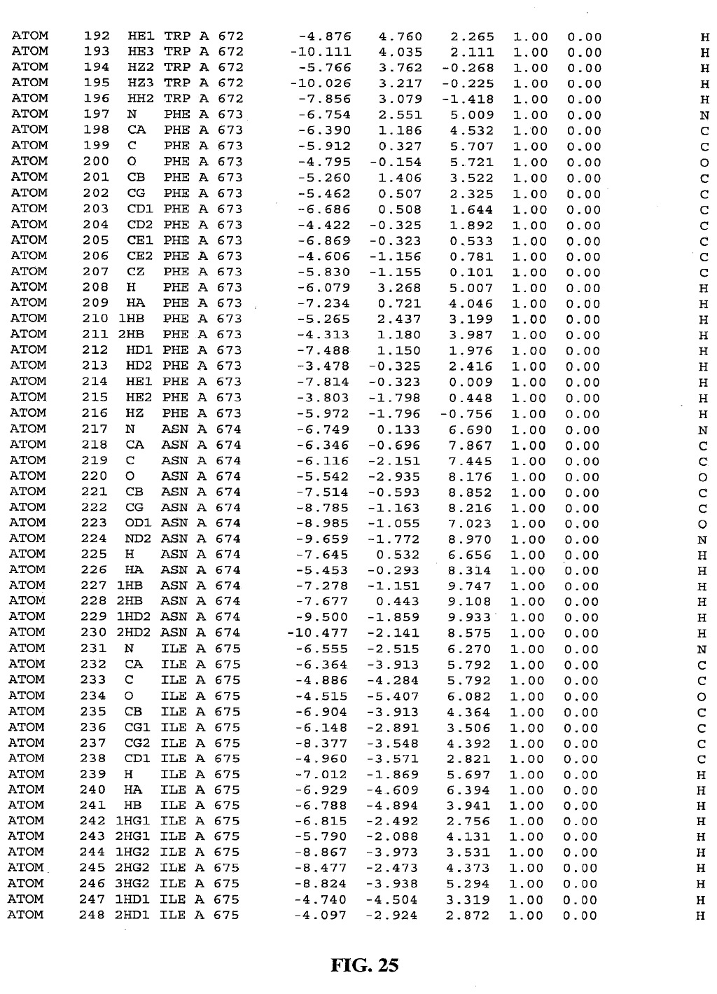

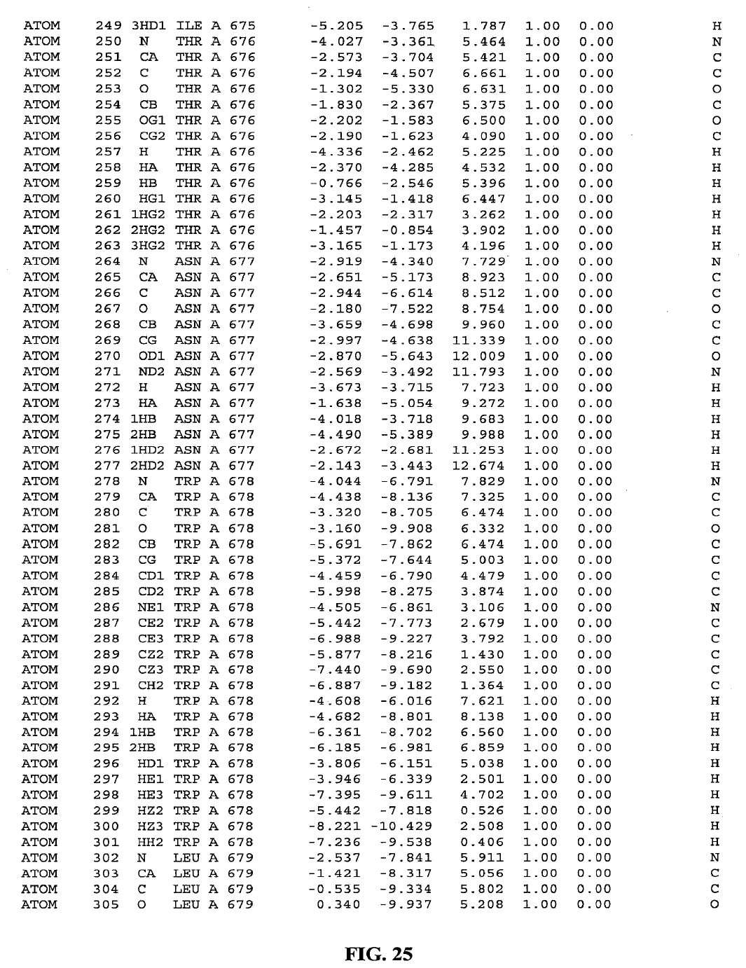

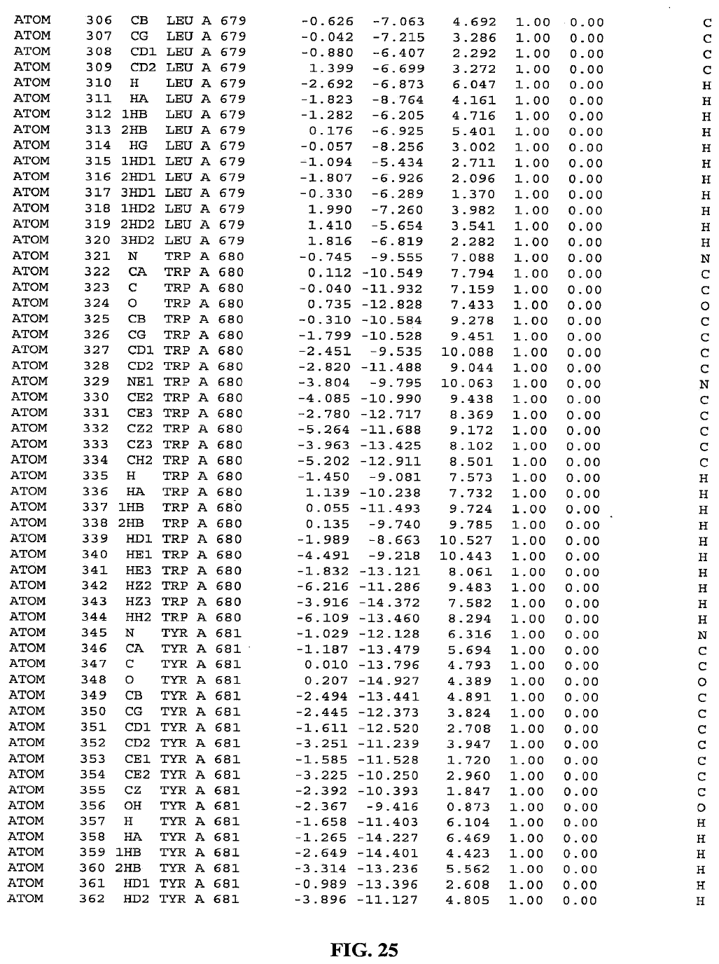

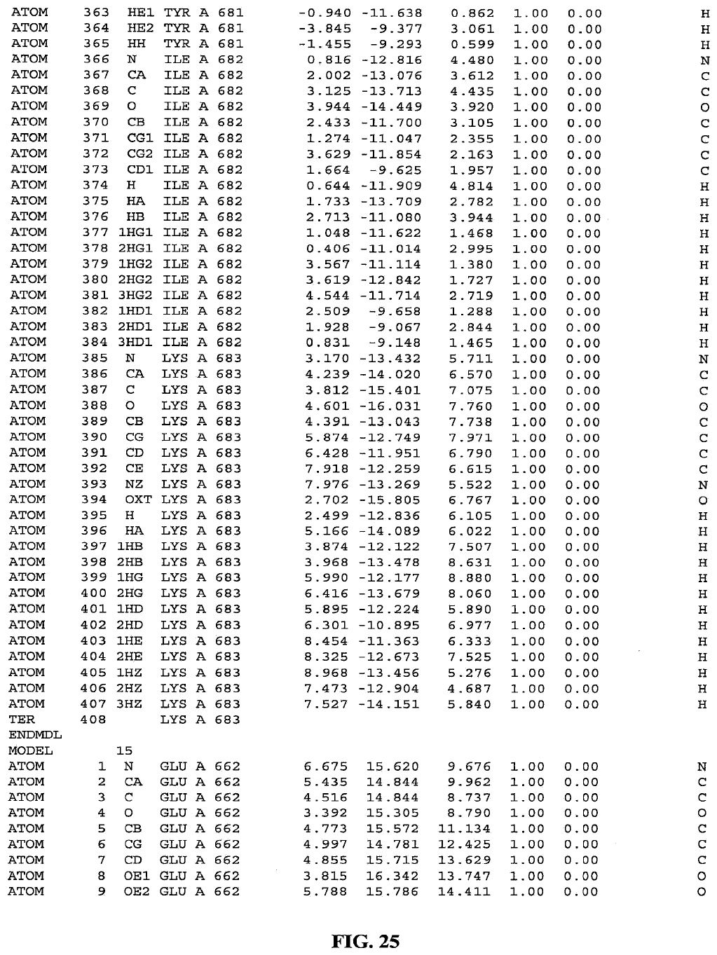

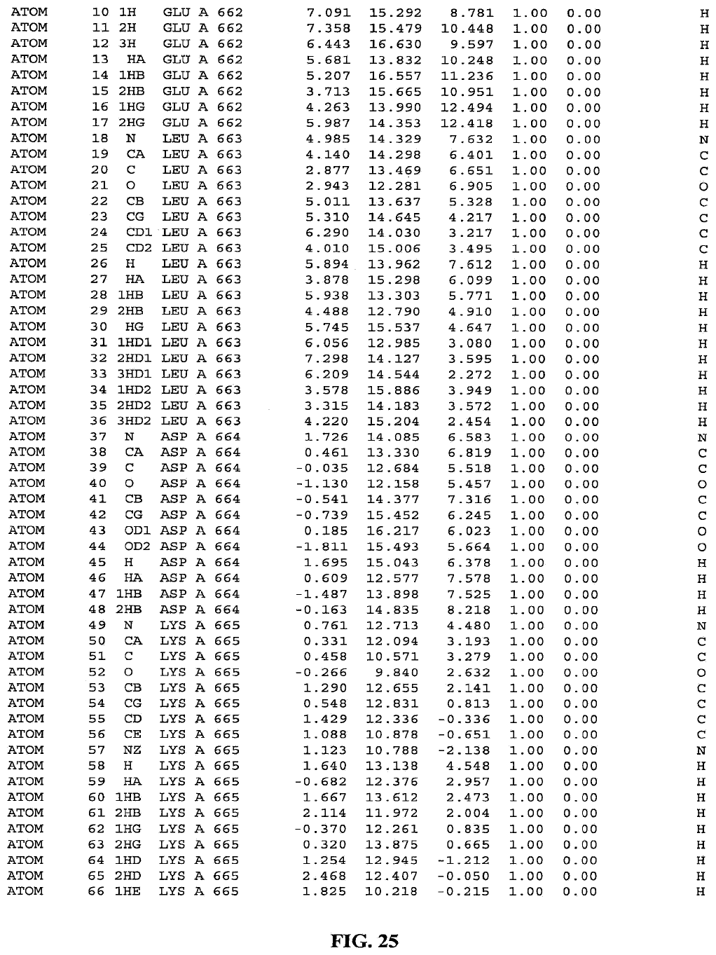

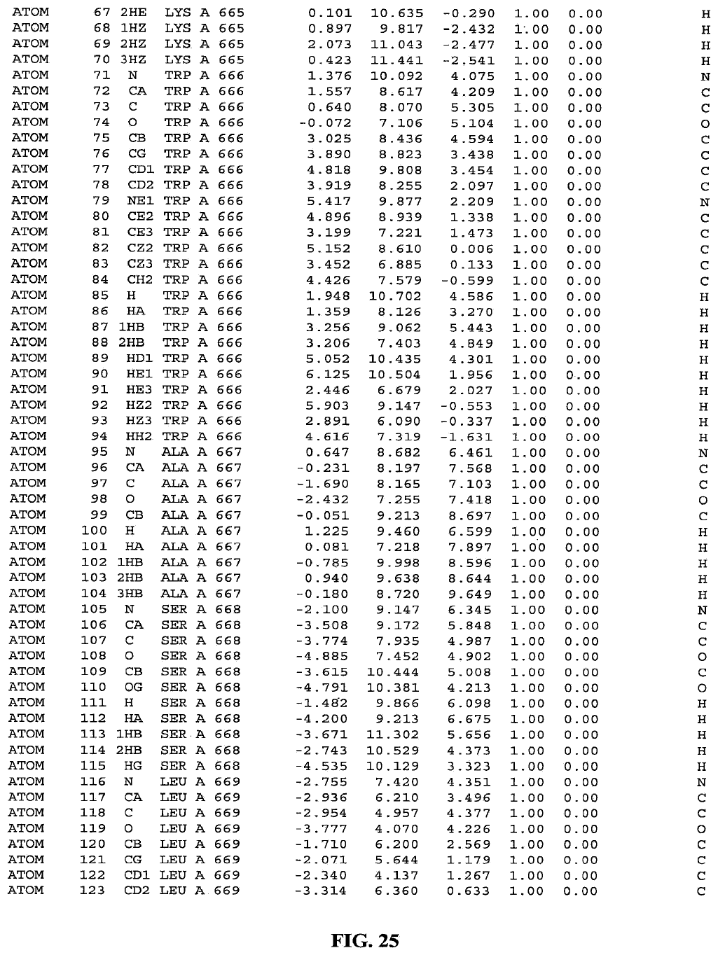

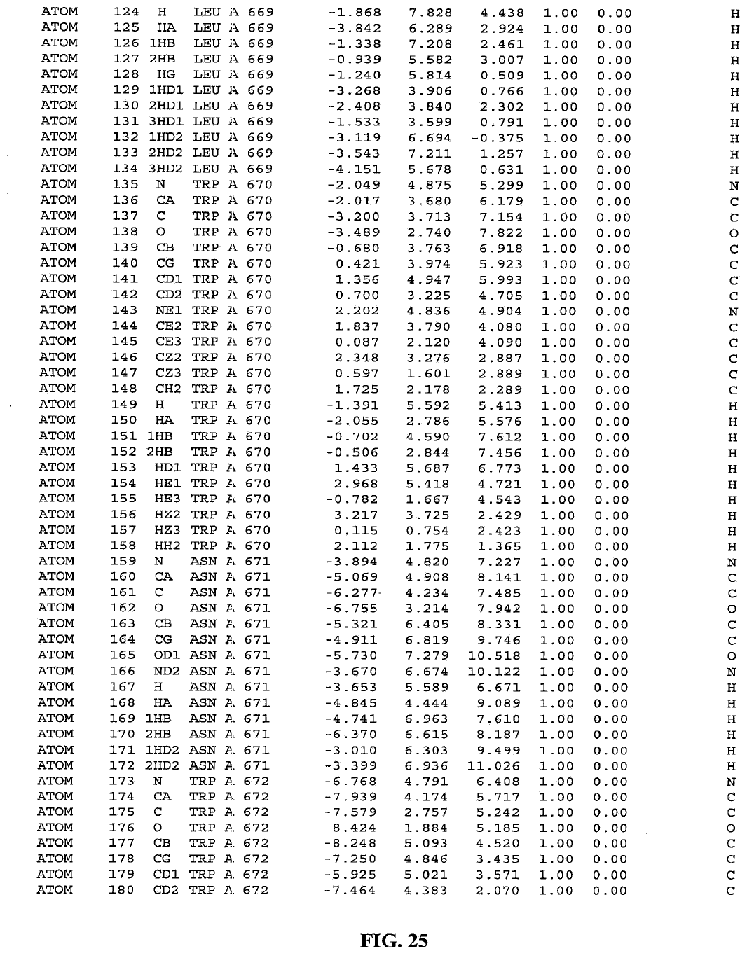

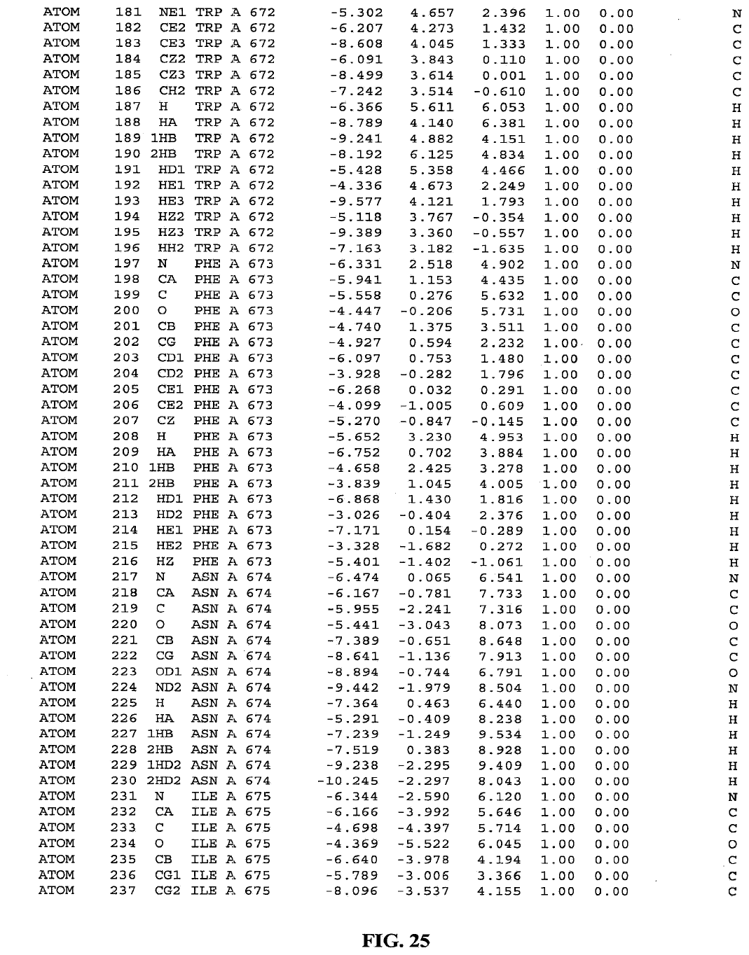

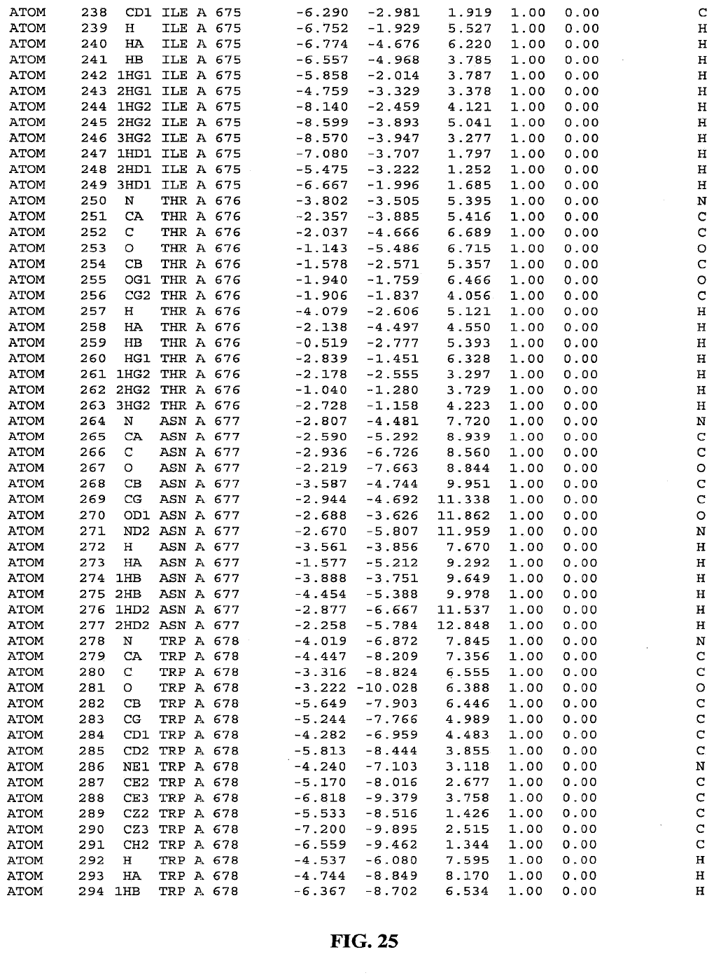

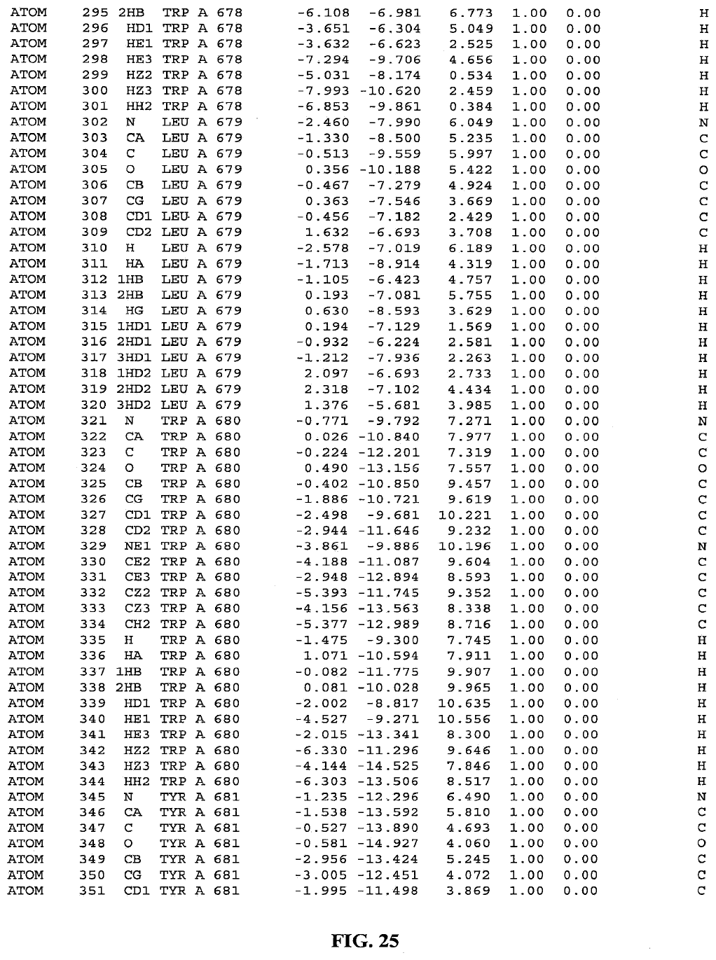

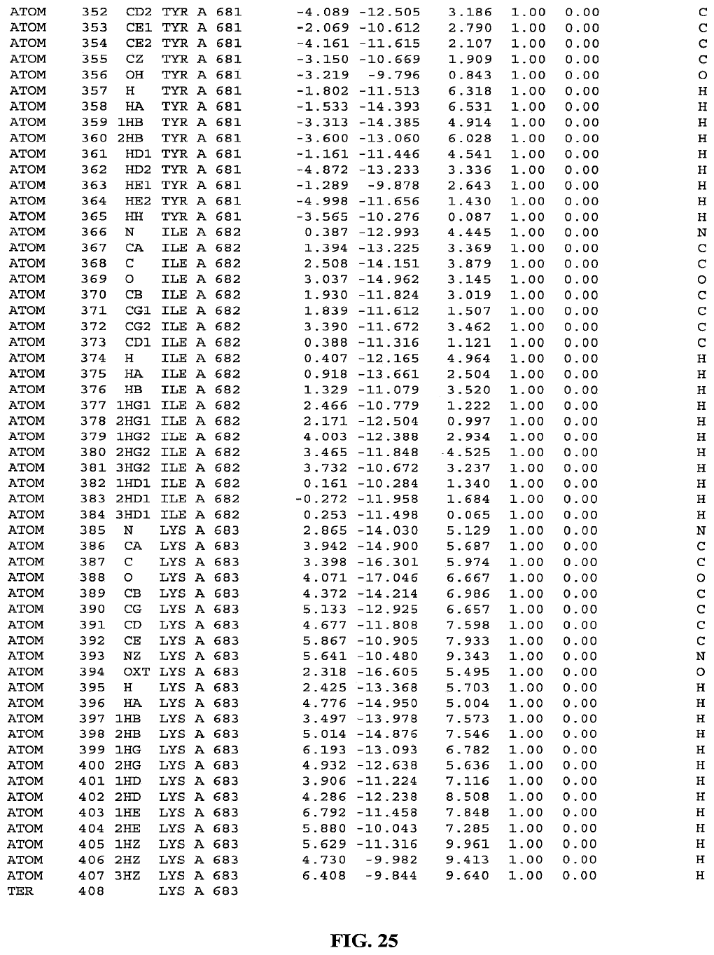

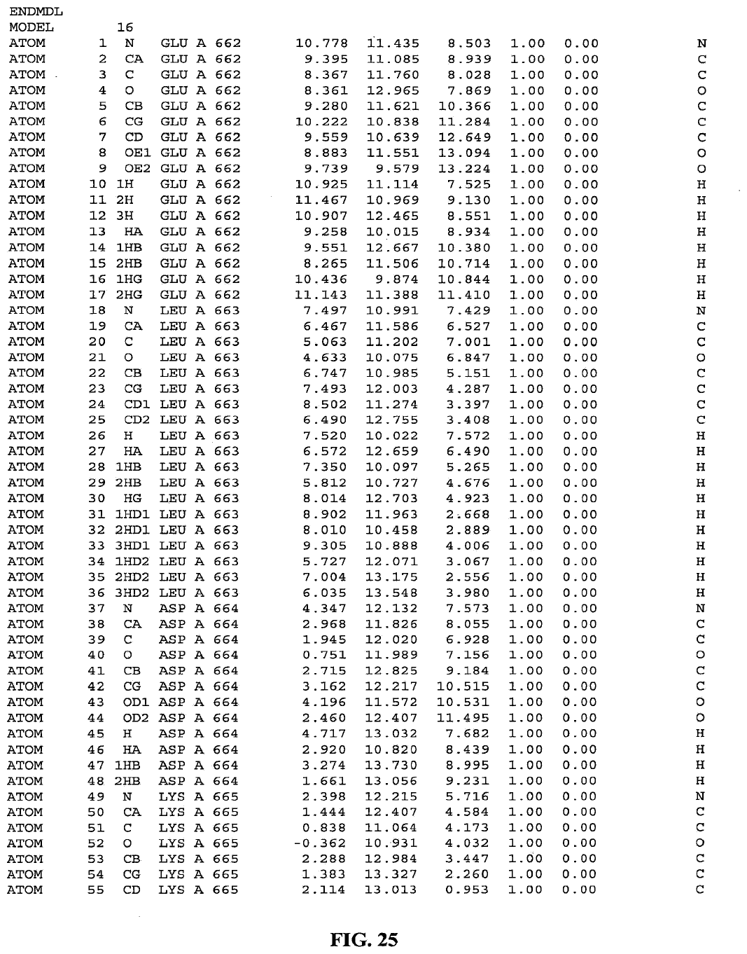

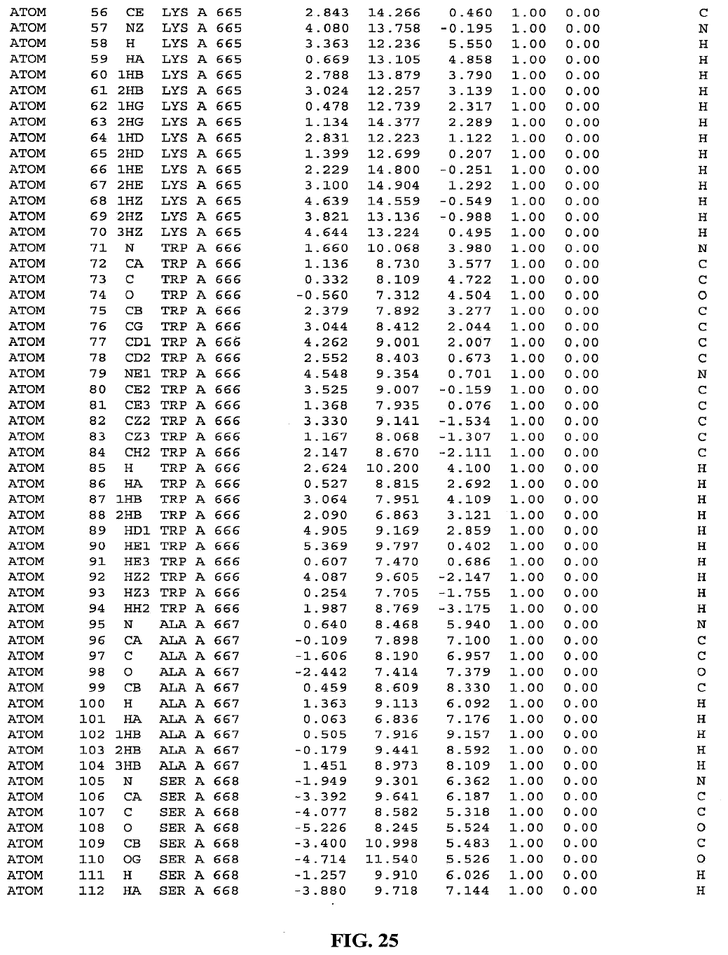

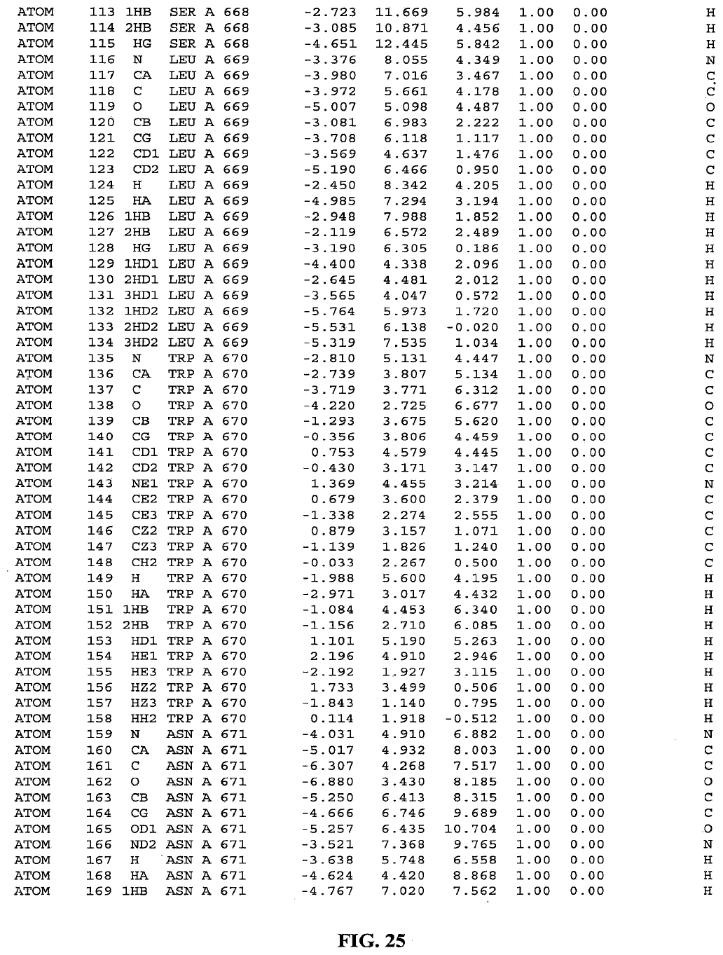

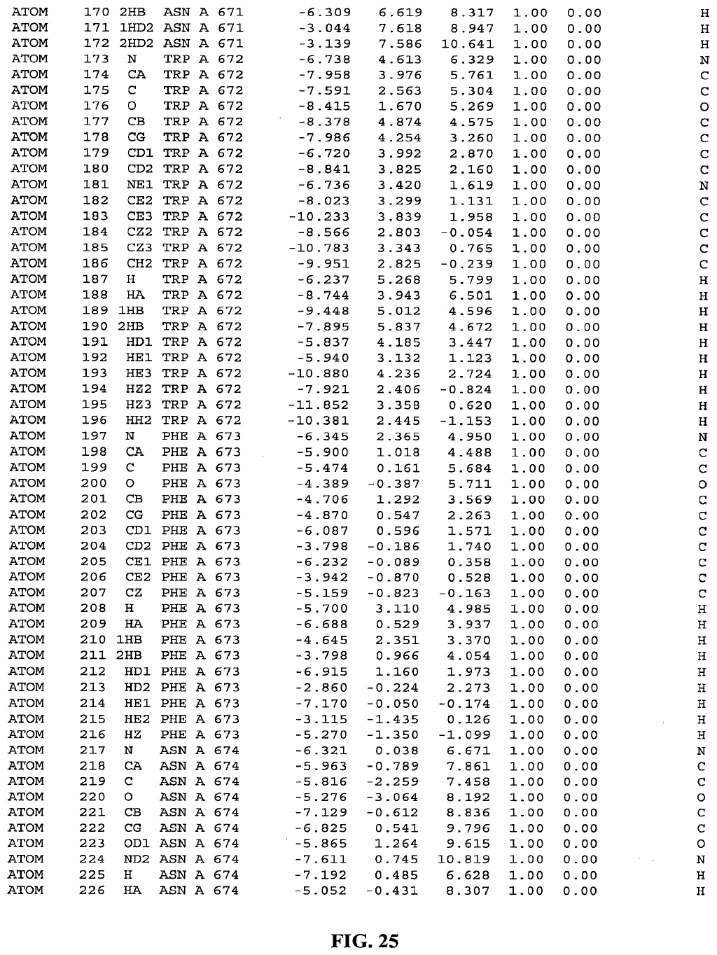

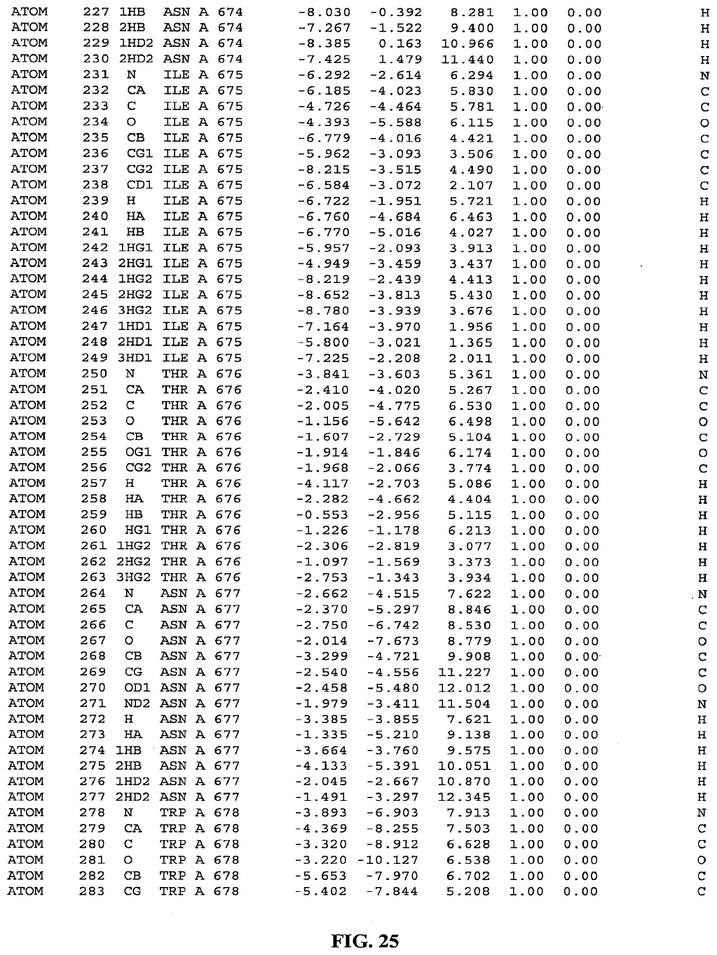

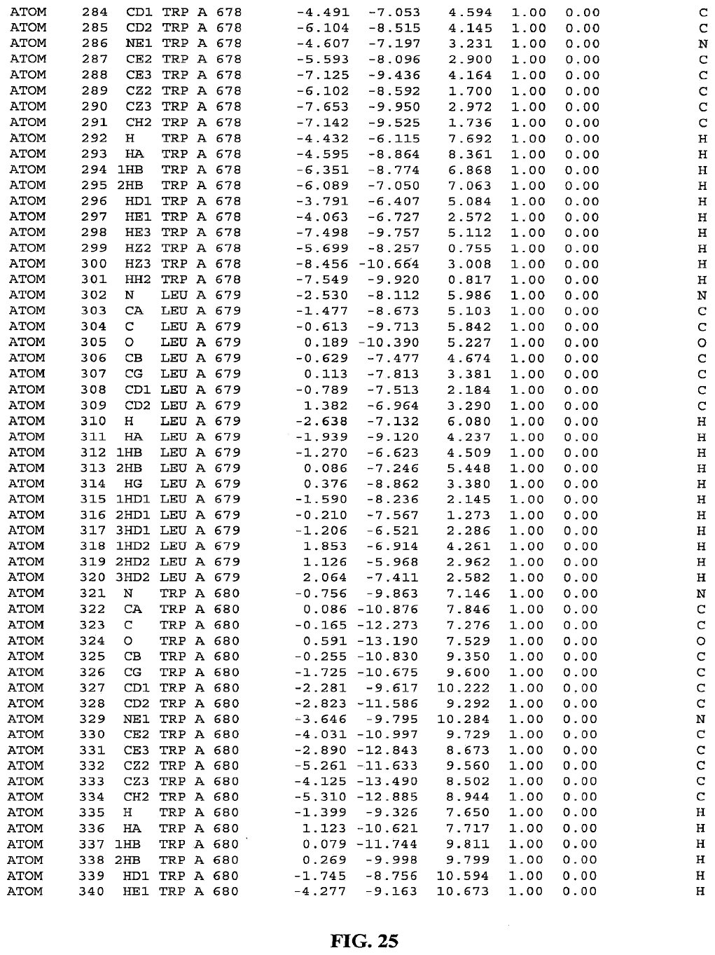

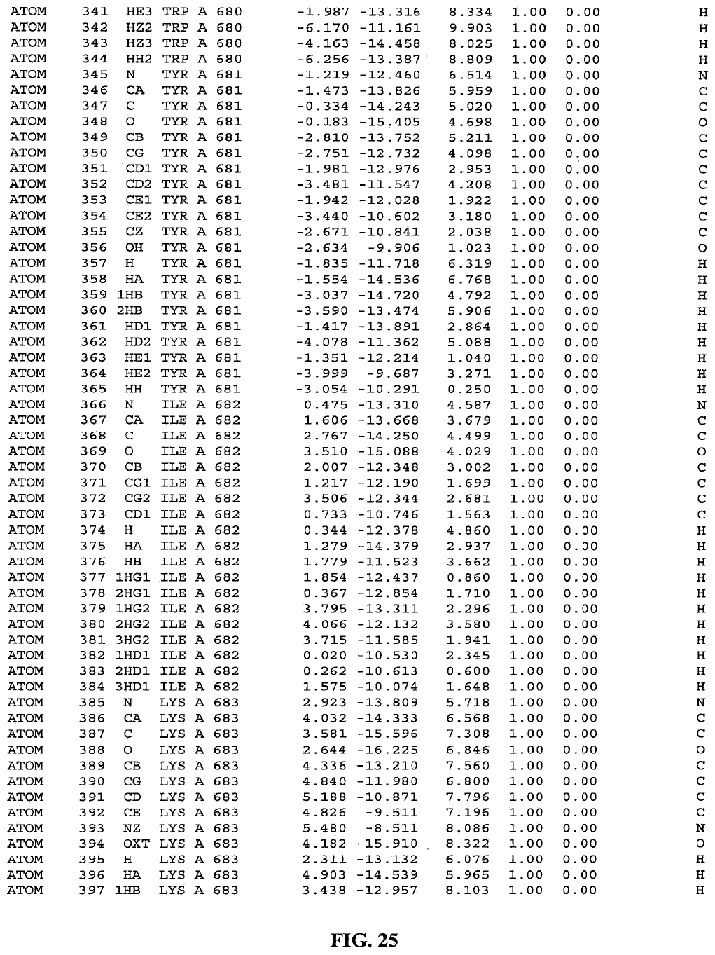

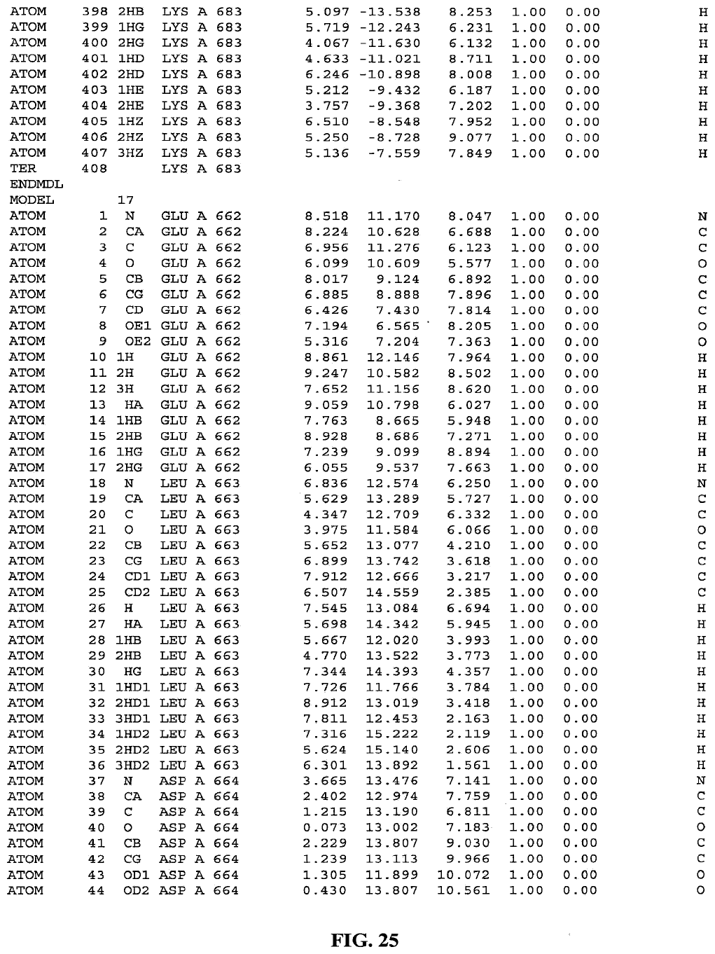

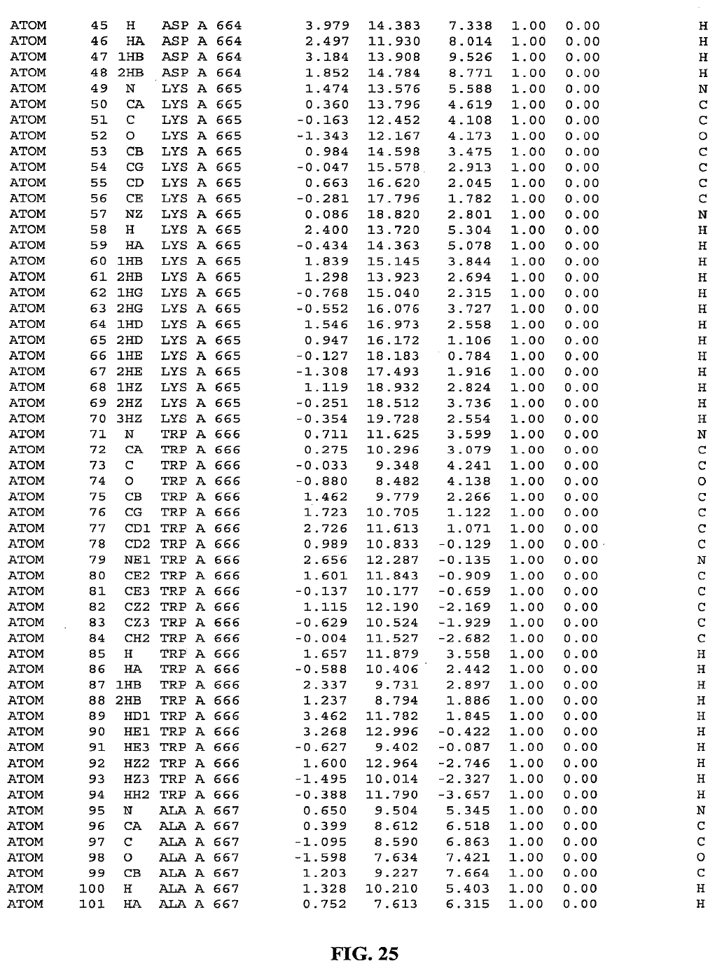

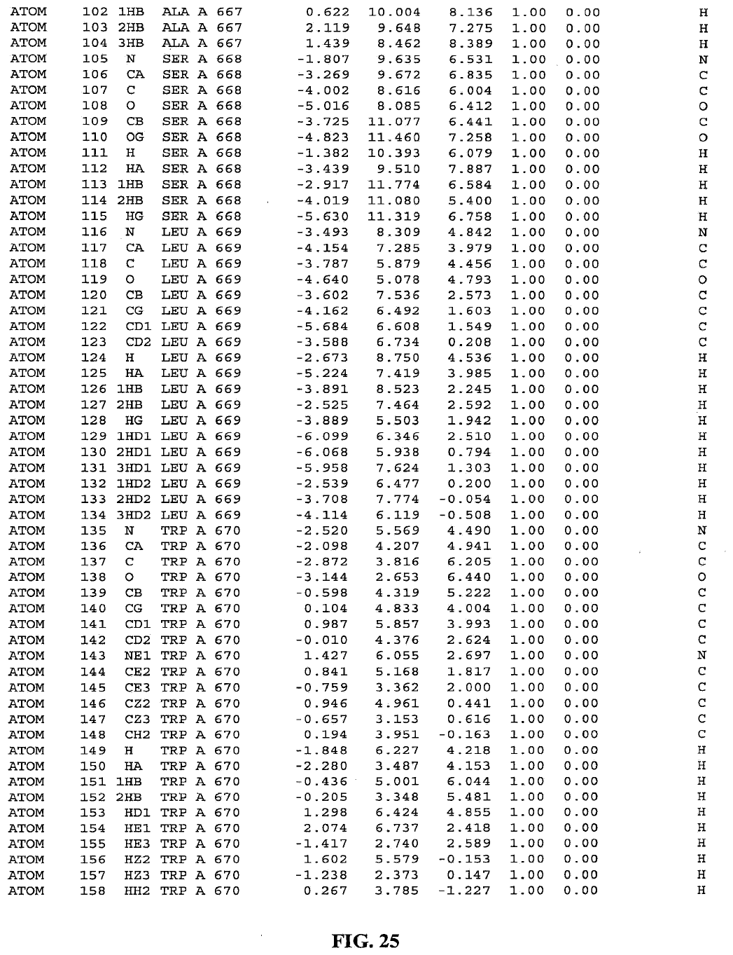

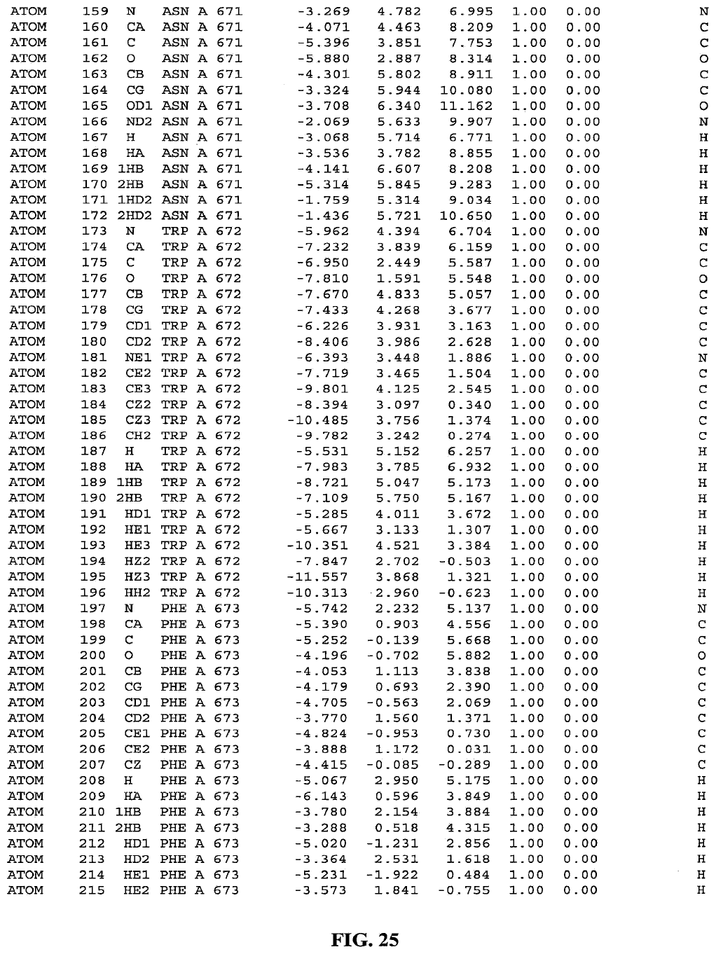

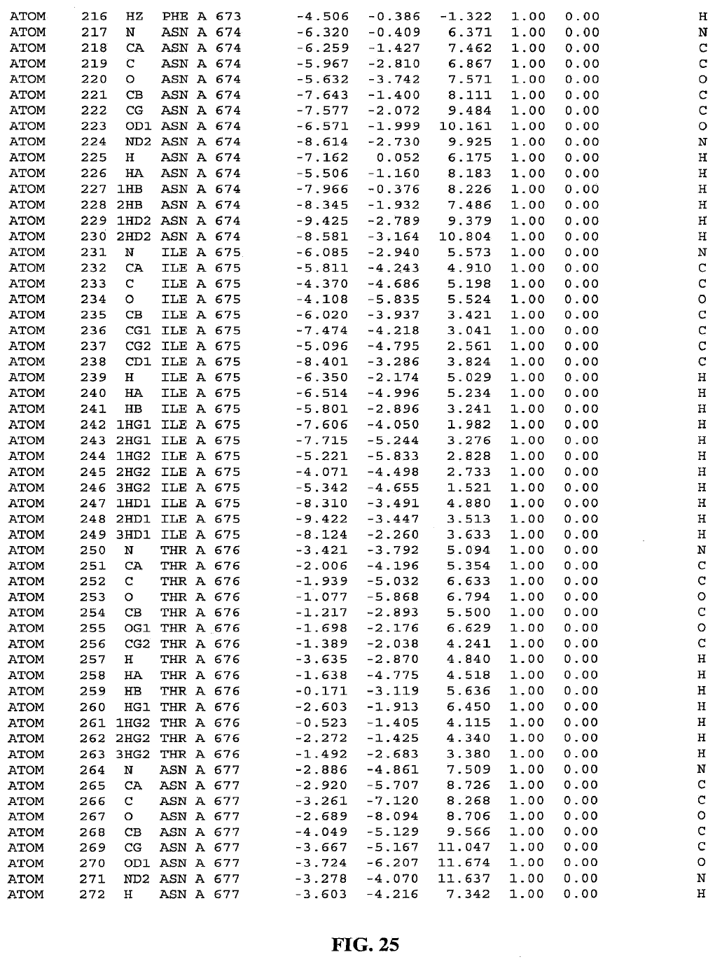

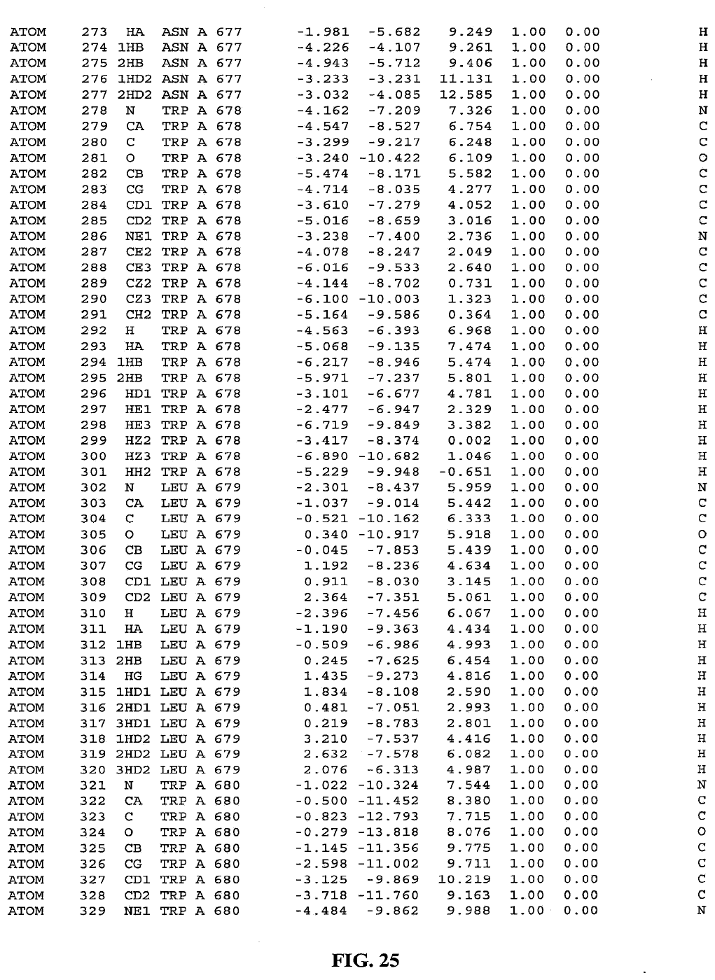

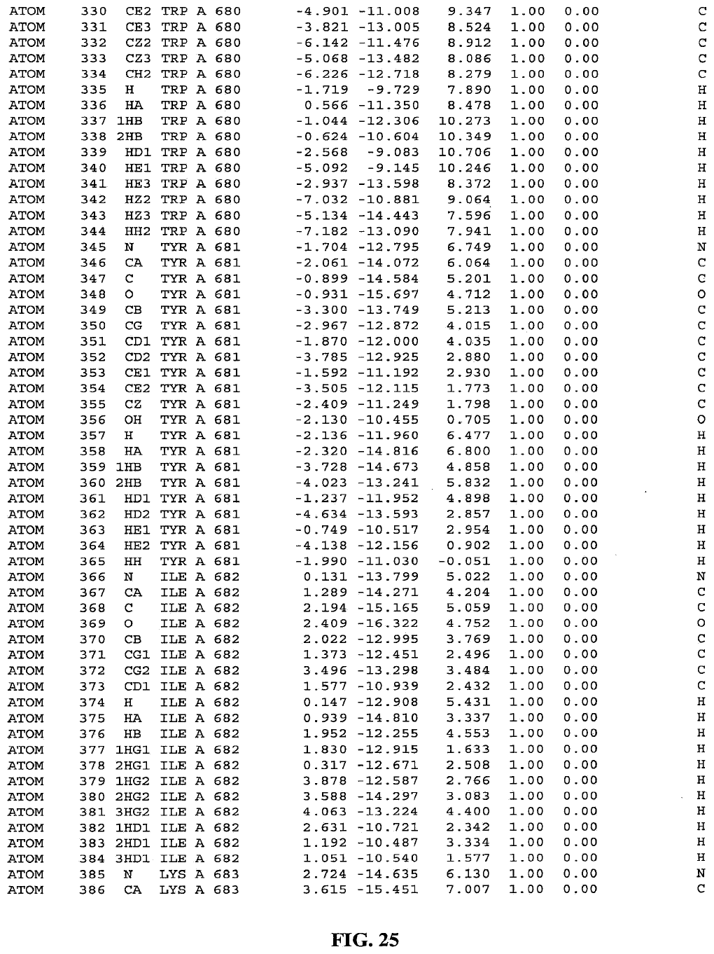

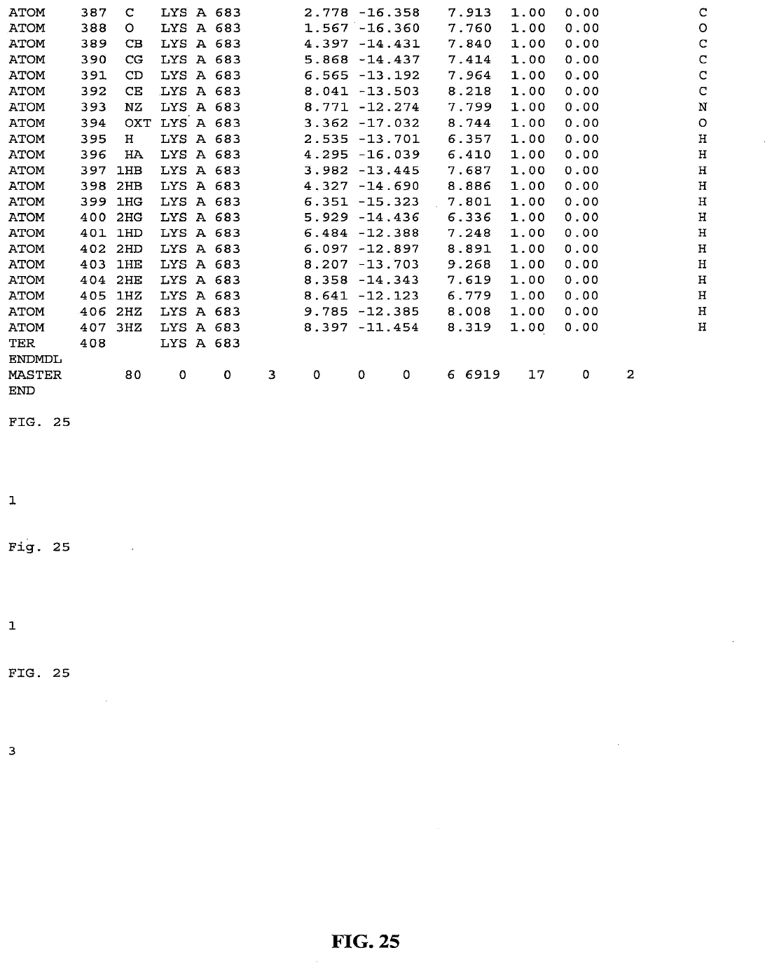

The disclosure also relates to a three dimensional structure of an MPER of an HIV-1 gp160 polypeptide in the context of lipid, that is, wherein at least one (e.g., two, three, four, five, six, seven, eight, nine, 10, 11, 12, 13, 14, 15, 16, 17, 18, 19, or 20 or more) amino acid of the MPER is embedded in the lipid. The three dimensional structure is determined by, for example, X-ray diffraction of a crystal of an MPER in the context of a lipid, or nuclear magnetic resonance (NMR) data from a solution containing the complex. In one example, the disclosure features a solution structure of an MPER as determined using NMR spectroscopy and various computer modeling techniques. Structural coordinates of an MPER (e.g., the solution structure coordinates disclosed herein at FIG. 25; also disclosed as Protein Data Bank (PDB) deposit rcsb042808 or PDB ID 2PV6) are useful for a number of applications, including, but not limited to, the characterization of a three dimensional structure of an MPER, as well as the visualization, identification and characterization of regions of the MPER that are involved in mediating fusion of an HIV-1 particle and a cell.

The MPER:lipid complex suitable for determining a three-dimensional structure can be formed by mixing an MPER polypeptide with, e.g., one or more lipids or lipid vesicles. MPER:lipid complexes formed by mixing an MPER polypeptide with POPC/POPG (4:1, w/w) large unilamellar lipid vesicles are described in the accompanying Examples.

As used herein, the MPER:lipid complex in solution comprises all or a fragment of an MPER polypeptide. The MPER polypeptide can include, for example, amino acid residues from about 660 to about 690 (e.g., from about 662 to about 683) of SEQ ID NO:37, and can be, e.g., the amino acid residues 662-683 set forth in FIG. 25, or conservative substitutions thereof.

The lipid can be any of those described herein and in any form. For example, the lipid can include one or more phospholipids and/or form a lipid monolayer or bilayer.

The MPER in solution can be either unlabeled, ¹⁵N enriched or ¹⁵N, ¹³C enriched. In addition, the secondary structure of the MPER in the solutions described herein can comprise two alpha (a) helices. In some embodiments, a first alpha helix corresponds to acid residue positions 662-672 of SEQ ID NO:37 and a second alpha helix corresponding to amino acid positions 676-682 of SEQ ID NO:37. For example, a first alpha helix comprises from about amino acid residues 662-672 as set forth in FIG. 25 and a second alpha helix comprises from about amino acid residues 675-682 as set forth in FIG. 25.

The solution structure of the MPER polypeptide can be characterized by a three dimensional structure comprising part of all of the relative structural coordinates of FIG. 25. For example, the solution structure of the MPER polypeptide can be characterized by a three dimensional structure comprising the relative structural coordinates of amino acid residues L669 to W680 according to FIG. 25, ±a root mean square deviation from the conserved backbone atoms of said amino acids of not more than 0.5 Å (e.g., not more than 1.0 Å or 1.5 Å). In some embodiments, the solution structure of the MPER can be characterized by a three dimensional structure comprising the complete structural coordinates of the amino acids according to FIG. 25, ±a root mean square deviation from the conserved backbone atoms of said amino acids of not more than 1.5 Å (e.g., not more than 1.0 Å or 0.5 Å).

In some embodiments, the solution structure of the MPER polypeptide can be characterized by a three dimensional structure comprising one or both of the two alpha helices characterized by amino acid residues 662 to 672 and/or 676 to 682 of SEQ ID NO:37 according to FIG. 25, ±a root mean square deviation from the conserved backbone atoms of said amino acids of not more than 1.5 Å (e.g., not more than 1.0 Å or 0.5 Å).

The solution structural coordinates provided herein can be used to characterize a three dimensional structure of the MPER of an HIV-1 gp160 polypeptide. From such a structure, putative antibody or agent binding sites can be computationally visualized, identified and characterized based on the surface structure of the molecule, surface charge, steric arrangement, the presence of reactive amino acids, regions of hydrophobicity or hydrophilicity, etc. Such putative sites can be further refined using chemical shift perturbations of spectra generated from various and distinct MPER/lipid complexes, competitive and non-competitive inhibition experiments, and/or by the generation and characterization of MPER mutants to identify critical residues or characteristics of an antibody or agent binding site.

These binding sites are particularly important for use in the design or selection of inhibitors (e.g., antibodies or agents) that affect the activity of the MPER (e.g., an inhibitor of the fusion between an HIV-1 particle and a cell). For example, an inhibitor designed using the three-dimensional structure of MPER in lipid can be capable of extracting part of an MPER polypeptide from a lipid membrane (e.g., in vitro or in vivo).

In order to use the structural coordinates generated for a solution structure described herein as set forth in FIG. 25, the relevant coordinates can be displayed as, or converted to, a three dimensional shape or graphical representation. For example, a three dimensional representation of the structural coordinates is often used in rational drug design, molecular replacement analysis, homology modeling, and mutation analysis. This is typically accomplished using any of a wide variety of commercially available software programs capable of generating three dimensional graphical representations of molecules or portions thereof from a set of structural coordinates. Examples of commercially available software programs include, without limitation, the following: GRID (Oxford University, Oxford, UK); MCSS (Molecular Simulations, San Diego, Calif.); AUTODOCK (Scripps Research Institute, La Jolla, Calif.); DOCK (University of California, San Francisco, Calif.); Flo99 (Thistlesoft, Morris Township, N.J.); Ludi (Molecular Simulations, San Diego, Calif.); QUANTA (Molecular Simulations, San Diego, Calif.); Insight (Molecular Simulations, San Diego, Calif.); SYBYL (TRIPOS, Inc., St. Louis, Mo.); and LEAPFROG (TRIPOS, Inc., St. Louis, Mo.).

For storage, transfer and use with such programs, a machine, such as a computer, is provided for that produces a three dimensional representation of the MPER (with or without the lipid context). The machine can contain a machine-readable data storage medium comprising a data storage material encoded with machine-readable data. Machine-readable storage media comprising data storage material include conventional computer hard drives, floppy disks, DAT tape, CD-ROM, and other magnetic, magneto-optical, optical, floptical and other media which may be adapted for use with a computer. The machine of the present invention also comprises a working memory for storing instructions for processing the machine-readable data, as well as a central processing unit (CPU) coupled to the working memory and to the machine-readable data storage medium for the purpose of processing the machine-readable data into the desired three dimensional representation.

The machine can also include a display connected to the CPU so that the three dimensional representation may be visualized by the user. Accordingly, when used with a machine programmed with instructions for using said data, e.g., a computer loaded with one or more programs of the sort identified above, the machine provided for herein is capable of displaying a graphical three-dimensional representation of any of the molecules or molecular complexes, or portions of molecules of molecular complexes, described herein.

The structural coordinates of the MPER described herein permit the use of various molecular design and analysis techniques in order to (i) solve the three dimensional structures of related molecules, molecular complexes or MPER analogues, and (ii) to design, select, and synthesize chemical agents capable of favorably associating or interacting with an MPER, wherein said chemical agents potentially act as inhibitors of the fusion of an HIV-1 particle and a cell.

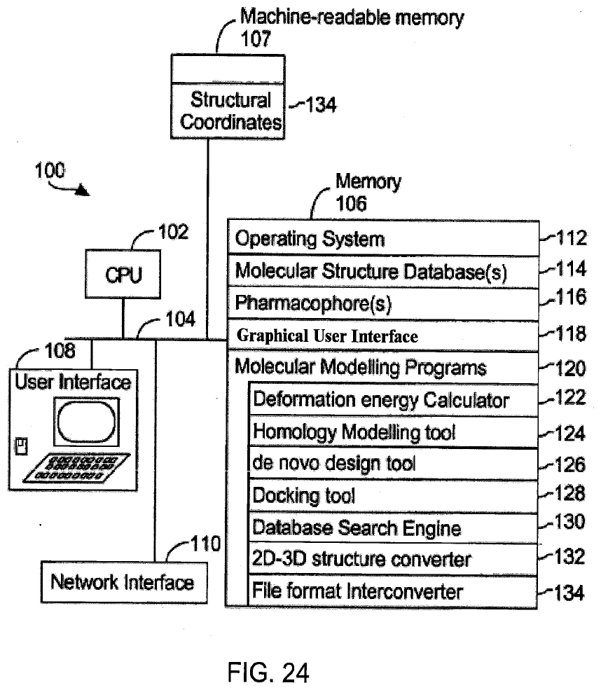

An exemplary computer system for use in the methods described herein is depicted in FIG. 24. According to FIG. 24, a computer system 100 on which methods described herein can be carried out, comprises: at least one central-processing unit 102 for processing machine readable data, coupled via a bus 104 to working memory 106, a user interface 108, a network interface 110, and a machine-readable memory 107.

Machine-readable memory 107 comprises a data storage material encoded with machine-readable data, wherein the data comprises the structural coordinates 134 of at least one MPER polypeptide (in a lipid environment such as DPC micelle), or a binding site on the MPER,; and

Working memory 106 stores an operating system 112, optionally one or more molecular structure databases 114, one or more pharmacophores 116 derived from structural coordinates 134, a graphical user interface 118 and instructions for processing machine-readable data comprising one or more molecular modelling programs 120 such as a deformation energy calculator 122, a homology modelling tool 124, a de novo design tool, 126, a “docking tool” 128, a database search engine 130, a 2D-3D structure converter 132 and a file format interconverter 134.

Computer system 100 can be any of the varieties of laptop or desktop personal computer, or workstation, or a networked or mainframe computer or super-computer, that would be available to one of ordinary skill in the art. For example, computer system 100 may be an IBM-compatible personal computer, a Silicon Graphics, Hewlett-Packard, Fujitsu, NEC, Sun or DEC workstation, or may be a supercomputer of the type formerly popular in academic computing environments. Computer system 100 may also support multiple processors as, for example, in a Silicon Graphics “Origin” system.

Operating system 112 may be any suitable variety that runs on any of computer systems 100. For example, in one embodiment, operating system 112 is selected from the UNIX family of operating systems, for example, Ultrix from DEC, AIX from IBM, or IRIX from Silicon Graphics. It can also be a LINUX operating system. In some embodiments, operating system 112 may be a VAX VMS system. In some embodiments, operating system 112 is a Windows operating system such as Windows 3.1, Windows NT, Windows 95, Windows 98, Windows 2000, or Windows XP. In some embodiments, operating system 112 is a Macintosh operating system such as MacOS 7.5.x, MacOS 8.0, MacOS 8.1, MacOS 8.5. MacOS 8.6, MacOS 9.x and MaxOS X.

The graphical user interface (“GUI”) 118 is preferably used for displaying representations of structural coordinates 134, or variations thereof, in 3-dimensional form on user interface 108. GUI 118 also preferably permits the user to manipulate the display of the structure that corresponds to structural coordinates 134 in a number of ways, including, but not limited to: rotations in any of three orthogonal degrees of freedom; translations; projecting the structure on to a 2-dimensional representation; zooming in on specific portions of the structure; coloring of the structure according to a property that varies amongst to different regions of the structure; displaying subsets of the atoms in the structure; coloring the structure by atom type; displaying tertiary structure such as .alpha.-helices and .beta.-sheets as solid or shaded objects; and displaying a surface of a small molecule, peptide, or protein, as might correspond to, for example, a solvent accessible surface, also optionally colored according to some property. Structural coordinates 134 are also optionally copied into memory 106 to facilitate manipulations with one or more of the molecular modelling programs 120.

Network interface 110 may optionally be used to access one or more molecular structure databases stored in the memory of one or more other computers.

The computational methods of the present invention may be carried out with commercially available programs which run on, or with computer programs that are developed specially for the purpose and implemented on, computer system 100. Commercially available programs typically comprise large integrated molecular modelling packages that contain at least two of the types of molecular modelling programs 120 shown in FIG. 24. Examples of such large integrated packages that are known to those skilled in the art include: Cerius2 (available from Accelrys, a subsidiary of Pharmacopeia, Inc.; see also www.accelrys.com/cerius2/index.html), Molecular Operating Environment (available from, Chemical Computing Group Inc., 1010 Sherbrooke Street West, Suite 910, Montreal, Quebec, Canada; see www.chemcomp.com/fdept/prodinfo.htm), Sybyl (available from Tripos, Inc., 1699 South Hanley Road, St. Louis, Mo.; see www.tripos.com/software-/sybyl.html) and Quanta (available from Accelrys, a subsidiary of Pharmacopeia, Inc.; see also www.accelrys.com/quanta/index.html).

Alternatively, the computational methods of the present invention may be performed with one or more stand-alone programs each of which carries out one of the functions performed by molecular modelling programs 120. In particular, certain aspects of the display and visualization of molecular structures may be accomplished by specialized tools, for example, GRASP (Nicholls, A.; Sharp, K.; and Honig, B., PROTEINS, Structure, Function and Genetics, (1991), Vol. 11 (No. 4), 281; available from Dept. Biochem., Room 221, Columbia University, Box 36, 630 W. 168th St., New York, N.Y.; see also trantor.bioc.columbia.edu/grasp/).

Also provided is a method for determining the molecular structure of a molecule or molecular complex whose structure is unknown, comprising the steps of obtaining a solution of the molecule or molecular complex whose structure is unknown, and then generating NMR data from the solution of the molecule or molecular complex. The NMR data from the molecule or molecular complex whose structure is unknown is then compared to the solution structure data obtained from the MPER/lipid solutions described herein. Then, 2D, 3D, and 4D isotope filtering, editing and triple resonance NMR techniques are used to conform the three dimensional structure determined from the MPER/lipid solution to the NMR data from the solution molecule or molecular complex.

Alternatively, molecular replacement may be used to conform the MPER solution structure of the present invention to x-ray diffraction data from crystals of the unknown molecule or molecular complex.

Molecular replacement uses a molecule having a known structure as a starting point to model the structure of an unknown crystalline sample. This technique is based on the principle that two molecules which have similar structures, orientations and positions will diffract x-rays similarly. A corresponding approach to molecular replacement is applicable to modeling an unknown solution structure using NMR technology. The NMR spectra and resulting analysis of the NMR data for two similar structures will be essentially identical for regions of the proteins that are structurally conserved, where the NMR analysis consists of obtaining the NMR resonance assignments and the structural constraint assignments, which may contain hydrogen bond, distance, dihedral angle, coupling constant, chemical shift and dipolar coupling constant constraints. The observed differences in the NMR spectra of the two structures will highlight the differences between the two structures and identify the corresponding differences in the structural constraints. The structure determination process for the unknown structure is then based on modifying the NMR constraints from the known structure to be consistent with the observed spectral differences between the NMR spectra.

Accordingly, in some embodiments, the resonance assignments for the MPER:lipid solution provide the starting point for resonance assignments of an MPER:lipid complex in a new MPER:lipid:“unsolved agent” complex. Chemical shift perturbances in two dimensional ¹⁵N/¹H spectra can be observed and compared between the MPER:lipid solution and the new MPER:lipid:agent complex. In this way, the affected residues may be correlated with the three dimensional structure of the MPER as provided by the relevant structural coordinates of FIG. 25. This effectively identifies the region of the MPER:lipid:agent complex that has incurred a structural change relative to the native MPER structure. The ¹H, ¹⁵N, ¹³C and ¹³CO NMR resonance assignments corresponding to both the sequential backbone and side-chain amino acid assignments of the MPER:lipid can then be obtained and the three dimensional structure of the new MPER:lipid:agent complex may be generated using standard 2D, 3D and 4D triple resonance NMR techniques and NMR assignment methodology, using the MPER:lipid solution structure, resonance assignments and structural constraints as a reference. Various computer fitting analyses of the new agent with the three dimensional model of the MPER can be performed in order to generate an initial three dimensional model of the new agent complexed with an MPER in the context of lipid, and the resulting three dimensional model may be refined using standard experimental constraints and energy minimization techniques in order to position and orient the new agent in association with the three dimensional structure of an MPER.

The structural coordinates described herein can be used with standard homology modeling techniques in order to determine the unknown three-dimensional structure of a molecule or molecular complex. Homology modeling involves constructing a model of an unknown structure using structural coordinates of one or more related protein molecules, molecular complexes or parts thereof. Homology modeling can be conducted by fitting common or homologous portions of the protein whose three dimensional structure is to be solved to the three dimensional structure of homologous structural elements in the known molecule, specifically using the relevant (i.e., homologous) structural coordinates provided by FIG. 25 herein. Homology may be determined using amino acid sequence identity, homologous secondary structure elements, and/or homologous tertiary folds. Homology modeling can include rebuilding part or all of a three dimensional structure with replacement of amino acids (or other components) by those of the related structure to be solved.

Accordingly, a three dimensional structure for the unknown molecule or molecular complex may be generated using the three dimensional structure of the MPER described herein, refined using a number of techniques well known in the art, and then used in the same fashion as the structural coordinates of the present invention, for instance, in applications involving molecular replacement analysis, homology modeling, and rational drug design.

Determination of the three dimensional structure of an MPER in the context of a lipid, and potential binding sites in the MPER for neutralizing antibodies, is useful for the targeted and rational identification and/or design of agents that can, e.g., inhibit the fusion of HIV-1 and a cell. This is advantageous over conventional drug assay techniques, which often requires screening thousands of test compounds.

X-ray, spectroscopic and computer modeling technologies allow for visualization of the three dimensional structure of a targeted compound (i.e., an MPER). Three dimensional structures can be used to identify putative binding sites and then identify or design agents to interact with these binding sites. These agents can then be screened for an inhibitory effect on the target molecule. By this method, the number of agents to be screened is typically less than that required for conventional drug assay techniques as described above.

Accordingly, the disclosure features a method for identifying a potential inhibitor of the fusion of an HIV-1 particle with a cell, which method includes the steps of using a three dimensional structure of an MPER, such as the structure defined by the relative structural coordinates of FIG. 25 to design or select an agent that binds to the MPER and potentially inhibits the fusion of an HIV-1 particle to a cell. The inhibitor can be selected by screening an appropriate database, can be designed de novo by analyzing the steric configurations and charge potentials of an MPER (or the amino acids exposed on the surface of an HIV-1 envelope) in conjunction with the appropriate software programs, or may be designed using characteristics of known fusion inhibitors in order to create “hybrid” inhibitors.

An agent that interacts or associates with an MPER can be identified by determining a putative binding site from the three dimensional structure of the MPER, and performing computer fitting analyses to identify an agent which interacts or associates with said binding site. Computer fitting analyses utilize various computer software programs that evaluate the “fit” between the putative binding site and the identified agent, by (a) generating a three dimensional model of the putative binding site of a molecule or molecular complex using homology modeling or the atomic structural coordinates of the binding site, and (b) determining the degree of association between the putative binding site and the identified agent. The degree of association can be determined computationally by any number of commercially available software programs, or may be determined experimentally using standard binding assays.

Three dimensional models of a binding site for an inhibitory agent (e.g., an MPER-specific antibody) can be generated using any one of a number of methods known in the art, and include, but are not limited to, homology modeling as well as computer analysis of raw structural coordinate data generated using crystallographic or spectroscopy techniques. Computer programs used to generate such three dimensional models and/or perform the necessary fitting analyses include, but are not limited to: GRID (Oxford University, Oxford, UK), MCSS (Molecular Simulations, San Diego, Calif.), AUTODOCK (Scripps Research Institute, La Jolla, Calif.), DOCK (University of California, San Francisco, Calif.), Flo99 (Thistlesoft, Morris Township, N.J.), Ludi (Molecular Simulations, San Diego, Calif.), QUANTA (Molecular Simulations, San Diego, Calif.), Insight (Molecular Simulations, San Diego, Calif.), SYBYL (TRIPOS, Inc., St. Louis, Mo.) and LEAPFROG (TRIPOS, Inc., St. Louis, Mo.).

The effect of such an agent identified by computer fitting analyses using the MPER structure can be further evaluated computationally, or experimentally by competitive binding experiments or by contacting the identified agent with an HIV-1 particle and measuring the effect of the agent on the ability of the HIV-1 particle to fuse to a target cell. Methods for detecting fusion of an HIV-1 particle to a cell are known in the art and described in, e.g., Zhou et al. (2004) Gene Therapy 11(23):1703-1712; Goudsmit et al. (1998) AIDS 2(3):157-64; Wells et al. (1991) J Virol. 65(11):6325-30; and Momota et al. (1991) Biochem Biophys Res Commun. 179(1):243-50, the disclosures of each of which are incorporated by reference in their entirety. Further tests can be performed to evaluate the selectivity of the binding of the identified agent to a particular MPER with regard to, e.g., other MPER regions or other regions of HIV-1 gp160.

An agent designed or selected to interact with an MPER can be capable of both physically and structurally associating with the MPER via various covalent and/or non-covalent molecular interactions, and of assuming a three dimensional configuration and orientation that complements the relevant binding site in the MPER.

Accordingly, the structural coordinates of the MPER as disclosed herein, through molecular replacement or homology modeling techniques, can be used to redesign known inhibitiors that increase either or both of the potency or selectivity of the known inhibitors, either by modifying the structure of known inhibitors or by designing new agents de novo via computational inspection of the three dimensional configuration and electrostatic potential of an MPER binding site.

The structural coordinates of FIG. 25, or structural coordinates derived therefrom using molecular replacement or homology modeling techniques as discussed above, can be used to screen a database for agents that can bind to the MPER and act as potential inhibitors of HIV-1 fusion. Specifically, the obtained structural coordinates described herein can be entered into a software package and the three dimensional structure analyzed graphically. A number of computational software packages may be used for the analysis of structural coordinates, including, but not limited to, Sybyl (Tripos Associates), QUANTA and XPLOR (Brunger, A. T., (1994) X-Plor 3.851: a system for X-ray Crystallography and NMR. Xplor Version 3.851 New Haven, Conn.: Yale University Press). Additional software programs check for the correctness of the coordinates with regard to features such as bond and atom types. If necessary, the three dimensional structure can be modified and then energy minimized using the appropriate software until all of the structural parameters are at their equilibrium/optimal values. The energy minimized structure is superimposed against the original structure to make sure, e.g., that there are no significant deviations between the original and the energy minimized coordinates.

The energy minimized coordinates of an MPER bound to a “solved” binding agent/inhibitor are then analyzed and the interactions between the solved ligand and MPER can be identified. The final MPER structure can be modified by graphically removing the solved inhibitor so that only the MPER and a few residues of the solved agent are left for analysis of the binding site cavity. QSAR and SAR analysis and/or conformational analysis can be carried out to determine how other inhibitors compare to the solved inhibitor. The solved agent can be docked into the uncomplexed structure's binding site to be used as a template for data base searching, using software to create excluded volume and distance restrained queries for the searches. Structures qualifying as hits are then screened for activity using standard assays and other methods known in the art.

Further, once the specific interaction is determined between the solved binding agent/inhibitor, docking studies with different inhibitors allow for the generation of initial models of new binding agents/inhibitors bound to an MPER. The integrity of these new models may be evaluated a number of ways, including constrained conformational analysis using molecular dynamics methods (i.e., where both the MPER and the bound binding agent/inhibitor are allowed to sample different three dimensional conformational states until the most favorable state is reached or found to exist between the protein and the bound agent). The final structure as proposed by the molecular dynamics analysis is analyzed visually to make sure that the model is in accord with known experimental SAR based on measured binding affinities. Once models are obtained of the original solved agent bound to the MPER and computer models of other molecules bound to an MPER, strategies are determined for designing modifications into the inhibitors to improve their activity and/or enhance their selectivity.

Once an MPER binding agent has been optimally selected or designed, as described above, substitutions may then be made in some of its atoms or side groups in order to improve or modify its selectivity and binding properties. Generally, initial substitutions are conservative, i.e., the replacement group will have approximately the same size, shape, hydrophobicity and charge as the original group. Suitable conservative substitutions for protein binding agents are described above. Such substituted chemical compounds may then be analyzed for efficiency of fit to the MPER by the same computer methods described in detail above.

Various molecular analysis and rational drug design techniques are further disclosed in U.S. Pat. Nos. 5,834,228, 5,939,528 and 5,865,116, as well as in PCT Application No. PCT/US98/16879, published as WO 99/09148, the contents of which are hereby incorporated by reference.

Methods for Identifying an Agent Capable of Extracting an MPER from Lipid

As described herein, the inventors have discovered that the HIV-1-specific broadly neutralizing antibody (BNAb), 4E10, upon binding to the MPER in a lipid environment, extracts key antibody epitope residues, W672 and F673, from the lipid. These observations provide important implications for vaccine design strategy and offer insight into how BNAbs perturb viral fusion in the case of HIV-1. Moreover, the observations allow for the identification of a wide variety of agents that, like the 4E10 antibody, are capable of extracting MPER amino acids from the lipid and thus potentially inhibiting HIV-1 fusion to a cell. Such agents are useful as therapy for, or prophylaxis against, HIV-1 infection in a subject.

Accordingly, the disclosure features a method of identifying an agent capable of extracting one or more amino acid residues of an MPER from lipid. The method includes the steps of: providing a composition comprising lipid and an MPER of an HIV-1 polypeptide, wherein one or more amino acids of the MPER are embedded in the lipid; contacting the composition with a candidate agent; and detecting whether the candidate agent extracts one or more amino acids of the MPER from the lipid. The extraction of one or more amino acids from the lipid indicates that the candidate agent is capable of extracting one or more amino acid residues of an MPER from lipid.

Methods for determining whether one or more amino acids of an MPER are extracted from lipid are set forth in the accompanying Examples. For example, the energetics of the binding of an agent to an MPER can be determined using NMR and EPR techniques. First, EPR membrane immersion depth data on spin-labeled MPER peptides can be obtained in the presence and absence of a candidate agent to measure the orientation of the MPER peptide in complex with or without the agent with respect to the membrane. A change in the immersion depth data in the presence of a candidate agent as compared to the absence of the agent indicates that the a portion or all of the MPER is lifted up toward the aqueous phase.

In some embodiments, e.g., where the candidate agent is found to change the membrane immersion status of one or more amino acids of the MPER, the methods can further include the step of determining whether conformational changes at specific residues of the MPER occurred. An MPER peptide in complex with the candidate agent can be prepared in deuterated lipid micelles and evaluated using NMR spectroscopy. Amide chemical shift perturbations of the MPER residues in the presence or absence of the candidate agent can be determined. In some embodiments, the amino acid residues of the MPER displaying the most significant chemical shift changes in the presence of the candidate agent are those preferentially affected by the candidate agent.

The methods can further include the step of determining the crystal or solution structure for the MPER bound to the candidate agent in a lipid environment. Methods for determining such a structure are described herein (see above and the accompanying Examples).

It is understood that in methods described above, the 4E10 BNAb can be used as a positive control for extraction of one or more MPER amino acids from the lipid.

In some embodiments, the method can also include the step of determining if the agent inhibits the fusion of an HIV-1 particle and a cell. Suitable methods for measuring or detecting fusion in the presence and absence of an agent are described above.

Additional methods for determining whether a candidate agent is capable of extracting one or more amino acids of an MPER from lipid are contemplated by the concepts described herein. That is, the disclosure embraces methods for determining whether one or more amino acids of an MPER are extracted from lipid, which are not expressly described.

It is understood that these methods can be applied to a wide variety of polypeptides (e.g., microbial polypeptides such as other viral polypeptides involved in fusion with a cell).

### Agents

Agents (e.g., binding agents or inhibitory agents) identified in any of the methods described herein can include various chemical classes, though typically small molecules (e.g., small organic molecules) having a molecular weight in the range of 50 to 2,500 daltons. These agents can comprise functional groups necessary for structural interaction with proteins (e.g., hydrogen bonding), and typically include at least an amine, carbonyl, hydroxyl, or carboxyl group, and preferably at least two of the functional chemical groups. These agents often comprise cyclical carbon or heterocyclic structures and/or aromatic or polyaromatic structures (e.g., purine core) substituted with one or more of the above functional groups.

In alternative embodiments, compounds can also include biomolecules including, but not limited to, peptides, polypeptides (e.g., antibodies or antigen binding fragments thereof; see above), peptidomimetics (e.g., peptoids), amino acids, amino acid analogs, saccharides, fatty acids, steroids, purines, pyrimidines, derivatives or structural analogues thereof, polynucleotides, and polynucleotide analogs.

In some embodiments, the agents can be small molecule compounds such as nucleic acid aptamers which are relatively short nucleic acid (DNA, RNA or a combination of both) sequences that bind with high avidity to a variety of proteins and inhibit the binding to such proteins of ligands, receptors, and other molecules. Aptamers are generally about 25-40 nucleotides in length and have molecular weights in the range of about 8-14 kDa. Aptamers with high specificity and affinity for targets can be obtained by an in vitro evolutionary process termed SELEX (systemic evolution of ligands by exponential enrichment) [see, for example, Zhang et al. (2004) Arch. Immunol. Ther. Exp. 52:307-315, the disclosure of which is incorporated herein by reference in its entirety]. For methods of enhancing the stability (by using nucleotide analogs, for example) and enhancing in vivo bioavailability (e.g., in vivo persistence in a subject's circulatory system) of nucleic acid aptamers see Zhang et al. (2004) and Brody et al. [(2000) Reviews in Molecular Biotechnology 74:5-13, the disclosure of which is incorporated herein by reference in its entirety].

Agents can be identified from a number of potential sources, including: chemical libraries, natural product libraries, and combinatorial libraries comprised of random peptides, oligonucleotides, or organic molecules. Chemical libraries consist of random chemical structures, some of which are analogs of known compounds or analogs or compounds that have been identified as “hits” or “leads” in other drug discovery screens, while others are derived from natural products, and still others arise from non-directed synthetic organic chemistry. Natural product libraries re collections of microorganisms, animals, plants, or marine organisms which are used to create mixtures for screening by: (1) fermentation and extraction of broths from soil, plant or marine microorganisms, or (2) extraction of plants or marine organisms. Natural product libraries include polypeptides, non-ribosomal peptides, and variants (non-naturally occurring) thereof. For a review, see Science 282:63-68 (1998). Combinatorial libraries are composed or large numbers of peptides, oligonucleotides, or organic compounds as a mixture. These libraries are relatively easy to prepare by traditional automated synthesis methods, PCR, cloning, or proprietary synthetic methods. Of particular interest are non-peptide combinatorial libraries. Still other libraries of interest include peptide, protein, peptidomimetic, multiparallel synthetic collection, recombinatorial, and polypeptide libraries. For a review of combinatorial chemistry and libraries created therefrom, see Myers, Curr. Opin. Bioechnol. 8:701-707 (1997) the disclosure of which are incorporated by reference in its entirety. Identification of test compounds through the use of the various libraries herein permits subsequent modification of the test compound “hit” or “lead” to optimize the capacity of the “hit” or “lead” to bind to an MPER or to inhibit the fusion of an HIV-1 particle and a cell.

The agents identified above can be synthesized by any chemical or biological method. The agents can be pure, or can be in a heterologous composition (e.g., a pharmaceutical composition), and can be prepared in an assay-, physiologic-, or pharmaceutically-acceptable diluent or carrier. This composition can also contain additional compounds or constituents which do not bind to an MPER or inhibit the fusion of an HIV-1 particle and a cell.

### Kits and Articles of Manufacture

Also provided herein are kits containing one or more of any of the reagents described herein and, optionally, instructions for administering the one or more reagents to a subject (e.g., a human or any of the subjects described herein). The subject can have, be at risk of having, or be suspected of having, an HIV-1 infection. The kits can also, optionally, include one or more pharmaceutically acceptable carriers or diluents.

In some embodiments, the kits can further include instructions and/or diagnostic components for determining if a subject has an HIV-1 infection.

In some embodiments, the kits can include instructions and or diagnostic components useful for determining whether an immune response to the reagent has occurred in a subject.

In some embodiments, the kits can include one or more reagents for processing a sample (e.g., a blood sample). For example, a kit can include reagents for isolating or detecting RNA (e.g., HIV-1 RNA), protein (e.g., HIV-1 proteins), or antibodies to an HIV-1 protein from a sample.

The disclosure also provides an article of manufacture containing: a container; and a composition contained within the container, wherein the composition comprises an active ingredient for inducing an immune response in a mammal, wherein the active ingredient comprises any of the reagents described herein, and wherein the container has a label indicating that the composition is for use in inducing an immune response in a mammal.

In some embodiments, the label can further indicate that the composition is to be administered to a mammal having, suspected of having, or at risk of developing, an HIV-1 infection. The article of manufacture can also contain instructions for administering the composition (e.g., the rehydrated composition) to the mammal.

In some embodiments, the composition can be dried or lyophilized. The composition can be ready to administer without need for rehydration or further formulation.

The following examples are intended to illustrate, not limit, the invention.

### EXAMPLES

### Example 1

### Materials and Methods

Lipids

Phospholipids 1,2-dioleoyl-sn-glycero-3-phosphatidylcholine (DOPC), 1,2-dioleoyl-sn-glycero-3-phosphatidylethanolamine (DOPE), 1,2-dioleoyl-sn-glycero-3-[phospho-rac-(1-glycerol)] (DOPG), egg sphingomyelin (SM) dissolved in chloroform and cholesterol (CHOL) in powder were purchased from Avanti Polar Lipids, Inc. (Alabaster, Ala.). 1-Palmitoyl-2-oleoyl-sn-glycero-3-phosphocholine (POPC), 1-palmitoyl-2-oleoyl-sn-glycero-3-[phospho-rac-(1-glycerol)] (POPG), 1-palmitoyl-2-oleoyl-sn-glycero-3-phosphotempocholine (PC tempo), 1-palmitoyl-2-stearoyl(5-doxyl)-sn-glycero-3-phosphocholine (5-doxyl PC), 1-palmitoyl-2-stearoyl(7-doxyl)-sn-glycero-3-phosphocholine (7-doxyl PC), 1-palmitoyl-2-stearoyl(10-doxyl)-sn-glycero-3-phosphocholine (10-doxyl PC), 1-palmitoyl-2-stearoyl(12-doxyl)-sn-glycero-3-phosphocholine (12-doxyl PC) were purchased from Avanti Polar Lipids, Inc. N-tempoylpalmitamide was synthesized (Shin et al. (1992) Biophys. J. 61:1443-1453). Dodecyl phosphatidylcholine (DPC) for the production of micelle structures, 1,2-diheptanoyl-sn-glycero-3-phosphocholine (DHPC) and 1,2-Dimyristoyl-sn-Glycero-3-Phosphocholine (DMPC) for the production of bicelle structures were purchased from Avanti Polar Lipids, Inc. Deuterated (d38-) DPC was purchased from Cambridge Isotope Laboratories (Andover, Mass.). The MPER segment 662-683 of HXB2 gp160 (ELDKWASLWNWFNITNWLWYIK; SEQ ID NO:2), the MPER segment of an ADA strain gp160 (ALDKWASLWNWFDISNWLWYIK; SEQ ID NO:3) or mutant variants were expressed as a GB1-MPER fusion protein in E. coli. Each peptide was released from the fusion protein using cyanogen bromide (CNBr) cleavage and subsequently purified by high performance liquid chromatography (HPLC) to greater than 95% homogeneity. For spin-labeling experiments, the MPER segment 662-683 of HXB2 gp160 containing a single cysteine substitution at various positions was synthesized and desalted. The N- and C-termini of all the peptides were modified by acetylation and amidation, respectively. Further description related to expression and purification of MPER polypeptides is set forth below.

Electron Paramagnetic Resonance (EPR) Spectroscopy

EPR spectra were obtained on a Bruker EMX spectrometer (Billerica, Mass.) using a Bruker High Sensitivity resonator at room temperature. All spectra were recorded at 2 mW incident microwave power using a field modulation of 1.0-2.0 G at 100 kHz. For power saturation experiments, NiEDDA was synthesized as described in, e.g., Altenbach et al. (1994) Proc. Natl. Acad. Sci. USA 91:1667-1671 and Oh et al. (2000) Methods Mol. Biol. 145:147-169. In order to measure the accessibility parameters, Π, of O₂ and NiEDDA, power saturation experiments were carried out with a loop-gap resonator (JAGMAR, Krakow, Poland) (see, e.g., Farahbakhsh et al. (1992) Photochem Photobiol. 56:1019-1033; Oh et al. (2000) Methods Mol. Biol. 145:147-169; and Shin et al. (1992) Biophys. J. 61:1443-1453). The source of oxygen (O₂) gas was air supplied in house and the concentration of NiEDDA was 5 mM. Nitrogen (N₂) gas was used to purge O₂ when necessary. In order to measure the immersion-depths of membrane-inserted spin-labeled residues, air O₂ and 50 or 100 mM NiEDDA were used as collision reagents. The range of the incident microwave power was 0.4 to 100 mW for power saturation experiments. Power saturation data were analyzed using the R program (version 1.5.1) (see, e.g., Ihaka et al. J Comput. Graph. Stat. 3:299-314). Depth calibration curves were determined using the large unilamellar vesicles consisting of POPC/POPG (4:1, w/w) containing spin labeled lipids (Altenbach et al. (1994) Proc. Natl. Acad. Sci. USA 91:1667-1671 and Farahbakhsh et al. (1992) Photochem Photobiol. 56:1019-1033) in the presence and absence of 4E10 antibody at 800:1 molar ratio of total phosphate to antibodies. In order to determine the number of spin labels attached to peptides, EPR spectra were taken after liberating the spin labels from the peptide molecules by incubating the labeled peptides with 100 mM tris-(2-carboxyethyl)phosphine (Molecular Probes, Inc.). The amount of spin label was calculated by double integration of the EPR spectra using 3-carboxy-proxyl (Sigma-Aldrich) as a standard.

Surface Plasmon Resonance (SPR) Measurements

BIAcore experiments were carried out with a BIAcore 3000 using the Pioneer L1 sensor chip composed of alkyl chains covalently linked to a dextran-coated gold surface (BIAcore AB, Uppsala, Sweden) at 25° C. The running buffer was 20 mM HEPES containing 0.15M NaCl, pH 7.4 (HBS-N). The BIAcore instrument was cleaned extensively and left running overnight using Milli-Q water to remove trace amounts of detergent. The large unilamillar vesicles (LUV) (30 μl, 5 mM) were applied to the sensor chip surface at a flow rate of 3 μl/min, and the liposomes were captured on the surface of the sensor chip and provided a supported lipid bilayer. To remove any multilamellar structures from the lipid surface, sodium hydroxide (20 μl, 25 mM) was injected at a flow rate of 100 μl/min, which resulted in a stable baseline corresponding to the immobilized liposome bilayer membrane with response units (RU) of 8000-11,000.

Peptide solutions (0.7 μM) were prepared by dissolving the polypeptides in running buffer right before injection and the solution (60 μl) was injected over the lipid surface at a flow rate of 5 μl/min. Antibody solution (20 μg/ml) was passed over peptide-liposome complex for 3 min at a flow rate of 5 μl/min. Since the peptide-lipid interactions are very hydrophobic, the regeneration of the liposome surface was not possible. The immobilized liposomes were therefore completely removed with an injection of 40 mM CHAPS (25 μl) at a flow rate of 5 μl/min, and each peptide injection was performed on a freshly prepared liposome surface.

For analysis of antibody binding to spin-labeled, membrane-bound MPER peptides, a volume of 30 μl of POPC/POPG (4:1, w/w) LUVs (10.5 mM phosphate) in HBS-N was layered onto an L1 Sensor Chip and followed by spin-labeled peptide and antibody injection as described above at a rate of 3 μl/min. The wild-type and mutant peptide with 672A/673A double alanine substitution mutations were prepared as described in Expression and purification of MPER segments.

Isothermal Calorimetry (ITC) Experiments

Samples for ITC experiments were prepared in HBS-N buffer. Twenty injections of 15 μl liposome/MPER peptide mixture were delivered to 1.35 ml of 10 μM 4E10 Fab. 4E10 Fab was prepared using the Pierce Fab digestion kit (Rockford, Ill.) according to the manufacturer's recommendations. Data were acquired at 25° C. using a MicroCal ITC instrument, and analyzed using the software Microcal Origin (Northampton, Mass.).

NMR Spectroscopy and Structure Modeling

Samples for NMR experiments were prepared by co-dissolving lyophilized MPER peptides with regular or deuterated DPC, and adjusted to pH 6.6. All NMR experiments were carried out at 35° C. on spectrometers equipped with cryogenic probes. The data for backbone assignment of MPER peptide in DPC micelle were acquired using a Varian Inova 600 MHz spectrometer. The 3D N15-noesy (Nuclear Overhauser Enhancement Spectroscopy; 60 ms mixing time) and 2D noesy (80 ms mixing time, in D20) data were acquired using Bruker 750 MHz and 600 MHz spectrometers respectively. The Transverse Relaxation Optimized Spectroscopy (TROSY) data of MPER peptide in complex with 4E10 Fab were acquired using a Bruker 900 MHz spectrometer. The cross-saturation experiment was performed on a Bruker 600 MHz spectrometer in an interleaved fashion using 250 ms WURST ¹H saturation pulses with 2.3 ppm bandwidth irradiating at 0 ppm (methyl region) and −40 ppm (empty region) for alternating FIDs (Shimada et al. (2005) Methods Enzymol. 394:483-506).

Data were processed by using the software PROSA (Guntert et al. (1992) J Biomol. NMR 2:619-629) and analyzed using the software CARA (see the “Computer Aided Resonance Assignment” website). Chemical shift assignments were carried out using conventional NMR techniques (Ferentz et al. (2000) Q Rev. Biophys. 33:29-65). The preliminary structures were calculated by using the software CYANA (Guntert et al. (2004) J. Biomol. NMR 2:619-629), and the final structures by XPLOR-NIH (Brunger (1992) X-PLOR Version 3.1: A System for X-ray Crystallography and NMR (New Haven, Conn.: Yale University Press) and Schwieters et al. (2003) J Magn. Res. 160:66-74). NMR constraints and structural statistics are listed in Table 2.

TABLE 2

| NOE restraints (total non-redundant) | 331 |
|---|---|
| intra-residue | 92 |
| medium range (i <= 4) | 239 |
| long range (i > 4) | 0 |
| Dihedral angle restraints (total) | 34 |
| Φ angle | 20 |
| Ψ angle | 14 |
| Hydrogen bonds | 5 |
| Backbone <RMSD> to mean structure | |
| 665-682 | 0.59 Å |
| 665-673 (N-terminal) | 0.24 Å |
| 674-682 (C-terminal) | 0.15 Å |
| Geometry | |
| bonds (Å) | 0.0037 +/− 0.0001 |
| angles (deg) | 0.63 +/− 0.02 |
| impropers (deg) | 0.49 +/− 0.02 |
| Ramachandran statistics | |
| most favored regions | 83.5% |
| additionally allowed regions | 15.3% |
| generously allowed regions | 0.9% |
| disallowed regions | 0.3% (L663 only) |

The antibody-bound MPER peptide was modeled based on the X-ray crystallographic structure of peptide mimics in complex with 4E10 Fab (PDB code: 2FX7, 1TZG), the solution NMR structure of the free peptide as well as structural information obtained from the TROSY NMR experiments (Pervushin et al. (2000) Q Rev Biophys. 33:161-197). The secondary structures were confirmed from TALOS (Cornilescu et al. (1999) J Biomol. NMR 13:289-302) analysis of the chemical shift data (Table 2).

Expression and Purification of MPER Segments

The MPER segment of HXB2, the ADA strain or mutant variants fused at the C-terminus of a protein G B1 was expressed as a GB1-MPER fusion protein in E. coli. DNA coding for MPER segment was amplified by polymerase chain reaction (PCR), digested with restriction enzymes BamH I and Xho I, and then ligated into the expression vector pET 30a at corresponding sites, which vector harbors a gene coding protein G B1 domain fused with His tag at the C-terminus. The sequences were verified by DNA sequencing. E. coli BL21 cells were grown either in complete media for BIAcore studies or in 15N-labeled and ¹⁵N/¹³C-labeled M9 media for NMR studies to a cell density of OD₅₉₅ 0.6. Expression was induced by adding 1 mM isopropyl β-D-1-thiogalactopyranoside (IPTG) followed by incubation for 3-6 hours at 37° C. The overexpressed fusion protein was isolated from the cells in the form of inclusion bodies. The inclusion bodies were gradually dissolved in 6 M guanidine containing 20 mM Tris (pH8.0), 0.5 M NaCl and 20 mM imidazole. The fusion protein was then purified by Ni2+ column, dialyzed extensively against water followed by lyophilization. The peptide was released from the fusion protein using cyanogen bromide (CNBr) cleavage. The fusion protein dissolved in 70% trifluoroacetic acid (TFA) was incubated with 150 mg of CNBr overnight at room temperature. Upon completion of the reaction 10 volumes of water was added to the sample, and it was then lyophilized to complete dryness. The product was dissolved in 0.1% TFA in water and purified by high performance liquid chromatography (HPLC) using a preparative VYDAC C5 reversed-phase column (10 μm, 10 mm×25 cm) to greater than 95% homogeneity. Amino acid analysis and mass spectrometry confirmed the composition and molecular weight of the peptide. The concentration of peptide was measured by amino acids composition analysis.

Spin Labeling of Synthetic Peptides

For spin labeling, 4-6 mg of desalted peptides containing single cysteine substitutions were dissolved in 150 μl dimethyl sulfoxide (DMSO) and mixed with appropriate volume of (1-oxyl-2,2,5,5,-tetramethylpyrroline-3-methyl)-methanethiosulfonate(MTSL) stock solution in acetonitrile (100 mg/ml). The MTSL was 2-3 times in excess of the peptides in molar ratio. After reaction for approximately 16 hours at room temperature, the spin-labeled peptides were purified by reverse phase high pressure liquid chromatography (HPLC) using a C5 column (Sigma-Aldrich, St. Louis, Mo.). The fractions containing spin labeled peptides were identified by electron paramagnetic resonance (EPR) spectroscopy as described. The concentrations of the spin-labeled peptides were determined as described above (EPR spectroscopy). The masses of the spin-labeled peptides were confirmed by mass spectrometry. The total concentrations of the peptides were determined by amino acid analysis. The spin labeling ratio of the peptides, defined as the ratio of the spin label concentration determined by EPR to the total peptide concentration by amino acid analysis, ranged from 0.39 to 1.32. Peptide solutions were stored at −80° C.

Preparation of Lipid Vesicles

Mixtures of lipids were prepared in chloroform, divided in 50 mg aliquots and dried as thin films in glass test tubes under nitrogen gas. These were further dried under vacuum for 16 hours and resuspended in a 1 ml volume of 20 mM Hepes, 150 mM KCl, pH 7.0 (buffer A, hereafter). The lipid suspensions were freeze-thawed 10-15 times and extruded 15 times through two sheets of polycarbonate membrane with a pore size of 100 nm (Avestin) using an extruder (Avanti Polar Lipids, Inc.), resulting in large unilamellar vesicles (LUVs) (Szoka et al. (1980) Biochim Biophys Acta 601:559-571). POPC/POPG vesicles were made of 80% POPC and 20% POPG by weight. For the immersion-depth measurements, POPC/POPG vesicles containing trace amounts (1/1000 by weight) of PC tempo, N-tempoylpalmitamide, 5-, 7-, 10-, or 12-doxyl PC were also prepared in buffer A. The LUV of DOPC/SM/DOPE/DOPG/CHOL was prepared at the molar ratio of 34:7:16:10:33 for T cell membrane mimic and at the molar ratio of 9:18:20:9:45 for virion membrane mimic, and was used in the indicated BIAcore experiments. The phosphate contents of the vesicles were determined as described (Böttcher et al. (1961) Anal Chim Acta 24:203-204).

NMR Structure Determination and Modeling

In addition to NOE distance constraints (Table 2), data for backbone dihedral angles were acquired using a Bruker 500 MHz spectrometer. Specifically, 20 backbone dihedral angle Φ restraints were determined from the HNHA experiment (Vuister et al. (1993) J Am Chem Soc. 115:7772-7777), and 14 backbone Ψ angle restraints were obtained from the modified HNHB experiment (Dux et al. (1997) J Biomol NMR 10:301-306) with ranges from −60° to 0° for 3JNHa>0.8 Hz, and 10° to 180° for 3JNHa<0.6 Hz (Wang et al. (1995) J Am Chem Soc. 117:1810-1813). For modeling of the MPER/4E10 complex, the residues C-terminal of N671 were taken from the crystal structure (PDB code: 2FX₇), N671 taken from a homologous crystal structure (PDB code: 1TZG), and residues N-terminal of W670 were taken from the current solution structure. The backbone orientation for residue W670 was adjusted manually based on the backbone angles predicted by TALOS to avoid steric hindrance. The overall orientation of the MPER/4E10 complex relative to the membrane surface was adjusted to fit the EPR immersion depth results. The side-chain of Y681 was rotated manually towards the membrane.

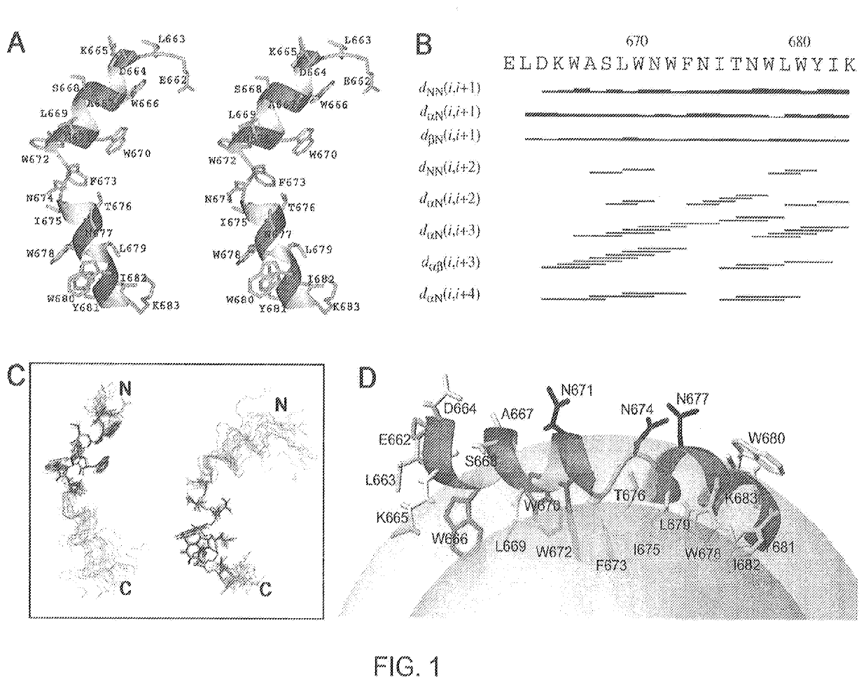

FIGS. 1A, 1C-1D, 3A, and 4D-4E were prepared by using the software MOLMOL (Koradi et al. (1996) J Mol Graphics 14:51-55).

Bioinformatics

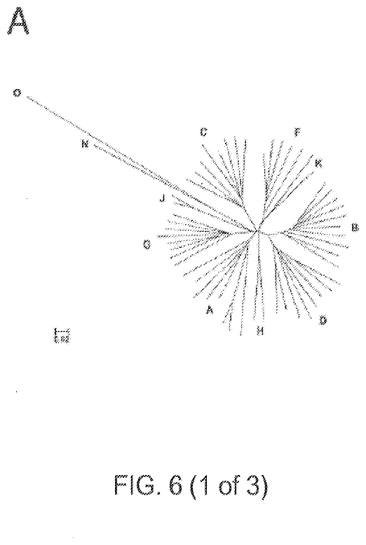

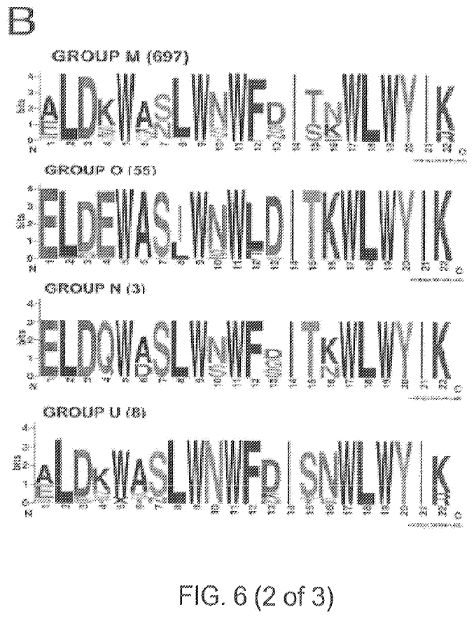

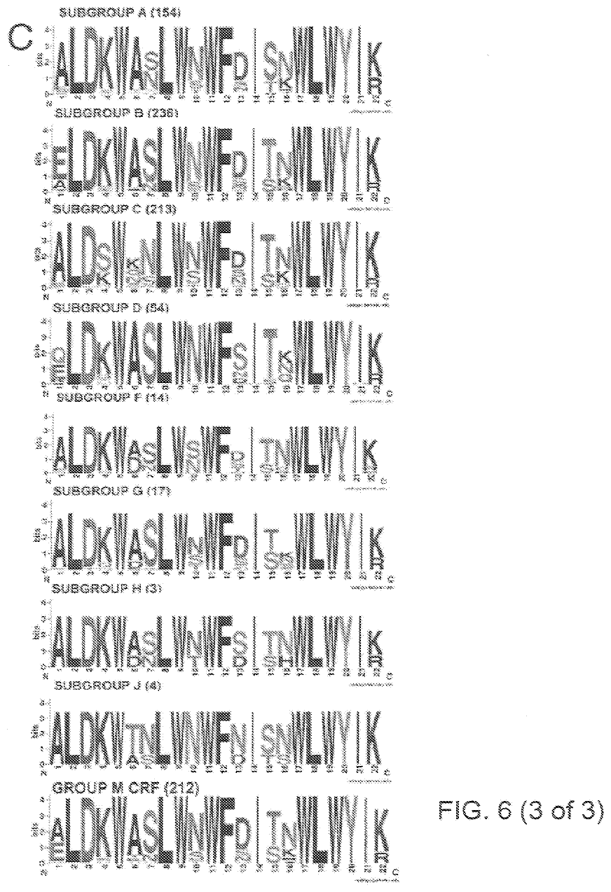

The initial data set (UniProt set) included sequences extracted from the UniProt Knowledgebase Controlled Vocabulary of Species, release 52.1 (www.expasy.org/cgibin/speclist). This set included 46 HIV-1, 13 HIV-2, and 15 SIV taxons whose database entries contain MPER sequence. The second data set (HIV database set) included 975 HIV-1 sequences extracted from the HIV Sequence Database (Kuiken et al. (2003) AIDS Rev 5: 52-61). The following HIV-1 groups were represented in the data sets: M (909 sequences), N (3), O (55), and unknown U(8). The group M contained sequence subgroups A (154), B (236), C (213), D (54), F (14), G (17), H (3), J (4), K (2), and circulating recombinant forms CRFs (212). The phylogenic guide tree file was generated by ClustalW (www.ebi.ac.uk/clustalw) and the graph in FIG. 6 was produced using MEGA4 (www.megasoftware.net).

The multiple sequence alignments of the full-length envelope sequences and of the MPER peptides were performed using the MAFFT program (Katoh et al. (2005) Genome Inform 16: 22-33). The reference HIV-1 envelope sequence is the standard HXB2 strain. The automatic strategy, moderately accurate option was selected for multiple sequence alignments. Patterns within the multiple sequence alignments were discerned using WebLogo tool for graphical representation of amino acid patterns within sequence alignments (Crooks et al. (2004) Genome Res 14: 1188-1190). Diversity analysis of HIV-1 envelope protein was performed using Sequence Variability Server (bio.dfci.harvard.edu/Tools/sys.html), which calculates Shannon entropy for multiple sequence alignments. The default values were used for sequence variability analysis.

The entropy analysis was performed on the multiple sequence alignment of the second data set of 975 full-length HIV-1 sequences. The alignment was done relative to the sequence and all the positions containing gaps were removed from the entropy calculations. Entropy was calculated as average values for windows of the length 10, 15, and 20 amino acids long. For window length of 20 amino acids, the mean value of entropy for MPER is 0.46 versus 0.85 for the envelope protein; minimal and maximal entropy within MPER are 0.27 and 0.63 versus 0.21 and 2.06 for envelope protein; SD for MPER entropy is 0.1 vs. 0.4 for envelope protein. The analysis of widow lengths of 10 and 15 amino acids agreed with the results for window length of 20. Hence, only the latter was used herein.

Conjugation of Cys-Modified MPER Peptides to Maleimide-Functionalized Nanoparticles.

Cys-modified MPER peptides are reconstituted in PBS pH 7.4 containing the reducing agent tris(2-carboxyethyl)phosphine hydrochloride (TCEP, Pierce Chemical Co.) to prevent intra-peptide disulfide bond formation. Cys-functionalized MPER peptides (0.1-10 μM) are incubated with 10 mg/mL maleimide-functionalized nanoparticles in PBS pH 7.4 containing 1 mM TCEP/25 mM EDTA at 20° C. for 1 hour to allow MPER adsorption/maleimide coupling. Nanoparticles are separated from unconjugated peptide by centrifugation (5 minutes at 14,000×g) and washing with buffer.

Encapsulation of T Helper Epitopes/CpG in Lipid-Enveloped Nanoparticle Core.

A 1 mL peptide solution (20 mg/mL in water) with or without CpG (1 mg/mL) is emulsified in 5 mL of dichloromethane containing 0.64 mg/mL lipid and 16 mg/mL PLGA using an Ika-Werke Ultra-Turrax T25 homogenizer at 13,500 rpm at 4° C. for 2 minutes. The peptide-in-PLGA emulsion is added to 100 mL deionized water with homogenization (13,500 rpm) at 4° C. for 2 minutes, followed by immediate sonication at 4° C. (2 minutes, 22 Watts with a Misonix Microson XL probe tip sonicator). The particles forming in the double emulsion are solidified by evaporating the organic solvent at atmospheric pressure with stirring at 20° C. for 12 hours, washed, and stored at 4° C. (short term storage) or lyophilized in the presence of trehalose and stored at 4° C. until used.

T Helper Epitope and CpG Release Kinetics.

One mL of Th peptide- and/or CpG-loaded nanoparticles (10 mg/mL) in RPMI 1640 medium containing 10% FCS or PBS pH 5.5 is incubated at 37° C. for 3-7 days. Peptide release is assessed by pelleting the nanoparticles at selected timepoints (e.g., 2 hours, 12 hours, 24 hours, or daily), collecting the supernatant, and resuspending the particles in fresh medium for further incubation. Peptide concentrations in the particle supernatants is assessed using the microBCA assay (Pierce Chem. Co.) following the manufacturer's instructions. Unlabeled CpG is used for experiments where peptide release is measured. To assess CpG release, FITC-conjugated CpG is encapsulated and its release quantified by fluorescence measurements on the supernatants, compared to a standard curve of FITC-CpG fluorescence.

Bone Marrow-Derived DC Culture.

Dendritic cells are prepared from bone marrow using the method described in Inaba et al. (1992) J Exp Med 176:1693-702. Briefly, marrow cells from the tibia and femur of C57Bl/6 mice are collected, red blood cells are lysed, and progenitors is cultured at 10⁶ cells/mL in the presence of 5 ng/mL GM-CSF in complete RPMI (RPMI 1640 medium supplemented with 10% FCS, 10 mM HEPES, 100 U/mL penicillin, 100 μg/mL streptomycin, 2 mM L-glutamine, and 50 μM 2-mercaptoethanol). Every 2 days, medium with GM-CSF is replenished; DCs will be used at days 6-7.

Targeting Protein Conjugation to Lipid-Enveloped Nanoparticles.

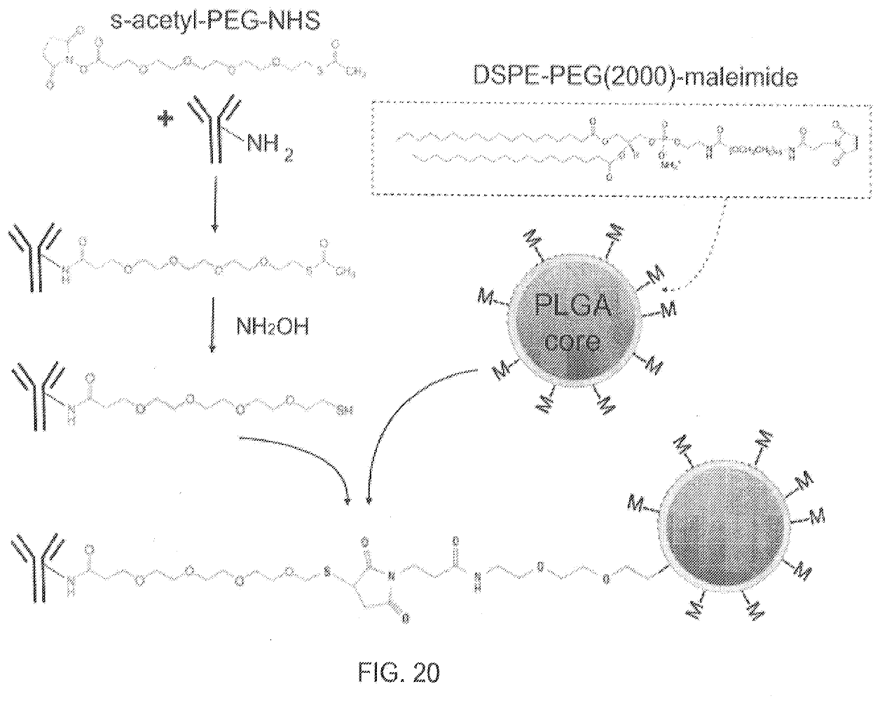

To conjugate flagellin or targeting antibodies to maleimide-bearing lipid-enveloped nanoparticles, the targeting proteins are first thiolated using a protected thiol, as outlined in FIG. 20. Targeting ligand (2 mg/mL) is mixed with s-acetyl-(PEO)4-NHS (1 mM) in PBS pH 7.4 and allowed to react for 30 minutes at 20° C. with agitation. Glycine is added to a final concentration of 35 mM to quench the reaction (15 min at 20° C. with agitation) followed by buffer exchange using a Zeba 0.5 mL desalting column (Pierce Chem. Co.) to remove unreacted glycine/SAT-PEO-NHS. The purified SAT-PEO-conjugated ligand is then deacetylated by incubated for 2 hours at 20° C. in PBS pH 7.4 containing 0.5 M hydroxylamine (Pierce), 25 mM EDTA. Deacetylated ligand is buffer exchanged into PBS pH 7.3 containing 10 mM EDTA and 10 mM TCEP using a desalting column. Maleimide-bearing nanoparticles is suspended (1 mg/mL) in this same buffer and the particles and ligand are mixed and reacted for 1 hour at 20° C. to allow maleimide coupling to the thiol-containing ligand. The ligand-functionalized nanoparticles are pelleted and washed by centrifugation and stored until use as before at 4° C. or lyophilized.

LeX-Polymer Conjugation to Lipid-Enveloped Nanoparticles.

LeX-PHEAAm (2 mg/mL) is activated with carbodiimidazole (CDI, 10 mM) in anhydrous DMSO under dry nitrogen for 1 hour at 20° C. The activated polymer is then diluted to 20 μg/mL in PBS pH 7.4 containing 1 mg/mL amine-PEG-functionalized lipid-enveloped nanoparticles to allowed to react at 20° C. for 4 hours. Unconjugated LeX-PHEAAm is removed by centrifugation and washing of the conjugated particles with PBS, followed by storage as described above.

### Example 2

### The Micelle-Bound MPER Adopts an L-Shaped Helical Structure

The HIV-1 MPER segment (amino acids 662-683 of HXB2 gp160) contains a large number of hydrophobic residues, and hence can only be solubilized in aqueous solutions in the presence of detergents or lipid vesicles. NMR spectroscopic studies of the HIV-1 strain HXB2 MPER in dodecyl phosphatidylcholine (DPC) micelles at pH 6.6 were carried out by using isotopically labeled peptide and multi-dimensional triple-resonance experiments. The solution structure was found to consist of two discrete helical segments with a central hinge, forming an L-shape (FIG. 1A). The N-terminal segment was found to contain a two-turn α-helix from D664 to W672, while the C-terminal segment was found to begin with a one-turn α-helix from 1675 to L679 followed by a 3₁₀ helix from W680 to K683. The characteristic α-helical 3-residue separated Hα to Hβ NOE and 4-residue separated Hα to HN NOE was clearly missing for residues F673 and N674 in the hinge region (FIG. 1B). The flexibility of the hinge region was found to result in an overall backbone <rmsd> of 0.59 Å when superimposed from residues 665 to 682 (Table 3).

TABLE 3

| | MPER (TALOS prediction) | | MPER in 2FX7 | |
|---|---|---|---|---|
| Amino acid | Φ (deg) | Ψ (deg) | Φ (deg) | Ψ (deg) |
| E662 | — | — | | |
| L663 | — | — | | |
| D664 | −68.84 +/− 15.64 | −34.81 +/− 11.59 | | |
| K665 | −66.11 +/− 11.60 | −37.3 +/− 10.50 | | |
| W666 | −64.13 +/− 15.42 | −43.72 +/− 13.45 | | |
| A667 | −59.76 +/− 7.97 | −42.9 +/− 6.24 | | |
| S668 | −75.29 +/− 10.59 | −34.58 +/− 15.88 | | |
| L669 | −78.07 +/− 11.70 | −22.94 +/− 6.38 | | |
| W670 | −95.98 +/− 25.27 | 127.29 +/− 22.86 | −102.333 (1TZG) | 92.289 (1TZG) |
| N671 | −87.45 +/− 14.57 | 135.67 +/− 23.65 | −82.932 (1TZG) | 111.923 (1TZG) |
| W672 | −55.59 +/− 4.50 | −40.97 +/− 11.20 | −53.277 | −34.380 |
| F673 | −65.30 +/− 8.82 | −29.44 +/− 18.94 | −69.321 | −3.972 |
| N674 | −93.21 +/− 20.14 | −7.92 +/− 18.37 | −103.721 | −8.330 (D674) |
| I675 | −57.53 +/− 8.22 | −42.79 +/− 11.00 | −58.791 | −48.138 |
| T676 | −65.16 +/− 7.09 | −36.05 +/− 9.05 | −63.423 | −28.778 |
| N677 | −66.76 +/− 5.46 | −42.63 +/− 8.08 | −66.757 | −44.019 |
| W678 | −62.78 +/− 12.09 | −41.66 +/− 9.36 | −61.844 | −45.028 |
| L679 | −65.98 +/− 5.09 | −40.27 +/− 12.43 | −59.312 | −40.615 |

However, the individual N- or C-terminal segments converged well, with backbone <rmsd> of 0.24 Å and 0.15 Å, respectively (FIG. 1C), excluding the two N-terminal residues, E662 and L663, and the C-terminal K683 which appear to be extended and unstructured. This structure was distinct from the straight α-helix of an earlier NMR model for the unabeled MPER peptide in DPC micelle at pH 3.5 (Schibli et al. (2001) Biochemistry 40:9570-9578), which does not present a single membrane-binding face. The kinked MPER structure, on the other hand, uniquely possessed a hydrophobic membrane-binding face containing 4 of the 5 W residues as well as the critical F673 residue described below, while 3 hydrophilic N residues within the 4E10 epitope are solvent exposed (FIG. 1D).

### Example 3

### Membrane Immersion-Depths of Individual MPER Residues

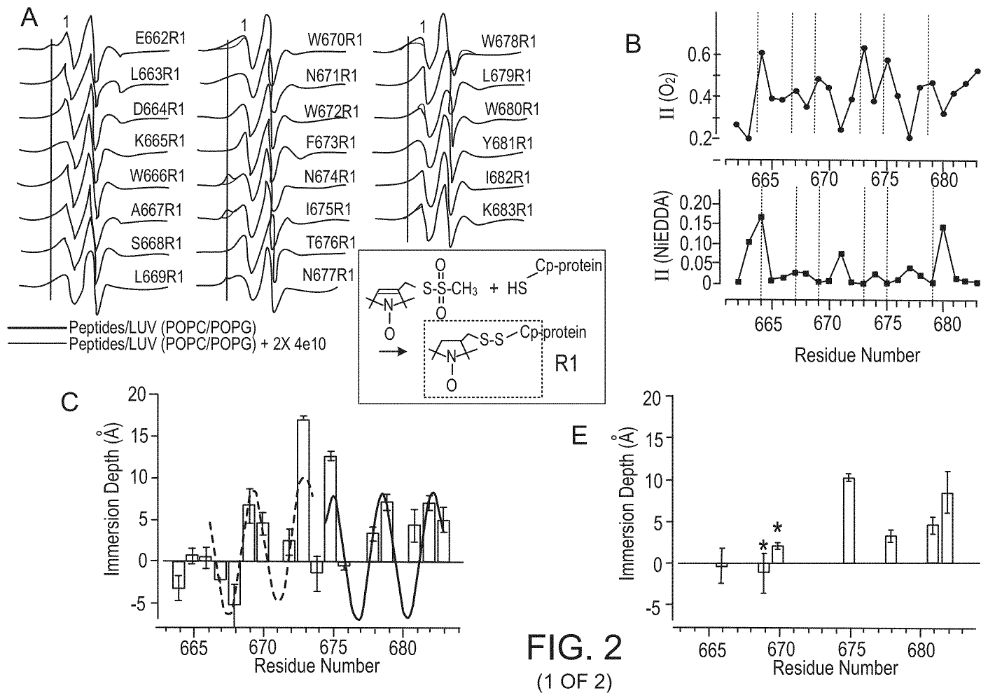

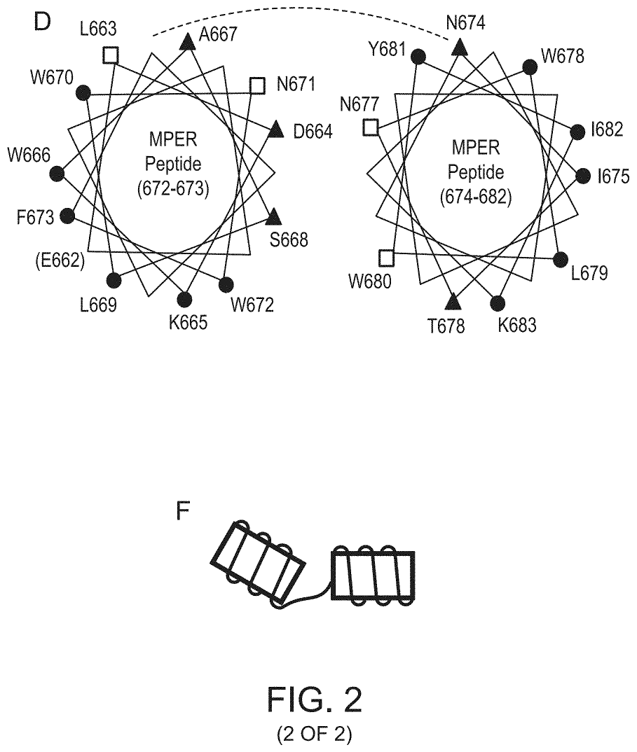

To experimentally determine the orientation of the MPER in the membrane-bound state, the site-directed spin labeling method (Hubbell et al. (1998) Curr Opin Struct Biol 8: 649-656) of electron paramagnetic resonance (EPR) spectroscopy was used to study 22 synthetic MPER peptides with spin-labels at different residue positions (FIG. 2A). The accessibility values of the nitroxide spin labeled sidechains (R1) to the relaxation agents, oxygen and NiEDDA, were measured by power saturation techniques (Altenbach et al. (1994) Proc Natl Acad Sci USA 91: 1667-1671) for each spin-labeled peptide bound to a lipid bilayer (liposome) consisting of POPC and POPG molecules. The plots of accessibility parameters Π(O₂) and Π(NiEDDA) (FIG. 2B) showed that the collision frequencies of the spin-labeled side chain R1 for the relaxation agents oscillate as a function of sequence position. Hence, the spin labels alternate between polar and nonpolar environments. Interestingly, the two curves oscillate approximately in the same phase for residues 662R1-667R1 but in the opposite phase (180°) for residues 668R1-683R1. The periodicity with local maxima (or minima) often occurs at every third or fourth sequence position, suggesting that most residues are in helical conformation in the presence of membrane. The membrane immersion-depths of MPER residues derived from the ratio of the accessibility parameters were determined by EPR as shown in FIG. 2C. The residues L669R1, W670R1, W672R1, F673R1, I675R1, W678R1, L679R1, Y681R1, I682R1 and K683R1 were found to be buried in the acyl chain region of the lipid bilayer (depth>0 Å) while residues K665R1, W666R1 and T676R1 were found to reside close to the interface between the acyl chain region and the lipid headgroup region. Residues D664R1, A667R1, S668R1 and N674R1 were found to be in the phospholipids headgroup region (−5≤depth≤0 Å). Other residues such as L663R1, N671R1, N677R1 and W680R1 are completely exposed to the aqueous phase so that the immersion-depths cannot be determined. The accessibility parameters and the immersion-depth data show that the membrane-interaction pattern can be best described by two out-of-phase amphipathic N- and C-terminal helices separated at residue N674 (FIG. 2D), which also supports the presence of the kink in the MPER helix.

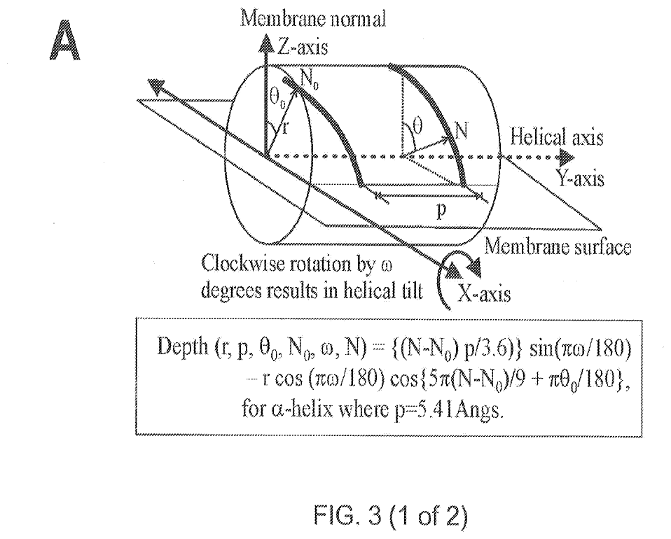

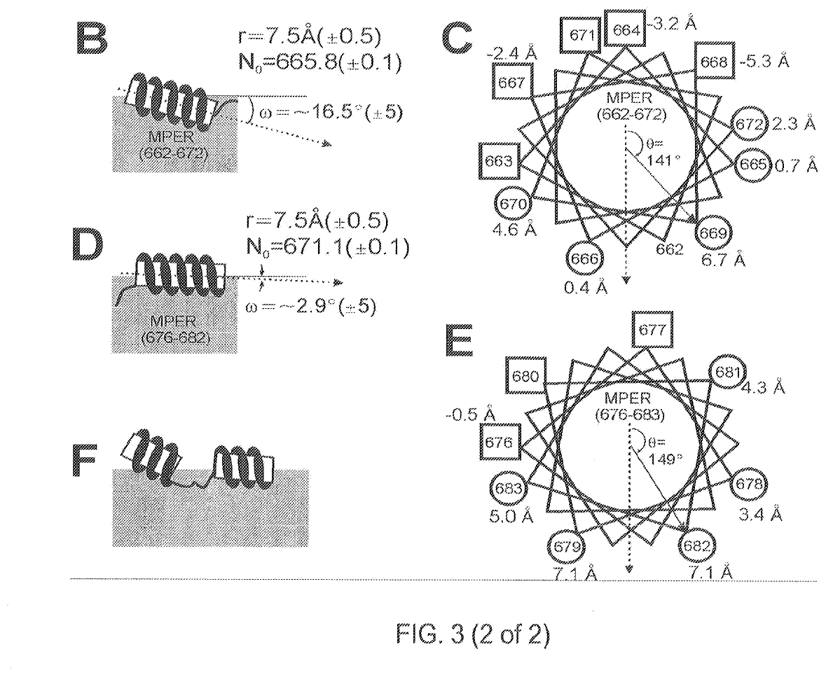

To provide a detailed structural basis for the EPR results, the orientation of the MPER peptide relative to the lipid bilayer was determined by fitting the membrane immersion-depth data by computer simulations using simple helical models (FIG. 3). As depicted in FIG. 2C, the N-terminal segment of the peptide (residues 664-672) is in α-helical conformation with a tilting angle of approximately 15° (upwards at the N-terminus) relative to the membrane surface (see also FIGS. 1D and 2F). The residues 662-666 in the N-terminal helical segment, however, did not fit well with the predicted depth pattern, for which the accessibility parameters Π(O₂) and Π(NiEDDA) oscillate approximately in the same phase (FIG. 2B). This discrepancy may originate from either altered spin label conformations or from high exposure to the aqueous phase, as often observed for helices on a soluble protein surface (Hubbell et al. (1998) Curr Opin Struct Biol 8: 649-656). The C-terminal segment (residues 675-683) lies essentially parallel to the membrane surface (tilt angle less than 5°, FIG. 2C and FIG. 3). The two helical segments form a kink (FIG. 2F) with angles ranging from 90° to 120° that are primarily defined by the peptide bonds between F673 and N674 (FIG. 1C). The pivot residue N674 resides in the membrane head-group region and points toward the aqueous phase. In contrast, F673 and I675, hydrogen-bonded within the N- and C-terminal helices respectively, anchoring deeply towards the hydrophobic region of the membrane (FIGS. 1D and 2C).

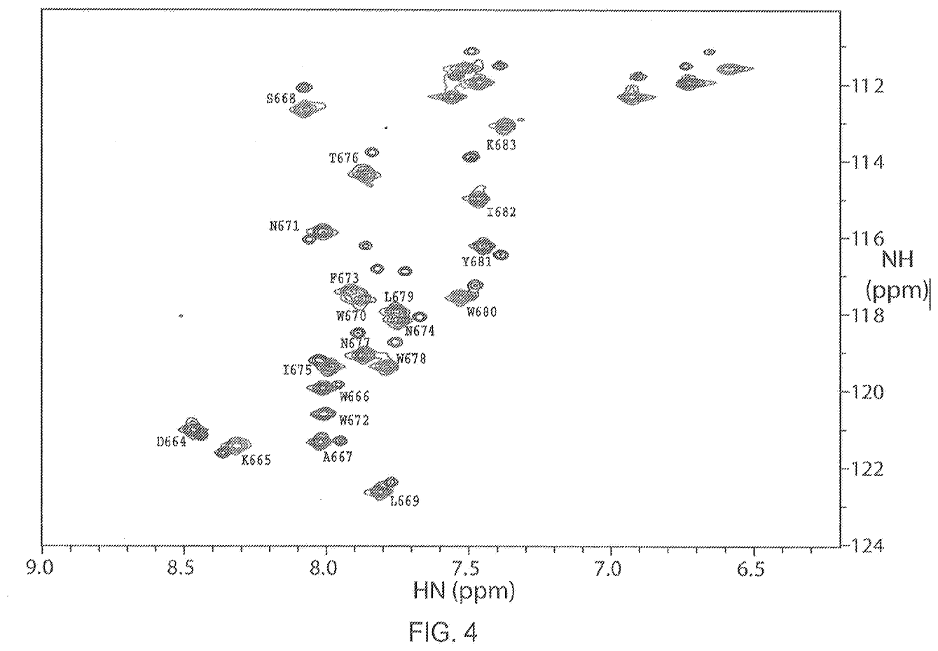

The NMR analyses of ¹⁵N-labeled MPER peptide in DPC micelle and disc-like DHPC-DMPC bicelle show similar spectral patterns (FIG. 4). Since the MPER peptide binds to the flat surfaces of lipid bicelle that resemble the membranes of much larger lipid vesicles, the conformations of the MPER peptides are expected to be similar in the membrane systems (Chou et al. (2002) J Am Chem Soc 124, 2450-2451) used in the EPR and NMR studies. The L-shaped structure was not caused by an adaptation of the peptide to the curvature of the micelle surface. Instead, the middle of the peptide forming the kink is immersed deepest into the micelle (FIG. 1D), while the N-terminus projects away from the micelle consistent with a trajectory connecting to the extracellular part of gp160 in the full-length protein. Overall, the N-terminal residues are predominantly exposed to the aqueous phase, whereas the C-terminal residues leading to the transmembrane helix are mostly immersed in the membrane.

### Example 4

### Exposed Residues Display Greatest Sequence Variability Within the Conserved MPER

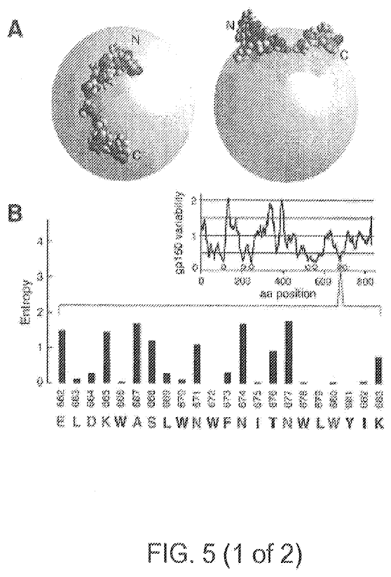

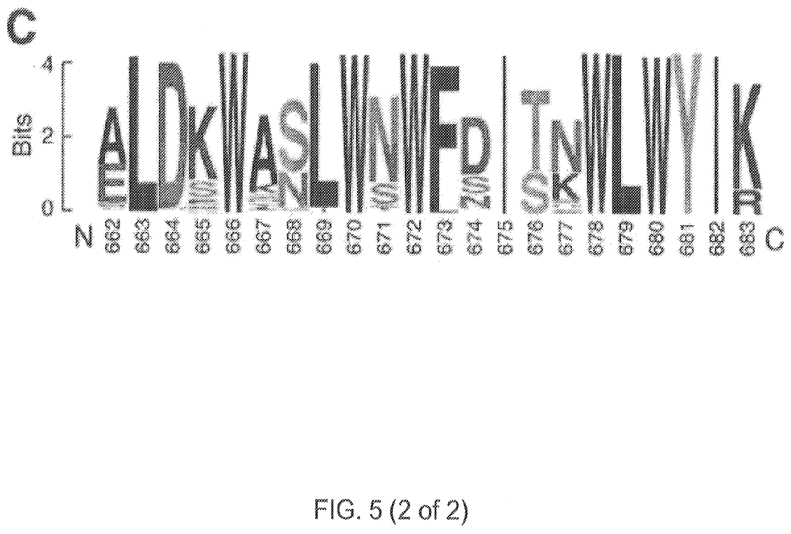

The space-filling models of the MPER revealed how it is largely immersed in a micelle (FIG. 5A). Remarkably, hydrophobic residues that were found to be buried in the lipid phase are the most conserved, in general, while those polar residues that were found to be exposed to the aqueous phase are the most variable. As shown by Shannon entropy analysis of 975 HIV-1 sequences compiled from M, O, N and U groups and available M subgroups (FIG. 5B and FIG. 6), the variability of amino acids at each of the 22 positions is limited, being among the least variable of all 20 amino acid segments probed within the gp160 molecule (FIG. 5B, insert). In particular, the 15 C-terminal residues of the MPER include only three positions, 671, 674 and 677 with values≥1. The other residues are either invariant or very restricted, primarily representing dimorphic variants (FIG. 5C). Nonetheless, the implications of even this limited variability for vaccine design, as discussed later, are remarkable since subtle sequence alterations at 671 and/or 674 affect 4E10 and Z13e1 binding. Immersion of conserved hydrophobic residues in lipid also facilitates evasion of immune attack.

### Example 5

### MPER Conformational Changes Upon 4E10 mAb Binding

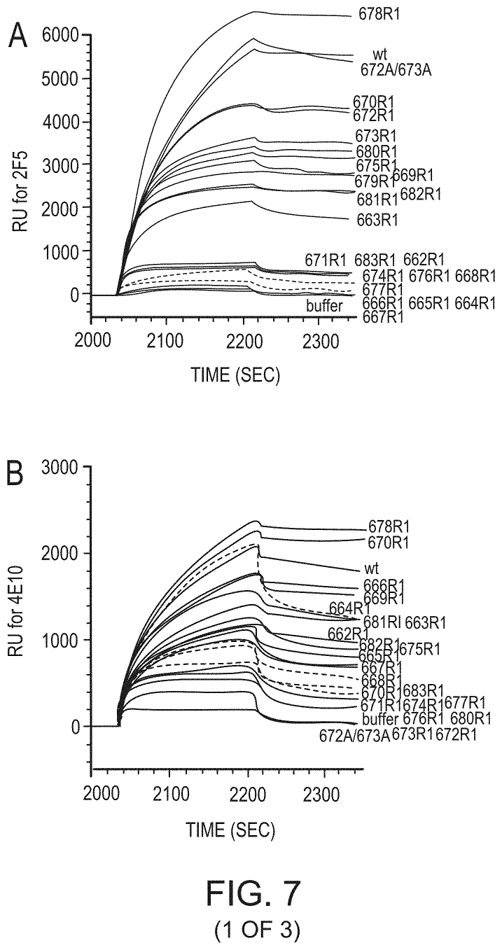

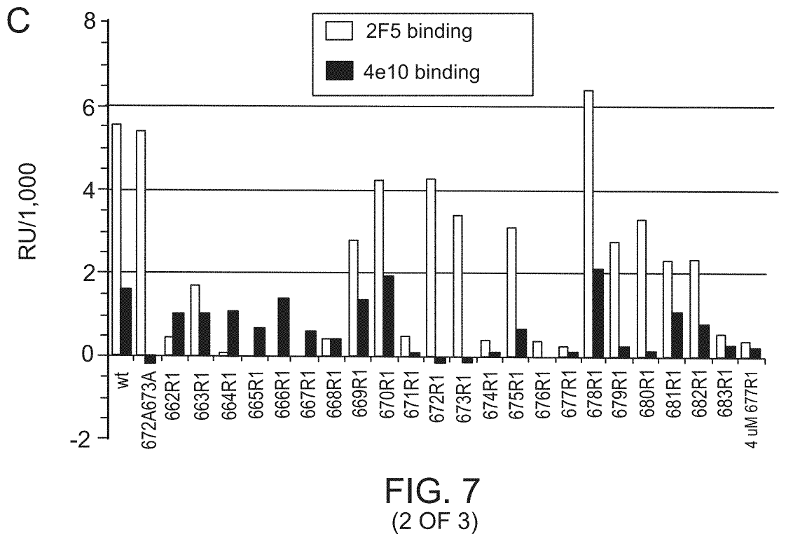

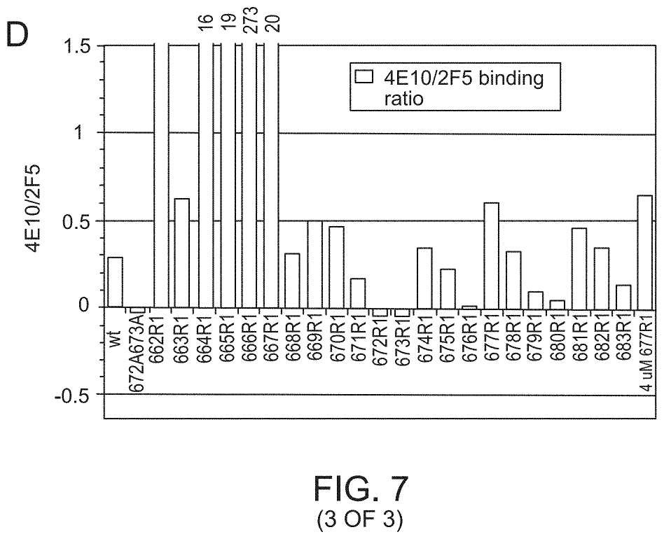

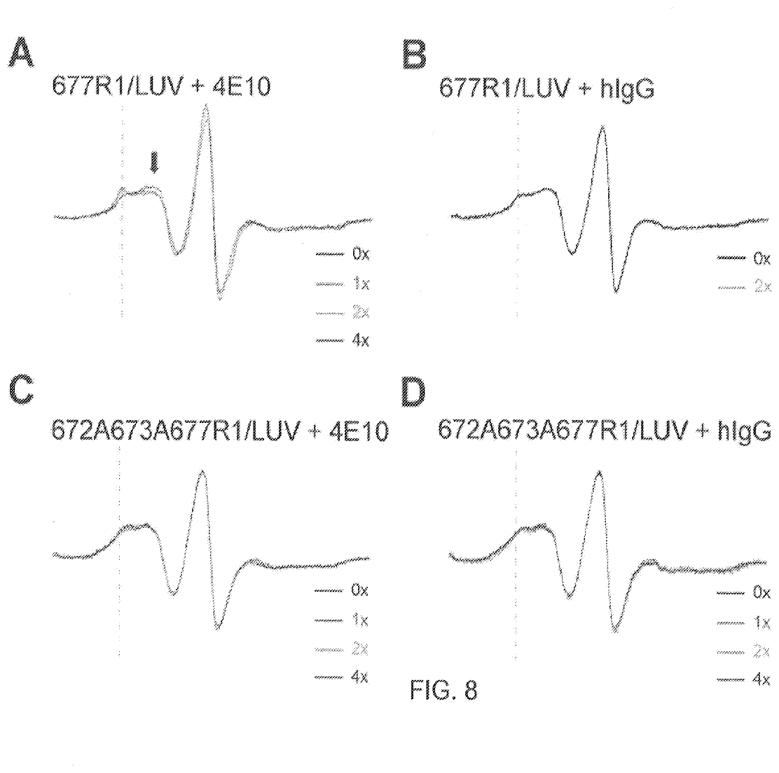

Unexpectedly, both EPR and NMR results showed that three hydrophobic residues (W672, F673, and L679) critical for neutralization of the HIV virus by 4E10 mAb (Zwick et al. (2005) J Virol 79:1252-1261) are buried in the lipid phase. Only the key polar T676 residue was found to be in the headgroup region. These findings suggest that the 4E10 mAb first attaches onto the membrane-bound MPER and subsequently induces a major conformational change in the peptide, exposing the complete epitope. To this end, EPR membrane immersion depth data on spin-labeled MPER peptides that retain affinity for 4E10 binding (FIG. 2A and FIG. 7) were obtained to confirm the orientation of the MPER peptide in complex with 4E10 mAb with respect to the membrane (FIG. 2E). Spectral decomposition of the spectra of 669R1, 679R1, 675R1, 678R1 and 681R1 in the presence of equimolar 4E10, which are essentially identical to those in FIG. 2A, suggest that the peptides are in equilibrium between the free and bound state, obscuring accurate determination of the immersion-depths of the antibody-bound peptide in the membrane. However, the change in the presence (FIG. 2E) and absence (FIG. 2C) of 4E10 could be used as an indicator of either the depth change or conformational change upon 4E10 binding for these residues. The trends in the change in the immersion depth data implied that the N-terminal segment is lifted up toward the aqueous phase while the C-terminal segment is little affected (FIG. 2E). The EPR spectral changes were highly specific to the 4E10 antibody and the MPER peptide sequence as shown by data derived from negative controls consisting of a 4E10-unreactive mutant peptide W672A/F673A/N677R1 and a non-binding control IgG antibody (FIG. 8). Notably, pronounced EPR spectral changes were observed in N674R1, I675R1, N677R1, W678R1 and Y681R1 (FIG. 2A), at or near the C-terminal end of the MPER peptide. On the other hand, the spin-labeling at positions W672, F673 and T676 completely abolished 4E10 antibody binding as determined by SPR experiments, and resulted in little or no EPR spectral changes in the presence of 4E10 (FIGS. 2A and 7).

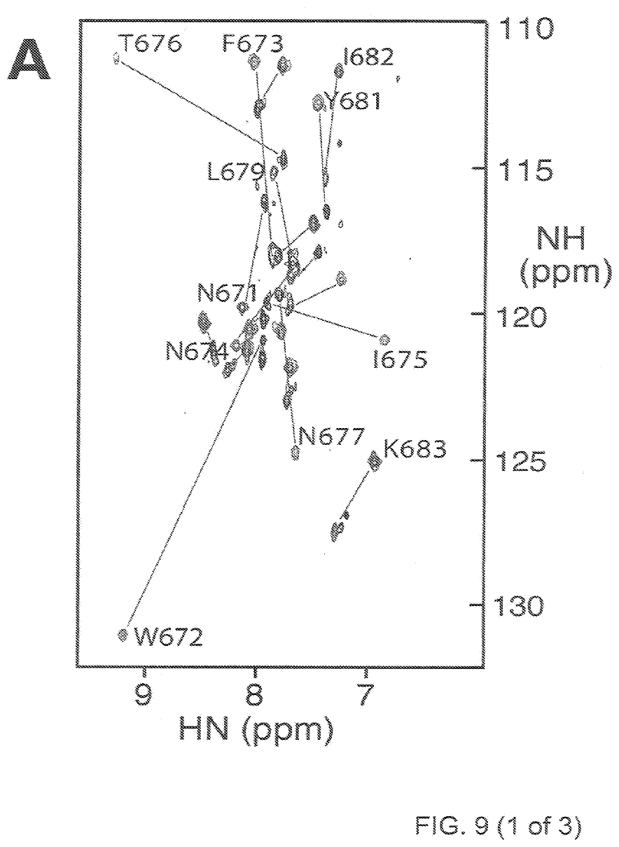

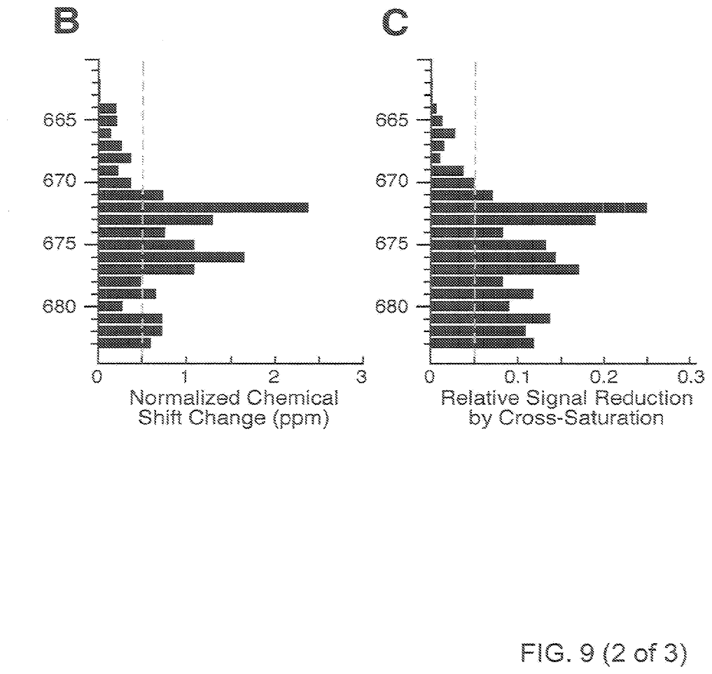

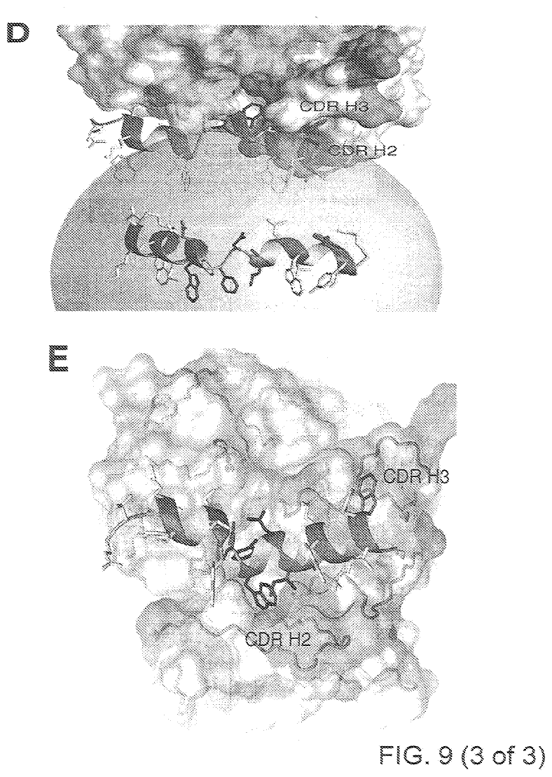

To confirm those structural changes and assess conformational alterations at all key binding residues, the MPER peptide in complex with the 4E10 antigen-binding fragment (Fab) in deuterated DPC micelles was investigated using NMR spectroscopy. The amide chemical shift perturbations of the MPER residues upon 4E10 binding are shown in FIGS. 9A and 9B. Whereas all residues that were measured manifest noticeable peak shifts, the residues displaying the most significant changes (>0.5 ppm of normalized chemical shifts) include the core 4E10 epitope residues WFNIT (672-676) (SEQ ID NO:44), plus residues N671, N677 and L679, and the three C-terminal residues Y681, I682 and K683. Results from NMR cross-saturation experiment further identify those residues in direct contact with the 4E10 antibody, as NMR magnetizations are transferred from the protonated methyl regions of 4E10 to the nearby amides of the per-deuterated MPER peptide. The residues in the MPER peptide that showed cross-saturation change (>5% reduction) include the C-terminal segment 671-683 (FIG. 9C). The region of MPER peptide responsible for 4E10 binding, therefore, is not restricted to the WFNIT core but comprises a segment spanning ˜18 Å, consistent with the width of the 4E10 Fab binding site. These results obtained for 4E10-binding in the presence of membrane are in general agreement with the recently published crystal structure of a soluble shorter (671-683) MPER peptide in complex with the 4E10 antibody (Cordoso et al. (2007) J Mol Biol 365, 1533-1544).

### Example 6

### Modeling 4E10 Interaction with the Micelle-Bound MPER

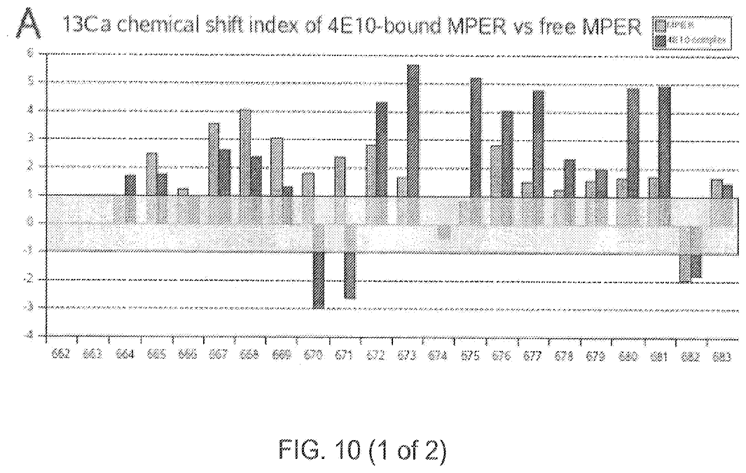

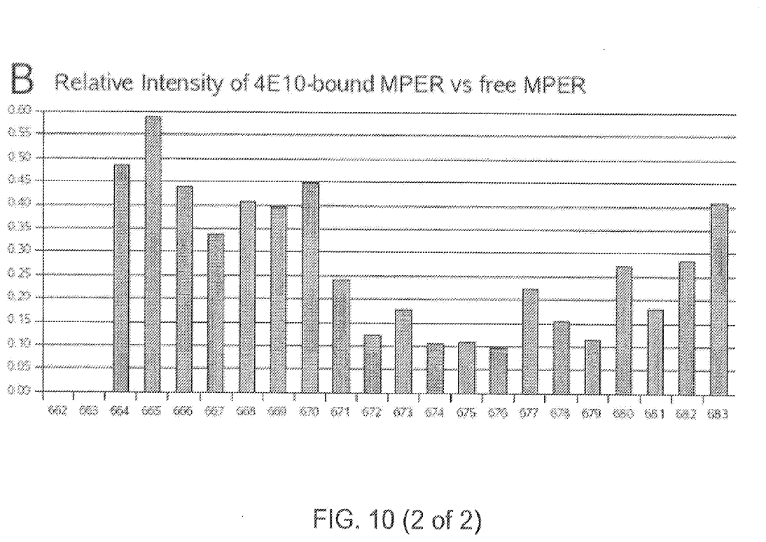

The combined NMR and EPR data refined the existing model of the 4E10 in complex with the full length MPER peptide. Secondary structure information was obtained from the ¹³C chemical shifts values of the per-deuterated MPER peptide in complex with 4E10 (FIG. 10 and Table 3). Upon binding, the hinge region in the kinked MPER peptide has become part of the C-terminal helix from W672 to K683 and residues W670 and N671 adopt an extended, non-helical conformation, in agreement with the crystal structure (Cordoso et al. (2007) J Mol Biol 365, 1533-1544 and Cordoso et al. (2005) Immunity 22, 163-173). The N-terminal segment was found to remain α-helical from residues D664 to L669, permitting this segment to be appended to the shorter MPER peptide from the crystal structure by overlapping the residues N671 and W672 in the model described herein (FIGS. 9D and 9E). The NWFNIT (SEQ ID NO:45) segment was found to make extensive interactions with antibody, with F673 swinging upward ˜15 Å (end-to-end) and inserting deeply into the 4E10 binding pocket. Additional contacts were found to be contributed by residues L679, W680, I682, and K683. Among the four MPER residues (N671, N674, N677, and W680) that are solvent accessible in the free form, N671 was found to be the most important for 4E10 interactions, by forming a hydrogen bond with the 4E10 light chain (Cordoso et al. (2007) J Mol Biol 365, 1533-1544 and Cordoso et al. (2005) Immunity 22, 163-173).

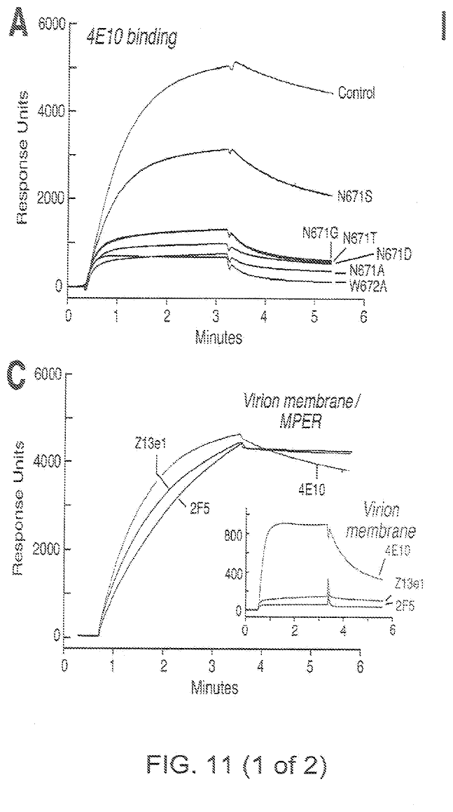

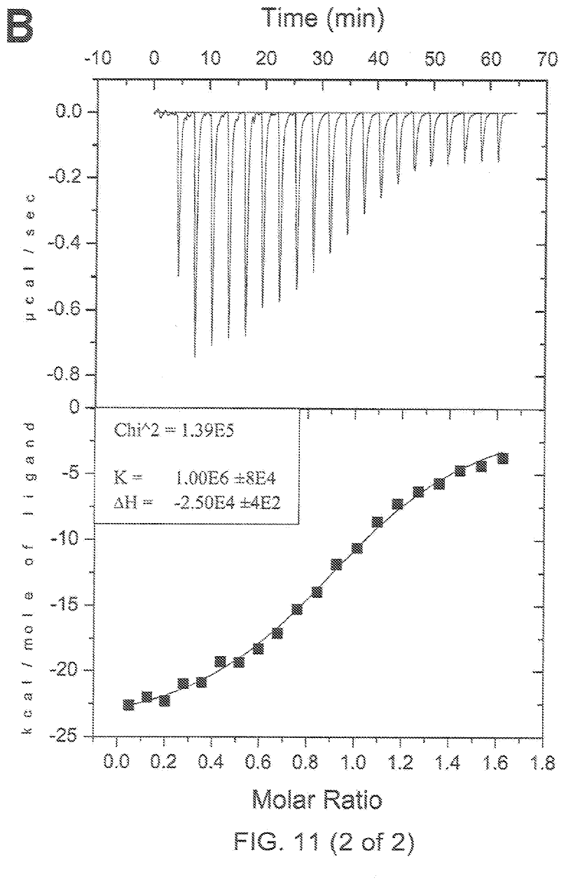

N671 likely participates in the initial contact between the 4E10 antibody and the lipid-embedded segment prior to MPER rearrangement as shown by the SPR data with a N671A mutant (FIG. 11A). Consistent with this notion, N671A was found to contribute little, if any, to 4E10 binding to MPER peptide in solution since other core residues including W672 and F673 are exposed (Brunel et al. (2006) J Virol 80:1680-1687. Furthermore, mutation of N671 to naturally occurring residues in other viral strains moderately (N671S) or severely (N671G, N671T, N671D) decreased 4E10 binding to the lipid-embedded MPER. Upon antibody binding, the N-terminal helix prior to N671 remained relatively mobile, although partially confined by the 4E10 light chain positioned above the membrane. Based on the EPR results, the orientation of the 4E10 antibody is such that it tilts away from the MPER peptide allowing the hydrophobic CDR2 loop of the heavy chain fragment to set anchor in the viral membrane (FIGS. 9D and 9E).

### Example 7

### Strong Lipid Binding is Not an Essential BNAb Requirement

To examine the energetics of 4E10 BNAb binding to the membrane-embedded MPER, ITC and SPR experiments were performed using liposomes whose lipid constituents mimic those found in HIV-1 virions (Bragger et al. (2006) Proc Natl Acad Sci USA 103, 2641-2646. The enthalpy change by ITC was determined to be −25 kcal/mole for the Fab form of 4E10, with a 1.0 μM Kd, suggesting a high entropic energy penalty (FIG. 11B). In addition, there was detectable monovalent binding of 4E10 Fab with the virion membrane-like liposome in the ITC experiment but was too weak to quantitate. As a consequence, intact BNAb IgG binding was examined using SPR. Consistent with a prior study (Alam et al. (2007) J Immunol 178:4424-4435), the best global curve fitting of 4E10 binding to the membrane-bound MPER involved a two-step conformational change model with Kd of ˜10 nM. FIG. 11C depicts the results of a comparison of the binding of 4E10, Z13e1, and 2F5 to the virion membrane-embedded MPER versus binding to the virion membrane alone. The 4E10, 2F5, and Z13e1 antibodies are described in, e.g., Zwick et al. (2005) J. Virol. 79(2):1252-1261; Ofek et al. (2004) J. Virol. 78(19):10724; Barbato et al. (2003) J. Virol. 330(5):1101-15; Zwick et al. (2001) J. Vorl. 75(22):10892-905; Joyce et al. (2002) J Biol. Chem. 277(48):45811-20; Parker et al. (2001) J. Virol. 75(22):10906-11; Zwick et al. (2004) J. Virol. 78(6):3155-61; and Nelson et al. (2007) J. Virol. 81(8):4033-43. As shown, specific binding of Z13e1 and 2F5 to the MPER is comparable to that of 4E10, but little or no direct binding to the membrane alone is observed. 4E10 mAb binds to the virion membrane mimic but with a much faster off-rate and consequently, a much weaker affinity (˜10 μM Kd). Thus strong membrane binding is not an essential BNAb characteristic.

### Example 8

### Fabrication of Lipid-Enveloped Micro- and Nano-Particles

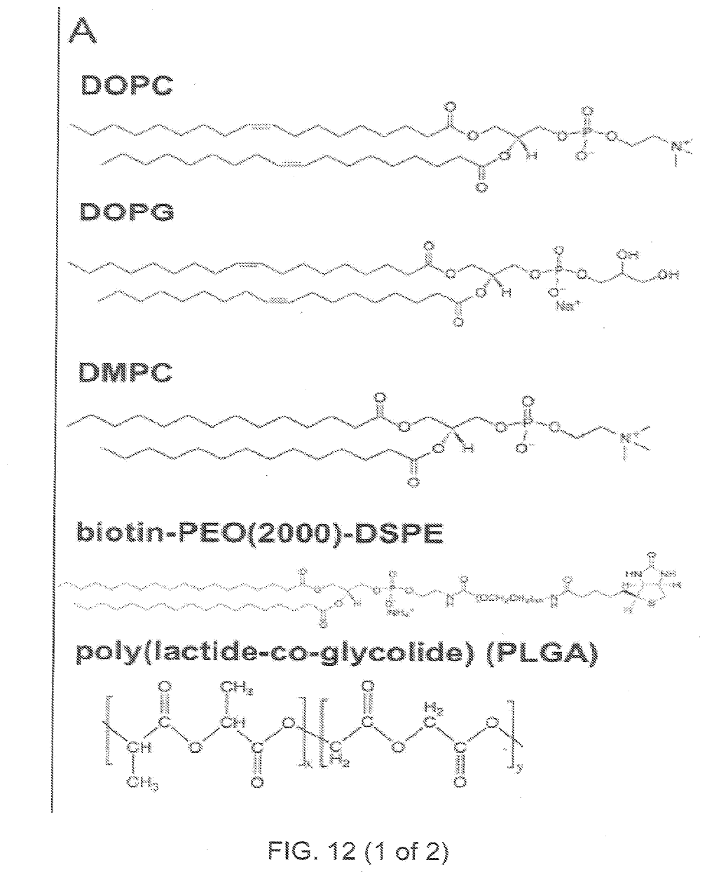

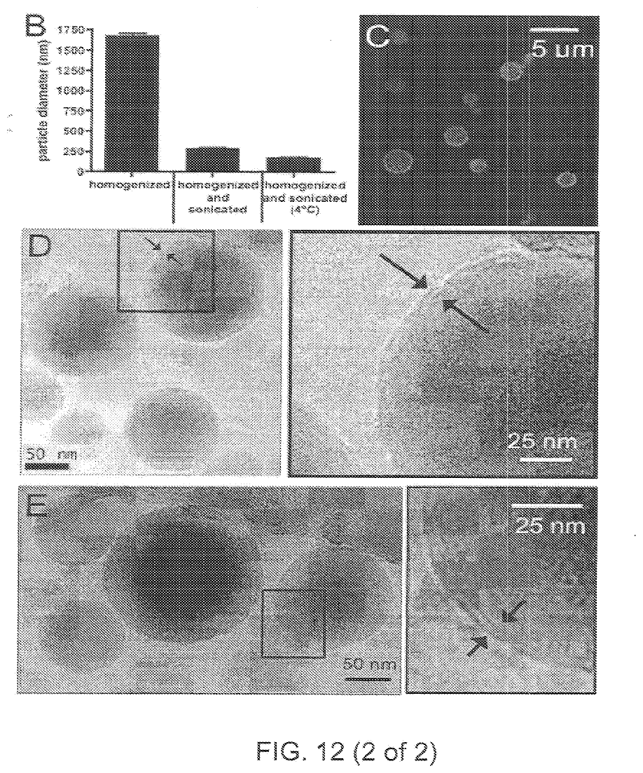

Phospholipid-enveloped nanoparticles were synthesized by an emulsion/solvent evaporation process: 5 mL of dichloromethane containing 0.64 mg/mL 1,2-dimyristoyl-sn-glycero-3-phoshpocholine (DMPC, Avanti Polar Lipids), 9.4 μg/mL 1,1′-dioctacdecyl-3,3,3′,3′-tetramethylindodicarbocyanine (DiD, fluorescent phospholipid analog, Invitrogen), and 16 mg/mL poly(lactide-co-glycolide) (PLGA, 50:50 lactide:glycolide by mass, MW 13 KDa, Medisorb) were added to 100 mL of deionized water with homogenization (13,500 rpm, Ika-Werke Ultra-Turrax T25 Basic homogenizer) at 20° C. for 2 minutes, forming an initial emulsion (FIG. 12A). Evaporation of the dichloromethane from this initial emulsion by stirring at 20° C. under atmospheric pressure for 6 hours led to the formation of micron-sized lipid-enveloped particles (FIG. 12B). Immediately sonicating the particles at 20° C. or 4° C. (2 minutes, 22 Watts with a Misonix Microson XL probe tip sonicator), after the initial homogenization and prior to organic solvent evaporation, lipid-coated PLGA nanoparticles were obtained with mean hydrodynamic diameters of ˜250 nm or ˜180 nm, as determined by dynamic light scattering (FIG. 12B). Simple changes to the processing scheme allow the mean particle size to be adjusted.

Particles containing 1 mole % rhodamine-labeled lipid, confocal microscopy were synthesized and an enrichment of lipid fluorescence at the surface of lipid/PLGA microparticles was observed (FIG. 12C). To provide more direct evidence for the structural organization at the surface of lipid-enveloped particles, cryo-electron microscopy (cryoEM): CryoEM imaging was used and revealed that many of the particles in preparations with mean hydrodynamic diameters of 150-180 nm were ˜100 nm in size (FIGS. 12D and 12E). Imaging of unstained preparations of the nanoparticles (FIGS. 12D and 12E) revealed a translucent polymer/lipid core with a clearly detectable surface layer of lipid, with electron-dense stripes defining the location of the lipid headgroups. These surface lipid layers often appeared to have a bilayer structure (FIGS. 12D and 12E insets/right panels). Particles were incubated for 10 days in PBS to partially hydrolyze the PLGA cores and exhibited further evidence of lipid bilayers at the surface of the particles using cryoEM. No free liposomes were observed in these preparations.

The composition of lipids included in the particle fabrication was readily varied, and inclusion of 1-10 mole % of biotinylated, fluorophore-conjugated, or maleimide-functionalized lipids in the lipid component of the particle synthesis did not significantly alter the particle sizes or lipid assembly as observed by cryoEM. In addition, use of an HIV envelope-mimicking lipid composition or T cell membrane-mimicking composition (DOPC/sphingomyelin/DOPE/DOPG/cholesterol at a 9:18:20:9:44 or 34:7:16:10:33 mole ratio, respectively) in the synthesis also gave lipid-coated particles of similar size.

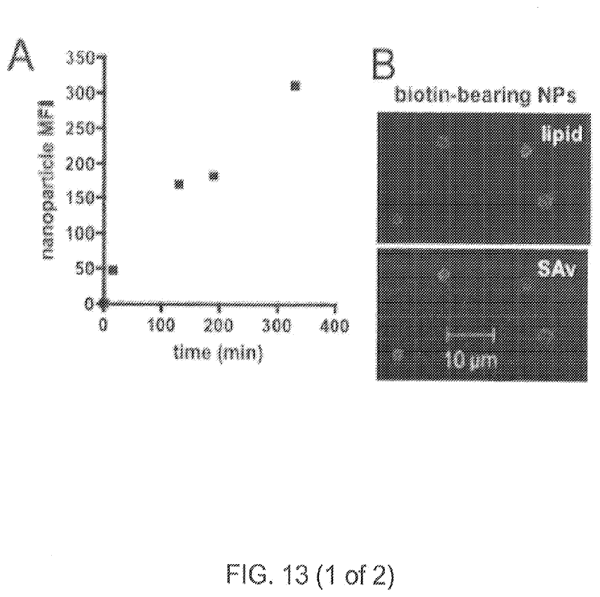

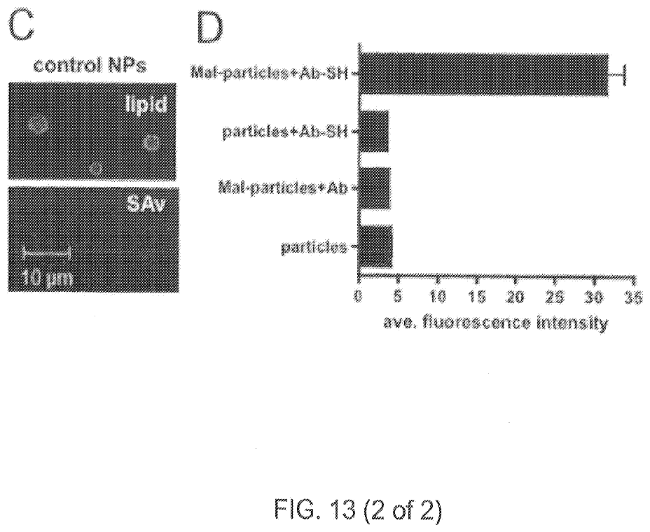

Incubation of lipid-enveloped nanoparticles with dendritic cells led to uptake of the particles of DCs over time in culture (FIG. 13A). However, to control the fate of particles following binding to DCs, and to preferentially target nanoparticles carrying MPER and HIV T cell epitopes to dendritic cells, targeting ligands are conjugated to the surface of the lipid-enveloped nanoparticles. By mixing small quantities of derivatized lipids with DMPC or DOPC base phospholipids in the particle synthesis, functional groups are introduced in the lipid envelope, as described above. Functionalized lipids incorporated in the synthesis were accessible at the surface of lipid-enveloped particles, as evidenced by the specific binding of fluorescent streptavidin to particles containing 1 mole % DSPE-PEG(2000)-biotin lipid (FIG. 12A, Avanti Polar Lipids) in the lipid component (FIG. 13B).

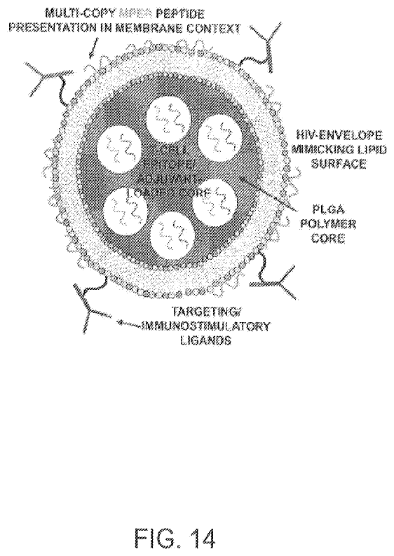

To demonstrate antibody functionalization, lipid-enveloped microparticles were synthesized including 1 mole % DSPE-PEG(2000)-maleimide (1,2-Distearoyl-sn-Glycero-3-Phosphoethanolamine-N-[Maleimide(Polyethylene Glycol)2000] (Avanti Polar Lipids) in the lipid component. Alexa fluor 488-conjugated rat IgG was thiolated with s-acetyl-PEO₄-NHS (a protected thiol crosslinker that reacts with primary amines; Pierce Chemical Co.) following the manufacturer's instructions, and then coupled to maleimide-functionalized nanoparticles by mixing thiolated antibody (400 μg/mL) with maleimide-bearing particles (10 mg/mL) in PBS pH 7.4 with 25 mM EDTA and 10 mM tris(2-carboxyethyl)phosphine hydrochloride (TCEP) for 1 hour at 20° C. with agitation. This coupling reaction anchored the antibody covalently through a thioether linkage to the lipid-anchored poly(ethylene glycol) spacer, as schematically illustrated in FIG. 14. Control reactions of particles lacking maleimide or using non-thiolated antibody were run in parallel. Following conjugation, the particles were collected by centrifugation and washed to remove unbound antibody, then imaged by confocal microscopy with a CCD camera. Surface fluorescence on particles qualitatively similar to that shown for SAv conjugation in FIG. 13B was only observed for reactions of maleimide-functionalized particles mixed with thiolated antibody; no Alexa fluorescence was observed for particles reacted under control conditions. This is quantitatively summarized by the mean fluorescence intensity of individual particles calculated from confocal images (fluorescence intensities collected from CCD pixel intensities) (FIG. 13D); only maleimide-particles mixed with thiolated Ab showed intensities above the background detected with untreated particles.

### Example 9

### MPER Peptide Binding to Lipid-Enveloped Nanoparticles and Neutralizing Antibody Recognition of Particle-Associated MPER

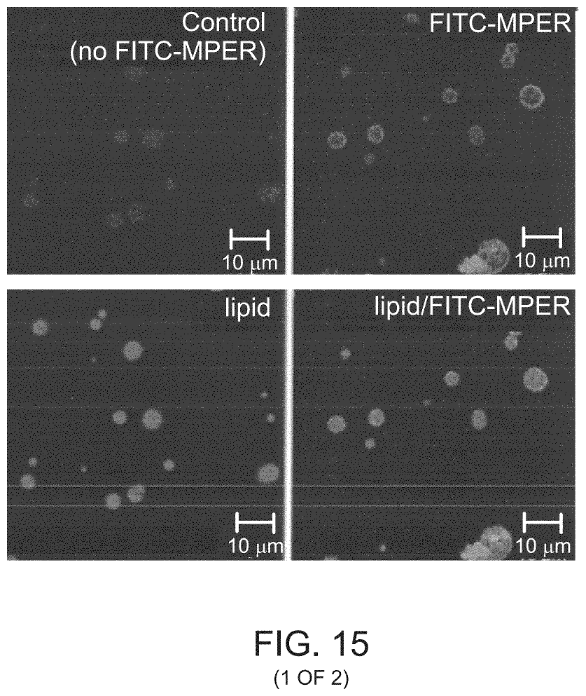

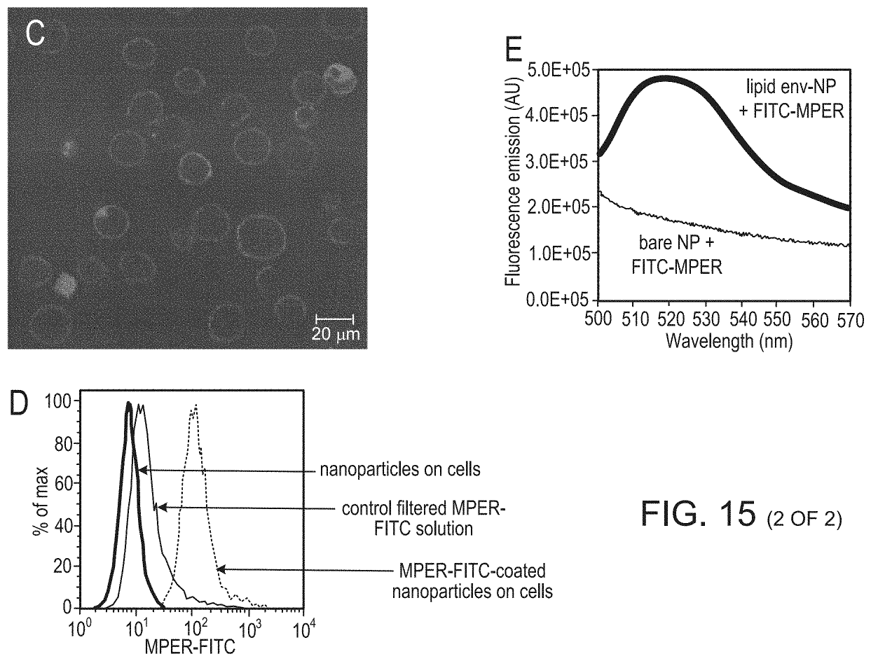

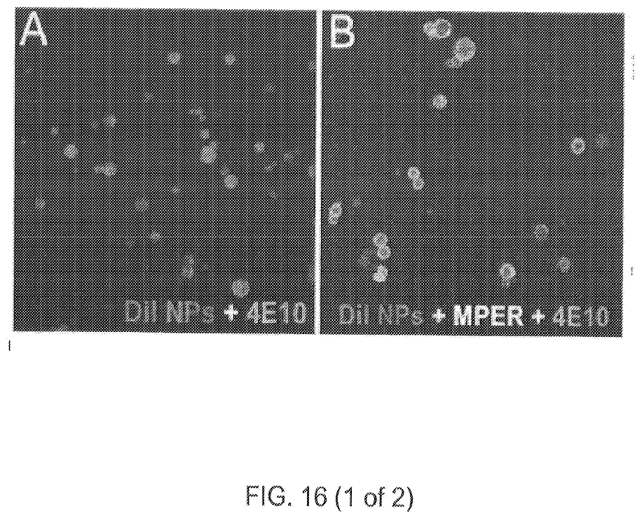

MPER peptides (residues 662-683 of the env protein) contacted to phospholipid membranes or micelles spontaneously adsorb to the phospholipid membranes and micelles, taking on a two-helix conformation partially buried in the lipid surface. To determine if MPER peptides would likewise bind to lipid-enveloped PLGA particles, MPER peptides (ELDKWASLWNWFNITNWLWYIK (SEQ ID NO:2)) FITC-labeled at the N-terminus were incubated with 10 mg/mL lipid-enveloped particles for 30 min at 37° C., testing a range of MPER concentrations. Following incubation, the particles were washed by centrifugal filtration to remove unbound FITC-MPER, and then imaged by confocal fluorescence microscopy. As shown in FIGS. 15A and 15B, MPER peptide readily adsorbed to lipid-coated PLGA microparticles. To analyze MPER adsorption to PLGA nanoparticles (which diffused too quickly in aqueous suspensions for direct confocal imaging), a flow cytometry-based assay was developed, where nanoparticles were ‘captured’ on the surface of cells for fluorescence analysis. First, lipid-enveloped nanoparticles bearing surface biotin groups were prepared by adding 1 mole % DSPE-PEG(2000)-biotin to the lipid component of the particle synthesis. The resulting biotinylated particles were incubated with 10 μM FITC-MPER and then washed as before to remove unbound MPER. As a control, a 10 μM solution of FITC-MPER was carried through the same washing steps, to ensure that no free MPER was detectable in the cytometry assay. To capture the biotinylated nanoparticles from solution, the murine dendritic cell line DC2.4 was biotinylated (using Sulfo-NHS-LC-LC-biotin, Pierce Chemical Co., per the manufacturer's instructions) at the surface of the cells, stained with streptavidin (5 μg/mL for 30 min at 4° C.), washed, then incubated with 10 mg/mL biotinylated nanoparticles (with or without adsorbed FITC-MPER) at 4° C. for 30 min. The cells were washed and then analyzed on a BD FACSCalibur flow cytometer to detect bound nanoparticles (DiD fluorescence) and MPER (FITC fluorescence). As shown in FIG. 15C, confocal microscopy of the nanoparticle-decorated DC2.4 cells revealed high densities of nanoparticles bound to each cell following this capture assay, forming dense punctate staining on the surface of each cell. Flow cytometry analysis of the nanoparticle-decorated cells showed clear binding of FITC-MPER to the biotinylated nanoparticles (FIG. 15D), well above the background autofluorescence of ‘blank’ nanoparticles bound to cells or the filtered MPER solution control. To determine if the lipid surface of the nanoparticles is important for MPER binding, lipid-enveloped PLGA nanoparticles or ‘bare’ PLGA nanoparticles were incubated with 10 μM FITC-MPER for 1 hour at 37° C., washed to remove unbound MPER, and then recorded fluorescence emission spectra from the dilute particle suspension in the FITC emission range using 450 nm excitation light. As shown in FIG. 15E, clear FITC emission indicating strong MPER binding to lipid-enveloped nanoparticles was observed, but bare PLGA particles showed no evidence for MPER binding. Thus, the lipid envelope is key to promoting MPER binding to the nanoparticles.

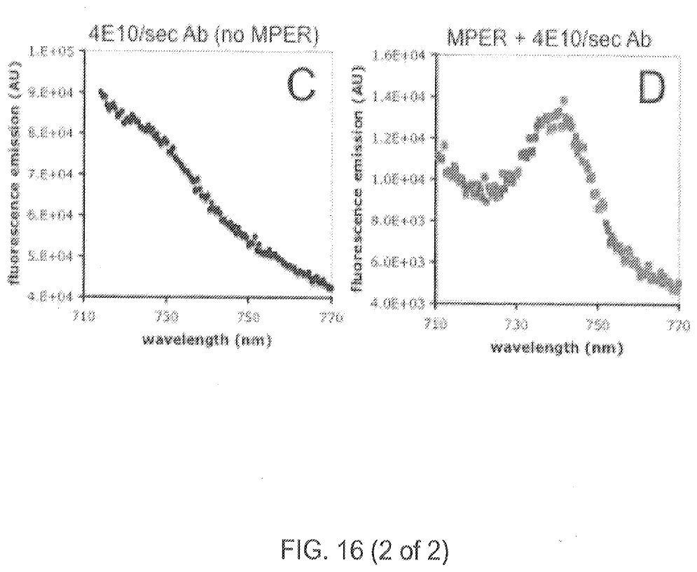

MPER adsorbed to lipid micelles or liposomes takes on a conformation recognized by the 4E10 broadly neutralizing anti-gp41 antibody. To determine if the 4E10 epitope is also accessible when MPER adsorbs to lipid-enveloped PLGA particles, 10 mg/mL DMPC lipid-coated PLGA microparticles was incubated with 10 μM MPER for 30 min at 37° C., washed by centrifugation to remove unbound MPER, and then stained the microspheres with 4E10 antibody and Alexafluor-labeled secondary antibody. Although 4E10 has been shown to interact with some lipids, DMPC-enveloped control particles (not exposed to MPER, FIG. 16A) showed no background 4E10/secondary Ab binding. In contrast, MPER-coated particles (FIG. 16B) were brightly stained, suggesting that 4E10 recognized MPER bound to the surface of lipid-enveloped PLGA particles. Control particles coated with MPER and stained with the secondary antibody alone showed no background secondary Ab staining. Lipid-enveloped nanoparticles were too small to directly observe in suspension by confocal microscopy. Thus, to determine if 4E10 also recognized MPER adsorbed to nanoparticles, 150 nm DMPC-enveloped nanoparticles was incubated with MPER (as described for the microparticles), then analyzed the fluorescence emission spectra of dilute suspensions of the nanoparticles stained with 4E10 and an Alexa 647-conjugated secondary antibody (emission peak ˜740 nm). Control nanoparticles that were not exposed to MPER showed no evidence for 4E10/secondary antibody binding (FIG. 16C), while MPER-coated nanoparticles exhibited a clear fluorescence emission peak.

### Example 10

### Nanoparticles in the 200 nm Size Range are Efficiently Transported to Lymph Nodes Following Intradermal Injection and Predominantly Localize in DCs and B Cells

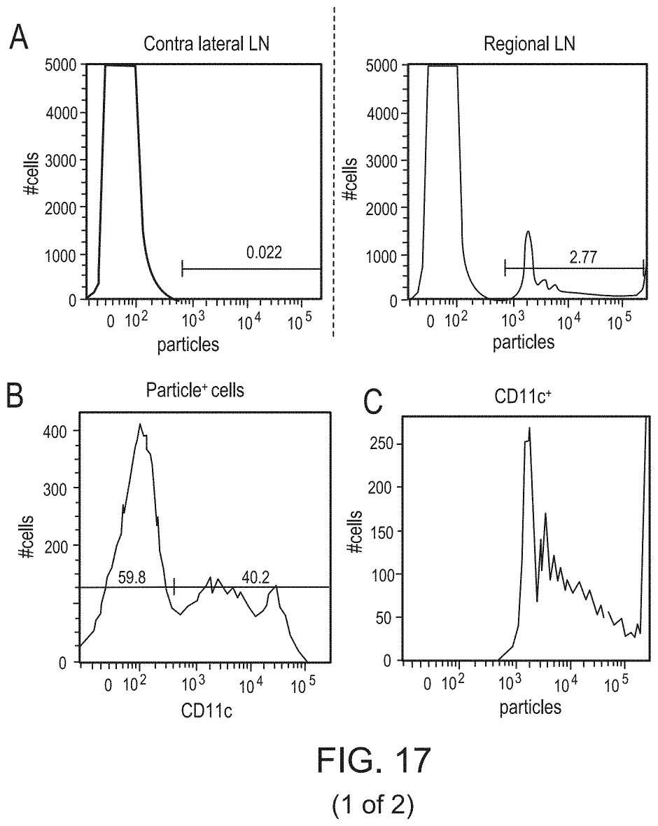

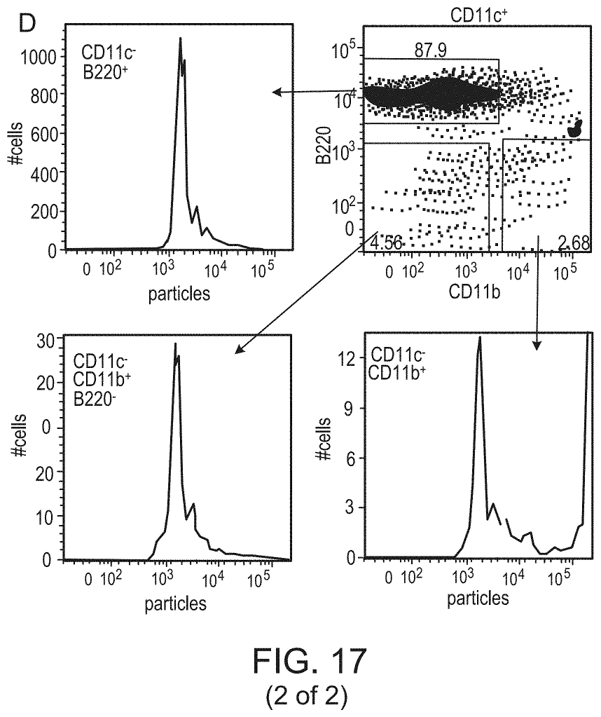

Tests were also conducted to determine the efficiency of nanoparticle transport to lymph nodes. Immunization through the intradermal (i.d.) route has been suggested to elicit immune responses at 10-fold lower doses of antigen as compared to other routes such as subcutaneous. In addition i.d. immunization elicits both systemic and mucosal immunity. To determine whether nanoparticles with sizes similar to the lipid-enveloped particles described here are transported to lymph nodes effectively following intradermal immunization, and what cell types take up nanoparticles following i.d. immunization, 8 week old C57Bl/6 mice (groups of 2) were immunized with fluorescent polystyrene nanoparticles 200 nm in diameter (Invitrogen Fluospheres, Invitrogen, Carlsbad, Calif.). Anesthetized mice received 2 mg of nanoparticles in 50 μL of sterile PBS i.d. Forty-eight hour post injection, the animals were sacrificed and the draining inguinal lymph nodes and contralateral control lymph nodes were recovered. Lymph nodes (LN) were digested with collagenase and the recovered cells were stained with fluorescent antibodies against CD11c, CD11b, and B220, and analyzed by flow cytometry. Nanoparticle fluorescence was clearly detected in ˜3% of the total LN cells of draining lymph nodes, but none were detected in contralateral LNs (FIG. 17A). Of the particle containing cells, ˜40% were CD11c+ dendritic cells (FIGS. 17B and 17C). Among the CD11c−particle+ cells, the majority (˜88%) were B220+CD11b− B cells (FIG. 17D). Thus, i.d. injection of nanoparticles in the same size range as the lipid-enveloped particles described above leads to substantial nanoparticle accumulation in lymph nodes by 48 hours, with both dendritic cells and B cells prominently taking up the particles. These results suggest that i.d. immunization is an appropriate choice for the in vivo tests of lipid-enveloped nanoparticle MPER delivery.

### Example 11

### Synthesis and Chemical Modification of Lipid-Enveloped Nanoparticles as MPER Carriers

Synthesis of Sub-50 nm-Diameter Lipid-Enveloped PLGA Nanoparticles.

To determine whether the antibody response elicited by MPER-carrying nanoparticle vaccination is more effectively triggered by direct delivery of the nanoparticles to the draining lymph nodes or by cell-mediated transport of the nanoparticles from injection sites to the lymph nodes, conditions in which to prepare sub-50 nm mean diameter (target diameter 30 nm) enveloped PLGA particles as well as larger 100 nm mean diameter nanoparticles are determined. The size of lipid-enveloped PLGA particles was readily modulated by varying emulsion formation conditions (FIG. 12A) or polymer/lipid concentration in the organic phase. Various parameters are adjusted to produce the particle size of interest. The amount of time of probe tip sonication at 4° C. following the initial homogenization is evaluated to enhance the formation of ultrasmall organic phase droplets in the emulsion. As a second approach, the concentration of polymer in the organic phase is reduced from 16 mg/mL to 5, 1, or 0.2 mg/mL PLGA, reducing the viscosity of the organic phase and facilitating smaller droplet formation.

Nanoparticles are washed post-synthesis using 100 kDa centrifugal filters, and stored at 4° C. (for short-term storage) or lyophilized in trehalose until used. Defined nanoparticle suspensions are prepared for studies by weighing lyophilized particles and resuspending in defined volumes of buffer before use.

Covalent Anchoring of MPER Peptide to Lipid-Enveloped Nanoparticles.

To ensure that MPER peptides remain associated with nanoparticles following immunization and increase the likelihood of presentation of these peptides in a correct membrane-mediated conformation for B cell priming in vivo, the optimal conditions to covalently conjugate MPER peptides to the surface of lipid-enveloped nanoparticles are determined. For example, lipid adsorbed and covalently-anchored MPER containing nanoparticles are compared.

Lipids carrying maleimide functional groups attached to the lipid headgroup via a poly(ethylene glycol) spacer are used to form covalent thioether linkages to cysteines introduced at the termini of the MPER peptide. Preliminary experiments of MPER interacting with lipid surfaces revealed that the N-terminal segment of the MPER sequence takes on a canted helix orientation extending out of the lipid headgroups while the C-terminal segment forms a helix more deeply buried in the lipid layer. The C-terminal segment of this peptide also formed a central part of the footprint of the 4E10 neutralizing antibody. Thus, it is expected that covalent tethering via linker residues at the N-terminus of the peptide are more likely to anchor the peptide without disrupting 4E10 recognition. MPER peptides (residues 662-683, ELDKWASLWNWFNITNWLWYIK (SEQ ID NO:2)) extended at the N-terminus, C-terminus, or both with a short cysteine linker sequence (CGGGS (SEQ ID NO:39), placing a free cysteine at one or both ends of the peptide) are obtained. For fluorescence tracking studies, peptides with a FITC tag on the N-terminus or following the Cys residue in the anchorable MPER are obtained.

Maleimide-functionalized nanoparticles are prepared by including 1 mole % mal-PEG-DHPE in the lipid component of the lipid-enveloped nanoparticle synthesis. Cys-functionalized MPER peptides are coupled to maleimide functionalized nanoparticles by incubation of particles and MPER in reaction buffer (detailed protocol in experimental methods section below). The efficiency of peptide conjugation and final coupling yields obtained by this reaction are assessed using FITC-labeled MPER peptides. An aliquot containing a known quantity of FITC-MPER-conjugated particles is and the particles/lipid/MPER are solubilized by treatment with 0.5M NaOH/1% SDS for 30 min, a treatment that we have confirmed hydrolyzes and dissolves the PLGA core of the particles. The solution is neutralized with HCl, and the solution concentration of FITC-MPER is determined by fluorescence spectrophotometry, relative to a FITC-MPER standard curve. This measurement is further confirmed by direct microBCA assay (Pierce Chem. Co.) to measure peptide concentration.

Encapsulation of T Cell Helper Epitopes and CpG Oligonucleotides in the Bioresorbable Core of Lipid-Enveloped Nanoparticles.

Peptides or adjuvant molecules can be encapsulated within the bioresorbable core of the lipid-enveloped nanoparticles, providing a means to co-deliver these factors to support the immune response elicited by the particles. Candidate T helper epitopes are identified using bioinformatics studies. To further augment the immune response, CpG oligonucleotides, ligands for TLR 9, are co-encapsulated in the core of the nanoparticles. Because TLR 9 is expressed in endosomal/phagosomal compartments, release of CpG from the particle cores following particle uptake should efficiently target this receptor while protecting CpG from extracellular DNAses prior to particle uptake. Synthesis schemes are developed to encapsulate pools of these candidate peptides in the cores of lipid-enveloped particles, with or without CpG oligos.

A peptide encapsulation protocol is validated using a pair of universal helper epitopes, pan HLA-DR-binding peptide (PADRE (SEQ ID NO:40); aK-Cha-VAAWTLKAAa (SEQ ID NO:41); where a is D-alanine, and Cha is L-cyclohexylalanine); and tetanus toxoid T-helper epitope (TT-Th; QYIKANSKFIGITEL (SEQ ID NO:42)); these peptides bind both HLA-DR and murine I-Ab/d and I-Eb/d class II MHC molecules and are used as positive controls in in vivo testing. These T helper peptides (1:1 mixtures of the two universal epitopes) are encapsulated in the core of lipid-enveloped nanoparticles using a double emulsion approach commonly employed for encapsulation of peptides in PLGA microparticles/nanoparticles. For example, a peptide solution (20 mg/mL in water) is emulsified in dichloromethane containing lipid and PLGA as before at 4° C. The resulting water-in-oil emulsion is added to deionized water, with homogenization followed by sonication at 4° C. to form the secondary water/oil/water emulsion. The particles are solidified by evaporating the organic solvent, washed, and stored at 4° C. (short term storage) or lyophilized in the presence of trehalose and stored at 4° C. until used.

The efficiency of peptide encapsulation is measured by incubating a sample of the particles in 0.5M NaOH/1% SDS for 30 minutes to hydrolyze the PLGA cores and solubilize the surface lipid layer, neutralizing the solution with HCl, and measuring the resulting concentration of released peptide using the microBCA protein/peptide assay (Pierce Chem. Co.) following the manufacturer's instructions. The kinetics of peptide release from the nanoparticles at extracellular pH or endolysosomal pH (mimicking release of peptides from particles within the phagosomes of APCs) are assessed by measuring the concentration of peptides released over time from 10 mg/mL particle suspensions incubated at 37° C. in pH 7.4 RPMI culture medium with 10% FCS or pH 5.5 PBS.

For co-encapsulation of CpG oligonucleotides, CpG (1 mg/mL) is mixed with T helper peptides and the mixed solution encapsulated as described above. For the planned murine in vivo studies, we will use CpG 1826 (5′-TCC ATG ACG TTC CTG ACG TT-3′ (SEQ ID NO:43), shown to strongly augment immune responses in mice; other immunostimulatory sequences are known for human cells. CpG encapsulation/release is assessed by using 3′-FITC labeled oligo, and measured by fluorescence spectrophotometry compared to a standard curve of labeled oligo.

To assess whether T helper peptides encapsulated in the core of lipid-enveloped nanoparticles are effectively released, processed, and presented by DCs following nanoparticle uptake, in vitro analyses of antigen presentation and T cell responses to the universal helper epitopes are performed. CpG is known to impact antigen processing/presentation as well as DC activation, and thus the impact of CpG co-encapsulation on CD4+ T cell priming in these assays is tested.

Groups of 4 C57Bl/6 mice are immunized subcutaneously with 50 μg of Th peptides mixed with 50 μL complete Freund's adjuvant or no peptide as a negative control. Nine days following immunizations, separate wells of bone marrow-derived DCs from C57Bl/6 mice are incubated with Th peptide-loaded nanoparticles (at doses ranging from 1 mg/mL down to 0.01 mg/mL) and 100 ng/mL LPS to mature the cells; Th peptide- and CpG-loaded nanoparticles (no LPS added); Th peptide-loaded nanoparticles (no LPS added); equivalent doses of soluble Th peptides, or Th peptides mixed with CpG as positive controls; empty nanoparticles and LPS, or LPS alone (as negative controls) for 12 hrs. The immunized mice are then sacrificed, and CD4+ T cells are isolated from spleens and lymph nodes by magnetic bead negative selection (Miltenyi). The isolated T cells are restimulated by culture with nanoparticle-, peptide-pulsed, or control DCs at a 10:1 T:DC ratio for 48 hours, and the culture supernatants from 6 hrs and 48 hrs are analyzed by ELISA for the production of IL-4, IFN-γ, and IL-10. T cell proliferation over the last 18 hours of the cultures are assessed by ³H-Thymidine incorporation. The prolonged restimulation culture time is used to allow time for sufficient peptide release from nanoparticles and processing by the DCs. These assays determine whether encapsulated T helper peptides are effectively processed/presented by DCs, and whether CpG co-delivery positively impacts presentation to CD4+ T cells.

Encapsulation of magnetic iron oxide particles in lipid-enveloped carriers to facilitate magnetic separation and MRI imaging.

In addition to encapsulation of T helper epitopes, the PLGA core of lipid-enveloped nanoparticles can be loaded with magnetic particles (sizes 4-10 nm, substantially smaller than the PLGA cores themselves). Encapsulation of such magnetic nanoparticles provides several opportunities with respect to in vitro/in vivo analyses: (1) magnetic lipid-enveloped nanoparticles (or cells that have taken up these particles) can be separated from tissue/cell suspensions using a magnet, (2) the high electron density of these particles makes the lipid-enveloped nanoparticles readily identifiable in TEM images, which allows ultrastructural analysis of particle localization in TEM sections of isolated cells or lymph nodes, and (3) magnetic labeling opens up the possibility of using MRI imaging to track the biodistribution of particles following immunization (in mice or humans).

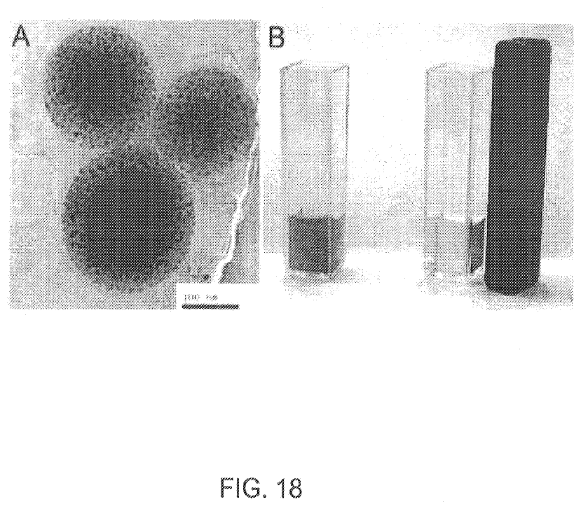

Prior studies have demonstrated that hydrophobically-capped paramagnetic iron oxide nanoparticles are readily encapsulated in PLGA by single-emulsion processes. In preliminary experiments, 10 nm-diameter CoFe2O4 iron oxide particles were encapsulated in lipid-enveloped PLGA nanoparticles (FIG. 18). These particles, which were synthesized by the method of Sun et al. (J Am Chem Soc 126, 273-9 (2004)) and stabilized with oleic acid were provided by Dr. Kimberly Hamad-Schifferli (Dept. of Biological Engineering at MIT). The iron oxide particles, synthesized in toluene, were precipitated by dilution with ethanol, then 59 mg were resuspended in DMPC/PLGA-containing dichloromethane solution and homogenized/sonicated in water to form lipid-enveloped nanoparticles as described above. CryoEM imaging of the resulting iron oxide-loaded nanoparticles revealed that high densities of the small magnetic particles could be encapsulated by this process (FIG. 18A). These highly-loaded particles were readily separated from macroscopic solutions by a bar magnet within 1-2 minutes (FIG. 18B).

To co-encapsulate both T cell epitopes and magnetic particles in the core of the PLGA carriers, first the minimal wt % loading of iron oxide nanoparticles required to easily isolate the lipid-enveloped PLGA particles with standard lab-size magnetic isolation columns/bar magnets is determined. Lipid-enveloped particles are prepared with 1, 5, 10 or 30 vol % iron oxide particles included in the initial organic phase, and the percentage of particles recovered from 1 mL of a 10 mg/mL enveloped particle suspension by a laboratory bar magnet within 5 minutes is quantified by measuring the absorbance of solutions before/after magnetic separation.

Next, to determine if T helper peptides can be co-encapsulated with magnetic nanoparticles in the core of lipid-enveloped PLGA particles, magnetic particles at the lowest dose sufficient for magnetic separation in the above assays are suspended in PLGA/lipid dichloromethane solution. This organic phase is used for formation of the aqueous peptide-in-dichloromethane emulsion as described above for T helper epitope encapsulation. The efficiency of peptide encapsulation and peptide release kinetics are determined as described above. If T helper epitope encapsulation efficiency is dramatically reduced, or peptide release kinetics are negatively influenced by the co-encapsulation of magnetic nanoparticles, magnetic lipid-enveloped particles are then used for mechanistic studies of lipid-enveloped nanoparticle behavior in the absence of T helper peptides, and/or immunize with mixtures of magnetic/T helper peptide-loaded particles to allow both nanoparticle tracking and T helper epitope delivery in vivo.

As shown above, intradermal immunization with nanoparticles leads to ˜3% of lymph nodes positive for nanoparticles by 48 hours. Magnetic lipid-enveloped nanoparticles are used as a tool to enrich the cells in draining lymph nodes internalizing nanoparticles for flow cytometric and in vitro analysis. Cell suspensions recovered from mice immunized with MPER-carrying nanoparticles are subjected to magnetic sorting using commercial magnetic separation columns, to positively select and isolate nanoparticle-loaded cells. Recovered cells will then be analyzed by flow cytometry for phenotype and/or analyzed biochemically for the detection of delivered MPER peptide as described above.

Structure/Compositional Characterization of Lipid-Enveloped Nanoparticles

A thorough understanding of the structure and composition of the lipid-enveloped nanoparticles will facilitate the design of particles that optimally bind and present MPER, support targeting ligand conjugation, and allow effective peptide encapsulation/release. Thus, in parallel with the experiments described above, the following studies are conducted to further elucidate the structure and physicochemical behavior of lipid-enveloped nanoparticles.

NMR and biochemical analysis of lipid surface composition. As described above, the composition of the lipid membrane was found to impact the affinity and amount of MPER binding to liposomes; thus it is expected that the membrane composition at the surface of lipid-enveloped particles to likewise control the amount and conformation of MPER binding to the nanoparticles. To assess the surface density of lipid enveloping PLGA nanoparticles of each size/composition, accessible phospholipid headgroups are measured using the Bottcher-modified Bartlett phosphate assay (Bottcher et al. (1961) Anal. Chim. Acta 24, 203-204). By combining measured phospholipid concentrations with known particle masses/sizes and the known dimensions of the phospholipids headgroups, this analysis can provide information about the quality of the lipid-enveloped particles: are particles all fully covered by a lipid surface, or do some particles exhibit “bald spots.” Separately, the surface densities of PEGylated lipids (used for MPER immobizliation and targeting ligand conjugation) are quantified using the HABA assay (Pierce Chem. Co.) to quantify biotin-PEG-lipids accessible at the surface of enveloped nanoparticles, per the manufacturer's instructions.

To quantify the actual composition of lipids self-assembled at the surface of lipid-enveloped nanoparticles, and determine whether the composition of lipids added to the synthesis matches the composition assembled at the surface of the lipid-enveloped particles (as opposed to preferential enrichment of certain lipid components), ¹H NMR analysis of lipid-enveloped nanoparticle suspensions are carried out. DOPC/DOPG, T cell membrane-mimicking, and HIV-mimetic lipid compositions are analyzed with or without 1 mole % mal-PEG-DHPE lipid. Particles are suspended in deuterated phosphate buffer and 1H-NMR spectra are collected on a Bruker Avance spectrometer operating at 600 MHz with 16K data points and a relaxation delay of 2 seconds. Analysis of relative peak intensities allows for the determination of mole ratios of surface-accessible lipid groups.

The encapsulation of T helper peptides or magnetic particles can influence the overall structure of lipid-enveloped nanoparticles. In addition, it is of interest to understand whether the surface lipid membrane maintains its integrity following exposure to the acidic pH expected in dendritic cell phagosomal compartments and/or following slow hydrolysis at extracellular pH. Cryoelectron microscopy is used to directly visualize how the internal and surface structure of lipid-enveloped nanoparticles is affected by peptide/iron oxide encapsulation, incubation in pH 5.5 PBS buffers at 37° C., or incubation in RPMI medium containing 10% FCS for 0-36 hours.

### Example 12

### Quantification of MPER Binding to Nanoparticles as a Function of Lipid Composition

Preliminary studies revealed that the affinity of MPER binding to liposomes varies with the membrane composition; when comparing liposomes composed of 4:1 DOPC:DOPG, DMPC, or an HIV membrane-mimicking composition (Brugger et al. (2006) Proc Natl Acad Sci USA 103, 2641-6) (DOPC/sphingomyelin/DOPE/DOPG/cholesterol at a 9:18:20:9:44 mole ratio), MPER binding affinity increased in the order DMPC<DOPC/DOPG<HIV mimic.

To determine whether the binding of MPER to lipid-enveloped nanoparticles occurs with the same hierarchy in binding affinity, binding curves are measured for MPER adsorption to nanoparticles prepared with different membrane compositions: lipid-enveloped nanoparticles prepared with 4:1 DOPC:DOPG, T cell membrane mimicking, or HIV-mimetic lipid coats (1 mg/mL, approximately 5.66×10¹¹ particles/mL) incubated with FITC-labeled MPER at concentrations ranging from 10 nm to 50 μM (10 to ˜5×10⁴-fold molar excess over nanoparticles) for 1 hour at 37° C. The nanoparticles are pelleted by centrifugation (5 minutes at 14,000 g) and washed to remove unbound MPER, resuspended in 0.5M NaOH/1% SDS for 30 minutes to lyse the PLGA cores of the particles and solubilize the lipids, neutralized with HCl, and the released MPER concentration determined by measuring the FITC fluorescence in solution compared to a standard curve of MPER-FITC fluorescence. To quantify the role of the lipid in regulating peptide binding, the MPER adsorption to ‘bare’, non-enveloped PLGA nanoparticles synthesized with no lipid coating are compared.

The MPER association with DOPC/DOPG, T cell-mimetic, and HIV membrane-mimetic liposomes, is also compared with one another to determine whether the PLGA particle core influences MPER association indirectly. MPER binding to liposomes are compared by comparing liposomes and lipid-enveloped nanoparticles with diameters as close to equal as experimentally feasible, with concentrations adjusted to ensure equivalent surface areas. This data will reveal what membrane composition promotes maximal MPER binding to enveloped nanoparticles.

Stability of MPER Binding in Presence of Serum.

For the nanoparticles to successfully deliver adsorbed MPER peptides, the HIV fragments will need to be stably bound to the particles in the presence of serum proteins that may compete for binding to the particle surfaces. In preliminary studies it was discovered that the MPER remains stably adsorbed to lipid-enveloped PLGA microparticles for at least a few hours in culture medium containing 10% FCS in the presence of DCs, based on qualitative confocal imaging results using FITC-tagged MPER. To quantitatively assess the stability of MPER association with lipid-coated nanoparticles over longer periods, 1 mg/mL nanoparticles with or without lipid surfaces (4:1 DOPC/DOPG mixture, T cell-mimetic, or HIV-mimetic) are incubated with saturating concentrations of FITC-MPER for 1 hr at 37° C., centrifuged/washed to remove unbound MPER, and resuspended in RPMI 1640 culture medium with or without 10% fetal calf serum for 1 hour, 6 hours,12 hours, or 24 hours. Particle samples (in triplicate) are recovered by centrifugation at the end of the incubation period, washed, and then lysed/analyzed for remaining MPER via FITC fluorescence as above.

Binding of 4E10 Neutralizing Antibody to Nanoparticles as a Function of Lipid Skin Composition.

In preliminary experiments, it was found that MPER peptides adsorbed to DMPC-enveloped nanoparticles were recognized by the HIV MPER-targeting 4E10 neutralizing antibody, as detected by fluorescence spectrophotometry (FIGS. 16C and 16D). The studies described above are useful to characterize the levels of MPER binding to nanoparticles with different lipid compositions. However, it is possible that the lipid composition providing the highest binding affinity for MPER adsorption will not leave the peptide in a conformation readily recognized by HIV-neutralizing antibodies. Thus, the binding of 4E10 to MPER peptides adsorbed to nanoparticles bearing DOPC/DOPG, T cell-mimetic, or HIV-mimetic lipid surfaces is measured. Binding is measured using a variation of the fluorescence assay described above for quantification of MPER adsorption to lipid-enveloped particles: FITC-MPER peptide (0.1 μM, 1 μM, or 10 μM; we may adjust these concentrations based on findings in the MPER adsorption studies) are incubated with 1 mg/mL DOPC/DOPG, T cell-mimic, or HIV-mimic lipid-coated nanoparticles for 30 min at 37° C. in PBS. Control particles without MPER are incubated for mock treatment in buffer. The nanoparticles are pelleted using centrifugation, washed to remove unbound MPER, then immunostained. That is, particles are incubated with 5 μg/mL unlabeled 4E10 primary antibody at 4° C. or 37° C. for 30 minutes, washed at 4° C., and then stained with Alexafluor 647-conjugated goat anti-human IgG antibody (5 μg/mL at 4° C. for 30 min). Control staining is performed using the secondary Ab only (no 4E10). Following secondary labeling, the particles are washed to remove unbound antibody, then lysed with 0.5M NaOH/1% SDS as described above for MPER binding quantification. 4E10 and MPER binding is determined from Alexafluor and FITC fluorescence in the solution, respectively, measured using a spectrofluorometer. 4E10 binding is carried out both at 37° C. and 4° C. to determine whether there are effects of temperature on lipid or MPER organization/mobility on 4E10 recognition. Relative 4E10 binding is normalized to the quantity of MPER bound to particles of each composition as a function of MPER concentration during the peptide adsorption step, to rank-order the relative efficiency of 4E10 recognition of MPER bound to each lipid-enveloped nanoparticle composition. The 4E10 binding experiments are repeated with unlabeled MPER peptide to ensure that the FITC label does not affect the 4E10 recognition results.

Conformation of MPER Peptides Adsorbed to Nanoparticles as a Function of Lipid Skin Composition.

In order to understand how MPER binding and 4E10 recognition on lipid-enveloped nanoparticles compares to the simpler model of MPER association with lipid micelles or liposomes, electron paramagnetic resonance (EPR) is used to analyze the conformation of MPER peptides associated with lipid-enveloped nanoparticles (4:1 DOPC:DOPG, T cell-mimetic, or HIV-mimetic membranes) and liposomes with the same membrane composition. MPER peptides with EPR spin labels attached at different residues are prepared as described above. Nanoparticles are incubated with 10 μM spin-labeled MPER peptide for 1 hour at 37° C., centrifuged and washed to remove unbound MPER, then analyzed by EPR.

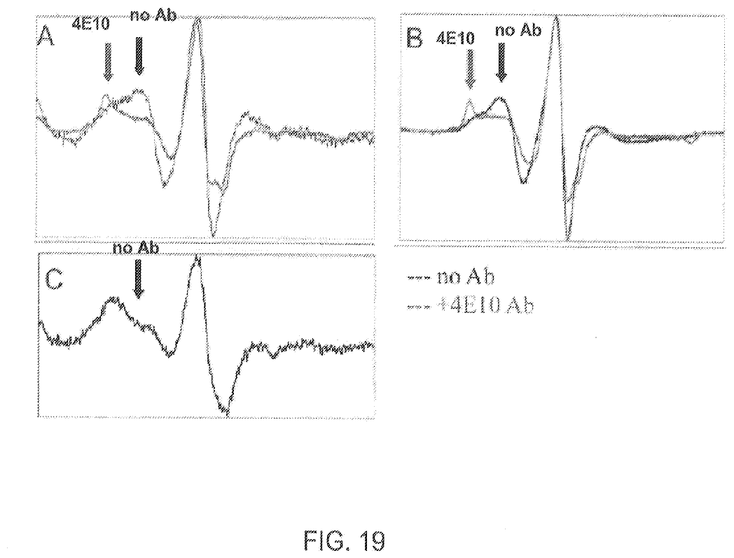

In preliminary experiments, the EPR spectrum of spin-labeled MPER associated with lipid-enveloped nanoparticles was found to be very similar to the spectrum of MPER associated with 4:1 DOPC:DOPG liposomes (FIGS. 19A and 19B). This EPR spectrum correlates with the peptide assuming a two-helix structure in the lipid membrane, as revealed by structural NMR studies. As shown in FIG. 19C, ‘bare’ PLGA nanoparticles lacking a lipid envelope exhibit an EPR spectrum with significantly altered features (compare region of No Ab in FIG. 19A and FIG. 19B vs. FIG. 19C), suggesting that the lipid membrane surface is required for this particular neutralizing antibody-recognized conformation of the MPER peptide. (This spectrum also exhibited substantially higher noise due to the low amount of MPER adsorbing to the ‘bare’ PLGA). Addition of 4E10 antibody to the MPER-coated nanoparticles at a 2:1 ratio elicited a change in the mobility of spin labels on the MPER matching that observed for MPER adsorbed to liposomes (FIGS. 19A and 19B, “4E10” spectra and arrows), indicating similar conformation changes in the peptide bound to liposomes or lipid-enveloped nanoparticles on 4E10 binding. These results are in accord with the 4E10 binding measurements shown in FIG. 16A and provides further evidence that lipid-enveloped nanoparticles can provide a proper membrane environment for MPER presentation to the immune system.

### Example 13

### Analysis of the Effect of Nanoparticle Targeting on MPER/Nanoparticle Binding to Dendritic Cells and MPER Fate Following Particle Binding to Cells

Linkage of Targeting/DC-Modulating Ligands to Lipid-Enveloped Nanoparticles.

Conjugation of targeting antibodies or flagellin to nanoparticles. Rat anti-murine DEC-205 monoclonal antibody (NLDC-145) are purified from hybridoma supernatants (ATCC). Agonistic anti-CD40 (1C10) are commercially available from R&D Systems and isotype control Abs ware available from BD Biosciences and R&D Systems. Anti-murine CD32b (K9.361) is be purified from hybridoma supernatants (Holmes et al. (1985) Proc Natl Acad Sci USA 82, 7706-10). Recombinant E. Coli-expressed monomeric flagellin is obtained from VaxInnate Inc. (New Haven, Conn.).

A generic strategy is developed for covalent conjugation of protein ligand to lipid-enveloped nanoparticles (FIG. 20). Lipid-enveloped nanoparticles are synthesized with 1 mole % DSPE-PEG(2000)-maleimide (1,2-Distearoyl-sn-Glycero-3-Phosphoethanolamine-N-[Maleimide(Polyethylene Glycol)2000], Avanti Polar Lipids) in the lipid component. Protein ligand (antibody or flagellin) is reacted with the heterobifunctional crosslinker s-acetyl-(PEO)₄-NHS (Pierce Chemical Co.), which reacts with free amines on the protein. Excess linker is removed by filtration. The free end of the crosslinker is a protected thiol; this thiol is deprotected using the mild reductant TCEP and the thiol-functionalized protein is mixed with maleimide-bearing nanoparticles in the presence of TCEP and EDTA to allow conjugation through formation of a thioether linkage. Nanoparticles are separated from unconjugated protein by centrifugation and washing. The yield of conjugation is controlled by varying the concentration of thiolated protein and maleimide-bearing nanoparticles during the conjugation step.

The surface densities of ligand typically needed for targeting of liposomes or particles to specific receptors in vivo are very low (e.g., from other published examples, ˜5-50 antibodies per particle, at densities as low as 0.4 ligands/μm²). To determine the ligand density achieved in the above coupling reaction, the yield of protein coupled (μg protein per mg nanoparticles) is quantified using the microBCA protein assay (Pierce Chemical Co.) following the manufacturer's instructions. Yields in maleimide coupling reactions are typically high, on the order of ˜80%. To limit the number of variables that need to be optimized, coupling conditions are developed that yield ˜500, 100, or 50 ligands per nanoparticle on 150 nm-diameter nanoparticles. If it is found that suitable targeting occurs at ligand densities too low to effectively characterize by microBCA, the particles are then solubolized with brief NaOH/SDS treatment as described above and quantify ligand in solution using ELISAs.

Lewis x sugars. While monoclonal antibodies can offer high specificity and affinity for targeting DC cell surface receptors, less costly targeting molecules that can be produced synthetically and that avoid the need for ‘humanization’ for clinical use are of interest. To this end, targeting C-type lectins to DCs with synthetic lewis x (Le^{x}) trisaccharides is evaluated. Lectins typically bind to lewis x (Le^{x}) and related sugars with relatively low affinity, but multivalent sugar motifs (as they are typically encountered on the surface of pathogens) can bind cell-surface lectins with high net avidity. Therefore, water-soluble poly(hydroxyethyl acrylamide) (PHEAAm) polymers bearing multiple Le^{x} trisaccharides (30 KDa PHEAAm with Le^{x} coupled to ˜20 mole % of the hydroxyl side chains, Glycotech, Rockville, Md.) are conjugated to lipid-enveloped nanoparticles, to obtain high-avidity Le^{x}-based targeting. Other sugar variants are available commercially and from the Consortium for Functional Glycomics, if these sugar-based ligands show promise in initial studies.

To conjugate Le^{X}-PHEAAm polymers to lipid-enveloped nanoparticles, free hydroxyl groups on PHEAAm are activated using carbodiimidazole (CDI) in DMSO. The activated polymer is then mixed with lipid-enveloped nanoparticles prepared with 1 mole % DSPE-PEG(2000)-amine as part of the lipid component, providing a free primary amine group at the end of a short poly(ethylene glycol) tether in the surface lipid layer of the particles. The activated Le^{x}-PHEAAm react with PEG-amines on the particle surface to covalently tether the Le^{X}-polymer to the nanoparticle. To avoid crossreactivity with MPER amines, Le^{x} conjugation are performed prior to MPER adsorption/binding to nanoparticles. Note that the CDI coupling chemistry does not interfere with the maleimide coupling used for MPER anchoring. The nanoparticles are centrifuged and washed to remove unbound Le^{x}-polymer. The yield and surface density of Le^{x} conjugated is determined by lysing and solubilizing an aliquot of the nanoparticles with 0.5M NaOH/1% SDS for 30 min, followed by anti-Le^{x} ELISA to detect the concentration of released Le^{x}-polymer (Covance/Signet Labs).

Co-conjugation of MPER and targeting ligands. As stated above, the density of targeting ligand needed is very low, and it is expected that co-conjugation of targeting ligand will not interfere with obtaining high densities of MPER conjugated to particles if desired. For particles bearing both covalently-bound MPER and targeting ligands, MPER and targeting proteins/lewis x are co-conjugated to particles simultaneously by adding Cys-functionalized MPER and thiolated targeting ligand to particles at low targeting ligand:MPER mole ratios in the presence of TCEP/EDTA; purification of particles from unconjugated MPER/ligand is performed as before. To determine targeting ligand coupling yields/surface densities on nanoparticles in this case, particles are solubolized with 0.5M NaOH/1% SDS and quantify targeting proteins using ligand-specific ELISAs.

To confirm the functionality of targeting ligands bound to nanoparticles and determine optimal targeting ligand densities, first the binding of targeted nanoparticles to DCs vs. untargeted control particles in vitro is measured. For these initial characterization experiments, particles lacking MPER are used. Murine bone marrow-derived DCs from C57Bl/6 mice (2×10⁵ cells in 200 μL medium) are cultured with fluorescent lipid-enveloped nanoparticles (1 μg/mL, 10 μg/mL, or 50 μg/mL) for 1, 2, or 6 hours at 37° C. Nanoparticles conjugated are tested with each targeting ligand (at the 3 target ligand densities described above) vs. non-targeted control particles. At the end of the incubation period, the cells are washed to remove unbound particles, fixed with paraformaldehyde, and then analyzed on a BD LSRII flow cytometer to quantify relative particle uptake. The relatively early times are focused on, in particular, since prolonged incubation of DCs with particles in vitro leads to eventual phagocytosis even in the absence of any targeting ligand, a well-known characteristic of highly phagocytic immature DCs and also observed in the above studies with lipid-enveloped nanoparticles (FIG. 13A). To confirm the specificity of targeting ligand effects, the inhibition of targeted particle binding with free soluble targeting ligands is tested.

The encapsulation of two different adenosine receptor inhibitors (caffeine, a preferential inhibitor of adenosine receptor A2AR (Sigma); and 1,3-diethyl-8-(3,4-dimethoxystyryl)-7-methyl-3,7-dhydro-1H-purine-2,6-dione (DMS-DEX), an inhibitor of A2AR and A2BR) is studied. For HIF-1α inhibition, a novel 5-aminosubstituted camptothecin derivative (5AC) is tested.

First, the encapsulation of the inhibitors alone in the core of lipid-enveloped nanoparticles (i.e. no co-encapsulation of T cell helper peptide antigens) is tested. Because the adenosine receptor and HIF-1α inhibitors target complementary pathways, the encapsulation of each drug alone or mixtures of the two types of inhibitor are tested (see the schedule in Table 4).

TABLE 4

| Adenosine receptor | HIF-1α |
|---|---|
| inhibitors | inhibitor |

| Caffeine | DMS-DEX | 5AC |
|---|---|---|
| X | | |
| | X | |
| | | X |
| X | | X |
| | X | X |

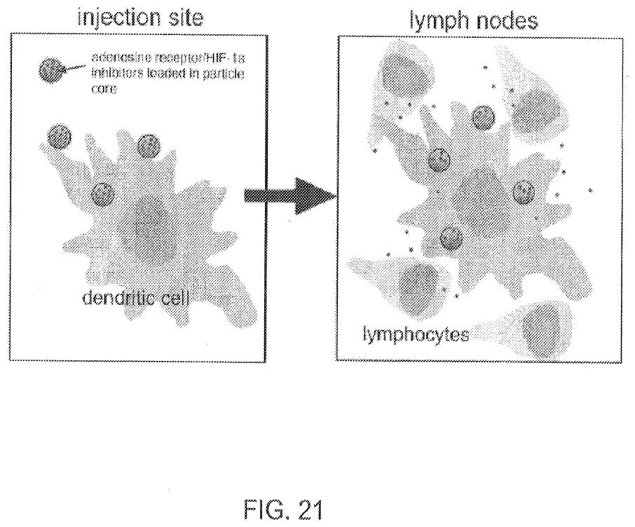

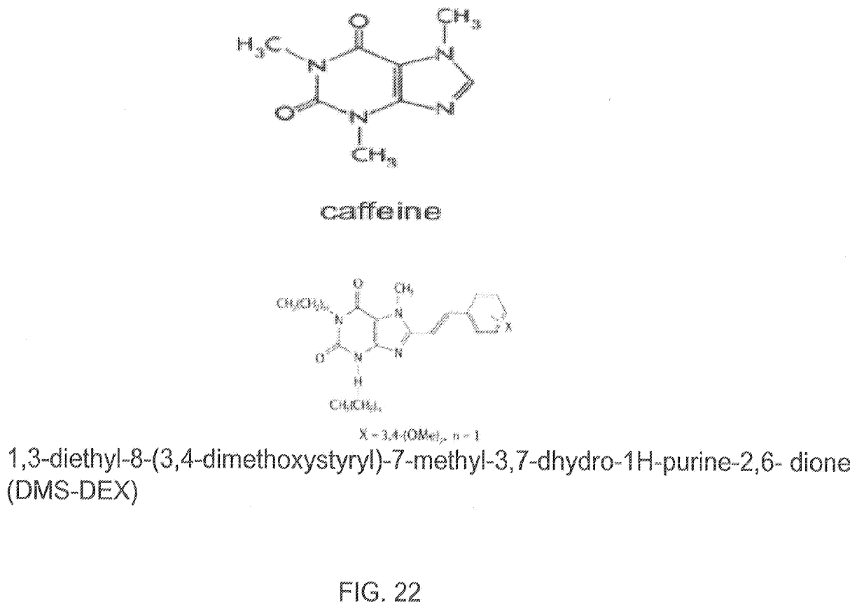

The hydrophobic nature of these compounds (FIG. 21) allows their direct addition to the organic polymer solution during nanoparticle synthesis: PLGA/lipid dichloromethane solutions are prepared as before, and inhibitors are co-dissolved at PLGA:inhibitor weight ratios of 99:1 to 90:10, to achieve target drug loading in the range of 1-10 wt % of the final particles. Based on much work in the field of drug delivery encapsulating lyophilic small molecule drugs in PLGA and related polyester microspheres, it is expected that inhibitor loading in lipid-enveloped nanoparticles are efficient. Whether or not ADR/HIF-1α inhibitors can be co-encapsulated with PADRE and TT-Th universal T helper cell epitopes in the core of lipid-enveloped nanoparticles is also tested. This is achieved by adding the inhibitors to the organic phase during nanoparticle synthesis as described above, while performing T cell peptide epitope encapsulation in an internal aqueous phase through the double emulsion process described above. The resulting particles are characterized by dynamic light scattering and scanning electron microscopy, to determine if particle size or morphology is impacted by inhibitor/T cell epitope encapsulation. Drug loading/encapsulation efficiency is determined by solubilizing the nanoparticles with 0.5M NaOH/1% SDS treatment for 30 minutes and measuring the quantity of released inhibitors by HPLC using UV-vis detection. T helper epitope co-encapsulation is assessed using the microBCA protein/peptide assay as described above.

Ideally, release of ADR/HIF-1α inhibitors would be sustained over the course of the induction of primary immune responses elicited by the vaccine, e.g., 7-14 days. Both the total drug loading per nanoparticle and particle size will influence the kinetics of inhibitor release. Thus, release kinetics of each of the inhibitors/inhibitor mixtures alone or co-encapsulated with T helper epitopes are characterized for sub 50 nm and 150 nm diameter nanoparticles. Release profiles are obtained by incubating the drug-loaded nanoparticles (10 mg/mL) in complete RPMI medium containing 10% FCS at 37° C., and measuring the concentration of released drugs and T helper epitopes in the supernatant of the particle suspensions as a function of time daily over 2 weeks in vitro by HPLC and microBCA assays, respectively. At each timepoint, nanoparticles are pelleted by centrifugation, the supernatant is removed for HPLC analysis, and the particles are then resuspended in fresh medium. In parallel with these measurements, the mass loss of nanoparticles incubated in medium over time is measured to determine how inhibitor/T helper epitope loading of the lipid-enveloped nanoparticles affects the hydrolysis rates and breakdown of the PLGA cores.

In the setting of prophylactic vaccination, it is likely that for ADR/HIF-1α inhibitors to enhance the antibody response, these drugs will need to be delivered to the lymph nodes where naive T cell and B cell priming is occurring. As described above, the synthesis of sub 50 nm-diameter inhibitor-loaded nanoparticles are tested to determine if they are capable of directly draining to lymph nodes from a peripheral injection site. However, it is also of interest to test whether dendritic cells could directly take up nanoparticles at the immunization site and carry the particles to the lymph nodes, followed by release of inhibitors from particles from within DCs and diffusion of these drugs into the surrounding microenvironment.

To determine whether inhibitors released from nanoparticles internalized by DCs effectively diffuse out of the carrying cell and into the surroundings, and whether the kinetics of drug release from within cells differs substantially from the release from nanoparticles into culture medium, inhibitor accumulation in the medium of nanoparticle-loaded DCs is tested in vitro. Bone marrow-derived DCs from C57Bl/6 mice are incubated with inhibitor-loaded nanoparticles (1 mg/mL) for 2 hours in triplicate to allow nanoparticle uptake (FIG. 13A), even non-targeted nanoparticles are taken up by DCs over a few hrs in culture), then washed thoroughly to remove non-internalized particles. Inhibitors released into the medium over time are quantified by analyzing aliquots of the culture supernatant by HPLC. Control wells are prepared where following nanoparticle uptake and washing of the DCs, the cells are lysed with non-denaturing cell lysis buffer (Chemicon) to free internalized nanoparticles and allow direct release of drug into the medium. To allow comparison with bulk drug release measurements described above, the amount of total drug-loaded nanoparticles internalized by cells is determined by lysing cells in additional control wells, followed by solubilization of nanoparticles by treatment with 0.1 NaOH/1% SDS, and measuring total released drug in the supernatant by HPLC.

### Example 14

### Immigration of PLGA-Lipid-Coated, DiD-Labeled Nanoparticles to Lymph Nodes After Uptake and Transport by Dermal Dendritic Cells

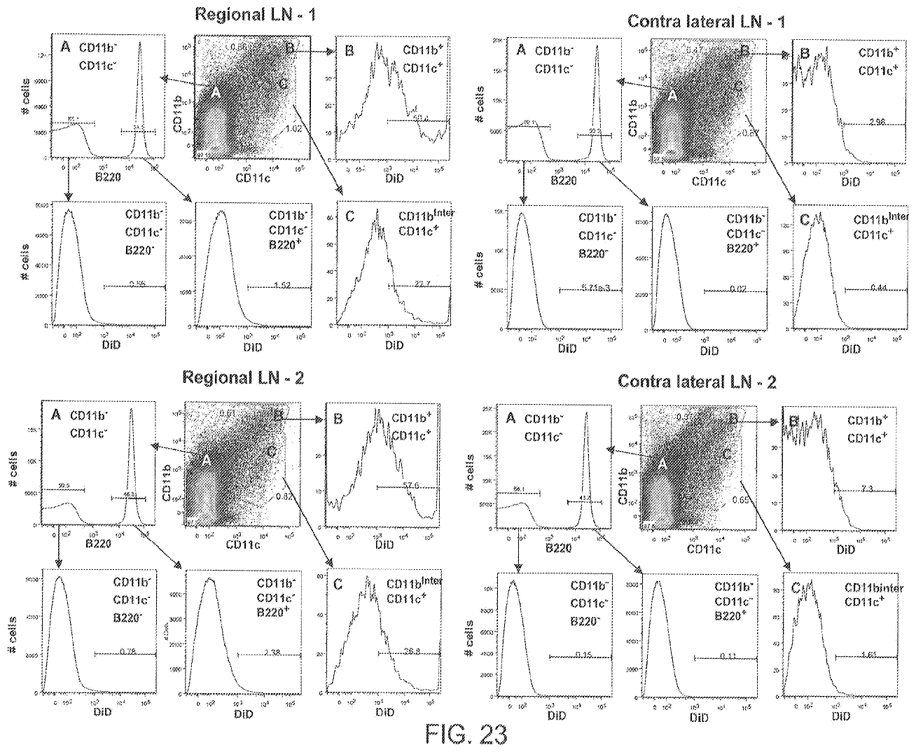

Mice were injected intradermally (i.d). with 1 mg of lipid-enveloped nanoparticles (200 nm diam). Lymph nodes from the injected (regional) side and control (contralateral) side were removed 48 hours after injection, stained with mAbs (specific to CD11b, Cd11c, or B220), and analyzed by multicolor flow cytometry (FIG. 23). As shown in gates B and C collectively, about 1.2-1.9% of cells were stained, with CD11chigh CD11b inter and CD11chigh CD11b high in both regional and contra lateral lymph nodes, representing dendritic cells. Of these dendritic cells, more than 50% of CD11chigh CD11b high and 23% of CD11chigh CD11b inter cells carried env-enveloped, DiD labeled nanoparticles in regional lymph nodes but virtually none in contra lateral lymph nodes. 1-2% of nanoparticles were taken up by CD11c− CD11b− B220+ B cells, while less than 1% of partilces were taken up by CD11c− CD11b− B220− cells including T cells in regional lymph nodes as shown A. These results indicate that lipid enveloped nanoparticles injected intradermally to a mammal can be delivered to draining lymph nodes.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for inducing an immune response in a subject, the method comprising administering to the subject a composition comprising:
a particle encapsulated in lipid, wherein the particle comprises a solid material; and
a protein consisting of:
a membrane proximal external region (MPER) of an HIV-1 gp160 polypeptide; or a fragment of the HIV-1 gp160 polypeptide comprising the MPER,
wherein the protein is no more than 100 amino acids in length and wherein at least one amino acid residue of the MPER that corresponds to position 669, 670, 672, 673, 675, or 678 of the HXB2 strain HIV-1 gp160 polypeptide is embedded in the lipid,
wherein the reagent is capable of inducing HIV-1 broadly neutralizing antibodies in the subject.

2. (canceled)

3. The method of claim 1, wherein the protein is no more than 60 amino acids in length.

4. The method of claim 1, wherein the protein is no more than 30 amino acids in length.

5. The method of claim 1, wherein the protein is no more than 22 amino acids in length.

6. The method of claim 1, wherein the MPER comprises the amino acid sequence X₁-L-X₂-X₃-W-X₄-X₅-X₆-W-X₇-W-X₈-X₉-I-X₁₀-X₁₁-W-L-W-Y-I-X₁₂ (SEQ ID NO:1), wherein X₁ is A, Q, G, or E; X₂ is D or S; X₃ is K, S, E, or Q; X₄ is A, S, T, D, E, K, Q, or N; X₅ is S, G, or N; X₆ is L or I; X₇ is F, N, S, or T; X₈ is F or S; X₉ is D, K, N, S, T, or G; X₁₀ is S or T; X₁₁ is N, K, S, H, R, or Q; and X₁₂ is K, E, or R;
the amino acid sequence ELDKWASLWNWFNITNWLWYIK (SEQ ID NO:2);
the amino acid sequence ALDKWASLWNWFDISNWLWYIK (SEQ ID NO:3); or
an amino acid sequence corresponding to amino acid positions 662 to 683 of the HXB2 strain HIV-1 gp160 polypeptide.

7. The method of claim 1, wherein the MPER consists of the amino acid sequence X₁-L-X₂-X₃-W-X₄-X₅-X₆-W-X₇-W-X₈-X₉-I-X₁₀-X₁₁-W-L-W-Y-I-X₁₂ (SEQ ID NO:1), wherein X₁ is A, Q, G, or E; X₂ is D or S; X₃ is K, S, E, or Q; X₄ is A, S, T, D, E, K, Q, or N; X₅ is S, G, or N; X₆ is L or I; X₇ is F, N, S, or T; X₈ is F or S; X₉ is D, K, N, S, T, or G; X₁₀ is S or T; X₁₁ is N, K, S, H, R, or Q; and X₁₂ is K, E, or R;
the amino acid sequence ELDKWASLWNWFNITNWLWYIK (SEQ ID NO:2);
the amino acid sequence ALDKWASLWNWFDISNWLWYIK (SEQ ID NO:3); or
an amino acid sequence corresponding to amino acid positions 662 to 683 of the HXB2 strain HIV-1 gp160 polypeptide.

8.-15. (canceled)

16. The method of claim 1, wherein the lipid is a lipid monolayer.

17.-26. (canceled)

27. The method of claim 1, wherein at least one amino acid of the MPER is not embedded within the lipid.

28. The method of claim 27, wherein the at least one amino acid of the MPER that is not embedded in the lipid corresponds to position 671, 674, 677, or 680 of the HXB2 strain HIV-1 gp160 polypeptide.

29. (canceled)

30. The method of claim 1, further comprising at least one additional polypeptidoprotein.

31. The method of claim 30, wherein the at least one additional protein is
a targeting polypeptide;
a dendritic cell activating polypeptide; or
a polypeptide comprising a T helper epitope.

32. (canceled)

33. The method of claim 31, wherein the targeting polypeptide targets the reagent to an antigen presenting cell.

34. (canceled)

35. The method of claim 1, wherein the composition further comprising comprises one or more additional therapeutic agents or one or more additional prophylactic agents.

36. The method of claim 35, wherein the at least one of the one or more additional therapeutic agents or at least one of the one or more prophylactic agents is lipophilic.

37. The method of claim 35, wherein at least one of the one or more additional therapeutic agents or at least one of the one or more prophylactic agents is embedded in the lipid.

38. The method of claim 35, wherein at least one of the one or more therapeutic agents is an immune modulator.

39.-42. (canceled)

43. The method of claim 1, wherein the MPER is:
a fragment of a Group M HIV-1 gp160 polypeptide;
a fragment of a Clade B HIV-1 gp160 polypeptide; or
a fragment of a Clade A, Clade C, or Clade D HIV-1 gp160 polypeptide.

44.-45. (canceled)

46. The method of claim 1, wherein the MPER is detectably labeled.

47. The method of claim 46, wherein the detectable label is a fluorescent label, a luminescent label, a radioactive label, or an enzymatic label.

48.-55. (canceled)

56. The method of claim 1, wherein the subject is a human.

57. The method of claim 1, further comprising, after administering the composition, determining whether an immune response in the subject has occurred.

58. The method of claim 1, wherein the subject has, is suspected of having, or is at risk of developing an HIV-1 infection.

59. The method of of claim 1, further comprising administering to the subject one or more anti-HIV-1 agents.

60.-95. (canceled)

96. The method of claim 1, wherein:
(i) the amino acid residue of the MPER that corresponds to position 673 of the HXB2 strain HIV-1 gp160 polypeptide is embedded in the lipid;
(ii) the amino acid residue of the MPER that corresponds to position 675 of the HXB2 strain HIV-1 gp160 polypeptide is embedded in the lipid; or
(iii) amino acid residues corresponding to positions 671, 674, 677, and 680 of the HXB2 strain HIV-1 gp160 polypeptide are not embedded in the lipid.

97. A method for inducing an immune response in a subject, the method comprising administering to the subject a composition comprising a reagent capable of inducing HIV-1 broadly neutralizing antibodies in the subject, wherein the reagent comprises:
a particle encapsulated in lipid, wherein the particle comprises a solid material; and
a protein consisting of:
a membrane proximal external region (MPER) of an HIV-1 gp160 polypeptide; or
a fragment of the HIV-1 gp160 polypeptide comprising the MPER,
wherein the MPER comprises a first a helix and a second α helix, wherein at least one amino acid residue in the first α helix is embedded in the lipid, and at least one amino acid residue in the second α helix is embedded in the lipid, and wherein the MPER comprises a kinked structure.

98. The method of claim 97, wherein the protein is no more than 100 amino acids in length.

99. The method of claim 97, wherein the lipid comprises sphingomyelin.

100. The method of claim 97, wherein the particle comprises a polymer, a resin, carbon, latex, a metal, or a glass.

101. The method of claim 1, wherein:
(i) at least one amino acid residue of the MPER that corresponds to position 669, 673, or 675 of the HXB2 strain HIV-1 gp160 polypeptide is embedded in the lipid;
(ii) at least one amino acid residue in the first a helix that corresponds to position 669, 670, or 672 of the HXB2 strain HIV-1 gp160 polypeptide is embedded in the lipid;
(iii) at least one amino acid residue in the second a helix that corresponds to position 675 or 678 of the HXB2 strain HIV-1 gp160 polypeptide is embedded in the lipid; or
(iv) the amino acid residue corresponding to position 680 of the HXB2 strain HIV-1 gp160 polypeptide is not embedded in the lipid.


SEQUENCE LISTING (43 sequences)

SEQ ID NO: 1 - protein, 22 aa, Artificial Sequence
  Synthetically generated peptide
  VARIANT 1: Xaa = Ala, Gln,  Gly or Glu
  VARIANT 3: Xaa = Asp or Ser
  VARIANT 4: Xaa = Lys, Ser, Glu or Gln
  VARIANT 6: Xaa = Ala, Ser, Thr, Asp, Glu, Lys, Gln, or Asn
  VARIANT 7: Xaa = Ser, Gly, or Asn
  VARIANT 8: Xaa = Leu or Ile
  VARIANT 10: Xaa = Phe, Asn, Ser or Thr
  VARIANT 12: Xaa = Phe or Ser
  VARIANT 13: Xaa = Asp, Lys, Asn, Ser, Thr or Gly
  VARIANT 15: Xaa = Ser or Thr
  VARIANT 16: Xaa = Asn, Lys, Ser, His, Arg or Gln
  VARIANT 22: Xaa = Lys, Glu or Arg
XLXXWXXXWXWXXIXXWLWYIX

SEQ ID NO: 2 - protein, 22 aa, Human immunodeficiency virus 1
ELDKWASLWNWFNITNWLWYIK

SEQ ID NO: 3 - protein, 22 aa, Human immunodeficiency virus 1
ALDKWASLWNWFDISNWLWYIK

SEQ ID NO: 4 - protein, 22 aa, Human immunodeficiency virus 1
ALDKWASLWTWFDISHWLWYIK

SEQ ID NO: 5 - protein, 22 aa, Human immunodeficiency virus 1
ALDKWASLWNWFDITQWLWYIK

SEQ ID NO: 6 - protein, 22 aa, Human immunodeficiency virus 1
ALDKWASLWNWFDITKWLWYIK

SEQ ID NO: 7 - protein, 22 aa, Human immunodeficiency virus 1
ALDKWASLWNWFDISNWLWYIR

SEQ ID NO: 8 - protein, 22 aa, Human immunodeficiency virus 1
ALDKWANLWNWFDISNWLWYIK

SEQ ID NO: 9 - protein, 22 aa, Human immunodeficiency virus 1
ELDKWASLWNWFSITNWLWYIK

SEQ ID NO: 10 - protein, 22 aa, Human immunodeficiency virus 1
ELDKWASLWNWFSITNWLWYIR

SEQ ID NO: 11 - protein, 22 aa, Human immunodeficiency virus 1
ELDKWASLWNWFDITNWLWYIR

SEQ ID NO: 12 - protein, 22 aa, Human immunodeficiency virus 1
ELDKWASLWNWFDITNWLWYIK

SEQ ID NO: 13 - protein, 22 aa, Human immunodeficiency virus 1
ELDKWASLWNWFSITQWLWYIK

SEQ ID NO: 14 - protein, 22 aa, Human immunodeficiency virus 1
ELDKWASLWNWFNITQWLWYIK

SEQ ID NO: 15 - protein, 22 aa, Human immunodeficiency virus 1
ELDKWASLWNWFSITKWLWYIK

SEQ ID NO: 16 - protein, 22 aa, Human immunodeficiency virus 1
QLDKWASLWNWFSITKWLWYIK

SEQ ID NO: 17 - protein, 22 aa, Human immunodeficiency virus 1
ELDKWASLWNWFGITKWLWYIK

SEQ ID NO: 18 - protein, 22 aa, Human immunodeficiency virus 1
ELDKWASLWNWFSISKWLWYIR

SEQ ID NO: 19 - protein, 22 aa, Human immunodeficiency virus 1
ELDEWASIWNWLDITKWLWYIK

SEQ ID NO: 20 - protein, 22 aa, Human immunodeficiency virus 1
ELDEWASLWNWFDITKWLWYIK

SEQ ID NO: 21 - protein, 22 aa, Human immunodeficiency virus 1
ELDQWASLWNWFDITKWLWYIK

SEQ ID NO: 22 - protein, 22 aa, Human immunodeficiency virus 1
ELDKWASLWNWFDISKWLWYIK

SEQ ID NO: 23 - protein, 22 aa, Human immunodeficiency virus 1
ELDKWANLWNWFDITQWLWYIR

SEQ ID NO: 24 - protein, 22 aa, Human immunodeficiency virus 1
ALDKWENLWNWFNITNWLWYIK

SEQ ID NO: 25 - protein, 22 aa, Human immunodeficiency virus 1
ALDKWTNLWNWFNISNWLWYIK

SEQ ID NO: 26 - protein, 22 aa, Human immunodeficiency virus 1
ALDKWANLWNWFSITNWLWYIR

SEQ ID NO: 27 - protein, 22 aa, Human immunodeficiency virus 1
ELDKWAGLWSWFSITNWLWYIR

SEQ ID NO: 28 - protein, 22 aa, Human immunodeficiency virus 1
ELDKWAGLWSWFSITNWLWYIR

SEQ ID NO: 29 - protein, 22 aa, Human immunodeficiency virus 1
ALDKWDSLWSWFSITNWLWYIK

SEQ ID NO: 30 - protein, 22 aa, Human immunodeficiency virus 1
ALDKWDNLWNWFSITRWLWYIE

SEQ ID NO: 31 - protein, 22 aa, Human immunodeficiency virus 1
ALDKWQNLWTWFGITNWLWYIK

SEQ ID NO: 32 - protein, 22 aa, Human immunodeficiency virus 1
ELDQWDSLWSWFGITKWLWYIK

SEQ ID NO: 33 - protein, 22 aa, Human immunodeficiency virus 1
QLDKWASLWTWSDITKWLWYIK

SEQ ID NO: 34 - protein, 22 aa, Human immunodeficiency virus 1
ALDKWDSLWNWFSITKWLWYIK

SEQ ID NO: 35 - protein, 5 aa, Artificial Sequence
  Synthetically generated peptide
WFNIT

SEQ ID NO: 36 - protein, 6 aa, Artificial Sequence
  Synthetically generated peptide
NWFNIT

SEQ ID NO: 37 - protein, 856 aa, Human immunodeficiency virus 1
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 38 - protein, 854 aa, Human immunodeficiency virus 1
MRVKEKYQHLWRWGWKWGTMLLGILMICSATEKLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDLRNVTNINNSSEGMRGEIKNCSFNITTSIRDKVKKDYALFYRLDVVPIDNDNTSYRLINCNTSTITQACPKVSFEPIPIHYCTPAGFAILKCKDKKFNGTGPCKNVSTVQCTHGIRPVVSTQLLLNGSLAEEEVVIRSSNFTDNAKNIIVQLKESVEINCTRPNNNTRKSIHIGPGRAFYTTGEIIGDIRQAHCNISRTKWNNTLNQIATKLKEQFGNNKTIVFNQSSGGDPEIVMHSFNCGGEFFYCNSTQLFNSTWNFNGTWNLTQSNGTEGNDTITLPCRIKQIINMWQEVGKAMYAPPIRGQIRCSSNITGLILTRDGGTNSSGSEIFRPGGGDMRDNWRSELYKYKVVKIEPLGVAPTKAKRRVVQREKRAVGTIGAMFLGFLGAAGSTMGAASITLTVQARLLLSGIVQQQNNLLRAIEAQQHLLQLTVWGIKQLQARVLALERYLRDQQLLGIWGCSGKLICTTAVPWNASWSNKTLDMIWDNMTWMEWEREIENYTGLIYTLIEESQNQQEKNEQDLLALDKWASLWNWFDISNWLWYIKIFIMIVGGLIGLRIVFTVLSIVNRVRQGYSPLSFQTHLPAPRGPDRPEGIEEEGGDRDRDRSVRLVDGFLALFWDDLRSLCLFSYHRLRDLLLIVARIVELLGRRGWEVLKYWWNLLQYWSQELRNSAVSLLNATAIAVAEGTDRVIEVVQRIYRAILHIPTRIRQGLERLLL

SEQ ID NO: 39 - protein, 5 aa, Artificial Sequence
  Synthetically generated peptide
CGGGS

SEQ ID NO: 40 - protein, 5 aa, Artificial Sequence
  Synthetically generated peptide
PADRE

SEQ ID NO: 41 - protein, 13 aa, Artificial Sequence
  Synthetically generated peptide
  VARIANT 1, 13: Xaa = D-alanine
  VARIANT 3: Xaa = L-cyclohexylalanine
XKXVAAWTLKAAX

SEQ ID NO: 42 - protein, 15 aa, Artificial Sequence
  Synthetically generated peptide
QYIKANSKFIGITEL

SEQ ID NO: 43 - DNA, 20 bp, Artificial Sequence
  Synthetically generated oligonucleotide
tccatgacgttcctgacgtt